(19) United States
(12) Patent Application Publication
(10) Pub. No.: US 2023/0165874 A1
(43) Pub. Date: Jun. 1, 2023

(54) ANTICOAGULANT COMPOUNDS AND METHODS AND DEVICES FOR THEIR PULMONARY USE

(71) Applicant: Elixir Medical Corporation, Milpitas (US)

(72) Inventors: John Yan, Los Gatos, CA (US); Xiaoxia Zheng, Fremont, CA (US); Vinayak D. Bhat, Cupertino, CA (US); Motasim Sirhan, Los Altos, CA (US); Blaine Bueche, San Mateo, CA (US)

(73) Assignee: Elixir Medical Corporation, Milpitas (US)

(21) Appl. No.: 18/160,519

(22) Filed: Jan. 27, 2023

Related U.S. Application Data
(63) Continuation of application No. PCT/US21/44414, filed on Aug. 3, 2021.
(60) Provisional application No. 63/195,824, filed on Jun. 2, 2021.
(60) Provisional application No. 63/193,017, filed on May 25, 2021.
(60) Provisional application No. 63/060,486, filed on Aug. 3, 2020.

(51) Int. Cl. A61K 31/5377; A61K 31/4709; A61K 45/06; A61K 31/4545; A61K 47/26; A61K 9/00; A61K 31/55; A61K 31/4706; A61K 47/40; A61K 47/12; A61K 31/496; A61K 31/4412; A61K 31/155; A61K 31/436; A61K 31/702

(52) U.S. Cl. CPC A61K 31/5377; A61K 31/4709; A61K 45/06; A61K 31/4545; A61K 47/26; A61K 9/0075; A61K 9/0078; A61K 31/55; A61K 31/4706; A61K 47/40; A61K 47/12; A61K 31/496; A61K 31/4412; A61K 31/155; A61K 31/436; A61K 31/702

(57) ABSTRACT

Devices, compositions, and methods are provided for inhibiting an inflammatory, fibrotic, and/or clot formation for pulmonary disease or condition in a patient. A therapeutic composition comprising a direct factor Xa inhibitor and/or a direct factor IIa inhibitor is provided. A therapeutically effective dose of the therapeutic composition is delivered to a site of the inflammatory, fibrotic, and/or clot formation pulmonary disease or condition in the patient's lung(s). The therapeutic composition may be formulated for delivery to the patient by inhalation, ventilation, instillation, ultrasound, vibration, injection, or the like. The therapeutic composition may include one or more additional therapeutically active substances and/or one or more additional pharmaceutical agents. The one or more additional therapeutically active substances and/or one or more additional pharmaceutical agents may be delivered together with the direct factor Xa inhibitor and/or direct factor IIa inhibitor or separately from the direct factor Xa inhibitor and/or factor IIa inhibitor.

### CROSS-REFERENCE

This application is a continuation of PCT Application No. PCT/US2021/044414 (Attorney Docket No. 32016-723.601), filed Aug. 3, 2021, which claims the benefit of each of U.S. Provisional Application No. 63/195,824, filed Jun. 2, 2021 (Attorney Docket No. 32016-723.103); U.S. Provisional Application No. 63/193,017, filed May 25, 2021 (Attorney Docket No. 32016-723.102); and U.S. Provisional Application No. 63/060,486 filed Aug. 3, 2020, (Attorney Docket No. 32016-723.101), each of which is incorporated herein by reference for all purposes in its entirety.

### BACKGROUND

### Field of the Disclosure

The present disclosure relates to anticoagulants and derivatives thereof and their use in therapeutic applications, and in particular with devices and uses in pulmonary applications.

Many viral and bacterial infections, and irritants (e.g., smoking, allergens) of the lung cause various lung conditions (e.g., pneumonia, bronchitis, emphysema, asthma, fibrosis, etc.). The conditions typically involve an immune reaction and/or inflammation leading to such conditions. In many cases, formation or increase of fluid build-up, fibrosis, cell apoptosis, and/or fibrin/clot formation occur, thereby damaging the lung tissues including the tissue in the alveolar sac where the blood oxygenation takes place. The condition and/or infection leading to inflammation and immune reaction upregulate the inflammatory pathway which in turn can cause fibrin formation and clot formation in the lung, leading to or increasing the likelihood of morbidities and mortality.

Systemic drugs had limited success in addressing these conditions or in long term effectiveness to address such conditions.

Coagulation is a process designed to stop bleeding from a damaged blood vessel. Disorders of coagulation can lead to obstructive clotting (thrombosis) or occlusion of the blood vessel.

Anticoagulant drugs act by inactivating thrombin and several other clotting factors that are required for a clot and/or fibrin to form.

Systemic administration of an anticoagulant may be ineffective in preventing or treating disorders associated with coagulation. For example, the concentration of the anticoagulant at or adjacent the site of injury may be insufficient at the appropriate time to prevent or treat disorders associated with coagulation. Furthermore, deficiencies of systemic administration of an anticoagulant can be exacerbated where the patient has a condition (e.g., cardiopulmonary disease, hypercholesterolemia, or diabetes) that renders the patient more susceptible to a vaso-occlusive event.

Previous attempts to provide local administration of an anticoagulant have had limited to no success in preventing coagulation disorders and/or preventing thrombus (clot) formation particularly after local tissue injury or infection. Furthermore, local injury to a tissue is commonly associated with additional injury to the tissue adjacent (e.g., proximal, distal, etc.) the site of the first injury or infection.

There is still a need to develop specialized therapeutic compositions that can rapidly reach local therapeutic dose and/or extended release of therapeutic agents, drugs, or bioactive materials directly into a localized tissue area during or following a medical procedure and/or inhalation of a drug for pulmonary condition, so as to treat pulmonary, or prevent pulmonary and nonpulmonary diseases or conditions such as pulmonary condition, tissue condition, or tissue thrombosis. The composition should release the therapeutic agent in an effective and efficient manner at the desired target location, where the therapeutic agent should rapidly permeate the target tissue at a local therapeutic level, preferably prior to the coagulation amplification cascade resulting in clot formation, and/or extended release to inhibit one or more of thrombin, fibrin, platelet aggregation, platelet activation, and/or clot formation.

There is still a need to develop specialized therapeutic compositions for pulmonary applications which can prevent, reduce, or eliminate fibrin and/or clot formation and reduce associate lung morbidities and mortalities.

There is still a need to develop specialized therapeutic compositions for pulmonary applications which can prevent, reduce, or eliminate fibrosis in the lung.

There is still a need to develop specialized therapeutic compositions for pulmonary applications which can prevent, reduce, or eliminate the ability of viral or bacterial infections from being able to invade the lung cells, replicate, or cause one or more of the lung conditions.

There is still a need to develop specialized therapeutic compositions for pulmonary applications which can prevent, reduce, or eliminate the ability of viral infections, bacterial infections, and/or irritants, from promoting a catastrophic immune response in the lung leading to lung tissue damage while maintaining a normal systemic immune response.

It would therefore be desirable to provide devices that locally deliver thrombin/clot formation-inhibiting agents, and optionally other types of biologically active agents (e.g., anti-fibrotic agents, anti-inflammatory agents, antiviral agents, antibiotic agents, immune suppressant agents, etc.), to the lungs to inhibit, reduce, and/or prevent coagulation, fibrin formation, and/or clot formation in the lung(s), and/or fibrosis of the lung tissue, and/or inflammation of the lung tissue, and/or to treat viral infections, and/or to treat bacterial infections.

It would therefore be desirable to provide devices that locally deliver thrombin/clot formation-inhibiting agents, deliver a clot inhibiting agent that additionally inhibits or promotes dissolution of one or more of inflammation, fibrin, injury, cell proliferation, platelet aggregation, platelet activation, and optionally other kinds of biologically active agents (e.g., anti-proliferative agents, anti-inflammatory agents, etc.), to the site of infection, inflammation, and/or injury of a lung or other body part or to an area adjacent thereto such as proximal or distal segments, before, during, and/or after infection, inflammation, and/or injury. The following inventions satisfy at least some of these desirable needs.

### Listing of Background Art

Relevant background art includes U.S. Pat. Nos. 10,668,015; 6,500,855; 8,409,272; 8,946,219; US2018/0008540; US2005/0169908; US2013/0199527; US2014/0083421; US2006/0014698; US2003/0017211; US2003/0158120; US2005/0064006; US2009/0075949; US2010/0003542; US2010/0130543; US2010/0184729; US2018/0000490; EP1849434; CA2464290; and WO2013/007840. The subject matter of this application is also related to that of commonly owned PCT applications PCT/US2021/34108, filed on May 25, 2021.

### SUMMARY OF THE DISCLOSURE

The present invention provides compositions and methods of using such compositions for one or more of the following therapies: inhibiting clotting, inhibiting clot formation, improving or promoting wound healing, inhibiting and/or resolving inflammation, inhibiting or attenuating vessel injury, inhibiting cell proliferation, inhibiting smooth cell proliferation, accelerate or promote fibrin dissolution, inhibiting platelet activation, inhibiting platelet aggregation, at the infection, inflammation, and/or injury site or at an area or segment adjacent thereto. While the compositions and methods when particularly use in the lungs, and more particular use after pulmonary delivery of the compositions, they are not so limited and would apply to treatments and therapies in other regions of the body as well.

In a first aspect, the present invention provides a method for inhibiting blood clotting in a patient's lung tissue, for example in the capillaries and more for particularly in the capillaries of the alveoli. The method comprises selecting a patient suffering from or at risk of suffering from blood clotting and/or fibrin formation in the patient's lung alveoli and/or other lung tissue and providing a therapeutic composition comprising each of a direct factor IIa inhibitor and a direct factor Xa inhibitor. The therapeutic composition is delivered to the patient's lung alveoli and/or other tissue at a dose sufficient to inhibit clot formation and/or fibrin formation therein. In one aspect, a method for inhibiting an inflammatory and/or condition for pulmonary condition or disease in a patient is provided. The method comprises providing a therapeutic composition comprising a direct factor Xa inhibitor; and delivering a therapeutically effective dose of the therapeutic composition to a site of the inflammatory and/or condition for pulmonary disease in the patient's lung.

In specific examples, the dose of the therapeutic composition may comprise one or more of inhalation, nebulization, ventilation, instillation, ultrasound dispersion, and injection.

In specific examples, the patient may be selected based upon blood clotting and/or fibrin formation caused by viral infection, bacterial infection, smoke inhalation, chemical exposure, genetic mutation, injury, smog and other air pollutions, inhalation of pollutants, work-related lung diseases, hypersensitivity pneumonitis, or a risk thereof. Usually, the selection comprise a diagnostic test run to determine the patient's status with respect to these conditions.

In other specific examples, the patient may be selected based upon blood clotting and/or fibrin formation caused by pneumonia, bronchitis, emphysema, asthma, pulmonary fibrosis, lung cancer, pulmonary edema, pulmonary embolism, sarcoidosis and granulomatosis with polyangiitis, chronic obstructive pulmonary disease, bronchiectasis, acute respiratory distress syndrome (ARDS), severe acute respiratory syndrome (SARS), and COVID-19. Usually, the selection comprise a diagnostic test run to determine the patient's status with respect to these conditions.

In specific examples, the direct factor Xa inhibitor may be selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), eribaxaban (PD 0348292), 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), (S)-3-(7-carbamimidoylnaphthalen-2-yl)-2-(44(S)-1-(1-iminoethyl)pyrrolidin-3-yl)oxy)phenyl)propanoic acid hydrochloride pentahydrate (DX9065a), letaxaban (TAK-442), GW813893, JTV-803, KFA-144, DPC-423, RPR-209685, MCM-09, or antistasin.

In preferred examples, the direct factor Xa inhibitor comprises rivaroxaban, or a salt, isomer, solvate, analog, derivative, metabolite, or prodrugs thereof

In other preferred examples, the direct factor Xa inhibitor comprises apixaban, or a salt, isomer, solvate, analog, derivative, metabolite, or prodrugs thereof.

In specific examples, a delivered dose of the direct factor Xa may be sufficient to generate a tissue concentration of the direct factor Xa inhibitor in the patient's lung alveoli of at least 0.2 ng/mg tissue measured 5 minutes after delivery of the therapeutic composition is completed, preferably being at least 0.5 ng/mg tissue, and more preferably being at least 1 ng/mg tissue. In these examples, the patient's blood concentration of the direct factor Xa inhibitor will typically be less than 200 ng/ml measured 5 minutes after delivery of the therapeutic composition is completed, often being less than 100 ng/ml, more often being less than 50 ng/ml, preferably being less than 40 ng/ml, and more preferably being less than 10 ng/ml.

In specific examples, the direct factor IIa inhibitor may be selected from the group consisting of argatroban, dabigatran, ximelagatran, melagatran, efegatran, inogatran, atecegatran metoxil (AZD-0837), hirudin, hirudin analogs, bivalirudin, desirudin, or lepirudin, preferably comprising argatroban or a salt, isomer, solvate, analog, derivative, metabolite, or prodrugs thereof. In an particularly preferred example, the direct factor Xa inhibitor comprises apixaban and the direct factor IIa inhibitor comprises argatroban.

In specific examples, the delivered dose of the direct factor IIa inhibitor is sufficient to generate a tissue concentration of the direct factor IIa inhibitor of at least 0.1 ng/mg tissue measured 5 minutes after delivery of the therapeutic composition is completed, often being at least 0.2 ng/mg tissue, more often being at least 0.5 ng/mg tissue, and preferably being at least 1 ng/mg tissue. In such examples, the patient's blood concentration of the direct factor IIa inhibitor is typically less than 100 ng/ml measured 30 minutes after delivery of the therapeutic composition is completed, sometimes being less than 50 ng/ml, often being less than 30 ng/ml, and preferably being less than 10 ng/ml.

In still other examples, the dose may be sufficient to generate a blood concentration of the direct factor Xa inhibitor which is smaller than a median maximum serum concentration (Cmax) of the direct factor Xa inhibitor generated by systemic delivery of the direct factor Xa inhibitor to achieve the same tissue concentration at the site of the inflammatory pulmonary disease.

In still other examples, the therapeutically effective dose may be sufficient to generate a blood concentration of the direct factor Xa inhibitor which does not exceed a median maximum serum concentration (Cmax) of the direct factor Xa inhibitor generated by systemic delivery of the direct factor Xa inhibitor to achieve the same tissue concentration at the site of the inflammatory pulmonary disease for more than about 26 hours to about 4 hours.

In further examples, the dose may be sufficient to generate a blood concentration of the direct factor IIa inhibitor which is smaller than a median maximum serum concentration (Cmax) of the direct factor IIa inhibitor generated by systemic delivery of the direct factor IIa inhibitor to achieve the same tissue concentration at the site of the inflammatory pulmonary disease.

In specific examples, the dose may sufficient to generate a blood concentration of the direct factor IIa inhibitor which does not exceed a median maximum serum concentration (Cmax) of the direct factor IIa inhibitor generated by systemic delivery of the direct factor IIa inhibitor to achieve the same tissue concentration at the site of the inflammatory pulmonary disease for more than about 2 hours to about 4 hours.

In specific examples, a weight ratio of the direct factor Xa inhibitor to the direct factor IIa inhibitor in the therapeutic composition is within a range of 3:1 to 1:10, for example being at a ratio of 1:5.

In specific examples, the therapeutic composition comprises one or more additional pharmaceutical agents, such as one or more anti-fibrotic agents, anti-platelet, antihistamine, anti-viral agents, anti-bacterial agents, metformin or its salt, steroids, interferons, anti-proliferative, anti-angiogenic, anti-VEGF, or combinations thereof.

In specific examples, the therapeutic composition may be delivered to the patient's lung alveoli by dispersing liquid droplets comprising the therapeutic composition into a breathing gas which is delivered to or inhaled by the patient, such droplets will typically have a mean droplet size in a range from 1 μm to 10 μm.

In specific examples, delivering the therapeutic composition to the patient's lung alveoli and/or other lung tissue may comprise dispersing dry particles comprising the therapeutic composition into a breathing gas which is delivered to or inhaled by the patient, where the particles typically have a mean diameter or width in a range from 1 μm to 10 μm.

In specific examples, a total dosage of apixaban from 1 mg to 5 mg and a total dosage of argatroban from 20 mg to 40 mg is delivered per day.

In a second aspect, the present invention provide a therapeutic composition for inhibiting an inflammatory pulmonary disease in a patient, the composition comprising each of a direct factor Xa inhibitor and a direct factor IIA inhibitor, wherein the composition is formulated for localized delivery to the patient's lungs. Such compositions are formulated for local delivery and therapeutic effect in a patient's lung tissue, for particularly within the lung tissue capillaries, and more particularly within the capillaries of the lung alveoli.

In specific examples, the compositions may be formulated for delivery via any one of inhalation, nebulization, ventilation, instillation, ultrasound dispersion, and injection.

Such formulations may be liquid formulations formulated to be dispersed into droplets in a breathing gas for delivery to the patient, typically being aqueous formulations and comprising a solubility enhancer to achieve therapeutic concentrations of the factor Xa and factor IIa.

Suitable solubility enhancers may comprise an oligosaccharide selected from the group consisting of cyclodextrins, 2-hydroxypropyl-13-cyclodextrin, methyl-P-cyclodextrin, randomly methylated-P-cyclodextrin, ethylated-P-cyclodextrin, triacety1[3-cyclodextrin, peracetylated-P-cyclodextrin, carboxymethyl-13-cyclodextrin, hydroxyethyl-I3-cyclodextrin, 2-hydroxy-3-(trimethylammonio)propyl-3-cyclodextrin, glucosyl-13-cyclodextrin, maltosyl-cyclodextrin, sulfobutyl ether-P-cyclodextrin, branched-P-cyclodextrin, hydroxypropyl-y-cyclodextrin, randomly methylated-y-cyclodextrin, trimethyl-y-cyclodextrin, or combinations thereof.

Preferred solubility enhancers comprise modified cyclodextrins, for example sulfoalkyl ether cyclodextrin derivatives such as those described in U.S. Pat. Nos. 5,134,127 and 5,376,645, the pull disclosures of which are incorporated herein by reference. One useful modified cyclodextrins is available under the tradename Captisol®, available from Ligand Phamaceuticals Inc.

Other suitable solubility enhancers comprise one or more of sodium carboxymethylcellulose, hydroxypropylmethylcellulose, sodium hyaluronate, sodium alginate, chitosan and its derivatives, polyethylene glycol, glycerin, propylene glycol, Triacetin, N,N-Dimethylacetamide, poly(vinyl pyrrolidone), pyrrolidone, dimethyl sulfoxide, ethanol, N-(-beta-Hydroxyethyl)-lactamide, 1-Methyl-2-pyrrolidinone, triglycerides, monothioglycerol, sorbitol, lecithin, methylparaben, propylparaben, or combinations thereof.

In specific examples, the direct factor Xa inhibitor may be present in such aqueous formulations including a solubility enhancer at from 1% to 20% by weight and the direct factor IIA inhibitor may be present in such aqueous formulations at from 25% to 75% by weight in the liquid formulation.

Another specific examples, the formulation may comprise a dry powder formulated for delivery via a dry powder inhaler.

Such dry powder formulations will usually comprise a stabilizing agent, such as a saccharide or a polysaccharide selected from a group consisting of mannose, sucrose, lactose, mannitol, and most commonly trehalose. Other suitable stabilizing agents may be selected from a group consisting of a citrate, a tartrate, methionine, vitamin A, vitamin E, zinc citrate, trisodium citrate, and zinc chloride. Still other suitable stabilizing agents include amino acids selected from a group consisting of glycine, L-leucine, isoleucine, and trileucine.

The dry powder formulations of the present invention may further comprise a surface modification agent, typically being selected from the group consisting of leucine, trileucine, ipalmitoylphosphatidylcholine, disteroylphosphatidylcholine, diarachidoylphosphatidylcholine dibehenoylphosphatidylcholine, diphosphatidyl glycerol, short-chain phosphatidylcholines, long-chain saturated phosphatidylethanolamines, long-chain saturated phosphatidylserines, long-chain saturated phosphatidylglycerols, and long-chain saturated phosphatidylinositols, sucrose tristearate, magnesium stearate or combinations thereof, often comprising leucine.

The direct factor Xa inhibitor in such dry powder formulations is typically selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl] acetic acid (YM-466 or YM-60828), or eribaxaban (PD 0348292), 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), (S)-3-(7-carbamimidoylnaphthalen-2-yl)-2-(4-(((S)-1-(1-iminoethyl)pyrrolidin-3-yl)oxy)phenyl)propanoic acid hydrochloride pentahydrate (DX9065a), letaxaban (TAK-442), GW813893, JTV-803, KFA-144, DPC-423, RPR-209685, MCM-09, or antistasin, usually being rivaroxaban, or a salt, isomer, solvate, analog, derivative, metabolite, or prodrugs thereof or being apixaban, or a salt, isomer, solvate, analog, derivative, metabolite, or prodrugs thereof.

In third aspect, the present invention provides a therapeutic composition for systemic delivery to a patient. The composition typically comprises each of (a) a direct factor Xa inhibitor and (b) a direct factor IIa inhibitor in combination with a penetration enhancer and formulated for delivery to and rapid absorption by the patient's lungs.

In specific examples, the penetration enhancer may be selected from a group consisting of sulphoxides, laurocaprams, alkanones, alkanol alcohols, fatty alcohols, propylene glycol, polyethylene glycol, ethylene glycol, diethylene glycol, triethylene glycol, dipropylene glycol, glycerol, propanediol, butanediol, pentanediol, hexanetriol, propylene glycol monolaurate and diethylene glycol monomethyl ether amides, pyrrolidone derivatives, pyrrolidone derivatives, cyclic amides, linear fatty acids, branched fatty acids, aliphatic fatty acid esters, alkyl fatty acid esters, anionic surfactants, cationic surfactants, zwitterionic surfactants, bile salts, Lecithin, terpenes, cyclodextrins, or combinations thereof, preferably being dimethylsulfoxide (DMSO).

Such systemic delivery compositions will often further comprise a surface modification agent. Exemplary surface modification agent may be selected from a group consisting of L-, D- or DL-forms of leucine, triluecine, isoleucine, lysine, valine, methionine, phenylalanine, lecithin, cysteine, phosphatidyl choline, sucrose tristearate, magnesium stearate, zwitterions, dipalmitoyl phosphatidylcholine, phosphatidylglycerol, dipalmitoyl phosphatidylethanolamine, dipalmitoyl phosphatidylinositol, zinc stearate, magnesium stearate, calcium stearate, sodium stearate or lithium stearate, derivatives, or combinations thereof. A preferred surface modification agent comprises leucine.

The systemic delivery compositions of the present invention are typically formulated for delivery via any one of inhalation, nebulization, ventilation, instillation, ultrasound dispersion, and injection.

The systemic delivery compositions may comprise a liquid formulation formulated to be dispersed into droplets in a breathing gas for delivery to the patient. Such liquid formulations typically comprise a solubility enhancer.

Suitable solubility enhancers may comprise an oligosaccharide selected from the group consisting of cyclodextrins, 2-hydroxypropyl-13-cyclodextrin, methyl-P-cyclodextrin, randomly methylated-P-cyclodextrin, ethylated-P-cyclodextrin, triacetyl[3-cyclodextrin, peracetylated-P-cyclodextrin, carboxymethyl-P-cyclodextrin, hydroxyethyl-I3-cyclodextrin, 2-hydroxy-3-(trimethylammonio)propyl-3-cyclodextrin, glucosyl-13-cyclodextrin, maltosyl-cyclodextrin, sulfobutyl ether-P-cyclodextrin, branched-P-cyclodextrin, hydroxypropyl-y-cyclodextrin, randomly methylated-y-cyclodextrin, trimethyl-y-cyclodextrin, or combinations thereof, with preferred solubility enhancers comprising a sulfoalkyl ether cyclodextrin derivative, as discussed previously.

Alternative solubility enhancers may comprise one or more of sodium carboxymethylcellulose, hydroxypropylmethylcellulose, sodium hyaluronate, sodium alginate, chitosan and its derivatives, polyethylene glycol, glycerin, propylene glycol, Triacetin, N,N-Dimethylacetamide, poly(vinyl pyrrolidone), pyrrolidone, dimethyl sulfoxide, ethanol, N-(-beta-Hydroxyethyl)-lactamide, 1-Methyl-2-pyrrolidinone, triglycerides, monothioglycerol, sorbitol, lecithin, methylparaben, propylparaben, or combinations thereof.

The systemic delivery compositions of the present invention may alternatively comprise a comprises a dry powder formulated for delivery via a dry powder inhaler.

In both liquid and dry powder formulations, the direct factor Xa inhibitor may be present at from 1% to 20% by weight and the direct factor IIa inhibitor may be present at from 25% to 75% by weight.

Suitable direct factor Xa inhibitors for the systemic delivery compositions may be selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl] acetic acid (YM-466 or YM-60828), or eribaxaban (PD 0348292), 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), (S)-3-(7-carbamimidoylnaphthalen-2-yl)-2-(4-(((S)-1-(1-iminoethyl)pyrrolidin-3-yl)oxy)phenyl)propanoic acid hydrochloride pentahydrate (DX9065a), letaxaban (TAK-442), GW813893, JTV-803, KFA-144, DPC-423, RPR-209685, MCM-09, or antistasin, typically comprising rivaroxaban, or a salt, isomer, solvate, analog, derivative, metabolite, or prodrugs thereof or comprising apixaban, or a salt, isomer, solvate, analog, derivative, metabolite, or prodrugs thereof.

Blood and tissue concentrations of the drugs delivered to a human patent's lungs may be measured in various conventional ways. Blood concentrations will be measured in blood sample drawn in a conventional manner, typically from venous circulation from a patient's arm. Non-invasive method to measurement of drug concentration in lung tissue after the drug has been delivered to a patient's lungs may be measured by several known techniques. For example, positron emission tomography (PET) with radiolabeled drug may be used to determine the amount or concentration of drug present in lung tissue at a desired time after inhalation or other delivery to the lung. In particular, a pulmonary formulation of 11^{C}-labeled and unlabeled apixaban and argatroban with a target dose may be inhaled or otherwise delivered into the lungs of a human patient or test animal (for example a mouse or rat) for target time point. The 11^{C}-labeled drug typically constitutes about 1-10 μg of the total drug formulation, which when given alone after administration, would lead to typical plasma concentrations of the order of 10-100 pM). A PET scan of lungs is performed immediately after inhalation and subsequent time points. Images are recorded. Each image is recorded by the color scale of radioactivity dose drug delivered to lungs. The representative results of the group sizes should be at least six to ten. The tissue concentration is then calculated as tissue radioactivity concentration normalized for inhaled total dose.

Alternatively, microdialysis (MD) may be used to determine free drug (unbounded) concentration in lung tissue. A microdialysis probe is introduced into a target region of the lungs of a human patient or animal model and diffusion of drugs across a semipermeable membrane at the tip of the microdialysis probe implanted into the interstitial fluid (ISF) of the lung tissue after a pulmonary formulation of apixaban and Argatroban with target dose is inhaled into lungs for target time point. The probe is constantly perfused with a physiological solution (perfusate) at a low flow rate, 1 to 10 μl/min. Once the probe is implanted into the lungs tissue, substances present in the ISF are filtered by diffusion out of the extracellular fluid into the probe, resulting in their presence in the perfusion medium (dialysate with Apixaban and Argatroban). To obtain concentrations in the ISF from concentrations in the dialysate, MD probes need to be calibrated. The collected microdialysates are kept in a −80° C. freezer until shipment to assay analysis lab. Apixaban and argatroban concentrations are measured using a Quantum Ultra LC-MS-MS system.

In another aspect, a method for inhibiting an inflammatory and/or condition for pulmonary condition or disease in a patient is provided. The method comprises providing a therapeutic composition comprising a direct factor IIa inhibitor; and delivering a therapeutically effective dose of the therapeutic composition to a site of the inflammatory and/or condition for pulmonary disease in the patient's lung.

In a preferred aspect, a method for inhibiting an inflammatory pulmonary disease in a patient is provided. The method comprises providing a therapeutic composition comprising a direct factor Xa inhibitor and a direct factor IIa inhibitor; and delivering a therapeutically effective dose of the therapeutic composition to a site of the inflammatory and/or condition for pulmonary condition or disease in the patient's lung.

In some examples, the therapeutically effective dose of the therapeutic composition is effective to suppress or prevent initiation, progression, or relapses of disease, including the progression of established disease.

In some examples, the composition is formulated for delivery via any one of the inhalation devices using aerosolization techniques by a jet nebulizer, a vibrating mesh nebulizer or an ultrasonic wave nebulizer. All these nebulizers can be configured for use on patients including ventilated patients. In other example, the composition is formulated for delivery via dry powder configured for inhalation via the lung.

In many examples, the inflammatory and/or condition for pulmonary disease is caused by viral infection, bacterial infection, smoke inhalation, chemical exposure, genetic mutation, injury, smog and other air pollutions, inhalation of environmental and occupational pollutants, work-related lung diseases, hypersensitivity pneumonitis, and combinations thereof.

In many examples, the inflammatory and/or condition for pulmonary disease comprises one or more of clot formation or fibrin formation.

In many examples, the inflammatory and/or condition for pulmonary condition or disease comprises pneumonia, bronchitis, emphysema, asthma, pulmonary fibrosis, lung cancer, pulmonary edema, pulmonary embolism, sarcoidosis and granulomatosis with polyangiitis, chronic obstructive pulmonary disease, bronchiectasis, acute respiratory distress syndrome (ARDS), SARS, or COVID-19.

In many examples, the method further comprises diagnosing the patient as having the inflammatory and/or condition for pulmonary disease prior to delivering the therapeutically effective dose of the therapeutic composition.

In one aspect, a therapeutic composition and/or a method for inhibiting a pulmonary condition or an inflammatory pulmonary condition or disease in a patient is provided. The therapeutic composition and/or method comprises providing a therapeutic composition comprising a direct factor Xa inhibitor; and delivering a therapeutically effective dose of the therapeutic composition to a site of the pulmonary condition or the inflammatory pulmonary condition or disease in the patient's lung. Optionally, the composition comprises one or more additional agents.

In another aspect, a therapeutic composition and/or a method for inhibiting a pulmonary condition or an inflammatory pulmonary condition or disease in a patient is provided. The therapeutic composition and/or method comprises providing a therapeutic composition comprising a direct factor IIa inhibitor; and delivering a therapeutically effective dose of the therapeutic composition to a site of the pulmonary condition or the inflammatory pulmonary disease in the patient's lung. Optionally, the composition comprises one or more additional agents.

In another aspect, a therapeutic composition and/or a method for inhibiting a pulmonary condition in a patient is provided. The therapeutic composition and/or method comprises providing a therapeutic composition comprising a direct factor IIa inhibitor and a direct factor Xa inhibitor; and delivering a therapeutically effective dose of the therapeutic composition to a site in the patient's lung. Optionally, the composition comprises one or more additional agents.

In another aspect, a therapeutic composition and/or a method for inhibiting a pulmonary condition in a patient is provided. The therapeutic composition and/or method comprises providing a therapeutic composition comprising at least one or at least both of a direct factor IIa inhibitor and a direct factor Xa inhibitor; and delivering a therapeutically effective dose of the therapeutic composition to the patient's lung, wherein the composition reaching the systemic circulation is below the systemic Cₘₐₓ therapeutic level (or below a mean therapeutic level) for at least one agent, preferably below the systemic Cₘₐₓ therapeutic levels (or below a mean therapeutic levels) for both agents. In a preferred example, the composition is configured to maintain a local therapeutic level for an extended period ranging from 1 hour to 24 hours, preferably ranging from 1 hour to 12 hours, more preferably ranging from 1 hour to 6 hours.

In another aspect, a therapeutic composition and/or a method for rapidly delivering a systemic therapeutic dose to a patient is provided. The therapeutic composition and/or method comprises providing a therapeutic composition comprising at least one or at least both of a direct factor IIa inhibitor and a direct factor Xa inhibitor; wherein the composition is delivered to the lung of a patient. In a preferred example, the composition is configured to rapidly provide a systemic therapeutic dose to a patient body after delivering said composition to the lung. The composition is configured to reach systemic therapeutic dose (levels) within 3 hours, 2 hours, 1 hour, 30 minutes, or 15 minutes, from delivering said composition to a patient's lung. Optionally, the composition comprises one or more agents consisting of anti-platelet agents and anti-proliferative agent.

In some examples, the direct factor Xa inhibitor is present in the therapeutic composition at a concentration within a range of about 0.1 nM to about 1,000,000 nM. For example, the direct factor Xa inhibitor may present in the therapeutic composition at a concentration within a range of about 1 nM to about 1,000,000 nM, about 10 nM to about 1,000,000 nM, or about 100 nM to about 1,000,000 nM.

In some examples, the therapeutically effective dose is sufficient to generate a tissue concentration of the direct factor Xa inhibitor of about 0.1 ng/mg tissue to about 10 ng/mg tissue. For example, the therapeutically effective dose may be sufficient to generate a tissue concentration of the direct factor Xa inhibitor of about 0.2 ng/mg tissue to about 10 ng/mg tissue, about 0.5 ng/mg tissue to about 5 ng/mg tissue, or about 1 ng/mg tissue to about 5 ng/mg tissue. In some examples, the therapeutically effective dose is sufficient to generate a blood concentration of the direct factor Xa inhibitor which is less than about 200 ng/ml, 100 ng/ml, 50 ng/ml, 40 ng/ml, or 10 ng/ml. In some examples, the therapeutically effective dose is sufficient to generate a blood concentration of the direct factor Xa inhibitor which is smaller than a median maximum serum concentration (Cₘₐₓ) of the direct factor Xa inhibitor generated by systemic delivery of the direct factor Xa inhibitor to achieve the same tissue concentration at the site of the inflammatory pulmonary disease. In some examples, the therapeutically effective dose is sufficient to generate a blood concentration of the direct factor Xa inhibitor which does not exceed a median maximum serum concentration (Cₘₐₓ) of the direct factor Xa inhibitor generated by systemic delivery of the direct factor Xa inhibitor to achieve the same tissue concentration at the site of the inflammatory pulmonary disease for more than about 6 hours to about 12 hours.

In some examples, the therapeutic composition further comprises at least one additional therapeutically active substance. In some examples, the at least one additional therapeutically active substance comprises a direct factor IIa inhibitor selected from the group consisting of Argatroban, dabigatran, ximelagatran, melagatran, efegatran, inogatran, atecegatran metoxil (AZD-0837), hirudin, hirudin analogs, bivalirudin, desirudin, or lepirudin. For example, the direct factor IIa inhibitor may comprise Argatroban. or a salt, isomer, solvate, analog, derivative, metabolite, or prodrugs thereof. In some examples, the direct factor Xa inhibitor comprises Apixaban and the direct factor IIa inhibitor comprises Argatroban. In some examples, the direct factor IIa inhibitor is present in the therapeutic composition at a concentration within a range of about 0.1 nM to about 1,000,000 nM. For example, the direct factor IIa inhibitor may present in the therapeutic composition at a concentration within a range of about 1 nM to about 1,000,000 nM, about 10 nM to about 1,000,000 nM, or about 100 nM to about 1,000,000 nM. In some examples, the therapeutically effective dose is sufficient to generate a tissue concentration of the direct factor IIa inhibitor of about 0.1 ng/mg tissue to about 10 ng/mg tissue. For example, the therapeutically effective dose may be sufficient to generate a tissue concentration of the direct factor IIa inhibitor of about 0.2 ng/mg tissue to about 10 ng/mg tissue, about 0.5 ng/mg tissue to about 5 ng/mg tissue, or about 1 ng/mg tissue to about 5 ng/mg tissue. In some examples, the therapeutically effective dose is sufficient to generate a blood concentration of the direct factor IIa inhibitor which is less than about 200 ng/ml, 100 ng/ml, 50 ng/ml, 40 ng/ml, or 10 ng/ml. In some examples, the therapeutically effective dose is sufficient to generate a blood concentration of the direct factor IIa inhibitor which is smaller than a median maximum serum concentration (Cₘₐₓ) of the direct factor IIa inhibitor generated by systemic delivery of the direct factor IIa inhibitor to achieve the same tissue concentration at the site of the inflammatory pulmonary disease. In some examples, the therapeutically effective dose is sufficient to generate a blood concentration of the direct factor IIa inhibitor which does not exceed a median maximum serum concentration (Cₘₐₓ) of the direct factor IIa inhibitor generated by systemic delivery of the direct factor IIa inhibitor to achieve the same tissue concentration at the site of the inflammatory pulmonary disease for more than about 6 hours to about 12 hours. In some examples, the weight compositional ratio of the direct factor Xa inhibitor to the direct factor IIa inhibitor in the therapeutic composition is within a range of about 3:1 to about 1:3. For example, the weight compositional ratio of the direct factor Xa inhibitor to the direct factor IIa inhibitor in the therapeutic composition may be about 1:1.

In some examples, the therapeutic composition is administered in combination with one or more additional pharmaceutical agents. In some examples, the one or more additional pharmaceutical agents comprises one or more anti-fibrotic agents, anti-viral agents, anti-bacterial agents, metformin or its salt, steroids, interferons, or combinations thereof. In some examples, the anti-fibrotic agent comprises pirfenidone and is present in the therapeutic composition at a concentration within a range of about 1,000 nM to about 10,000,000 nM. For example, prifenidone may present in the therapeutic composition at a concentration within a range of about 10,000 nM to about 10,000,000 nM or about 100,000 nM to about 10,000,000 nM. In some examples, the anti-fibrotic agent comprises nintedanib and is present in the therapeutic composition at a concentration within a range of about 0.1 nM to about 1,000,000 nMFor example, nintedanib may present in the therapeutic composition at a concentration within a range of about 1 nM to about 1,000,000 nM, about 10 nM to about 1,000,000 nM, or about 100 nM to about 1,000,000 nM. In some examples, the anti-viral/anti-diabetic agent comprises metformin or its salt and is present in the therapeutic composition at a concentration within a range of about 1,000 nM to about 100,000,000 nM. For example, metformin or its salt may present in the therapeutic composition at a concentration within a range of about 100,000 nM to about 100,000,000 nM or about 1,000,000 nM to about 100,000,000 nM.

In some examples, the therapeutic composition comprises one or more of a pharmaceutically acceptable carrier, a propellant, an excipient, a surfactant, a binding agent, an adjuvant agent, a flavoring agent or taste masking agent, a coloring agent, an emulsifying agent, a stabilizing agent, an isotonic agent, and targeting co-molecules.

In some examples, the therapeutic composition is atomized, nebulized, aerosolized, pressurized, micronized, nanosized, in the form of a dry powder, or combinations thereof.

In some examples, the therapeutically effective dose of the therapeutic composition is effective to inhibit thrombosis, inhibit clot formation, or inhibit fibrin formation in the patient's lung.

In another aspect, a therapeutic composition for inhibiting an inflammatory pulmonary disease in a patient is provided. The composition comprises a direct factor Xa inhibitor formulated for delivery to the patient by any one of inhalation, ventilation, instillation, ultrasound, vibration, and injection.

In some examples, delivery of a therapeutically effective dose of the therapeutic composition is effective to suppress or prevent initiation, progression, or relapses of disease, including the progression of established disease.

In many examples, the inflammatory pulmonary disease is caused by a viral infection, bacterial infection, smoke inhalation, chemical exposure, genetic mutation, injury, smog and other air pollutions, inhalation of environmental and occupational pollutants, work-related lung diseases, hypersensitivity pneumonitis, and combinations thereof.

In some examples, the inflammatory pulmonary disease comprises pneumonia, bronchitis, emphysema, asthma, pulmonary fibrosis, lung cancer, pulmonary edema, pulmonary embolism, sarcoidosis and granulomatosis with polyangiitis, chronic obstructive pulmonary disease, bronchiectasis, acute respiratory distress syndrome (ARDS), SARS, or COVID-19.

In another aspect, a method for inhibiting clot formation or fibrin formation in a lung of a patient is provided. The method comprises: providing a therapeutic composition comprising a direct factor Xa inhibitor; and delivering a therapeutically effective dose of the therapeutic composition to a site of the clot formation or fibrin formation in the patient's lung.

In some examples, the therapeutic composition comprises one or more anticoagulant agents that has an IC50 to inhibit factor Xa and factor II at a dose ranging from 0.0001 nM to 1000 nM, preferably at a dose ranging from 0.0001 nM to 100 nM, more preferably at a dose ranging from 0.0001 nM to 10 nM, and most preferably at a dose ranging from 0.0001 nM to 1 nM.

In some instances, the compositions of the present invention may comprise a first drug formulation formulated to provide a rapid drug release and a second drug formulation formulated to provide an extended drug release. The rapid release of the first drug formulation and extended release of the second drug formulation will typically act in combination to accelerate inhibition and/or dissolution of clot or thrombus in the 1 ng. In addition, such formulations may also inhibitor control one or more of inflammation, cell proliferation, thrombin, fibrin formation, platelet aggregation, platelet activation, and clot or thrombus formation, and/or increase or prolong time before blood forms clot or thrombus.

In specific instances, at least one of the first drug formulation and the second drug formulation may comprise both a direct factor IIa inhibitor and a direct factor Xa inhibitor. In other instances, the first drug formulation and the second drug formulation may each comprise both a direct factor IIa inhibitor and a direct factor Xa inhibitor.

In specific instances, the at least one drug of the first (rapid release) drug formulation is released from the first therapeutic composition over a first time period (duration) is in a range from 3 hours to 28 days after implantation, usually from 3 hours to 7 days after implantation, and preferably from 3 hours to 3 days after implantation. The first therapeutic composition is typically configured to release the at least one drug of the first drug formulation at a mean rate in the range from 1 μg/hour to 10 μg/hour, usually from 1 μg/hour to 5 μg/hour, preferably from 2 μg/hour to 4 μg/hour over a 24 hour period following exposure to the pulmonary environment, where the mean rate may be determined based on the amount (weight) of drug released over the total duration of the release.

In specific instances, the at least one drug of the second drug formulation (sustained release) is released from the second therapeutic composition over a second time period is in a range from 30 days to 12 months after implantation, usually from 30 days to 9 months after implantation, and preferably from 30 days to 6 months after implantation. The second therapeutic composition is typically configured to delay release the at least one drug of the second drug formulation for at least one 24-hour period following exposure to the pulmonary environment. The second therapeutic composition is typically configured to release the at least one drug of the second drug formulation at a mean rate not exceeding 2 μg/hour, usually 1 μg/hour, preferably 0.5 μg/hour, and more preferably 0.1 μg/hour after the 24 hour period following exposure to the pulmonary environment, where the mean rate may be determined based on the amount (weight) of drug released over the total duration of the release.

The first and second therapeutic composition will typically but not necessarily comprise a polymer to sequester and control the release rate and duration of the drugs. In some instances the drugs may be coated, layered, or otherwise deposited on or in surfaces or receptacles on the implantable structure without a polymer but optionally with excipients, carriers, coating agents, and other conventional drug coating materials.

In some instances, one of the first and second therapeutic compositions may comprise a polymer while the other is free from polymer. For example, the first therapeutic (rapid release) composition may free from polymer and the second (sustained release) therapeutic composition may comprises a polymer to maintain or control the release rate and duration. For example, the first therapeutic composition may comprise a core region of a dry particle coated by the second therapeutic composition to effect a burst release.

In instances where the first and second therapeutic compositions each comprise a polymer, the first therapeutic composition will have a first drug-to-polymer weight ratio and the second therapeutic composition will a second drug-to-polymer weight ratio. The ratios may be the same but will more often be different. For example, the first drug-to-polymer weight ratio may be in a range from 5:1 to 1:3, usually from 5:2 to 1:2, and preferably from 5:3 to 1:1, and the second drug-to-polymer weight ratio may in a range from 5:2 to 1:5, usually from 5:3 to 2:5, and preferably from 1:1 to 1:2. The first drug-to-polymer weight ratio is usually greater than the second drug-to-polymer weight ratio (greater loading can enhance the burst effect in the first therapeutic composition), but in some instances the first drug-to-polymer weight ratio may less than the second drug-to-polymer weight ratio (greater loading can also enhance duration of release).

While drug release from the first and second therapeutic compositions may commence simultaneously, in many instances the first therapeutic composition and the second therapeutic composition are configured to delay start of release of the second drug formulation for a time period after release of the first drug formulation has started. For example, the first therapeutic composition may be layered over the second therapeutic composition to delay release of the second drug formulation, e.g. the first therapeutic composition may initially cover at least a portion of the second therapeutic composition and may be configured to dissolve over the time period in the pulmonary environment to expose the second therapeutic composition and allow release of the second drug formulation.

Alternatively, a sacrificial layer may present over at least one of the first therapeutic composition and the second therapeutic composition or between the first therapeutic composition and the second therapeutic composition to delay release of one or more drugs from either or both of the first therapeutic composition and the second therapeutic compositions.

Alternatively, a diffusion-rate controlling layer may be present over at least one of the first therapeutic composition and the second therapeutic composition or between the first therapeutic composition and the second therapeutic composition to control a release rate of one or more drugs from either or both of the first therapeutic composition and the second therapeutic compositions.

The polymer(s) may be configured to release the first and/or second drug formulation at least partly by dissolution of the polymer when exposed to the pulmonary environment. For example, the polymer of the first therapeutic composition may dissolve at a faster rate than dissolution of the second therapeutic composition in the pulmonary environment. Alternatively, the polymer may be configured to release the first and/or second drug formulation at least partly by a diffusion mechanism through the polymer when exposed to the pulmonary environment. Alternatively, the polymer may be configured to release the first and/or second drug formulation through a combination of dissolution of and diffusion through the polymer when exposed to the pulmonary environment.

Usually, but not necessarily, one or more polymer will be porous where the first and/or second drug formulation are sequestered in pores of the polymer(s). Often, a release rate of the first and/or second drug formulation may at least partly determined by a pore size of the polymer. In some instances, the polymers of the first and second drug formulations may have different pore sizes which provide different release rates.

In other instances, the first and second drug formulations may be at least partially separated in different regions within the porous polymer. Alternatively or additionally, the first and second drug formulations may at least partially present in overlapping regions of the porous polymer.

Exemplary anti-proliferative agents of the present invention include mTOR inhibitors selected from a group consisting of Sirolimus, biolimus, everolimus, myolimus, novolimus, ridaforolimus, temsirolimus, zotarolimus, or salts, isomers, solvates, analogs, derivatives, metabolites, or prodrugs thereof, which may be used individually or in combination. Preferred anti-mTOR proliferative agents comprise Sirolimus, or a salt, isomer, solvate, analog, derivative, metabolite, or prodrugs thereof.

Exemplary anti-proliferative agents of the present invention also include paclitaxel, or a salts, isomer, solvate, analog, derivative, metabolite, or prodrug thereof.

In specific examples of the present invention, the direct factor IIa inhibitor comprises Argatroban and the direct factor Xa inhibitor comprises Apixaban or Rivaroxaban. In other specific examples of the present invention, the direct factor IIa inhibitor comprises Argatroban or an analogue of Argatroban, the direct factor Xa inhibitor comprises Apixaban or Rivaroxaban or an analogue of Apixaban or Rivaroxaban, and the anti-proliferative agent comprises Sirolimus or an analogue of Sirolimus.

In some instances, at least one of the therapeutic compositions may comprises an excipient, an adjuvant, a carrier, a wetting agent. In some instances, the first and second therapeutic compositions may be formed contiguously. In some instances, the first and second therapeutic compositions are separated by barrier, for example a polymer layer.

In some examples, a third therapeutic composition comprises a third drug formulation including at least one drug selected from the group consisting of a direct factor IIa inhibitor and a direct factor Xa inhibitor. The third drug formulation may comprise any one of the previously discussed drugs and/or an additional drug. The third therapeutic composition may be disposed at least partially over the first therapeutic composition which may disposed at least partially over the second therapeutic composition, where the third therapeutic composition may be configured to effect a burst release which is more rapid than the release of either the first or second therapeutic compositions.

In specific examples, the first and second therapeutic compositions may comprise polymer and the third therapeutic composition may be free from polymer and coated or otherwise deposited over at least a portion of the first therapeutic composition.

In specific examples, the additional drug may be unique, i.e. not found in either the first or second drug formulations. Often, the additional drug wll have the same release rate as at least one of the other drugs but alternatively may have a different release rate than at least one of the other drugs

In other examples, the third drug formulation may comprise at least one polymer, where at least one polymer in the third formulation may be the same and/as or different from at least one polymer in the first and second drug formulations. For example, the at least one polymer in the third formulation may provide a different release rate than provided by at least one polymer in the first and second drug formulations. In other examples, the at least one polymer in the third formulation provides substantially the same release rate as provided by at least one polymer in the first and second drug formulations.

In specific instances, the first, second, or optional third therapeutic compositions may comprise a plurality of drug different formulations for at least one drug. For example, a single drug type may be sequestered in formulations with polymers have different release rates and/or drug loadings, allowing further control of the drug release characteristics.

The polymer comprises will typically be biodegradable polymer, for example being selected from the group consisting of polyesters, including polylactic acids, polyglycolic acids, polylactic acid-co-glycolic acids, polylactic acid-co-caprolactones, polyethylene glycol-block-poly caprolactone, and polyurethanes; poly(methyl methacrylate) (PMMA); poly N-(2-Hydroxypropyl) methacrylamides; polyethylenimine (PEI), dextran, dextrin, chitosans, poly(L-lysine); poly(aspartamides), polyethylenes; polypropylenes; polyamides; polyethylene glycols (PEG); silicones; poly(anhydrides); and poly ortho esters.

Alternatively, the polymer may comprises a non-degradable polymer, for example being selected from the group consisting of polyacrylates, polymethacrylates, poly(n-butyl methacrylate), poly(hydroxyethylmethacrylate), polyamides, nylons, nylon 12, dacron, polyethylene terephthalate, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate), and copolymers thereof.

In specific examples, the argatroban, at least one of apixaban and rivaroxaban, and the Sirolimus may be sequestered in a porous structure of the PLGA, and the release of the Argatroban, the direct factor Xa inhibitor including at least one of apixaban and rivaroxaban, and the Sirolimus into the pulmonary environment occurs through a combination of diffusion and dissolution.

In specific instances, at least one of the first drug formulation and the second drug formulation may comprise either or both a direct factor IIa inhibitor and a direct factor Xa inhibitor.

In specific instances, the first (rapid release) drug formulation may release drug from the first therapeutic composition over a first time period is in a range from 3 hours to 28 days after implantation, usually from 3 hours to 7 days after implantation, preferably from 3 hours to 3 days after implantation, where the at least one drug of the first drug formulation is typically at a mean rate in the range from 1 μg/hour to 10 μg/hour, usually from 1 μg/hour to 5 μg/hour, preferably from 2 μg/hour to 4 μg/hour over a 24 hour period following exposure to the pulmonary environment, where the mean rate may be determined based on the amount (weight) of drug released over the total duration of the release.

In specific instances, the at least one drug of the second drug formulation is released from the second (sustained release) therapeutic composition over a second time period is in a range from 30 days to 12 months after implantation, usually from 30 days to 9 months after implantation, preferably from 30 days to 6 months after implantation, where the second therapeutic composition is typically configured to release the at least one drug of the second drug formulation for at a mean rate not exceeding 2 μg/hour, usually 1 μg/hour, preferably 0.5 μg/hour, and more preferably 0.1 μg/hour after the 24 hour period following exposure to the pulmonary environment, where the mean rate may be determined based on the amount (weight) of drug released over the total duration of the release.

In preferred instances, the therapeutic compositions are formulated to locally release the first and second drug formulation Xa to the injury site at a rate or a concentration sufficient to begin to inhibit one or more of inflammation, cell proliferation, fibrin formation, and clot formation within about 3 hours to about 7 days after the drugs are delivered.

In some examples, therapeutic composition in accordance with the principles of the present invention may be formulated to release the one or more active substances at a rate sufficient to generate a tissue concentration at the injury site within a range of about 2 ng/mg to about 800 ng/mg within about 3 hours. In some examples, the therapeutic composition is formulated to release the one or more active substances at a rate sufficient to generate a tissue concentration at the injury site within a range of about 10 ng/mg to about 100 ng/mg within about 3 hours.

In some examples, the therapeutic composition of the present invention may be formulated to release the one or more active substances at a rate sufficient to generate a tissue concentration at the injury site within a range of about 2 ng/mg to about 100 ng/mg within about 24 hours. In some examples, the therapeutic composition is formulated to release the one or more active substances at a rate sufficient to generate a tissue concentration at the injury site within a range of about 3 ng/mg to about 50 ng/mg within about 24 hours. In some examples, the therapeutic composition is formulated to release the one or more active substances at a rate sufficient to generate a tissue concentration at the injury within a range of about 4 ng/mg to about 25 ng/mg within about 24 hours.

In some examples, the therapeutic composition of the present invention may be formulated to release the one or more active substances at a rate sufficient to generate a tissue concentration at a target site within a range of about 1 ng/mg to about 30 ng/mg within about 7 days. In some examples, the therapeutic composition is formulated to release the one or more active substances at a rate sufficient to generate a tissue concentration at the injury site within a range of about 1.5 ng/mg to about 20 ng/mg within about 7 days. In some examples, the therapeutic composition is formulated to release the one or more active substances at a rate sufficient to generate a tissue concentration at the injury site within a range of about 2 ng/mg to about 25 ng/mg within about 7 days.

In some examples, the therapeutic composition of the present invention may be formulated to release the one or more active substances at a rate sufficient to generate a tissue concentration at the injury site within a range of about 0.5 ng/mg to about 30 ng/mg within about 28 days. In some examples, the therapeutic composition is formulated to release the one or more active substances at a rate sufficient to generate a tissue concentration at the injury site within a range of about 1 ng/mg to about 20 ng/mg within about 28 days. In some examples, the therapeutic composition is formulated to release the one or more active substances at a rate sufficient to generate a tissue concentration at the injury site within a range of about 1.5 ng/mg to about 25 ng/mg within about 28 days.

In some examples, the therapeutic composition of the present invention may be formulated to release the one or more active substances at a rate sufficient to generate a tissue concentration at the injury site within a range of about 0.1 ng/mg to about 10 ng/mg within about 90 days or about 180 days.

In some examples, the therapeutic composition of the present invention may be formulated to release the one or more active substances at a rate sufficient to generate a tissue concentration at a location proximal or distal a proximal end of the structure or a distal end of the structure, respectively, within a range of about 0.5 ng/mg to about 500 ng/mg within about 3 hours. In some examples, the therapeutic composition is formulated to release the one or more active substances at a rate sufficient to generate a tissue concentration at the location proximal or distal the proximal end of the structure or the distal end of the structure, respectively, within a range of about 1 ng/mg to about 35 ng/mg within about 3 hours. In some examples, the therapeutic composition is formulated to release the one or more active substances at a rate sufficient to generate a tissue concentration at the location proximal or distal the proximal end of the structure or the distal end of the structure, respectively, within a range of about a range of about 1.5 ng/mg to about 30 ng/mg within about 3 hours. In some examples, the therapeutic composition is formulated to release the one or more active substances at a rate sufficient to generate a tissue concentration at the location proximal or distal to the proximal end of the structure or the distal end of the structure (e.g., within ±5 mm proximal or distal to an end of the structure), respectively, within a range of about 0.1 ng/mg to about 50 ng/mg, about 0.25 ng/mg to about 20 ng/mg, about 1 ng/mg to about 50 ng/mg, or about 3 ng/mg to about 50 ng/mg within about 3 hours.

In some examples, the therapeutic composition is formulated to release the one or more active substances at a rate sufficient to generate a tissue concentration at a location proximal or distal a proximal end of the structure or a distal end of the structure, respectively, within a range of about 0.2 ng/mg to about 25 ng/mg, about 2 ng/mg to about 25 ng/mg, or about 4 ng/mg to about 25 ng/mg within about 24 hours. In some examples, the therapeutic composition is formulated to release the one or more active substances at a rate sufficient to generate a tissue concentration at a proximal segment (proximal to the proximal end of the device or device structure) or distal segment (distal to the distal end of the device or device structure) (e.g., within ±5 mm proximal or distal to an end of the structure), to the injury site respectively, ranging from about 0.1 ng/mg to about 50 ng/mg, from about 0.25 ng/mg to about 20 ng/mg, from about 1 ng/mg to about 50 ng/mg, or from about 3 ng/mg to about 50 ng/mg within about 24 hours. In some examples, the therapeutic composition is formulated to release the one or more active substances at a rate sufficient to generate a tissue concentration at a segment proximal or distal to the device (within ±5 mm from the device end), respectively, within a range of about 0.3 ng/mg to about 10 ng/mg within about 24 hours.

In some examples, the therapeutic composition is formulated to release a larger dose of the direct factor Xa inhibitor than the anti-proliferative agent. In some examples, the dose of the direct factor Xa inhibitor is about 1 to about 6 times larger, about 1.25 to about 5 times larger, about 1.5 to about 3 times larger, or about 1.5 to about 2.5 times larger than a dose of the anti-proliferative agent.

In some examples, the therapeutic composition is formulated to release a dose of the direct factor Xa inhibitor sufficient to generate a blood concentration of the direct factor Xa inhibitor which is smaller than a median maximum serum concentration (Cₘₐₓ) of the direct factor Xa inhibitor generated by systemic delivery of the direct factor Xa inhibitor to achieve the same tissue concentration at the injury site. In other examples, the therapeutic composition is formulated to release a dose of the direct factor Xa inhibitor sufficient to generate a blood concentration of the direct factor Xa inhibitor which is smaller than a median maximum serum concentration (Cₘₐₓ) of the direct factor Xa inhibitor generated by systemic delivery of the direct factor Xa inhibitor when taking one or more oral dose of said factor Xa inhibitor. In some examples, the systemic delivery comprises a single oral dose, a daily oral dose, or a smallest oral dose of the direct factor Xa inhibitor. In some examples, the blood concentration is larger than a median minimum serum concentration (Cₘᵢₙ) of the direct factor Xa inhibitor generated by systemic delivery of a single oral dose. In some examples, the blood concentration is smaller than a median minimum serum concentration (Cₘᵢₙ) of the direct factor Xa inhibitor generated by systemic delivery of a single oral dose. In some examples, the Cₘₐₓ is measured using one of plasma blood, serum blood, or whole blood. In other examples, the median Cₘₐₓ is 80 ng/ml, or 123 ng/ml, or 171 ng/ml, or 321 ng/ml, or 480 ng/ml of blood.

In some examples, the therapeutic composition is formulated to release a dose of the direct factor Xa inhibitor sufficient to generate a plasma drug level area under the curve (AUC (0-24) or AUC (0-∞)) in ng·h/ml which is smaller than a median AUC (AUC (0-24) or AUC (0-∞)) in ng·h/ml of the direct factor Xa inhibitor generated by systemic delivery of the direct factor Xa inhibitor to achieve the same tissue concentration at the injury site. In other examples, the therapeutic composition is formulated to release a dose of the direct factor Xa inhibitor sufficient to generate a plasma drug level area under the curve (AUC (0-24) or AUC (0-∞)) in ng·h/ml which is less than a median (AUC (0-24) or AUC (0-∞)) in ng·h/ml of the direct factor Xa inhibitor generated by systemic delivery of the direct factor Xa inhibitor when taking one or more oral dose of said factor Xa inhibitor. In some examples, the systemic delivery comprises a single oral dose, a daily oral dose, or a smallest oral dose of the direct factor Xa inhibitor. In some examples, the (AUC (0-24) or AUC (0-∞)) is measured using one of plasma blood, serum blood, or whole blood. In other examples, the median (AUC (0-24) or AUC (0-∞cc)) is 724 ng·h/ml, or 1437 ng·h/ml, or 2000 ng·h/ml, or 4000 ng·h/ml.

In some examples, the therapeutic composition is formulated to release a dose of the anti-proliferative agent sufficient to generate a blood concentration of the anti-proliferative agent which is smaller than a median maximum serum concentration (Cₘₐₓ) of the anti-proliferative agent generated by systemic delivery of the anti-proliferative agent to achieve the same tissue concentration at the injury site. In other examples, the therapeutic composition is formulated to release a dose of the direct factor Xa inhibitor sufficient to generate a blood concentration of the anti-proliferative agent which is smaller than a median maximum serum concentration (Cₘₐₓ) of the anti-proliferative agent generated by systemic delivery of the anti-proliferative agent when taking one or more oral or IV dose of said anti-proliferative agent. In some examples, the systemic delivery comprises a single oral or IV dose, a daily oral dose, or a smallest oral dose of the anti-proliferative agent. In some examples, the blood concentration is larger than a median minimum serum concentration (Cₘᵢₙ) of the anti-proliferative agent generated by systemic delivery of such agent. In some examples, the blood concentration is smaller than a median minimum serum concentration (Cₘᵢₙ) of the anti-proliferative agent generated by systemic delivery of an oral or I.V systemic therapeutic dose. In some examples, the therapeutic composition is formulated to release a dose of the anti-proliferative agent sufficient to generate a plasma drug level area under the curve (AUC (0-∞)) in ng·h/ml which is smaller than a median AUC (0-∞) in ng·h/ml of the anti-proliferative agent generated by systemic delivery of the anti-proliferative agent to achieve the same tissue concentration at the injury site.

In some examples, the therapeutic composition is formulated to release the one or more active substances at a rate within a range of about 2 μg/mm device to about 100 μg/mm device, about 5 μg/mm device to about 100 μg/mm device, about 7 μg/mm device to about 100 μg/mm device, or about 10 μg/mm device to about 100 μg/mm device within about 3 hours, 12 hours, 1 day, 3 days, 7 days, 28 days, 90 days, or 180 days. In some examples, the therapeutic composition is formulated to release the one or more active substances at a rate within a range of about 5 μg/mm device to about 100 μg/mm device within about 3 hours. In some examples, the therapeutic composition is formulated to release the one or more active substances at a rate within a range of about 5 μg/mm device to about 100 μg/mm device within about 12 hours. In some examples, the therapeutic composition is formulated to release the one or more active substances at a rate within a range of about 5 μg/mm device to about 100 μg/mm device within about 7 days. In some examples, the therapeutic composition is formulated to release the one or more active substances at a rate within a range of about 5 μg/mm device to about 100 μg/mm device within about 28 days.

In some examples, the therapeutic composition is formulated to release the one or more active substances at a dose within a range of about 0.5 μg/mm² device to about 15 μg/mm² device, or of about 1 μg/mm² device to about 12 μg/mm² device, or of about 2 μg/mm² device to about 12 μg/mm² device, or of about 5 μg/mm² device to about 12 μg/mm² device, or of about 7 μg/mm² device to about 12 μg/mm² device, within about 3 hours or about 12 hours or about 1 day or about 3 days or about 7 days. In some examples, the therapeutic composition is formulated to release the one or more active substances at a dose within a range of about 1 μg/mm² device to about 12 μg/mm² device within about 3 hours. In some examples, the therapeutic composition is formulated to release the one or more active substances at a dose within a range of about 1 μg/mm² device to about 12 μg/mm² device within about 12 hours. In some examples, the therapeutic composition is formulated to release the one or more active substances at a dose within a range of about 1 μg/mm² device to about 12 μg/mm² device within about 7 days. In some examples, the therapeutic composition is formulated to release the one or more active substances at a dose within a range of about 1 μg/mm² device to about 12 μg/mm² device within about 28 days.

In some examples, the therapeutic composition is formulated to release the one or more active substances at a rate sufficient to generate a tissue concentration of about 1 ng/mg at about 14 mm from the external surface of the structure within about 28 days.

In some examples, the therapeutic composition is formulated to release the one or more active substances at a rate sufficient to generate a tissue concentration of about 0.5 ng/mg to about 10 ng/mg of tissue adjacent to the device structure within about 28 days or about 90 days or about 180 days.

In some examples, the therapeutic composition is formulated to release the direct factor Xa inhibitor and the anti-proliferative agent at the same rate. In some examples, the therapeutic composition is formulated to release the direct factor Xa inhibitor, and the anti-proliferative agent at different rates. In other examples, the therapeutic composition is formulated to release the direct factor Xa inhibitor at a faster rate than the anti-proliferative agent within the first 3 hours, 1 day, or 72 hour. In yet another example, the therapeutic composition is formulated to release the direct factor Xa inhibitor at a slower rate than the anti-proliferative agent within the first 3 hours, 1 day, or 72 hour.

In some examples, the release rate ratio of the direct factor Xa inhibitor to the anti-proliferative agent is within a range of about 3:2 to about 6:1, or about 2.2:2 to about 6:1, or about 2.5:2 to about 6:1. In some examples, the release rate ratio of the direct factor Xa inhibitor to the anti-proliferative agent is within a range of about 3:2 to about 6:1, about 2.2:2 to about 6:1, or about 2.5:2 to about 6:1 within about 3 hours, about 24 hours, about 7 days, or about 28 days. In some other examples, the release rate ratio of the direct factor Xa inhibitor to the anti-proliferative agent is within a range of about 1:1 to about 2:1 within about 3 hours, 1 day, about 3 days, about 7 days, or about 28 days.

In some examples, the therapeutic composition is formulated to release the anti-proliferative agent at a rate of about 1 μg/second/mm device to about 50 μg/day/mm device, of about 1 μg/min/mm device to about 10 μg/day/mm device, or of about 1 μg/hour/mm device to about 7 μg/day/mm device within about 3 hours, about 1 day, or about 3 days.

In some examples, the therapeutic composition is formulated to release the anti-proliferative agent at a rate of about 1 μg/hour/mm device to about 4 μg/day/mm device.

In some examples, the weight compositional ratio of the direct factor Xa inhibitor to the anti-proliferative agent in the therapeutic composition is about 5:2, about 2:1, about 1.25:1, or about 1:1. In some examples, the weight compositional ratio of the direct factor Xa inhibitor to the anti-proliferative agent in the therapeutic composition is within a range of about 5:1 to about 3:1 or about 5:1 to about 1:1.

In some examples, the therapeutic composition comprises a coating disposed on one or more surfaces of the device structure, and the coating comprises a first layer and a second layer. In some examples, the first layer comprises the direct factor Xa inhibitor. In some examples, the first layer comprises the anti-proliferative agent and the second layer comprises the direct factor Xa inhibitor. In some examples, the therapeutic composition further comprises a top layer or coat of the same or different material as the first layer or the second layer. In some examples, the first layer comprises the direct factor Xa inhibitor and the anti-proliferative agent. In some examples, the second layer comprises a top layer or coat of the same or different material as the first layer. In some examples, the therapeutic composition comprises a coating disposed on one or more surfaces the device structure, and the coating further comprises a biodegradable polymer carrier. In some examples, the first and/or second layer comprise a drug/polymer matrix of the one or more agents. In one example, the first layer is configured for a burst release of the one or more agents, while the second layer is configured for an extended release of the one or more agents. In yet another example, the first and/or second layer are topcoat covering one or more drug agents wherein the one or more drug agents are formulated with an excipient or are formulated in a drug polymer matrix under said first and/or second layer coating. The coating of the matrix and the first or second layers maybe the same or different.

In some examples, the weight compositional ratio of the biodegradable polymer carrier to the one or more active substances is about 1:5 to about 3:2, about 0.5:1 to about 1:1, or about 1:5 to about 1.25:1. In a preferred example, the polymer is biodegradable.

In some other examples, the weight compositional ratio of the carrier to the one or more active substances is about 1:5 to about 3:2, about 0.5:1 to about 1:1, or about 1:5 to about 1.25:1. In one example the carrier is one or more excipients.

In some examples, the therapeutic composition is disposed on at least one surface of the device, preferably on at least the external and/or the inner surfaces of the structure. In some examples, the therapeutic composition is disposed on the external surface (abluminal) of the structure, on the interior surface (luminal) of the structure, and on the side surfaces of the structure. In yet other examples, the therapeutic composition is disposed on one or more surfaces of the structure. In yet other examples, the therapeutic composition is disposed on all surfaces of the structure. In yet other examples, the therapeutic composition is disposed in a reservoir on or in the structure. In some examples, the therapeutic composition is disposed on the external surface of the structure.

In some examples, the therapeutic composition further comprises an anti-proliferative agent. In some examples, the direct factor IIa inhibitor comprises Argatroban, dabigatran, ximelagatran, melagatran, efegatran, inogatran, atecegatran metoxil (AZD-0837), hirudin, hirudin analogs, bivalirudin, desirudin, or lepirudin. In some examples, the direct factor IIa inhibitor comprises Argatroban, or a salt, isomer, solvate, analog (including deuterated analog), derivative, metabolite, or prodrugs thereof. In some examples, the direct factor IIa inhibitor comprises dabigatran, or a salt, isomer, solvate, analog (including deuterated analog), derivative, metabolite, or prodrugs thereof.

In some examples, the direct factor IIa inhibitor and the direct factor Xa inhibitor combined dose synergistically increase clotting time as measured by ACT by a range of about 3-7 times the ACT of the factor IIa at a dose equal to the combined dose, or by a range of about 1.5 to 2 times the ACT of the factor Xa inhibitor at a dose equal to the combined dose.

In some examples, the direct factor IIa inhibitor and the direct factor Xa inhibitor combined dose synergistically increase clotting time as measured by ACT by a range of about 2-3 times the ACT of the factor IIa at a dose equal to the combined dose and the ACT of the factor Xa inhibitor at a dose equal to the combined dose.

In other examples, the therapeutic composition comprising a direct factor IIa inhibitor and a direct factor Xa inhibitor is formulated to release said agents at a rate and/or concentration sufficient to accelerate dissolution or to inhibit one or more of inflammation, smooth muscle cell proliferation, cell proliferation, thrombin formation, fibrin formation, platelet aggregation, platelet activation, vessel injury, or clot formation, within about 3 hours to about 28 days or longer, or within about 3 hours to about 3 months or longer.

In other examples, the therapeutic composition comprising a direct factor IIa inhibitor and a direct factor Xa inhibitor is formulated to release said agents to accelerate dissolution of or to inhibit one or more of inflammation, smooth muscle cell proliferation, cell proliferation, thrombin formation, fibrin formation, platelet aggregation, platelet activation, vessel injury, or clot formation, within about 3 hours to about 28 days or longer, or within about 3 hours to about 3 months or longer.

In other examples, the therapeutic composition comprising a direct factor IIa inhibitor and a direct factor Xa inhibitor formulated to have a weight composition ratio of factor Xa inhibitor to factor IIa inhibitor in the ratio ranging from about 1:1 to about 10:1. In other examples, the therapeutic composition comprising a direct factor IIa inhibitor and a direct factor Xa inhibitor formulated to have a weight composition ratio of factor Xa inhibitor to factor IIa inhibitor in the ratio ranging from about 0.5:1 to about 5:1.

In some examples, the therapeutic composition is formulated to reduce one or more of cell proliferation or fibrin formation within 7 days or longer.

In some examples, the therapeutic composition is formulated to release the two or more active substances at a rate of 1 μg/second/mm device to about 50 μg/day/mm device, preferably at a rate of 1 μg/min/mm device to about 30 μg/day/mm device, more preferably at a rate of 1 μg/hour/mm device to about 30 μg/day/mm device. In some examples, the therapeutic composition is formulated to begin releasing the two or more active substances prior to positioning of the device adjacent to the injury site, or immediately after, or within about 5, about 15, or about 30 minutes after the at least one surface of the structure is positioned adjacent the injury site. In some examples, the therapeutic composition is formulated to begin releasing the two or more active substances before the external surface of the structure is positioned adjacent the injury site. In some examples, the therapeutic composition is formulated to release substantially all of the two or more active substances within about 1 to about 90 days or more. In some examples, the therapeutic composition is formulated to release substantially all of the two or more active substances within about 90 to about 180 days or more. In some examples, the therapeutic composition is formulated to release substantially all of the two or more active substances within about 7 days or about 28 days. In some examples, the therapeutic composition is formulated to release substantially all of the two or more active substances within about 3 hours or about 6 hours or about 12 hours or about 1 day or about 3 days. In some examples, the therapeutic composition is formulated to release at least 50% or at least 60% or at least 70% of the two or more active substances within about 3 hours or about 6 hours or about 12 hours or about 1 day or about 3 days or about 7 days or about 28 days.

In some examples, the therapeutic composition is formulated to release the two or more active substances at a rate sufficient to generate a tissue concentration at the injury site within a range of about 2 ng/mg to about 100 ng/mg within about 24 hours. In some examples, the therapeutic composition is formulated to release the two or more active substances at a rate sufficient to generate a tissue concentration at the injury site within a range of about 3 ng/mg to about 50 ng/mg within about 24 hours. In some examples, the therapeutic composition is formulated to release the two or more active substances at a rate sufficient to generate a tissue concentration at the injury within a range of about 4 ng/mg to about 25 ng/mg within about 24 hours.

In some examples, the therapeutic composition is formulated to release the two or more active substances at a rate sufficient to generate a tissue concentration at the injury site within a range of about 1 ng/mg to about 30 ng/mg within about 7 days. In some examples, the therapeutic composition is formulated to release the two or more active substances at a rate sufficient to generate a tissue concentration at the injury site within a range of about 1.5 ng/mg to about 20 ng/mg within about 7 days. In some examples, the therapeutic composition is formulated to release the two or more active substances at a rate sufficient to generate a tissue concentration at the injury site within a range of about 2 ng/mg to about 25 ng/mg within about 7 days.

In some examples, the therapeutic composition is formulated to release the two or more active substances at a rate sufficient to generate a tissue concentration at the injury site within a range of about 0.5 ng/mg to about 30 ng/mg within about 28 days. In some examples, the therapeutic composition is formulated to release the two or more active substances at a rate sufficient to generate a tissue concentration at the injury site within a range of about 1 ng/mg to about 20 ng/mg within about 28 days. In some examples, the therapeutic composition is formulated to release the two or more active substances at a rate sufficient to generate a tissue concentration at the injury site within a range of about 1.5 ng/mg to about 25 ng/mg within about 28 days.

In some examples, the therapeutic composition is formulated to release the two or more active substances at a rate sufficient to generate a tissue concentration at the injury site within a range of about 0.1 ng/mg to about 10 ng/mg within about 90 days or about 180 days.

In some examples, the therapeutic composition is formulated to release the two or more active substances at a rate sufficient to generate a tissue concentration at a location proximal or distal a proximal end of the structure or a distal end of the structure, respectively, within a range of about 0.5 ng/mg to about 500 ng/mg within about 3 hours. In some examples, the therapeutic composition is formulated to release the two or more active substances at a rate sufficient to generate a tissue concentration at the location proximal or distal the proximal end of the structure or the distal end of the structure, respectively, within a range of about 1 ng/mg to about 35 ng/mg within about 3 hours. In some examples, the therapeutic composition is formulated to release the two or more active substances at a rate sufficient to generate a tissue concentration at the location proximal or distal the proximal end of the structure or the distal end of the structure, respectively, within a range of about a range of about 1.5 ng/mg to about 30 ng/mg within about 3 hours. In some examples, the therapeutic composition is formulated to release the two or more active substances at a rate sufficient to generate a tissue concentration at the location proximal or distal to the proximal end of the structure or the distal end of the structure (e.g., within ±5 mm proximal or distal to an end of the structure), respectively, within a range of about 0.1 ng/mg to about 50 ng/mg, about 0.25 ng/mg to about 20 ng/mg, about 1 ng/mg to about 50 ng/mg, or about 3 ng/mg to about 50 ng/mg within about 3 hours.

In some examples, the therapeutic composition is formulated to release the two or more active substances at a rate sufficient to generate a tissue concentration at a location proximal or distal a proximal end of the structure or a distal end of the structure, respectively, within a range of about 0.2 ng/mg to about 25 ng/mg, about 2 ng/mg to about 25 ng/mg, or about 4 ng/mg to about 25 ng/mg within about 24 hours. In some examples, the therapeutic composition is formulated to release the two or more active substances at a rate sufficient to generate a tissue concentration at the location proximal or distal to the proximal end of the structure or the distal end of the structure (e.g., within ±5 mm proximal or distal to an end of the structure), respectively, within a range of about 0.1 ng/mg to about 50 ng/mg, about 0.25 ng/mg to about 20 ng/mg, about 1 ng/mg to about 50 ng/mg, or about 3 ng/mg to about 50 ng/mg within about 24 hours. In some examples, the therapeutic composition is formulated to release the two or more active substances at a rate sufficient to generate a tissue concentration at the location proximal or distal the proximal end of the structure or the distal end of the structure, respectively, within a range of about 0.3 ng/mg to about 10 ng/mg within about 24 hours.

In some examples, the therapeutic composition is formulated to release a dose of the direct factor Xa inhibitor sufficient to generate a plasma drug level area under the curve (AUC (0-24) or AUC (0-∞)) in ng·h/ml which is smaller than a median AUC (AUC (0-24) or AUC (0-∞)) in ng·h/ml of the direct factor Xa inhibitor generated by systemic delivery of the direct factor Xa inhibitor to achieve the same tissue concentration at the injury site. In other examples, the therapeutic composition is formulated to release a dose of the direct factor Xa inhibitor sufficient to generate a plasma drug level area under the curve (AUC (0-24) or AUC (0-∞)) in ng·h/ml which is less than a median (AUC (0-24) or AUC (0-∞)) in ng·h/ml of the direct factor Xa inhibitor generated by systemic delivery of the direct factor Xa inhibitor when taking one or more oral dose of said factor Xa inhibitor. In some examples, the systemic delivery comprises a single oral dose, a daily oral dose, or a smallest oral dose of the direct factor Xa inhibitor. In some examples, the (AUC (0-24) or AUC (0-∞)) is measured using one of plasma blood, serum blood, or whole blood. In other examples, the median (AUC (0-24) or AUC (0-∞)) is 724 ng·h/ml, or1437 ng·h/ml, or 2000 ng·h/ml, or 4000 ng·h/ml.

In some examples, the therapeutic composition is formulated to release a dose of the direct factor IIa inhibitor (of the two or more active substances) sufficient to generate a blood concentration of the direct factor IIa inhibitor which is smaller than a median maximum serum concentration (Cₘₐₓ) of the direct factor IIa inhibitor generated by systemic delivery of the direct factor IIa inhibitor to achieve the same tissue concentration at the injury site. In other examples, the therapeutic composition is formulated to release a dose of the direct factor IIa inhibitor sufficient to generate a blood concentration of the direct factor IIa inhibitor which is smaller than a median maximum serum concentration (Cₘₐₓ) of the direct factor IIa inhibitor generated by systemic delivery of the direct factor IIa inhibitor when taking one or more oral dose of said factor IIa inhibitor. In some examples, the systemic delivery comprises a single oral dose, a daily oral dose, or a smallest oral dose of the direct factor IIa inhibitor. In some examples, the blood concentration is larger than a median minimum serum concentration (Cₘᵢₙ) of the direct factor IIa inhibitor generated by systemic delivery. In some examples, the blood concentration is smaller than a median minimum serum concentration (Cₘᵢₙ) of the direct factor IIa inhibitor generated by systemic delivery. In some examples, the Cₘₐₓ is measured using one of plasma blood, serum blood, or whole blood. In other examples, the median Cₘₐₓ is 80 ng/ml, or 123 ng/ml, or 171 ng/ml, or 321 ng/ml, or 480 ng/ml of blood.

In some examples, the therapeutic composition is formulated to release a dose of the direct factor IIa inhibitor sufficient to generate a plasma drug level area under the curve (AUC (0-24) or AUC (0-∞)) in ng·h/ml which is smaller than a median AUC (AUC (0-24) or AUC (0-∞)) in ng·h/ml of the direct factor IIa inhibitor generated by systemic delivery of the direct factor IIa inhibitor to achieve the same tissue concentration at the injury site. In other examples, the therapeutic composition is formulated to release a dose of the direct factor IIa inhibitor sufficient to generate a plasma drug level area under the curve (AUC (0-24) or AUC (0-∞)) in ng·h/ml which is less than a median (AUC (0-24) or AUC (0-∞)) in ng·h/ml of the direct factor IIa inhibitor generated by systemic delivery of the direct factor IIa inhibitor when taking one or more oral dose of said factor IIa inhibitor. In some examples, the systemic delivery comprises a single oral dose, a daily oral dose, or a smallest oral dose of the direct factor IIa inhibitor. In some examples, the (AUC (0-24) or AUC (0-∞)) is measured using one of plasma blood, serum blood, or whole blood. In other examples, the median (AUC (0-24) or AUC (0-∞)) is 724 ng·h/ml, or 1437 ng·h/ml, or 2000 ng·h/ml, or 4000 ng·h/ml.

In some examples, the therapeutic composition is formulated to release a dose of the anti-proliferative agent (of the two or more active substances) sufficient to generate a blood concentration of the anti-proliferative agent which is smaller than a median maximum serum concentration (Cₘₐₓ) of the anti-proliferative agent generated by systemic delivery of the anti-proliferative agent to achieve the same tissue concentration at the injury site.

In some examples, the systemic delivery comprises a single oral dose, a daily oral dose, or a smallest oral dose of the anti-proliferative agent. In some examples, the blood concentration is larger than a median minimum serum concentration (Cₘᵢₙ) of the anti-proliferative agent generated by systemic delivery. In some examples, the blood concentration is smaller than a median minimum serum concentration (Cₘᵢₙ) of the anti-proliferative agent generated by systemic delivery.

In some examples, the therapeutic composition is formulated to release a dose of the anti-proliferative agent sufficient to generate a plasma drug level area under the curve (AUC (0-∞)) in ng·h/ml which is smaller than a median AUC (0-∞) in ng·h/ml of the anti-proliferative agent generated by systemic delivery of the anti-proliferative agent to achieve the same tissue concentration at the injury site.

In some examples, the therapeutic composition is formulated to release the two or more active substances at a rate sufficient to generate a tissue concentration of about 1 ng/mg at about 14 mm from the external surface of the structure within about 28 days. In some examples, the therapeutic composition is formulated to release the two or more active substances at a rate sufficient to generate a tissue concentration of about 0.5 ng/mg to about 10 ng/mg of tissue adjacent to the device structure within about 28 days or about 90 days or about 180 days.

In some examples, the therapeutic composition is formulated to release the direct factor IIa inhibitor and the direct factor Xa inhibitor at about the same rate. In some examples, the therapeutic composition is formulated to release the direct factor IIa inhibitor and the direct factor Xa inhibitor at different rates. In some examples, the release rate ratio of the direct factor IIa inhibitor to the direct factor Xa inhibitor is within a range of about 0.7:1 to about 2:1. In some examples, the release rate ratio of the direct factor IIa inhibitor to the direct factor Xa inhibitor is within a range of about 0.7:1 to about 2:1 within about 3 hours, about 24 hours, or about 7 days.

In some examples, the therapeutic composition is formulated to release the direct factor IIa inhibitor, the direct factor Xa inhibitor, and the anti-proliferative agent at about the same rate. In some examples, the therapeutic composition is formulated to release the direct factor Xa inhibitor and the direct factor IIa inhibitor faster than the anti-proliferative agent. In some examples, the dose of the direct factor Xa inhibitor or the direct factor IIa inhibitor is about 1 to about 6 times larger, about 1.25 to about 5 times larger, about 1.5 to about 3 times larger, or about 1.5 to about 2.5 times larger than a dose of the anti-proliferative agent. In some examples, the therapeutic composition is formulated to release the direct factor IIa inhibitor, the direct factor Xa inhibitor, and the anti-proliferative agent at different rates.

In some examples, the therapeutic composition is formulated to release the anti-proliferative agent at a rate of about 1 μg/second/mm device to about 50 μg/day/mm device, of about 1 μg/min/mm device to about 10 μg/day/mm device, or of about 1 μg/hour/mm device to about 7 μg/day/mm device within about 3 hours, about 1 day, or about 3 days.

In some examples, the release rate ratio of the direct factor IIa inhibitor to the direct factor Xa inhibitor to the anti-proliferative agent is within a range of about 1:1:1 to about 4:4:1. In some examples, the therapeutic composition is formulated to release the direct factor IIa inhibitor at a rate of about 4 μg/hour/mm device to about 14 μg/day/mm device. In some examples, the therapeutic composition is formulated to release the direct factor Xa inhibitor at a rate of about 4 μg/hour/mm device to about 14 μg/day/mm device. In some examples, the therapeutic composition is formulated to release the anti-proliferative agent at a rate of about 1 μg/hour/mm device to about 4 μg/day/mm device.

In some examples, the weight compositional ratio of the direct factor IIa inhibitor to the direct factor Xa inhibitor in the therapeutic composition is about 1:1. In some examples, the weight compositional ratio of the direct factor IIa inhibitor to the direct factor Xa inhibitor in the therapeutic composition is within a range of about 3:1 to about 1:3, for example about 1:1. In some examples, the weight compositional ratio of the direct factor IIa inhibitor to the direct factor Xa inhibitor to the anti-proliferative agent in the therapeutic composition is about 5:5:2. In some examples, the weight compositional ratio of the direct factor IIa inhibitor to the direct factor Xa inhibitor to the anti-proliferative agent in the therapeutic composition is within a range of about 6:6:1 to about 1:3:1.

In some other examples, the weight compositional ratio of the carrier to the two or more active substances is about 1:5 to about 3:1, about 0.5:1 to about 1:1, or about 1:5 to about 1.25:1. In one example the carrier is one or more excipients.

In some examples, the coating further comprises a third layer. In some examples, the first layer comprises the direct factor IIa inhibitor, the second layer comprises the direct factor Xa inhibitor, and the third layer comprises the anti-proliferative agent. In some examples, the therapeutic composition further comprises a top layer or coat of the same or different material as the first layer, the second layer, or the third layer.

In some examples, the two or more active substances are present in the polymeric material at weight ratios of about 1:3:1; about 3:2:1; about 2:2:1; about 2:3:1; about 3:3:1; about 5:5:1; or about 6:6:1 of direct factor IIa inhibitor to direct factor Xa inhibitor to anti-proliferative agent.

In some examples, the polymeric material is porous. In some examples, the polymeric material has a porosity within a range of about 10 nm to about 10 μm. In some examples, the polymeric material is non-degradable. In some examples, the polymeric material is biodegradable. In some examples, the polymeric material has a degradation rate within a range of about 1 month to about 36 months. In some examples, the polymeric material comprises a material selected from a group consisting of polyesters, polylactide, polyglycolide, poly(F-caprolactone), polydioxanone, poly(hydroxyalkanoates), poly(L-lactide-co-D-lactide), poly(L-lactide-co-D,L-lactide), poly(D-lactide-co-D,L-lactide), poly(lactide-co-glycolide) (including 70:30 to 99:1 PLA-co-PGA, such as 85:15 PLA-co-PGA), poly(lactide-co-F-caprolactone) (including 70:30 to 99:1 PLA-co-PCL, such as 90:10 PLA-co-PCL), poly(glycolide-co-F-caprolactone), poly(lactide-co-dioxanone), poly(glycolide-co-dioxanone), poly(lactide-co-trimethylene carbonate), poly(glycolide-co-trimethylene carbonate), poly(lactide-co-ethylene carbonate), block polymers and copolymers and combinations thereof, wherein lactide includes L-lactide, D-lactide and D,L-lactide. In some examples, the polymeric material comprises a material selected from a group of non-degradable polymeric materials consisting of polyacrylates, polymethacrylates, poly(n-butyl methacrylate), poly(hydroxyethylmethacrylate), poly(styrene-b-isobutylene-b-styrene), phosphorylcholine polymer, poly(ethylene-co-vinyl acetate), poly(n-butyl methacrylate), blend of thermoplastic Silicone-Polycarbonate-urethane with poly n-butyl methacrylate, poly(vinylidene-co-hexafluoropropylene), Blend of polyvinylpyrrolidone, poly(hexylmethacrylate)-co-polyvinylpyrrolidone-co-poly vinyl acetate, and poly(n-butyl methacrylate)-co-poly(vinyl acetate), Poly(styrene-butylene styrene), poly(tyrosine-derived polycarbonate), polyamides, nylons, nylon 12, Dacron, Polyethylene terephthalate, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate), polyvinylpyridine block with poly methyl methacrylate (PMMA), poly N-(2-Hydroxypropyl) methacrylamide, Polyethylenimine (PEI), dextran, dextrin, chitosans, poly(L-lysine), and poly(aspartamides), polyamides, Polyethylene glycol (PEG), Silicones, poly(anhydride), poly ortho esters, polystyrene-b-polyvinylpyridine, poly(styrene)-poly(butadiene)-poly(vinyl pyridine), poly(styrene-poly(methacrylic acid), poly(styrene)-poly(ethylene oxide), poly(vinyl pyridine)-poly(butadiene)-poly(vinyl pyridine), and poly(styrene)-poly(vinyl pyridine)-poly(ethylene oxide) and copolymers and combinations thereof.

In other examples, the therapeutic compositions of the present invention may comprise a direct factor IIa inhibitor and a direct factor Xa inhibitor and an anti-proliferative is formulated to release said agents at a rate sufficient to inhibit one or more of inflammation, smooth muscle cell proliferation, cell proliferation, thrombin formation, fibrin formation, or clot formation, within about 3 hours to about 28 days or longer, or within about 3 hours to about 3 months or longer.

In another example, the therapeutic composition of the present invention is formulated to release the two or more active substances, wherein the two or more substances comprise a direct IIa inhibitor, a direct Xa inhibitor, and an antiproliferative, to an injury site in a body lumen. In another example, the therapeutic composition is formulated to release the two or more active substances, wherein the two or more substances include a direct IIa inhibitor, a direct Xa inhibitor, and an antiproliferative, to a target site in a body lumen. In another example, the therapeutic composition is formulated to release the two or more active substances, wherein the two or more substances include a direct IIa inhibitor, and an antiproliferative, to a target site in a body lumen. In another example, the therapeutic composition is formulated to release the two or more active substances, wherein the two or more substances include a direct IIa inhibitor, a direct Xa inhibitor, to a target site in a body lumen. In another example, the therapeutic composition is formulated to release the two or more active substances, wherein the two or more substances include a direct Xa inhibitor and an anti-proliferative, to a target site in a body lumen.

In some examples, the injury is at least partially caused before deployment of the structure. In some examples, deployment of the structure causes the injury and the therapeutic composition is formulated to release the direct factor IIa inhibitor, the direct factor Xa inhibitor, or the anti-proliferative agent before the injury occurs.

In yet other examples, an implant comprising a therapeutic composition, wherein said composition is formulated to release a dose of a direct factor Xa inhibitor and a dose of a factor IIa inhibitor, at a target site sufficient to inhibit or resolve one or more of inflammation, injury, smooth muscle cell proliferation, cell proliferation, clot formation, platelet activation, or platelet aggregation, wherein said clot formation comprises one or more of clot at the injured tissue site, clot at a blood vessel adjacent to said tissue site, clot at the implant surface (exterior and/or interior), or clot in the systemic blood circulation resulting from said tissue injury.

In some examples, the therapeutic composition comprises a first and/or second layer comprise a drug/polymer matrix of the one or more agents. In one example, the first layer is configured for a burst release of the one or more agents, while the second layer is configured for an extended release of the one or more agents. In yet another example, the first and/or second layer are topcoat covering one or more drug agents wherein the one or more drug agents are formulated with an excipient or are formulated in a drug polymer matrix under said first and/or second layer coating. The coating of the matrix and the first or second layers maybe the same or different.

In another example of any of the examples in this application, a therapeutic composition comprising two or more active substances on at least one surface of the device is configured to be positioned adjacent to a target site in the patient's body, wherein adjacent to comprises one or more of the following: next to, touching, deployed at, expanded at, pushing against, placed against, or other. In a preferred example, the active substances are a direct factor IIa inhibitor and a direct factor Xa inhibitor. In another example the active substances are a direct factor IIa inhibitor, a direct factor Xa inhibitor and an anti-proliferative. In yet another example, the active substances are one of Argatroban, Rivaroxaban or Apixaban, and Sirolimus or Sirolimus analogue.

In one example, several anticoagulants delivered locally were tested in-vivo in an animal model including Heparin, Rivaroxaban (factor Xa inhibitor), and Argatroban (factor IIa inhibitor). It was an unexpected result that only Rivaroxaban formulation was shown to inhibit fibrin formation at 7 days.

In another example, two formulations of Rivaroxaban were tested in a local delivery in-vivo animal model, wherein one formulation comprised a faster release dose release within 7 days versus control within 7 days. It was unexpected result that the composition comprising faster release dose released within 7 days was more effective than control, within 7 days. The composition comprising faster dose formulation inhibited fibrin more effectively through 28 days compared to the 7 days slower release dose.

A surprising finding was that composition comprising the fast release of Rivaroxaban in combination with m-TOR inhibitor released locally was more effective at inhibiting fibrin at 7 days and 28 days as compared to control while a slower release formulation of Rivaroxaban in combination with m-TOR inhibitor was less effective at inhibiting fibrin formation at 28 days from implant.

A surprising finding was that a composition comprising Rivaroxaban released locally in combination with m-TOR inhibitor inhibits fibrin formation after injury. Many attempts using heparin, and other anticoagulants have failed to show such effects when combined with m-TOR inhibitors.

A surprising finding was that a composition comprising Rivaroxaban released locally in combination with Argatroban inhibited fibrin formation after injury.

A surprising finding was that a composition comprising Rivaroxaban released locally in combination with Argatroban inhibited smooth muscle cell proliferation after injury.

A surprising finding was that a composition comprising Rivaroxaban released locally in combination with Argatroban and an m-TOR inhibitor further inhibited smooth muscle cell proliferation after injury.

In one example, a device for use in a body lumen is configured to release locally a composition comprising factor Xa inhibitor, preferably Rivaroxaban, more preferably Apixaban, wherein said device is configured to release at least 89 μg, preferably at least 150 μg (micro-grams) of said factor Xa inhibitor, within 3 hours, within 12 hours, within 1 day, within 3 days, or within 7 days from time of injury.

In another example, a device for use in a body lumen is configured to release locally a composition comprising factor Xa inhibitor, preferably Rivaroxaban, more preferably Apixaban, wherein said device is configured to release at least 6.36 μg per millimeter of device length, preferably release at least 10.7 μg per millimeter of device length, of said factor Xa inhibitor within 3 hours, within 12 hours, within 1 day, within 3 days, or within 7 days from time of injury.

In another example, a device for use in a body lumen is configured to release locally a composition comprising factor Xa inhibitor, preferably Rivaroxaban, more preferably Apixaban, wherein said device from time of injury to 3 hours, 12 hours, 1 day, 3 days, or to 7 days is configured to release 89 μg or more or 6.36 μg or more/mm of device length of said drug, preferably configured to release 150 μg or more or 10.7 μg/mm of device length or more of said drug.

In another unexpected finding that the combination of factor Xa inhibitor Apixaban and Argatroban combination was shown to enhance the SMC proliferation inhibition when released together with m-TOR inhibitor, Sirolimus. Further finding showed Apixaban or Rivaroxaban and Argatroban combination had synergistic effects of one or more of extending time before clotting of blood, anti-fibrin formation, or anti clot formation effects that was better than either alone.

In an unexpected finding, composition comprising of a factor Xa inhibitor Apixaban, factor IIa inhibitor Argatroban, and the M-Tor inhibitor Sirolimus exhibited more efficacy at inhibiting one or more of the following at 28 days and/or 90 day time points: cell proliferation, inflammation, injury, fibrin formation inhibition, clot formation, and fibrin dissolution acceleration; and/or extending time before clotting of blood, and/or increasing ACT.

The composition comprising a combination of factor Xa inhibitor Apixaban, a factor II inhibitor Argatroban and an anti-proliferative (M-tor) formulation was surprisingly more effective than an anti-proliferative (M-tor) alone.

The composition comprising a combination of factor Xa inhibitor Apixaban and a factor II inhibitor Argatroban was effective at inhibiting clot/thrombus formation.

The composition comprising a combination of factor Xa inhibitor and a factor IIa inhibitor had surprisingly synergistic effect in extending time before clotting, an/or enhance anticoagulation effect, and/or inhibit clot formation, at a concentration of 0.025 ng/mg for each drug and higher. In a preferred example, a composition comprising a combination of factor Xa inhibitor and factor IIa inhibitor configured to release over a period ranging from 7 days to 1 year, preferably ranging from 21 day to 1 year, more preferably ranging from 30 days to one year, wherein the tissue concentration adjacent to said composition ranges from 0.025 ng/mg for each of said drugs to 10 ng/mg over said period.

In some examples, the therapeutic composition comprises one or more anticoagulant agents that has an IC50 to inhibit factor Xa and factor II at a dose ranging from 0.0001 nM to 1000 nM, preferably at a dose ranging from 0.0001 nM to 100 nM, more preferably at a dose ranging from 0.0001 nM to 10 nM, and most preferably at a dose ranging from 0.0001 nM to 1 nM.

Other aspects and features of the present invention are set forth in the following numbered clauses.

Clause 1. A method for inhibiting an inflammatory pulmonary disease in a patient, the method comprising: providing a therapeutic composition comprising at least one of a direct factor IIa and a direct factor Xa inhibitor; and delivering a therapeutically effective dose of the therapeutic composition to a site of the inflammatory pulmonary disease in the patient's lung.

Clause 2. The method of clause 1, wherein the therapeutically effective dose of the therapeutic composition is effective to suppress or prevent initiation, progression, or relapses of disease, including the progression of established disease.

Clause 3. The method of clause 1 or 2, wherein delivering the therapeutically effective dose of the therapeutic composition comprises any one or more of inhalation, ventilation, instillation, ultrasound, vibration, and injection.

Clause 4. The method of any one of the preceding clauses, wherein the inflammatory pulmonary disease comprises one or more of clot formation or fibrin formation.

Clause 5. The method of any one of the preceding clauses, wherein the inflammatory pulmonary disease is caused by viral infection, bacterial infection, smoke inhalation, chemical exposure, genetic mutation, injury, smog and other air pollutions, inhalation of environmental and occupational pollutants, work-related lung diseases, hypersensitivity pneumonitis, and combinations thereof.

Clause 6. The method of any one of the preceding clauses, wherein the inflammatory pulmonary disease comprises pneumonia, bronchitis, emphysema, asthma, pulmonary fibrosis, lung cancer, pulmonary edema, pulmonary embolism, sarcoidosis and granulomatosis with polyangiitis, chronic obstructive pulmonary disease, bronchiectasis, acute respiratory distress syndrome (ARDS), SARS, or COVID-19.

Clause 7. The method of any one of the preceding clauses, further comprising diagnosing the patient as having the inflammatory pulmonary disease prior to delivering the therapeutically effective dose of the therapeutic composition.

Clause 8. The method of any one of the preceding clauses, wherein the direct factor Xa inhibitor is selected from the group consisting of Apixaban, betrixaban, edoxaban, otamixaban, razaxaban, Rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), eribaxaban (PD 0348292), 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), (S)-3-(7-carbamimidoylnaphthalen-2-yl)-2-(4-(((S)-1-(1-iminoethyl)pyrrolidin-3-yl)oxy)phenyl)propanoic acid hydrochloride pentahydrate (DX9065a), letaxaban (TAK-442), GW813893, JTV-803, KFA-144, DPC-423, RPR-209685, MCM-09, or antistasin.

Clause 9. The method of clause 8, wherein the direct factor Xa inhibitor comprises Rivaroxaban, or a salt, isomer, solvate, analog, derivative, metabolite, or prodrugs thereof.

Clause 10. The method of clause 8, wherein the direct factor Xa inhibitor comprises Apixaban, or a salt, isomer, solvate, analog, derivative, metabolite, or prodrugs thereof.

Clause 11. The method of any one of the preceding clauses, wherein the direct factor Xa inhibitor is present in the therapeutic composition at a concentration within a range of about 0.1 nM to about 1,000,000 nM.

Clause 12. The method of clause 11, wherein the direct factor Xa inhibitor is present in the therapeutic composition at a concentration within a range of about 1 nM to about 1,000,000 nM.

Clause 13. The method of clause 11, wherein the direct factor Xa inhibitor is present in the therapeutic composition at a concentration within a range of about 10 nM to about 1,000,000 nM.

Clause 14. The method of clause 11, wherein the direct factor Xa inhibitor is present in the therapeutic composition at a concentration within a range of about 100 nM to about 1,000,000 nM.

Clause 15. The method of any one of the preceding clauses, wherein the therapeutically effective dose is sufficient to generate a tissue concentration of the direct factor Xa inhibitor of about 0.1 ng/mg tissue to about 10 ng/mg tissue.

Clause 16. The method of clause 15, wherein the therapeutically effective dose is sufficient to generate a tissue concentration of the direct factor Xa inhibitor of about 0.2 ng/mg tissue to about 10 ng/mg tissue.

Clause 17. The method of clause 15, wherein the therapeutically effective dose is sufficient to generate a tissue concentration of the direct factor Xa inhibitor of about 0.5 ng/mg tissue to about 5 ng/mg tissue.

Clause 18. The method of clause 15, wherein the therapeutically effective dose is sufficient to generate a tissue concentration of the direct factor Xa inhibitor of about 1 ng/mg tissue to about 5 ng/mg tissue.

Clause 19. The method of clause 15, wherein the therapeutically effective dose is sufficient to generate a blood concentration of the direct factor Xa inhibitor which is less than about 200 ng/ml, 100 ng/ml, 50 ng/ml, 40 ng/ml, or 10 ng/ml.

Clause 20. The method of clause 15, wherein the therapeutically effective dose is sufficient to generate a blood concentration of the direct factor Xa inhibitor which is smaller than a median maximum serum concentration (Cmax) of the direct factor Xa inhibitor generated by systemic delivery of the direct factor Xa inhibitor to achieve the same tissue concentration at the site of the inflammatory pulmonary disease.

Clause 21. The method of clause 15, wherein the therapeutically effective dose is sufficient to generate a blood concentration of the direct factor Xa inhibitor which does not exceed a median maximum serum concentration (Cmax) of the direct factor Xa inhibitor generated by systemic delivery of the direct factor Xa inhibitor to achieve the same tissue concentration at the site of the inflammatory pulmonary disease for more than about 6 hours to about 12 hours.

Clause 22. The method of any one of the preceding clauses, wherein the therapeutic composition further comprises at least one additional therapeutically active substance.

Clause 23. The method of clause 22, wherein the at least one additional therapeutically active substance comprises a direct factor IIa inhibitor selected from the group consisting of Argatroban, dabigatran, ximelagatran, melagatran, efegatran, inogatran, atecegatran metoxil (AZD-0837), hirudin, hirudin analogs, bivalirudin, desirudin, or lepirudin.

Clause 24. The method of clause 23, wherein the direct factor IIa inhibitor comprises Argatroban. or a salt, isomer, solvate, analog, derivative, metabolite, or prodrugs thereof.

Clause 25. The method of clause 23, wherein the direct factor Xa inhibitor comprises Apixaban and the direct factor IIa inhibitor comprises Argatroban.

Clause 26. The method of clause 23, wherein the direct factor IIa inhibitor is present in the therapeutic composition at a concentration within a range of about 0.1 nM to about 1,000,000 nM.

Clause 27. The method of clause 26, wherein the direct factor IIa inhibitor is present in the therapeutic composition at a concentration within a range of about 1 nM to about 1,000,000 nM.

Clause 28. The method of clause 26, wherein the direct factor IIa inhibitor is present in the therapeutic composition at a concentration within a range of about 10 nM to about 1,000,000 nM.

Clause 29. The method of clause 26, wherein the direct factor IIa inhibitor is present in the therapeutic composition at a concentration within a range of about 100 nM to about 1,000,000 nM.

Clause 30. The method of clause 23, wherein the therapeutically effective dose is sufficient to generate a tissue concentration of the direct factor IIa inhibitor of about 0.1 ng/mg tissue to about 10 ng/mg tissue.

Clause 31. The method of clause 30, wherein the therapeutically effective dose is sufficient to generate a tissue concentration of the direct factor IIa inhibitor of about 0.2 ng/mg tissue to about 10 ng/mg tissue.

Clause 32. The method of clause 30, wherein the therapeutically effective dose is sufficient to generate a tissue concentration of the direct factor IIa inhibitor of about 0.5 ng/mg tissue to about 5 ng/mg tissue.

Clause 33. The method of clause 30, wherein the therapeutically effective dose is sufficient to generate a tissue concentration of the direct factor IIa inhibitor of about 1 ng/mg tissue to about 5 ng/mg tissue.

Clause 34. The method of clause 30, wherein the therapeutically effective dose is sufficient to generate a blood concentration of the direct factor IIa inhibitor which is less than about 200 ng/ml, 100 ng/ml, 50 ng/ml, 40 ng/ml, or 10 ng/ml.

Clause 35. The method of clause 30, wherein the therapeutically effective dose is sufficient to generate a blood concentration of the direct factor IIa inhibitor which is smaller than a median maximum serum concentration (Cmax) of the direct factor IIa inhibitor generated by systemic delivery of the direct factor IIa inhibitor to achieve the same tissue concentration at the site of the inflammatory pulmonary disease.

Clause 36. The method of clause 30, wherein the therapeutically effective dose is sufficient to generate a blood concentration of the direct factor IIa inhibitor which does not exceed a median maximum serum concentration (Cmax) of the direct factor IIa inhibitor generated by systemic delivery of the direct factor IIa inhibitor to achieve the same tissue concentration at the site of the inflammatory pulmonary disease for more than about 6 hours to about 12 hours.

Clause 37. The method of clause 30, wherein the weight compositional ratio of the direct factor Xa inhibitor to the direct factor IIa inhibitor in the therapeutic composition is within a range of about 3:1 to about 1:3.

Clause 38. The method of clause 30, wherein the weight compositional ratio of the direct factor Xa inhibitor to the direct factor IIa inhibitor in the therapeutic composition is about 1:1.

Clause 39. The method of any one of the preceding clauses, wherein the therapeutic composition is administered in combination with one or more additional pharmaceutical agents.

Clause 40. The method of clause 39, wherein the one or more additional pharmaceutical agents comprises one or more anti-fibrotic agents, anti-viral agents, anti-bacterial agents, metformin or its salt, steroids, interferons, anti-proliferative, anti-angiogenic, anti-VEGF, or combinations thereof.

Clause 41. The method of clause 40, wherein the anti-fibrotic agent comprises pirfenidone and is present in the therapeutic composition at a concentration within a range of about 1,000 nM to about 10,000,000 nM, about 10,000 nM to about 10,000,000 nM, or about 100,000 nM to about 10,000,000 nM.

Clause 42. The method of clause 40, wherein the anti-fibrotic agent comprises nintedanib and is present in the therapeutic composition at a concentration within a range of about 0.1 nM to about 1,000,000 nM, about 1 nM to about 1,000,000 nM, about 10 nM to about 1,000,000 nM, or about 100 nM to about 1,000,000 nM.

Clause 43. The method of clause 40, wherein the anti-viral age or anti-diabetic agent comprises metformin or its salt and is present in the therapeutic composition at a concentration within a range of about 1,000 nM to about 100,000,000 nM, about 100,000 nM to about 100,000,000 nM, or about 1,000,000 nM to about 100,000,000 nM.

Clause 44. The method of any one of the preceding clauses, wherein the therapeutic composition comprises one or more of a pharmaceutically acceptable carrier, a propellant, an excipient, a surfactant, a binding agent, an adjuvant agent, a flavoring agent or taste masking agent, a coloring agent, an emulsifying agent, a stabilizing agent, an isotonic agent, and targeting co-molecules.

Clause 45. The method of any one of the preceding clauses, wherein the therapeutic composition is atomized, nebulized, aerosolized, pressurized, micronized, nanosized, in the form of a dry powder, or combinations thereof.

Clause 46. The method of any one of the preceding clauses, wherein the therapeutically effective dose of the therapeutic composition is effective to inhibit thrombosis, inhibit clot formation, or inhibit fibrin formation in the patient's lung.

Clause 47. A therapeutic composition for inhibiting an inflammatory pulmonary disease in a patient, the composition comprising: a direct factor Xa inhibitor formulated for delivery to the patient by any one of inhalation, ventilation, instillation, ultrasound, vibration, and injection.

Clause 48. The composition of clause 47, wherein delivery of a therapeutically effective dose of the therapeutic composition is effective to suppress or prevent initiation, progression, or relapses of disease, including the progression of established disease.

Clause 49. The composition of any one of the preceding clauses, wherein the inflammatory pulmonary disease is caused by a viral infection, bacterial infection, smoke inhalation, chemical exposure, genetic mutation, injury, smog and other air pollutions, inhalation of environmental and occupational pollutants, work-related lung diseases, hypersensitivity pneumonitis, and combinations thereof.

Clause 50. The composition of any one of the preceding clauses, wherein the inflammatory pulmonary disease comprises pneumonia, bronchitis, emphysema, asthma, pulmonary fibrosis, lung cancer, pulmonary edema, pulmonary embolism, sarcoidosis and granulomatosis with polyangiitis, chronic obstructive pulmonary disease, bronchiectasis, acute respiratory distress syndrome (ARDS), SARS, or COVID-19.

Clause 51. The composition of any one of the preceding clauses, wherein the direct factor Xa inhibitor is selected from the group consisting of Apixaban, betrixaban, edoxaban, otamixaban, razaxaban, Rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), (S)-3-(7-carbamimidoylnaphthalen-2-yl)-2-(4-(((S)-1-(1-iminoethyl)pyrrolidin-3-yl)oxy)phenyl)propanoic acid hydrochloride pentahydrate (DX9065a), letaxaban (TAK-442), GW813893, JTV-803, KFA-144, DPC-423, RPR-209685, MCM-09, or antistasin.

Clause 52. The composition of clause 51, wherein the direct factor Xa inhibitor comprises Rivaroxaban, or a salt, isomer, solvate, analog, derivative, metabolite, or prodrugs thereof.

Clause 53. The composition of clause 51, wherein the direct factor Xa inhibitor comprises Apixaban, or a salt, isomer, solvate, analog, derivative, metabolite, or prodrugs thereof.

Clause 54. The composition of any one of the preceding clauses, wherein the direct factor Xa inhibitor is present in the therapeutic composition at a concentration within a range of about 0.1 nM to about 1,000,000 nM.

Clause 55. The composition of clause 54, wherein the direct factor Xa inhibitor is present in the therapeutic composition at a concentration within a range of about 1 nM to about 1,000,000 nM.

Clause 56. The composition of clause 54, wherein the direct factor Xa inhibitor is present in the therapeutic composition at a concentration within a range of about 10 nM to about 1,000,000 nM.

Clause 57. The composition of clause 54, wherein the direct factor Xa inhibitor is present in the therapeutic composition at a concentration within a range of about 100 nM to about 1,000,000 nM.

Clause 58. The composition of any one of the preceding clauses, wherein the direct factor Xa inhibitor is present at a concentration effective to achieve a tissue concentration of the direct factor Xa inhibitor of about 0.1 ng/mg tissue to about 10 ng/mg tissue.

Clause 59. The composition of clause 58, wherein the direct factor Xa inhibitor is present at a concentration effective to achieve a tissue concentration of the direct factor Xa inhibitor of about 0.2 ng/mg tissue to about 10 ng/mg tissue.

Clause 60. The composition of clause 58, wherein the direct factor Xa inhibitor is present at a concentration effective to achieve a tissue concentration of the direct factor Xa inhibitor of about 0.5 ng/mg tissue to about 5 ng/mg tissue.

Clause 61. The composition of clause 58, wherein the direct factor Xa inhibitor is present at a concentration effective to achieve a tissue concentration of the direct factor Xa inhibitor of about 1 ng/mg tissue to about 2 ng/mg tissue.

Clause 62. The composition of clause 58, wherein the direct factor Xa inhibitor is present at a concentration effective to achieve a blood concentration of the direct factor Xa inhibitor which is less than about 200 ng/ml, 100 ng/ml, 50 ng/ml, 40 ng/ml, or 10 ng/ml.

Clause 63. The composition of clause 58, wherein the direct factor Xa inhibitor is present at a concentration effective to achieve a blood concentration of the direct factor Xa inhibitor which is smaller than a median maximum serum concentration (Cmax) of the direct factor Xa inhibitor generated by systemic delivery of the direct factor Xa inhibitor to achieve the same tissue concentration at the site of the inflammatory pulmonary disease.

Clause 64. The composition of clause 58, wherein the direct factor Xa inhibitor is present at a concentration effective to achieve a blood concentration of the direct factor Xa inhibitor which does not exceed a median maximum serum concentration (Cmax) of the direct factor Xa inhibitor generated by systemic delivery of the direct factor Xa inhibitor to achieve the same tissue concentration at the site of the inflammatory pulmonary disease for more than about 6 hours to about 12 hours.

Clause 65. The composition of any one of the preceding clauses, wherein the therapeutic composition further comprises at least one additional therapeutically active substance.

Clause 66. The composition of clause 65, wherein the at least one additional therapeutically active substance comprises a direct factor IIa inhibitor selected from the group consisting of Argatroban, dabigatran, ximelagatran, melagatran, efegatran, inogatran, atecegatran metoxil (AZD-0837), hirudin, hirudin analogs, bivalirudin, desirudin, or lepirudin.

Clause 67. The composition of clause 66, wherein the direct factor IIa inhibitor comprises Argatroban. or a salt, isomer, solvate, analog, derivative, metabolite, or prodrugs thereof.

Clause 68. The composition of clause 66, wherein the direct factor Xa inhibitor comprises Apixaban and the direct factor IIa inhibitor comprises Argatroban.

Clause 69. The composition of clause 66, wherein the direct factor IIa inhibitor is present in the therapeutic composition at a concentration within a range of about 0.1 nM to about 1,000,000 nM.

Clause 70. The composition of clause 69, wherein the direct factor IIa inhibitor is present in the therapeutic composition at a concentration within a range of about 1 nM to about 1,000,000 nM.

Clause 71. The composition of clause 69, wherein the direct factor IIa inhibitor is present in the therapeutic composition at a concentration within a range of about 10 nM to about 1,000,000 nM.

Clause 72. The composition of clause 69, wherein the direct factor IIa inhibitor is present in the therapeutic composition at a concentration within a range of about 100 nM to about 1,000,000 nM.

Clause 73. The composition of clause 66, wherein the direct factor IIa inhibitor is present at a concentration effective to achieve a tissue concentration of the direct factor IIa inhibitor of about 0.1 ng/mg tissue to about 10 ng/mg tissue.

Clause 74. The composition of clause 73, wherein the direct factor IIa inhibitor is present at a concentration effective to achieve a tissue concentration of the direct factor IIa inhibitor of about 0.2 ng/mg tissue to about 10 ng/mg tissue.

Clause 75. The composition of clause 73, wherein the direct factor IIa inhibitor is present at a concentration effective to achieve a tissue concentration of the direct factor IIa inhibitor of about 0.5 ng/mg tissue to about 5 ng/mg tissue.

Clause 76. The composition of clause 73, wherein the direct factor IIa inhibitor is present at a concentration effective to achieve a tissue concentration of the direct factor IIa inhibitor of about 1 ng/mg tissue to about 2 ng/mg tissue.

Clause 77. The composition of clause 73, wherein the direct factor IIa inhibitor is present at a concentration effective to achieve a blood concentration of the direct factor IIa inhibitor which is less than about 200 ng/ml, 100 ng/ml, 50 ng/ml, 40 ng/ml, or 10 ng/ml.

Clause 78. The composition of clause 73, wherein the direct factor IIa inhibitor is present at a concentration effective to achieve a blood concentration of the direct factor IIa inhibitor which is smaller than a median maximum serum concentration (Cmax) of the direct factor IIa inhibitor generated by systemic delivery of the direct factor IIa inhibitor to achieve the same tissue concentration at the site of the inflammatory pulmonary disease.

Clause 79. The composition of clause 73, wherein the direct factor IIa inhibitor is present at a concentration effective to achieve a blood concentration of the direct factor IIa inhibitor which does not exceed a median maximum serum concentration (Cmax) of the direct factor IIa inhibitor generated by systemic delivery of the direct factor IIa inhibitor to achieve the same tissue concentration at the site of the inflammatory pulmonary disease for more than about 6 hours to about 12 hours.

Clause 80. The composition of clause 73, wherein the weight compositional ratio of the direct factor Xa inhibitor to the direct factor IIa inhibitor in the therapeutic composition is within a range of about 3:1 to about 1:3.

Clause 81. The composition of clause 73, wherein the weight compositional ratio of the direct factor Xa inhibitor to the direct factor IIa inhibitor in the therapeutic composition is about 1:1.

Clause 82. The composition of any one of the preceding clauses, wherein the therapeutic composition is administered in combination with one or more additional pharmaceutical agents.

Clause 83. The composition of clause 82, wherein the one or more additional pharmaceutical agents comprises one or more anti-fibrotic agents, anti-viral agents, anti-bacterial agents, metformin or its salt, steroids, interferons, or combinations thereof.

Clause 84. The composition of clause 83, wherein the anti-fibrotic agent comprises pirfenidone and is p Clause resent in the therapeutic composition at a concentration within a range of about 1,000 nM to about 10,000,000 nM, about 10,000 nM to about 10,000,000 nM, or about 100,000 nM to about 10,000,000 nM.

Clause 85. The composition of clause 83, wherein the anti-fibrotic agent comprises nintedanib and is present in the therapeutic composition at a concentration within a range of about 0.1 nM to about 1,000,000 nM, about 1 nM to about 1,000,000 nM, about 10 nM to about 1,000,000 nM, or about 100 nM to about 1,000,000 nM.

Clause 86. The composition of clause 83, wherein the anti-viral age or anti-diabetic agent comprises metformin or its salt and is present in the therapeutic composition at a concentration within a range of about 1,000 nM to about 100,000,000 nM, about 100,000 nM to about 100,000,000 nM, or about 1,000,000 nM to about 100,000,000 nM.

Clause 87. The composition of any one of the preceding clauses, wherein the therapeutic composition further comprises one or more of a pharmaceutically acceptable carrier, a propellant, a blowing agent, an excipient, a surfactant, a binding agent, an adjuvant agent, a flavoring agent or taste masking agent, a coloring agent, an emulsifying agent, a stabilizing agent, an isotonic agent, and targeting co-molecules.

Clause 88. The composition of any one of the preceding clauses, wherein the therapeutic composition is atomized, nebulized, aerosolized, pressurized, micronized, nanosized, in the form of a dry powder, or combinations thereof.

Clause 89. The composition of any one of the preceding clauses, wherein delivery of a therapeutically effective dose of the therapeutic composition is effective to inhibit thrombosis, inhibit clot formation, or inhibit fibrin formation in the patient's lung.

Clause 90. A method for inhibiting clot formation or fibrin formation in a lung of a patient, the method comprising: providing a therapeutic composition comprising a direct factor Xa inhibitor; and delivering a therapeutically effective dose of the therapeutic composition to a site of the clot formation or fibrin formation in the patient's lung.

The illustrative examples described are not meant to be limiting. Other examples may be utilized, and other changes may be made, or combined in whole or in part, without departing from the scope of the subject matter presented herein. It will be readily understood that the aspects of the present disclosure, as generally described herein, and illustrated in the figures, and detailed description, and in the examples, can be arranged, substituted, combined, separated, and designed in a wide variety of different configurations, all of which are explicitly contemplated herein.

The illustrative aspects, examples, or embodiments describes are not meant to be limiting. For example, the examples provided for an implantable scaffold comprising a scaffold structure having a surface configured to be expanded in the patient's body, can also apply to other devices described in this application. Other examples may be utilized, and other changes may be made, or combined in whole or in part, without departing from the scope of the subject matter presented herein. These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows a plot of HAoSMC cell proliferation in the presence of rapamycin and varying concentrations of Apixaban, in accordance with examples;

FIG. 1B shows a plot of HAoSMC cell proliferation in the presence of rapamycin and varying concentrations of Argatroban, in accordance with examples;

FIG. 1C shows a plot of HAoSMC cell proliferation in the presence of rapamycin and varying concentrations of Apixaban and Argatroban, in accordance with examples;

FIG. 1D shows a plot of HAoSMC cell proliferation in the presence of difference concentrations of Apixaban, in accordance with examples;

FIG. 1E shows a plot of HAoSMC cell proliferation in the presence of difference concentrations of Argatroban, in accordance with examples;

FIG. 2A shows a plot of activated clotting time (ACT) versus drug concentration, in accordance with examples;

FIG. 2B shows a plot of activated clotting time (ACT) versus drug concentration, in accordance with examples;

FIG. 2C shows a plot of activated clotting time (ACT) versus drug concentration, showing the synergistic effects of Apixaban in combination with Argatroban, in accordance with examples;

FIG. 2D shows a plot of various synergistic effects of drug combination ratios between Apixaban and Argatroban, in accordance with examples;

FIG. 3 shows a reaction scheme of Argatroban with poly N-(2-Hydroxypropyl) methacrylamide, in accordance with examples.

FIG. 4 shows a plot of solubility of Apixaban vs Captisol, in accordance with examples;

FIG. 5 shows a plot of the liquid output rate of Aerogen Solo Nebulizer vs percentage of Cyclodextrin, in accordance with examples;

FIG. 6 shows a plot of the solubility limit of Argatroban and Apixaban vs solubility enhance concentration, in accordance with examples;

FIG. 7 shows a test set up of the nebulized aerosol delivered dose testing, in accordance with examples;

FIG. 8 shows a plot of Argatroban and Apixaban concentration in rat whole blood vs time, in accordance with examples;

FIG. 9 shows a plot of Argatroban and Apixaban concentration in rat lungs tissue vs time, in accordance with examples;

FIG. 10 shows a plot of Argatroban, Apixaban, Azelastine hydrochloride and Hydroxychloroquine sulfate concentration in rat whole blood vs time, in accordance with examples;

FIG. 11 shows a plot of Argatroban, Apixaban, Azelastine hydrochloride and Hydroxychloroquine sulfate concentration in rat lungs tissue vs time, in accordance with examples.

### DETAILED DESCRIPTION OF THE DISCLOSURE

In the following detailed description, reference is made to the accompanying figures, which form a part hereof. In the figures, similar symbols typically identify similar components, unless context dictates otherwise. The illustrative examples described in the detailed description, figures, and claims are not meant to be limiting. Other examples may be utilized, and other changes may be made, or combined in whole or in part, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain examples and examples are disclosed below, inventive subject matter extends beyond the specifically disclosed examples to other alternative examples and/or uses, and to modifications and equivalents thereof. Thus, the scope of the claims appended hereto is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples, however, the order of description should not be construed to imply that these operations are order dependent. Additionally, the structures, systems, and/or devices described herein may be embodied as integrated components or as separate components.

For purposes of comparing various examples, certain aspects and advantages of these examples are described. Not necessarily all such aspects or advantages are achieved by any particular example or embodiment. Thus, for example, various examples may be carried out in a manner that achieves or optimizes one advantage or group of advantages as taught herein without necessarily achieving other aspects or advantages as may also be taught or suggested herein.

Every example of the present invention may optionally be combined with any one or more of the other examples described herein. Every patent literature, and every non-patent literature, cited herein is incorporated herein by reference in its entirety.

As used herein, the term coagulation comprises one or more of thrombin formation, fibrin formation, platelet activation, platelet aggregation, and/or thrombus/clot formation. Coagulation typically arises in response to a body part injury and/or to a foreign body such as a device. This may lead to one or more of inflammation, injury, blockage of a lumen or vessel partially or fully, degradation of the device function, formation of clot, and/or adverse clinical events. In some examples, any of the devices described herein may, at least partially, cause an injury to the tissue which may initiate the coagulation cascade.

As used herein, the term anti-coagulant refers to an agent that inhibits one or more of thrombin formation, fibrin formation, platelet activation (typically indirectly), platelet aggregation (typically indirectly), thrombus (clot) formation, thrombin dissolution, fibrin dissolution, or thrombus dissolution, thereby inhibiting one or more of blockage of a lumen or vessel partially or fully, degradation of the device function, formation of clot, and/or adverse clinical events.

Inhibiting one or more of thrombin formation, fibrin formation, platelet activation, and/or platelet aggregation enables the inhibition of one or more of blockage of a lumen or vessel partially or fully, degradation of the device function, formation of thrombus (clot) formation, inflammation, and/or adverse clinical events.

Described herein are systems and methods for locally delivering a therapeutic composition to a patient, particularly to a patient's lungs via a pulmonary delivery route. The therapeutic composition includes one or more agents which inhibit one or more of thrombin, fibrin, and/or thrombus formation or promote one or more of thrombin, fibrin, and/or thrombus dissolution. In preferred examples, the therapeutic composition includes one or both of a direct Xa inhibitor and a direct IIa inhibitor. In another preferred example, an anti-proliferative agent may be added to the therapeutic composition of the direct Xa inhibitor and/or the direct IIa inhibitor. As described herein, it was surprisingly found that fast release formulation of factor Xa inhibitor (alone or in combination with release of an anti-proliferative agent) resulted in prolonged anti-coagulant effects (e.g., one or more of inhibition of fibrin, inhibition of thrombin formation, enhanced fibrin dissolution, and/or enhancing thrombin inhibition) compared to control and/or a slower release composition profile. The combination of a direct Xa inhibitor and a direct IIa inhibitor formulation was also surprisingly found to improve inhibition of fibrin and/or inhibition of clot formation compared to either agent alone. Additionally, it was surprisingly found that the combination of a direct Xa inhibitor and a direct IIa inhibitor formulation resulted in unexpected anti-proliferative effects (e.g., reduced cell proliferation) in combination, while each agent alone had little to no anti-proliferative effect. Furthermore, surprisingly, and unexpectedly, direct Xa inhibitor and a direct IIa inhibitor combination with an anti-proliferative agent formulation and improved or enhanced the anti-proliferative effect compared to the anti-proliferative agent formulation alone. It was also surprisingly found that the combination of an anti-proliferative agent with a direct Xa inhibitor and a direct IIa inhibitor formulation enhanced inhibition or enhanced dissolution of one or more of the following: fibrin, clot formation, thrombin, platelet aggregation, platelet activation, inflammation, and injury; acutely, and/or within 3 hours to 7 days, and/or within 28 days, and/or within 90 days. It was surprisingly found extending release of factor IIa inhibitor and/or a factor Xa inhibitor, inhibited one or more of clot formation, SMC proliferation, inflammation, and injury, wherein the extended release of the one or more drugs extended beyond 7 days, extended beyond 14 days, extended beyond 21 days, extended beyond 28 days, or extended beyond 3 months. It was surprisingly found extended release formulation comprising a factor IIa inhibitor and/or a factor Xa inhibitor, inhibited one or more of clot formation, SMC proliferation, inflammation, and injury, wherein the extended release of the one or more drugs extended beyond 7 days, extended beyond 14 days, extended beyond 21 days, extended beyond 28 days, or extended beyond 3 months.

In some examples, the compositions described herein can be configured to release a factor Xa inhibiting agent to a mammalian body, lumen, tissue, and/or device surface prior to an injury to said tissue, concurrent with injury to said tissue, or after an initial injury to said tissue. The composition is introduced into said mammalian body and advanced to said tissue site or body lumen. In specific examples, the composition releases said agent to a tissue segment adjacent to the device in the amount ranging from 0.01 ng/mg of tissue to 1000 ng/mg of tissue, preferably ranging from 0.1 ng/mg tissue to 500 ng/mg of tissue, more preferably ranging from 1 ng/mg of tissue to 150 ng/mg of tissue. In some other specific examples, the agent molecular weight ranges from 200 g/mol to 1500 g/mol, preferably ranges from 300 g/mol to 1000 g/mol, more preferably ranges from 350 g/mol to 500 g/mol. In some other examples, the composition releases said agent prior to engaging (or coupling or contacting) of the composition to the tissue site. In some specific examples, the composition locally releases said agent to a tissue segment in the amount ranging from about 10 ng/mg to 200 ng/mg within about 3 hours from tissue injury and/or release of the agent to the tissue segment. In a preferred example, the adjacent tissue segment drug (e.g., tissue 5 mm proximal and 5 mm distal to the tissue segment) concentration ranges from about 0.1 ng/mg of tissue to about 100 ng/mg of tissue, preferably ranges from about 1 ng/mg of tissue to 100 ng/mg of tissue, at about 3 hours from tissue injury and/or release of the agent to the tissue segment. In a preferred example, the tissue concentration in the tissue segment at 3 hours after injury and/or release of said agent to the tissue segment ranges from about 100,000 times the IC₅₀ of factor Xa inhibition to 10,000,000 times the IC₅₀ of factor Xa inhibition, preferably ranges from 500,000 times to 5,000,000 times the IC₅₀ of factor Xa inhibition. The tissue concentration in the adjacent tissue segment (e.g., ±5 mm) at 3 hours after release of said agent to the tissue segment ranges from 100 times the IC₅₀ of factor Xa inhibition to 1,000,000 times the IC₅₀ of factor Xa inhibition, preferably ranges from 1,000 times to 100,000 times the IC₅₀ of factor Xa inhibition. In a preferred example, the tissue concentration in the tissue segment at about 24 hours after injury and/or release of said agent to the tissue segment ranges from 100,000 times the IC₅₀ of factor Xa inhibition to 1000,000 times the IC₅₀ of factor Xa inhibition, preferably ranges from 1000 times to 20,000 times the IC₅₀ of factor Xa inhibition. The tissue concentration in the adjacent tissue segment (e.g., ±5 mm) at 24 hours after injury and/or release of said agent to the tissue segment ranges from 100 times the IC₅₀ of factor Xa inhibition to 1,000,000 times the IC₅₀ of factor Xa inhibition, preferably ranges from 1,000 times to 50,000 times the IC₅₀ of factor Xa inhibition. In another preferred example, the tissue concentration in the tissue segment at about 28 days after injury and/or release of said agent to the tissue segment ranges from 100 times the IC₅₀ of factor Xa inhibition to 100,000 times the IC₅₀ of factor Xa inhibition, preferably ranges from 500 times to 10,000 times the IC₅₀ of factor Xa inhibition. The tissue concentration in the adjacent tissue segment (e.g., ±5 mm) at 28 days after injury and/or release of said agent to the tissue segment ranges from zero times the IC₅₀ of factor Xa inhibition to 100 times the IC₅₀ of factor Xa inhibition, preferably ranges from 10 times to 1,000 times the IC₅₀ of factor Xa inhibition. In a preferred specific example, the composition releases a factor Xa inhibitor to a tissue site at about 3 hours after injury and/or release of agent to the tissue, wherein the tissue concentration in the tissue segment and in the adjacent tissue segment (e.g., ±5 mm from the tissue segment) is greater than the IC to inhibit factor Xa, preferably greater than 10 times the IC₅₀ to inhibit factor Xa, and more preferably greater than 1000 times the IC₅₀ to inhibit factor Xa. In a preferred specific example, the composition releases a factor Xa inhibitor to a tissue site at about 24 hours after injury and/or release of agent to the tissue, wherein the tissue concentration in the tissue segment and in the adjacent tissue segment (±5 mm from the tissue segment) are greater than the IC₅₀ to inhibit factor Xa, preferably greater than 10 times the IC₅₀ to inhibit factor Xa, and more preferably greater than 1000 times the IC 50 to inhibit factor Xa. In a preferred example, the agent is Rivaroxaban, Apixaban, and/or analogs, derivatives, or salts thereof. In a most preferred example, the agent is Apixaban.

In another example, the combination of Apixaban and Argatroban have an additive effect on thrombin formation inhibition or dissolution.

In some examples, a combination of factor IIa inhibitor and factor Xa inhibitor are released from a composition to a mammalian body, lumen, tissue, and/or composition surface after injury at sufficient concentrations in the tissue segment and adjacent tissue segments within about 3 hours after injury to inhibit thrombus (clot) formation. In a preferred example, the agents are Apixaban and Argatroban.

In some examples, the combination of Apixaban and Argatroban released from a composition containing an mTOR inhibitor such as Sirolimus maintains or enhances the antiproliferative effect of said mTOR at the tissue segment site while inhibiting thrombus formation at the said tissue segment site.

In some examples, the combination of Apixaban and Argatroban released from a composition containing an mTOR inhibitor inhibits thrombus formation on the composition surface.

In some examples, the composition comprises or is coated or loaded with one or more agents comprising Apixaban, Argatroban and an mTOR inhibitor. The coating coats one or more surfaces of the device, preferably coating all surfaces of the device including the abluminal and luminal surfaces of the device. Alternatively, or in combination, structural elements of the device are loaded with the one or more agents. In a specific example, the one or more agents are contained in a drug polymer matrix, or contained in a polymer top layer or coat, or is coated as a top layer or coat. In a preferred example of a device configured to release two or more agents, the agents are contained in the same polymer matrix or a different polymer matrix, or one agent is in a polymer matrix while the other agent is under a top polymer coat. In yet another preferred example, the device contains three agents in the same polymer matrix. In another example, each of the drugs is contained in a separate polymer matrix. In yet another example, two of the agents are contained in one polymer matrix while the third agent is contained in a separate polymer matrix or a top layer or coat. In yet another example, the one or more agents are contained in the same polymer matrix and a top layer or coat of a polymer material covers the surface of the device.

In some examples, the composition is contained in a polymer matrix, contained in micro or nano spheres, contained in hydrogels, or the like. In a preferred example, the one or more agents are Apixaban and Argatroban. In another example the agents are Rivaroxaban and Argatroban.

It is an objective of this invention to utilize factor Xa inhibitor and factor IIa inhibitor, and optionally in combination with an antiproliferative, to inhibit or enhance dissolution of one or more of thrombin formation, fibrin formation, clot formation, inflammation, blockage of a body lumen or vessel in a patient's lungs or in other anatomy. Anticoagulants have been successfully used in systemic application. Despite such success, anticoagulants had limited to no success when delivered locally.

When the therapeutic composition comprises a direct factor IIa inhibitor, a direct factor Xa inhibitor, and an anti-proliferative agent, the therapeutic composition may be present in the carrier material at weight ratios of 1:3:1, 3:2:1, 2:2:1, 2:3:1, 3:3:1, 5:5:1, or 6:6:1, respectively. In some examples, the weight compositional ratio of the direct factor IIa inhibitor to the direct factor Xa inhibitor to the anti-proliferative agent in the therapeutic composition may be about 5:5:2. In some examples, the weight compositional ratio of the direct factor IIa inhibitor to the direct factor Xa inhibitor to the anti-proliferative agent in the coating may be within a range of about 6:6:1 to 1:3:1.

When the therapeutic composition comprises a direct factor IIa inhibitor, a direct factor Xa inhibitor, and an anti-proliferative agent, the release rate ratio of the direct factor IIa inhibitor to the direct factor Xa inhibitor to the anti-proliferative agent may be about 1:1:1 to about 4:4:1. In some examples, the coating may be configured to release the direct factor IIa inhibitor, the direct factor Xa inhibitor, and the anti-proliferative agent at the same rate. In other examples, the coating may be configured to release the direct factor IIa inhibitor, the direct factor Xa inhibitor, and the anti-proliferative agent at different rates.

In some examples, the coating may be configured to release the direct factor IIa inhibitor at a rate of about 4 μg/hour/mm device 100 to about 14 μg/day/mm device 100.

In some examples, the coating may be configured to release the direct factor Xa inhibitor at a rate of about 4 μg/hour/mm device 100 to about 14 μg/day/mm device 100.

In some examples, the coating may be configured to release the anti-proliferative agent at a rate of about 1 μg/hour/mm device 100 to about 4 μg/day/mm device 100.

In some examples, the direct factor IIa inhibitor may have an inhibition potency for factor IIa ranging from about 0.001 nM to about 100 nM.

In some examples, the direct factor Xa inhibitor may have an inhibition potency for factor Xa ranging from about 0.001 nM to about 50 nM.

In some examples, the direct factor IIa inhibitor may comprise Argatroban, or a salt, isomer, solvate, analog (including deuterated analog), derivative, metabolite, or prodrugs thereof.

In some examples, the direct factor Xa inhibitor may comprise Apixaban, or a salt, isomer, solvate, analog (including deuterated analog), derivative, metabolite, or prodrugs thereof.

In some examples, the direct factor Xa inhibitor may comprise Rivaroxaban, or a salt, isomer, solvate, analog (including deuterated analog), derivative, metabolite, or prodrugs thereof.

In some examples, the anti-proliferative agent may comprise rapamycin, or a salt, isomer, solvate, analog (including deuterated analog), derivative, metabolite, or prodrugs thereof.

In some examples, the direct factor Xa inhibitor may comprise Apixaban, the direct factor IIa inhibitor may comprise Argatroban, and the anti-proliferative agent may comprise rapamycin.

In some examples, the therapeutic composition is disposed on the external surface of the structure and on the internal surface of the structure. In some examples, the therapeutic composition is disposed on the external surface (abluminal) of the structure, on the interior surface (luminal) of the structure, and on the side surfaces of the structure. In yet other examples, the therapeutic composition is disposed on one or more surfaces of the structure. In yet other examples, the therapeutic composition is disposed on all surfaces of the structure. In yet other examples, the therapeutic composition is disposed in a reservoir on or in the structure. In some examples, the therapeutic composition is disposed on the external surface of the structure.

In some examples, the drug coated balloon is to facilitate rapid and efficient uptake of drug by target tissue during transitory device deployment at a target site. The coated layers may be more than one.

In some examples, the layer may include a therapeutic agent and more than one excipient. For example, one excipient may serve to improve balloon adhesion of another excipient or excipient that are superior at promoting tissue uptake of drug and facilitate its rapid movement off the medical device during deployment and into target tissues.

In some examples, the therapeutic agent is rapidly released after the medical device is brought into contact with tissue and is readily absorbed.

In a further example, the balloon can optionally adopt carrier excipient to coat to facilitate drug transfer to the vessel wall and control release rate. A variety of carrier excipients and techniques can be used. The selected excipient could be contrast agent (i.e. iopromide), urea, dextrane, shellac, shelloic acid, keratosis (a naturally derived protein), Plasticizer (i.e. butyryl-tri-hexyl citrate, acetyl tributyl citrate, citrate ester, glycerol, other organic ester), hydrophilic space, Polyvinylpyrrolidone (PVP) and its hydrogels, Surfactants, Non-ionic surfactant Polysorbate/sorbitol (i.e. Tween20, Tween60 or Tween80), nordihydroguaiaretic acid (NDGA), hydrophibic excipient such as phospholipid, amphiphilic polymer such as Poly(ethylene glycol) (i.e PEG 8000), poly(ethylene oxide) (PEO) (molecular weight range from 100,000 to 10,000,000), Polyethylenimine (PEI) or polyaziridine linear or branched, amphiphilic block co-polymers composed of poly(ethylene oxide) (PEO) as the hydrophilic block and poly(ether)s, poly(amino acid)s), hydrophobic polymer space, biodegradable polymers such as Poly DL lactide-co-glycolide, Poly L Lactide-co-caprolactone, durable polymers, individually or combinations thereof.

In some examples, the therapeutic agent in the coating solution is mTOR, such as novolimus or rapamycin.

In some examples, the therapeutic agent in the coating solution is a factor Xa inhibitor such as Rivaroxaban or Apixaban.

In another example, a factor Xa inhibitor, preferably Rivaroxaban, more preferably Apixaban released locally in combination with Argatroban from a balloon catheter to inhibit smooth muscle proliferation after vessel injury.

In some examples, the therapeutic agent may release to coronary Artery or Superficial Femoral Artery (SFA) or below the knee (BTK).

In further examples, each of the one or more agents that inhibit or enhance dissolution of fibrin formation and/or thrombus formation or promote fibrin dissolution and/or thrombus dissolution is released from a temporary device such as drug coated balloon, and optionally is administered locally, over a period of at least about 1 sec., 10 sec. 30 sec., 1 min., 2 min, or up to 10 minutes continuously or intermittently.

In still further examples, a substantial amount, or substantially all, of each of the fibrin formation inhibition, thrombus formation-inhibiting or fibrin or thrombus dissolution-promoting agent(s) is released from the device within about 1 min., 15 min., 30 min., 1 hr, 6 hr, 12 hr, 1 day, 2 days, 3 days, 1 week, 2 weeks, 3 weeks, 1 month, 2 months, 3 months, 4 months, 5 months, 6 months, 9 months, or 1 years. In a preferred example, the one or more agents comprising factor IIa inhibitor or factor Xa inhibitor are configured to substantially release over at least 28 day, preferably over at least 90 days, over at least 6 months, or over at least 1 year.

In some examples, the therapeutic composition may be formulated to release one or more of the agents at a dose substantially below a systemic therapeutic dose of each agent to minimize off-target effects. Preferably, the dose is at least about 5 times lower than the systemic dose or more preferably about 10 times lower than the systemic dose.

In many examples, a tissue segment is composed of the tissue segment coupled to the device releasing agent. For example, if the stent or balloon catheter is 20 mm in length, the tissue segment is 20 mm in length. In another specific example, the agent is released beyond the tissue segment. For example, when the tissue segment coupled to a device is 20 mm in length, the tissue adjacent to the tissue segment is called the adjacent tissue segment. In many cases the adjacent tissue segment ranges from 1 mm to 10 mm, preferably within a range from 1 mm to 5 mm, more preferably about 5 mm proximal and/or distal to the tissue segment, and most preferably is about 5 mm proximal and distal to the tissue segment.

As used herein, the term “coating” refers to a layer of polymer and/or drug (or therapeutic agent or active agent) disposed on a surface of a device structure. The layer may comprise a polymer, a drug, or a combination of a drug and a polymer.

As used herein, the term “top layer or coat” refers to an outer-most layer of a coating. The top layer or coat may comprise a polymer, a drug (or therapeutic agent or active agent), or a combination of a drug and a polymer. The top layer or coat may comprise the same polymer or a different polymer as layers of coating disposed therebelow. The top layer or coat may comprise the same drug or a different drug(s) as layers of coating disposed therebelow.

As used herein, the term “matrix” refers to a mixture of a drug (or therapeutic agent or active agent) and a polymer.

The terms anti-thrombin, thrombin inhibiter, and thrombin formation inhibitor are used interchangeably herein. Also, the terms anti-fibrin, fibrin inhibitor, and fibrin formation inhibitor are used interchangeably herein.

As used herein, a direct factor Xa inhibitor refers to a direct, selective inhibitor of factor Xa that acts directly on factor Xa without using antithrombin as a mediator. The term “direct factor Xa inhibitor” is used herein interchangeably with the term “factor Xa inhibitor” or “anti-factor Xa”. Direct factor Xa inhibitors inhibit thrombin formation and/or fibrin formation, thereby inhibiting clot formation. Direct factor Xa inhibitors include, but are not limited to, Apixaban, betrixaban, edoxaban, otamixaban, razaxaban, Rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), or 2-[(7-carbamimidoylnaphthalen-2-yl)methyl-[4-(1-ethanimidoylpiperidin-4-yl)oxyphenyl]sulfamoyl]acetic acid (YM-466 or YM-60828), or eribaxaban (PD 0348292), or 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052). Preferred direct Xa inhibitors include Apixaban and Rivaroxaban.

As used herein, a direct factor IIa inhibitor refers to a direct, selective inhibitor of factor IIa (also referred to herein as thrombin) which acts directly on factor IIa/thrombin. The term “direct factor IIa inhibitor” is used herein interchangeably with the term “factor IIa inhibitor” or “anti-factor IIa”. Direct factor IIa inhibitors inhibit thrombin formation and/or fibrin formation, thereby inhibiting clot formation. Direct thrombin/factor IIa inhibitors include, but are not limited to, Argatroban, dabigatran, ximelagatran, melagatran, efegatran, inogatran, atecegatran metoxil (AZD-0837), hirudin, hirudin analogs, bivalirudin, desirudin, and lepirudin. Preferred direct factor IIa inhibitors include Argatroban.

As used herein, an anti-proliferative agent refers to anti-proliferative agents, anti-mitotic agents, cytostatic agents and anti-migratory agents which suppress cell growth, proliferation, and/or metabolism. Examples of anti-proliferative agents include without limitation inhibitors of mammalian target of rapamycin (mTOR), rapamycin (also called Sirolimus), deuterated rapamycin, rapamycin prodrug TAFA93, 40-O-alkyl-rapamycin derivatives, 40-O-hydroxyalkyl-rapamycin derivatives, everolimus {40-O-(2-hydroxyethyl)-rapamycin}, 40-O-(3-hydroxy)propyl-rapamycin, 40-O-[2-(2-hydroxy)ethoxy]ethyl-rapamycin, 40-O-alkoxyalkyl-rapamycin derivatives, biolimus {40-O-(2-ethoxyethyl)-rapamycin}, 40-O-acyl-rapamycin derivatives, temsirolimus {40-(3-hydroxy-2-hydroxymethyl-2-methylpropanoate)-rapamycin, or CCI-779 (temsirolimus), 40-O-phospho-containing rapamycin derivatives, ridaforolimus (40-dimethylphosphinate-rapamycin, or AP23573 (ridaforolimus, formerly known as deforolimus), 40(R or S)-heterocyclyl- or heteroaryl-containing rapamycin derivatives, zotarolimus {40-epi-(N1-tetrazolyl)-rapamycin, or ABT-578 (zotarolimus), 40-epi-(N2-tetrazolyl)-rapamycin, 32(R or S)-hydroxy-rapamycin, myolimus (32-deoxo-rapamycin), novolimus (16-O-desmethyl-rapamycin), taxanes, paclitaxel, docetaxel, cytochalasins, cytochalasins A through J, latrunculins, and salts, isomers, solvates, analogs (including deuterated analogs), derivatives, metabolites, and prodrugs thereof. The IUPAC numbering system for rapamycin is used herein. Preferred anti-proliferative agents include mTOR inhibitors and/or taxanes, or salts, isomers, solvates, analogs, derivatives, metabolites, or prodrugs thereof.

Table A provides non-limiting examples of derivatives of each of rapamycin, everolimus, biolimus, temsirolimus, ridaforolimus, zotarolimus, myolimus and novolimus.

TABLE A
Derivatives of rapamycin-type compounds
Derivatives of Each of Rapamycin, Everolimus,
Biolimus, Temsirolimus, Ridaforolimus,
Zotarolimus, Myolimus and Novolimus

N7-oxide
2-hydroxy
3-hydroxy
4-hydroxy
5-hydroxy
6-hydroxy
11-hydroxy
12-hydroxy
13-hydroxy
14-hydroxy
23-hydroxy
24-hydroxy
25-hydroxy
31-hydroxy
35-hydroxy
43-hydroxy (11-hydroxymethyl)
44-hydroxy (17-hydroxymethyl)
45-hydroxy (23-hydroxymethyl)
46-hydroxy (25-hydroxymethyl)
47-hydroxy (29-hydroxymethyl)
48-hydroxy (31-hydroxymethyl)
49-hydroxy (35-hydroxymethyl)
17,18-dihydroxy
19,20-dihydroxy
21,22-dihydroxy
29,30-dihydroxy
10-phosphate
28-phosphate
40-phosphate
16-O-desmethyl
27-O-desmethyl
39-O-desmethyl
16,27-bis(O-desmethyl)
16,39-bis(O-desmethyl)
27,39-bis(O-desmethyl)
16,27,39-tris(O-desmethyl)
16-desmethoxy
27-desmethoxy
39-O-desmethyl-14-hydroxy
17,18-epoxide
19,20-epoxide
21,22-epoxide
29,30-epoxide
17,18-29,30-bis-epoxide
17,18-19,20-21,22-tris-epoxide
19,20-21,22-29,30-tris-epoxide
16-O-desmethyl-17,18-19,20-bis-epoxide
16-O-desmethyl-17,18-29,30-bis-epoxide
16-O-desmethyl-17,18-19,20-21,22-tris-epoxide
16-O-desmethyl-19,20-21,22-29,30-tris-epoxide
27-O-desmethyl-17,18-19,20-21,22-tris-epoxide
39-O-desmethyl-17,18-19,20-21,22-tris-epoxide
16,27-bis(O-desmethyl)-17,18-19,20-21,22-tris-epoxide
16-O-desmethyl-24-hydroxy-17,18-19,20-bis-epoxide
16-O-desmethyl-24-hydroxy-17,18-29,30-bis-epoxide
12-hydroxy and opened hemiketal ring

It will be understood by one of ordinary skill in the art that the devices and methods described herein may be used in combination with one or more additional bioactive agents. Such agents optionally include anti-mitotic agents, cytostatic agents, anti-migratory agents, immunomodulators, immunosuppressants, anti-inflammatory agents, anti-ischemia agents, anti-hypertensive agents, vasodilators, anti-hyperlipidemia agents, anti-diabetic agents, anti-cancer agents, anti-tumor agents, anti-angiogenic agents, angiogenic agents, anti-chemokine agents, healing-promoting agents, anti-bacterial agents, anti-fungal agents, and combinations thereof. It is understood that a bioactive agent may exert more than one biological effect.

Use of anti-coagulants, or fibrin/thrombus formation-inhibiting agent(s) have surprisingly been found to also enhance or aid in inhibiting cell proliferation, smooth muscle cell proliferation, hyperplasia or restenosis (e.g., smooth muscle cell proliferation or hyperplasia), when two agents factor Xa inhibitor and factor IIa inhibitor (Apixaban and Argatroban) were tested in combination or additionally in combination with a third antiproliferative agent.

In a preferred example, a device releasing one or more factor Xa inhibitors, and/or one or more factor IIa inhibitors, and/or one or more antiproliferative agents, wherein said one or more agents inhibit thrombin formation and/or fibrin formation thereby inhibiting clot formation and smooth muscle cell proliferation.

In some examples, the injury to a tissue, surface, vessel/lumen wall, or other body part is the first substantial injury resulting from a surgery or intervention. In certain examples, the surgery or intervention is selected from the group consisting of pulmonary surgeries and interventions, cardiopulmonary surgeries and interventions, peripheral pulmonary surgeries and interventions, pulmonary grafting, pulmonary replacement, pulmonary angioplasty, thrombectomy, pulmonary stent placement, pulmonary laser therapy, coronary by-pass surgery, coronary angiography, coronary stent placement, carotid artery procedures, peripheral stent placement, organ transplants, artificial heart transplant, and plastic and cosmetic surgeries and interventions. In additional examples, the injury is the first substantial injury caused by the device delivering the one or more active substances, and optionally one or more other kinds of bioactive agents (e.g., anti-proliferative agents, anti-inflammatory agents, etc.). In some examples, a substantial injury to a tissue, surface, vessel/lumen wall or other body part results from contact of a device with the tissue, surface, vessel/lumen wall or other body part in a surgery or intervention (e.g., contact of the device causing damage to the endothelium lining a blood vessel, a surgical cutting instrument cutting a tissue, a deployed stent embedding into the wall of a blood vessel, etc.). In further examples, a substantial injury to a tissue, surface, vessel/lumen wall or other body part has a potential to elicit fibrin/thrombus formation, cell migration, cell proliferation or inflammation, or a combination thereof, at the site of injury or at an area adjacent thereto.

In some examples, the therapeutic composition is formulated to release the one or more active substances at a rate of 1 μg/second/mm device to about 50 μg/day/mm device, preferably at a rate of 1 μg/min/mm device to about 30 μg/day/mm device, more preferably at a rate of 1 μg/hour/mm device to about 30 μg/day/mm device.

In some examples, each of the one or more active substances is released from a temporary or non-temporary device at a rate within a range of about 1 μg/hour/mm device length to about 30 μg/day/mm device length, for example about 1 μg/hour/mm device length to about 20 μg/day/mm device length. In some examples, the therapeutic composition is formulated to release the one or more active substances at a rate of 1 μg/hour/mm device to about 20 μg/day/mm device. In some examples, the therapeutic composition may be formulated to release the one or more active substances at a rate within a range of about 1 μg/hour/mm device length to about 14 μg/hour/mm device length. In some examples, the therapeutic composition may be formulated to release the one or more active substances at a rate within a range bounded by any two of the following values: about 1 μg/hour/mm device length, about 2 μg/hour/mm device length, about 3 μg/hour/mm device length, about 4 μg/hour/mm device length, about 5 μg/hour/mm device length, about 6 μg/hour/mm device length, about 7 μg/hour/mm device length, about 8 μg/hour/mm device length, about 9 μg/hour/mm device length, about 10 μg/hour/mm device length, about 11 μg/hour/mm device length, about 12 μg/hour/mm device length, about 13 μg/hour/mm device length, about 14 μg/hour/mm device length, about 15 μg/hour/mm device length, about 16 μg/hour/mm device length, about 17 μg/hour/mm device length, about 18 μg/hour/mm device length, about 19 μg/hour/mm device length, about 20 μg/hour/mm device length, about 21 μg/hour/mm device length, about 22 μg/hour/mm device length, about 23 μg/hour/mm device length, about 24 μg/hour/mm device length, about 25 μg/hour/mm device length, about 26 μg/hour/mm device length, about 27 μg/hour/mm device length, about 28 μg/hour/mm device length, about 29 μg/hour/mm device length, or about 30 μg/hour/mm device length.

In some examples, the therapeutic composition may be formulated not to release the one or more active substances until a predetermined time period has elapsed in order to ensure that the one or more active substances are released to the target tissue of interest and not during delivery of the structure to the target tissue. In some examples, the therapeutic composition may be formulated not to release the one or more active substances until the external surface of the structure is positioned adjacent the injury site. In some examples, the therapeutic composition is formulated not to release the one or more active substances for at least about 1 minute, about 5 minutes, about 30 minutes, about 1 hour, about 12 hours, or about 24 hours after introduction into the patient's body. In some examples, a removable cover or sheath may be disposed about the external surface of the structure in order to prevent release of the one or more active substances until the predetermined time period has elapsed. When the predetermined time period (e.g., 30 minutes, 1 hour, 12 hours, 24 hours, etc.) has elapsed, the cover or sheath may be removed and the therapeutic composition may be exposed, thereby beginning release of the one or more active substances.

In some examples, the therapeutic composition is formulated to begin releasing the one or more active substances within about 1 minute, 5, 10, 15, 20, 25, or 30 minutes after the external surface of the structure is positioned adjacent the injury site.

In some examples, substantially all of each of the one or more active substances is released from a temporary or non-temporary device within about 1 day to about 180 days or more, for example within about 1 day to about 90 days. In some examples, the therapeutic composition is formulated to release substantially all of the one or more active substances within about 7 days or about 28 days. In some examples, the therapeutic composition may be formulated to release substantially all of the one or more active substances within a range bounded by any two of the following values: 1 day, 3 days, 7 days, 14 days, 21 days, 28 days, 45 days, 90 days, 180 days, or more.

In some examples, the therapeutic composition is formulated to release substantially all of the one or more active substances within about 3 hours, about 6 hours, about 12 hours, about 1 day, or about 3 days. In some examples, the therapeutic composition is formulated to release at least 50%, at least 60%, or at least 70% of the one or more active substances within about 3 hours, about 6 hours, about 12 hours, about 1 day, about 3 days, about 7 days, or about 28 days.

In some examples, each of the one or more active substances is released from a temporary or non-temporary device at a rate sufficient to generate a tissue concentration of each of the agents within a range of about 5 ng/mg tissue to about 200 nm/mg tissue at the injury site within about 3 hours of tissue contact.

In some examples, the therapeutic composition is formulated to locally release the one or more active substances to the injury site at a rate sufficient to generate a tissue concentration of about 2 ng/mg tissue to about 800 ng/mg tissue, about 2 ng/mg tissue to about 200 ng/mg tissue, preferably at about 20 ng/mg tissue to about 200 ng/mg tissue, more preferably at about 40 ng/mg tissue to about 200 ng/mg tissue, of the one or more active substances at the injury site within about 3 hours after the external surface of the structure is positioned adjacent the injury site. In some examples, the therapeutic composition is formulated to release the one or more active substances at a rate sufficient to generate a tissue concentration at the injury site within a range of about 10 ng/mg tissue to about 100 ng/mg tissue. The therapeutic composition may be formulated to locally release the one or more active substances to the injury site at a rate sufficient to generate a tissue concentration of the one or more active substances at the injury site within about 3 hours after placement adjacent the injury site within a range bounded by any two of the following values: 2 ng/mg tissue, 5 ng/mg tissue, 10 ng/mg tissue, 20 ng/mg tissue, 30 ng/mg tissue, 40 ng/mg tissue, 50 ng/mg tissue, 60 ng/mg tissue, 70 ng/mg tissue, 80 ng/mg tissue, 90 ng/mg tissue, 100 ng/mg tissue, 110 ng/mg tissue, 120 ng/mg tissue, 130 ng/mg tissue, 140 ng/mg tissue, 150 ng/mg tissue, 160 ng/mg tissue, 170 ng/mg tissue, 180 ng/mg tissue, 190 ng/mg tissue, or 200 ng/mg tissue.

In another example, the device releases the one or more active substances from 1 microgram per mm of device length to 25 micrograms per mm of device length, and preferably releases said agent from 5 micrograms per mm of device length to 20 micrograms per mm of device length.

In some examples, the therapeutic composition is formulated to release the one or more active substances at a rate within a range of about 2 μg/mm device to about 100 μg/mm device, about 5 μg/mm device to about 100 μg/mm device, about 7 μg/mm device to about 100 μg/mm device, or about 10 μg/mm device to about 100 μg/mm device within about 3 hours, 12 hours, 1 day, 3 days, 7 days, 28 days, 90 days, or 180 days. In some examples, the therapeutic composition is formulated to release the one or more active substances at a rate within a range of about 5 μg/mm device to about 100 μg/mm device within about 3 hours. In some examples, the therapeutic composition is formulated to release the one or more active substances at a rate within a range of about 5 μg/mm device to about 100 μg/mm device within about 12 hours. In some examples, the therapeutic composition is formulated to release the one or more active substances at a rate within a range of about 5 μg/mm device to about 100 μg/mm device within about 7 days. In some examples, the therapeutic composition is formulated to release the one or more active substances at a rate within a range of about 5 μg/mm device to about 100 μg/mm device within about 28 days.

In some examples, the therapeutic composition is formulated to release the one or more active substances at a dose within a range of about 0.5 μg/mm² device to about 15 μg/mm² device, or of about 1 μg/mm² device to about 12 μg/mm² device, or of about 2 μg/mm² device to about 12 μg/mm² device, or of about 5 μg/mm² device to about 12 μg/mm² device, or of about 7 μg/mm² device to about 12 μg/mm² device, within about 3 hours or about 12 hours or about 1 day or about 3 days or about 7 days. In some examples, the therapeutic composition is formulated to release the one or more active substances at a dose within a range of about 1 μg/mm² device to about 12 μg/mm² device within about 3 hours. In some examples, the therapeutic composition is formulated to release the one or more active substances at a dose within a range of about 1 μg/mm² device to about 12 μg/mm² device within about 12 hours. In some examples, the therapeutic composition is formulated to release the one or more active substances at a dose within a range of about 1 μg/mm² device to about 12 μg/mm² device within about 7 days. In some examples, the therapeutic composition is formulated to release the one or more active substances at a dose within a range of about 1 μg/mm² device to about 12 μg/mm² device within about 28 days, about 90 days, or about 180 days.

In some examples, each of the one or more agents is released from a temporary or non-temporary device at a rate sufficient to generate a tissue concentration of each of the agents within a range of about 1 ng/mg tissue at about 100 ng/mg tissue within about 28 days of tissue contact.

In some examples, the therapeutic composition is formulated to release the one or more active substances at a rate sufficient to generate a tissue concentration of about 0.5 ng/mg to about 10 ng/mg within the tissue adjacent to the device structure within about 28 days, about 90 days, or about 180 days.

In some examples, the therapeutic composition is formulated to release the one or more active substances at a rate sufficient to generate a tissue concentration at the injury site within a range of about 0.5 ng/mg to about 30 ng/mg within about 28 days. In some examples, the therapeutic composition is formulated to release the one or more active substances at a rate sufficient to generate a tissue concentration at the injury site within a range of about 1 ng/mg to about 20 ng/mg within about 28 days. In some examples, the therapeutic composition is formulated to release the one or more active substances at a rate sufficient to generate a tissue concentration at the injury site within a range of about 1.5 ng/mg to about 25 ng/mg within about 28 days.

In some examples, the therapeutic composition is formulated to release the one or more active substances at a rate sufficient to generate a tissue concentration at the injury site within a range of about 0.1 ng/mg to about 10 ng/mg within about 90 days or about 180 days.

In some examples, each of the one or more agents is released from a temporary or non-temporary device at the same rate. In other examples, one or more of the one or more agents that inhibit fibrin/thrombus formation or promote fibrin/thrombus dissolution and/or other bioactive agents is released from a temporary or non-temporary device at a different rate.

In some examples, the therapeutic composition is formulated to release the direct factor Xa inhibitor and/or the direct factor IIa inhibitor faster than the anti-proliferative agent.

In some examples, the therapeutic composition is formulated to release a larger dose of the direct factor Xa inhibitor than the anti-proliferative agent. In some examples, the dose of the direct factor Xa inhibitor is about 1.25 to about 5 times larger, about 1.5 to about 3 times larger, or about 1.5 to about 2.5 times larger than a dose of the anti-proliferative agent.

In some examples, the therapeutic composition is formulated to release the one or more active substances at a rate sufficient to generate a tissue concentration at a location proximal or distal a proximal end of the structure or a distal end of the structure, respectively (e.g., an adjacent tissue segment), within a range of about 0.5 ng/mg to about 500 ng/mg within about 3 hours. In some examples, the therapeutic composition is formulated to release the one or more active substances at a rate sufficient to generate a tissue concentration at the location proximal or distal the proximal end of the structure or the distal end of the structure, respectively (e.g., an adjacent tissue segment), within a range of about 1 ng/mg to about 35 ng/mg within about 3 hours. In some examples, the therapeutic composition is formulated to release the one or more active substances at a rate sufficient to generate a tissue concentration at the location proximal or distal the proximal end of the structure or the distal end of the structure, respectively (e.g., an adjacent tissue segment), within a range of about a range of about 1.5 ng/mg to about 30 ng/mg within about 3 hours. In some examples, the therapeutic composition is formulated to release the one or more active substances at a rate sufficient to generate a tissue concentration at the location proximal or distal to the proximal end of the structure or the distal end of the structure (e.g., within ±5 mm proximal or distal to an end of the structure), respectively, within a range of about 0.1 ng/mg to about 50 ng/mg, about 0.25 ng/mg to about 20 ng/mg, about 1 ng/mg to about 50 ng/mg, or about 3 ng/mg to about 50 ng/mg within about 3 hours.

In some examples, the therapeutic composition is formulated to release the one or more active substances at a rate sufficient to generate a tissue concentration at a location proximal or distal a proximal end of the structure or a distal end of the structure, respectively, within a range of about 0.2 ng/mg to about 25 ng/mg, about 2 ng/mg to about 25 ng/mg, or about 4 ng/mg to about 25 ng/mg within about 24 hours. In some examples, the therapeutic composition is formulated to release the one or more active substances at a rate sufficient to generate a tissue concentration at the location proximal or distal to the proximal end of the structure or the distal end of the structure (e.g., within ±5 mm proximal or distal to an end of the structure), respectively, within a range of about 0.1 ng/mg to about 50 ng/mg, about 0.25 ng/mg to about 20 ng/mg, about 1 ng/mg to about 50 ng/mg, or about 3 ng/mg to about 50 ng/mg within about 24 hours. In some examples, the therapeutic composition is formulated to release the one or more active substances at a rate sufficient to generate a tissue concentration at the location proximal or distal the proximal end of the structure or the distal end of the structure, respectively, within a range of about 0.3 ng/mg to about 10 ng/mg within about 24 hours.

In some examples, the therapeutic composition is formulated to release a dose of the direct factor Xa inhibitor sufficient to generate a blood concentration of the direct factor Xa inhibitor which is smaller than a median maximum serum concentration (Cₘₐₓ) of the direct factor Xa inhibitor generated by systemic delivery of the direct factor Xa inhibitor to achieve the same tissue concentration at the injury site. In other examples, the therapeutic composition is formulated to release a dose of the direct factor Xa inhibitor sufficient to generate a blood concentration of the direct factor Xa inhibitor which is smaller than a median maximum serum concentration (Cₘₐₓ) of the direct factor Xa inhibitor generated by systemic delivery of the direct factor Xa inhibitor when taking one or more oral dose of said factor Xa inhibitor. In some examples, the systemic delivery comprises a single oral dose, a daily oral dose, or a smallest oral dose of the direct factor Xa inhibitor. In some examples, the blood concentration is larger than a median minimum serum concentration (Cₘᵢₙ) of the direct factor Xa inhibitor generated by systemic delivery. In some examples, the blood concentration is smaller than a median minimum serum concentration (Cₘᵢₙ) of the direct factor Xa inhibitor generated by systemic delivery. In some examples, the Cₘₐₓ is measured using one of plasma blood, serum blood, or whole blood. In other examples, the median Cₘₐₓ is 80 ng/ml, or 123 ng/ml, or 171 ng/ml, or 321 ng/ml, or 480 ng/ml of blood.

In some examples, the therapeutic composition is formulated to release a dose of the direct factor Xa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor Xa inhibitor generated by systemic delivery of the direct factor Xa inhibitor to achieve the same tissue concentration at the injury site. In other examples, the therapeutic composition is formulated to release a dose of the direct factor Xa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor Xa inhibitor generated by systemic delivery of the direct factor Xa inhibitor when taking one or more oral dose of said factor Xa inhibitor. In some examples, the systemic delivery comprises a single oral dose, a daily oral dose, or a smallest oral dose of the direct factor Xa inhibitor. In some examples, the (AUC (0-24) or AUC (0-∞)) is measured using one of plasma blood, serum blood, or whole blood. In other examples, the median (AUC (0-24) or AUC (0-∞)) is 724 ng·h/ml, or 1437 ng·h/ml, or 2000 ng·h/ml, or 4000 ng·h/ml.

In some examples, the therapeutic composition is formulated to release a dose of the anti-proliferative agent sufficient to generate a blood concentration of the anti-proliferative agent which is smaller than a median maximum serum concentration (Cₘₐₓ) of the anti-proliferative agent generated by systemic delivery of the anti-proliferative agent to achieve the same tissue concentration at the injury site. In some examples, the systemic delivery comprises a single oral dose, a daily oral dose, or a smallest oral dose of the anti-proliferative agent. In some examples, the blood concentration is larger than a median minimum serum concentration (Cₘᵢₙ) of the anti-proliferative agent generated by systemic delivery. In some examples, the blood concentration is smaller than a median minimum serum concentration (Cₘᵢₙ) of the anti-proliferative agent generated by systemic delivery. In some examples, the therapeutic composition is formulated to release a dose of the anti-proliferative agent sufficient to generate a plasma drug level area under the curve (AUC (0-∞)) in ng·h/ml which is smaller than a median AUC (0-∞) in ng·h/ml of the anti-proliferative agent generated by systemic delivery of the anti-proliferative agent to achieve the same tissue concentration at the injury site.

In some examples, the therapeutic composition is formulated to release a dose of the direct factor IIa inhibitor sufficient to generate a blood concentration of the direct factor IIa inhibitor which is smaller than a median maximum serum concentration (Cₘₐₓ) of the direct factor IIa inhibitor generated by systemic delivery of the direct factor IIa inhibitor to achieve the same tissue concentration at the injury site. In other examples, the therapeutic composition is formulated to release a dose of the direct factor IIa inhibitor sufficient to generate a blood concentration of the direct factor IIa inhibitor which is smaller than a median maximum serum concentration (Cₘₐₓ) of the direct factor IIa inhibitor generated by systemic delivery of the direct factor IIa inhibitor when taking one or more oral dose of said factor IIa inhibitor. In some examples, the systemic delivery comprises a single oral dose, a daily oral dose, or a smallest oral dose of the direct factor IIa inhibitor. In some examples, the blood concentration is larger than a median minimum serum concentration (Cₘᵢₙ) of the direct factor IIa inhibitor generated by systemic delivery. In some examples, the blood concentration is smaller than a median minimum serum concentration (Cₘᵢₙ) of the direct factor IIa inhibitor generated by systemic delivery. In some examples, the Cₘₐₓ is measured using one of plasma blood, serum blood, or whole blood. In other examples, the median Cₘₐₓ is 80 ng/ml, or 123 ng/ml, or 171 ng/ml, or 321 ng/ml, or 480 ng/ml of blood.

In some examples, the therapeutic composition is formulated to release a dose of the direct factor IIa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor IIa inhibitor generated by systemic delivery of the direct factor IIa inhibitor to achieve the same tissue concentration at the injury site. In other examples, the therapeutic composition is formulated to release a dose of the direct factor IIa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor IIa inhibitor generated by systemic delivery of the direct factor IIa inhibitor when taking one or more oral dose of said factor IIa inhibitor. In some examples, the systemic delivery comprises a single oral dose, a daily oral dose, or a smallest oral dose of the direct factor IIa inhibitor. In some examples, the (AUC (0-24) or AUC (0-∞)) is measured using one of plasma blood, serum blood, or whole blood. In other examples, the median (AUC (0-24) or AUC (0-∞)) is 724 ng·h/ml, or 1437 ng·h/ml, or 2000 ng·h/ml, or 4000 ng·h/ml.

In some examples, local delivery of one or more of the active substances may reduce the time a patient needs to spend on oral medications and/or obviate the need for such medications entirely.

In some examples, the dose of each of the one or more active substances for optional systemic administration on a one-time basis or over a certain time period described herein (e.g., 6 hr, 12 hr, 1 day, 2 days, 3 days, 1 week, 2 weeks, 3 weeks, 1 month, etc.) independently is at least about 1, 5, 10, 20, 50, 100 or 500 mg, or at least about 1, 5 or 10 g. In further examples, the amount of each of the one or more active substances loaded in and/or on a temporary or non-temporary device, or the amount of each such agent released from the device, independently is at least about 1, 10, 50, 100 or 500 μg, or at least about 1, 5, 10 or 20 mg. In certain examples, the amount of each of the fibrin/thrombus formation-inhibiting or fibrin/thrombus dissolution-promoting agent(s) loaded in and/or on the device, or the amount of each such agent released from the device, independently is about 1 μg to about 20 mg, or about 10 μg to about 10 mg, or about 50 μg to about 5 mg, or about 100 μg to about 1 mg, or about 100 μg to about 500 μg, or about 500 μg to about 1 mg.

In further examples, the concentration of each of the one or more active substances released from a temporary or non-temporary device, and optionally administered systemically in addition to locally, in blood or tissue at the site of injury or at an area adjacent thereto, and/or in blood or tissue adjacent to the device, independently is at least about 0.001, 0.01, 0.1, 1, 10, 50, 100 or 500 nM, or at least about 1, 10, 50, 100, 500 or 1000 μM. In certain examples, the concentration of each of the fibrin/thrombus formation-inhibiting or fibrin/thrombus dissolution-promoting agent(s) released from a temporary or non-temporary device, and optionally administered systemically in addition to locally, in blood or tissue at the site of injury or at an area adjacent thereto, and/or in blood or tissue adjacent to the device, independently is about 0.01 or 0.1 nM to about 1000 μM, or about 0.1 or 1 nM to about 500 μM, or about 1 or 10 nM to about 100 μM, or about 50 nM to about 50 μM, or about 10 or 100 nM to about 10 μM, or about 100 nM to about 1 μM, or about 1 μM to about 10 μM.

In still further examples, the concentration of each of the one or more active substances released from a temporary or non-temporary device, and optionally administered systemically in addition to locally, in tissue at the site of injury or at an area adjacent thereto, and/or in tissue adjacent to the device, independently is at least about 0.01, 0.1, 1, 10, 50, 100 or 500 ng/gm tissue, or at least about 1, 10, 50, 100, 500 or 1000 μg/gm tissue. In certain examples, the concentration of each of the fibrin/thrombus formation-inhibiting or fibrin/thrombus dissolution-promoting agent(s) released from a temporary or non-temporary device, and optionally administered systemically in addition to locally, in tissue at the site of injury or at an area adjacent thereto, and/or in tissue adjacent to the device, independently is about 0.01 or 0.1 ng/gm tissue to about 1000 μg/gm tissue, or about 0.1 or 1 ng/gm tissue to about 500 μg/gm tissue, or about 1 or 10 ng/gm tissue to about 100 μg/gm tissue, or about 50 ng/gm tissue to about 50 μg/gm tissue, or about 10 or 100 ng/gm tissue to about 10 μg/gm tissue, or about 100 ng/gm tissue to about 1 μg/gm tissue, or about 1 μg/gm tissue to about 10 μg/gm tissue.

In additional examples, the concentration of each of the one or more active substances released from a temporary or non-temporary device (that may or may not cause an injury to a tissue, surface, vessel/lumen wall or other body part), and optionally administered systemically in addition to locally, in blood or tissue at the site of injury or at an area adjacent thereto, and/or in blood or tissue adjacent to the device, independently is at least about 0.001, 0.01, 0.1, 1, 10, 50, 100 or 500 nM, or at least about 1, 10, 50 or 100 μM, within about 1 day, 12 hr, 6 hr, 3 hr, 2 hr, 1 hr, 30 min., 15 min., 5 min. or 1 min. before, during and/or within about 1 day, 12 hr, 6 hr, 3 hr, 2 hr, 1 hr, 30 min., 15 min., 5 min. or 1 min. after delivery or deployment of the device and/or the injury. In further examples, the concentration of each of the one or more active substances released from a temporary or non-temporary device (that may or may not cause an injury to a tissue, surface, vessel/lumen wall or other body part), and optionally administered systemically in addition to locally, in tissue at the site of injury or at an area adjacent thereto, and/or in tissue adjacent to the device, independently is at least about 0.01, 0.1, 1, 10, 50, 100 or 500 ng/gm tissue, or at least about 1, 10, 50 or 100 μg/gm tissue, within about 1 day, 12 hr, 6 hr, 3 hr, 2 hr, 1 hr, 30 min., 15 min., 5 min. or 1 min. before, during and/or within about 1 day, 12 hr, 6 hr, 3 hr, 2 hr, 1 hr, 30 min., 15 min., 5 min. or 1 min. after delivery or deployment of the device and/or the injury.

In some examples, the dose of each of the one or more optional other kinds of bioactive agents (e.g., anti-proliferative agents, anti-inflammatory agents, etc.) for optional systemic administration on a one-time basis or over a certain time period described herein (e.g., 6 hr, 12 hr, 1 day, 2 days, 3 days, 1 week, 2 weeks, 3 weeks, 1 month, etc.) independently is at least about 1, 5, 10, 20, 50, 100 or 500 mg, or at least about 1, 5 or 10 g. In additional examples, the amount of each of the one or more optional other kinds of bioactive agents (e.g., anti-proliferative agents, anti-inflammatory agents, etc.) loaded in and/or on a temporary or non-temporary device, or the amount of each such agent released from the device, independently is at least about 1, 10, 50, 100 or 500 μg, or at least about 1, 5, 10 or 20 mg. In certain examples, the amount of each of the optional other kind(s) of bioactive agent(s) loaded in and/or on the device, or the amount of each such agent released from the device, independently is about 1 μg to about 20 mg, or about 10 μg to about 10 mg, or about 50 μg to about 5 mg, or about 100 μg to about 1 mg, or about 100 μg to about 500 μg, or about 500 μg to about 1 mg, or about 50 μg to about 200 μg.

In further examples, the concentration of each of the one or more optional other kinds of bioactive agents (e.g., anti-proliferative agents, anti-inflammatory agents, etc.) released from a temporary or non-temporary device, and optionally administered systemically in addition to locally, in tissue at the site of injury or at an area adjacent thereto, and/or in tissue adjacent to the device, independently is at least about 0.01, 0.1, 1, 10, 50, 100 or 500 ng/gm tissue, or at least about 1, 10, 50, 100, 500 or 1000 μg/gm tissue. In certain examples, the concentration of each of the optional other kind(s) of bioactive agent(s) released from a temporary or non-temporary device, and optionally administered systemically in addition to locally, in tissue at the site of injury or at an area adjacent thereto, and/or in tissue adjacent to the device, independently is about 0.01 or 0.1 ng/gm tissue to about 1000 μg/gm tissue, or about 0.1 or 1 ng/gm tissue to about 500 μg/gm tissue, or about 1 or 10 ng/gm tissue to about 100 μg/gm tissue, or about 50 ng/gm tissue to about 50 μg/gm tissue, or about 10 or 100 ng/gm tissue to about 10 μg/gm tissue, or about 100 ng/gm tissue to about 1 μg/gm tissue, or about 1 μg/gm tissue to about 10 μg/gm tissue.

In some examples, the patient receiving one or more active substances (e.g, anti-coagulants) has a condition or is susceptible to a condition that renders the subject more susceptible to a vaso-occlusive event. In further examples, the subject has pulmonary disease or is susceptible to pulmonary disease. In certain examples, the pulmonary disease is selected from the group consisting of arteriosclerosis, cardiopulmonary disease, cerebropulmonary disease, peripheral pulmonary disease, renopulmonary disease, mesenteric pulmonary disease, pulmonary pulmonary disease, and ocular pulmonary disease.

In additional examples, the patient has a condition or is susceptible to a condition selected from the group consisting of hyperlipidemia, hypercholesterolemia, hypertension, atherosclerosis, and diabetes. In certain examples, the patient is diabetic.

In some examples, measurements of blood or tissue described herein comprise one or more of mammalian blood or tissue, porcine blood or tissue, human blood or tissue, rabbit blood or tissue, rat blood or tissue, mouse blood or tissue, or the like.

One or more bioactive substances or agents can be delivered from any suitable medical device as described herein. The device can be a temporary device (e.g., a balloon, a catheter, a needle, a surgical knife or other surgical tool, a patch, etc.) or a non-temporary device (e.g., an implant, such as a stent, a graft, etc.). In some examples, the device is selected from the group consisting of temporary devices, non-temporary devices (including permanent devices), access devices, infusion devices, tools, surgical instruments and tools, implants, bodily implants, organ implants, hip implants, shoulder implants, knee implants, luminal implants, pulmonary implants, stent-delivery systems, stents (including pulmonary stents, coronary stents and peripheral stents), stent-grafts, catheters (including infusion catheters, diffusion catheters, balloon-catheters, weeping catheters, and electrode catheters), balloons, graft implants, grafts (including aortic grafts, arterio-venous grafts and by-pass grafts), aneurysm coils (including abdominal aortic aneurysm coils and cerebral aneurysm coils), valves (including artificial heart valves), valve implants, shunts (including axius coronary shunts and cerebrospinal fluid shunts), left atrial appendage implants, foramen implants, leads (including endocardial leads), closure devices (including arterial and patent foramen ovale closure devices), clips (including anastomotic clips), wound-closure devices and implants, sutures, patches, injection devices, needles inserted in the body of a subject, and needles inserted from outside the body.

Non-limiting examples of surgical instruments and tools include surgical knives and mechanical cutters (e.g., scalpels, lancets, drill bits, rasps, scissors); other cutting instruments (e.g., microtomes, dermatomes, cryotomes, cutting laser guides) and ultrasound tissue disruptors; graspers (e.g., forceps); clamps, occluders and compressors (e.g., hemostats) for organs and tubular structures (e.g., blood vessels and other lumens); sealing devices (e.g., surgical staplers, LigaSure™ tissue-fusion devices); dilators and specula; retractors (e.g., those used to spread open skin, ribs and other tissues and body parts) and tyndallers (e.g., those used to wedge open brain tissue and other tissues); needles, tips and tubes (e.g., trocars) for introducing or removing material (e.g., fluids); scopes and probes (e.g., endoscopes, tactile probes); distractors, positioners and stereotactic devices; powered devices (e.g., drills); carriers and appliers for optical, electronic and mechanical devices; and measurement devices (e.g., rulers, calipers).

In some examples, the device contains the bioactive agent(s) in the body and/or on at least one surface of the device. In certain examples, the bioactive agent(s) are contained in one or more layers in the body and/or at the surface of the device.

In further examples, the bioactive agent(s) are contained in one or more coatings disposed over the body of the device. The coating(s) can be disposed over any desired portion(s) and any desired surface(s) of the body of the device. As a non-limiting example, for a tubular pulmonary device such as a stent, the coating(s) can be disposed over the luminal (lumen-facing) surface, the abluminal (tissue-facing) surface or the side surface(s) of the stent, or a combination thereof (e.g., all surfaces of the stent).

In additional examples, the device comprises the bioactive agent(s) in the body of the device and in one or more coatings disposed over the body of the device.

A temporary or non-temporary device can comprise openings in and/or on the body (including at the surface) of the device, and/or in one or more coatings disposed over the body structure of the device. Examples of openings include without limitation pores (including partial pores and through pores), holes (including partial holes and through holes), voids, recesses, pits, cavities, trenches, reservoirs and channels. In some examples, a temporary or non-temporary device contains one or more anti-coagulant, and optionally one or more other kinds of bioactive agents (e.g., anti-proliferative agents, anti-inflammatory agents, etc.) in openings in and/or on the body (including at the surface) of the device, and/or in one or more coatings disposed over the body of the device.

The device may comprise one or more coatings disposed over an exterior surface of a structure of the device, as described herein. In some embodiments, the coating(s) may comprise a homopolymer, a copolymer, a mixture of homopolymers, a mixture of copolymers, or a mixture of a homopolymer and a copolymer. In some examples, the coating(s) comprise a soft or hydrophilic, or a softer or more hydrophilic, polymeric material. In further examples, the coating(s) comprise a polymeric material and an additive (e.g., a monomer of the polymeric material) that softens the polymeric material.

In some examples, the device has a first coating that comprises a biodegradable or non-degradable polymeric material, or one or more bioactive agents, or both a biodegradable or non-degradable polymeric material and one or more bioactive agents. In further examples, the device has a second coating that comprises a biodegradable or non-degradable polymeric material, or one or more bioactive agents, or both a biodegradable or non-degradable polymeric material and one or more bioactive agents, wherein the second coating optionally is disposed over the first coating. In additional examples, the device has a third coating that comprises a biodegradable or non-degradable polymeric material, wherein the third coating is disposed over the first coating and/or the second coating. In some examples, the third coating serves as a top layer or coat or diffusion barrier that controls release of one or more bioactive agents from inner coating(s) and/or the body of the device.

In some examples, a bioactive agent that is intended to have an earlier or shorter time of action can be contained in an outer coating, on a surface uncovered by a coating, and/or in the body of the device closer to the surface, and a bioactive agent that is intended to have a later or longer time of action can be contained in an inner coating, in a coating covered by a barrier coating, on a surface covered by a coating, and/or in the body of the device farther from the surface. In further examples, a bioactive agent that is intended to have an earlier or shorter time of action is contained on a surface of the device, or contained in a coating on the device or in a layer of the body of the device which comprises a faster-degrading polymeric material, and a bioactive agent that is intended to have a later or longer time of action is contained within the device, or contained in a coating on the device or in a layer of the body of the device which comprises a slower-degrading or non-degrading polymeric material. In additional examples, a bioactive agent that is intended to have an earlier or shorter time of action is more soluble, and a bioactive agent that is intended to have a later or longer time of action is less soluble.

In certain examples, the concentration of a bioactive agent [e.g., anti-coagulant, anti-proliferative, etc.] in a coating comprising a polymeric material is at least about 10%, 20%, 30%, 40%, 50% or 60% by weight relative to the weight of the bioactive agent and the polymeric material.

In further examples, the thickness (e.g., average thickness) of each of the coating(s) independently is no more than about 20, 15, 10, 5, 3 or 1 micron.

In some examples, the coating(s) may comprise carrier material. Non-limiting examples of carrier materials include biodegradable polymeric materials, non-degradable polymeric materials, and other matrix materials.

In some examples, the carrier material may be porous. In certain examples, the porosity of each of the coating(s) of the carrier material may be within a range of about 10 nm to about 10 μm.

In some examples, the carrier material may be biodegradable. In certain examples, the carrier material may have a depredation rate within a range of about 1 month to about 36 months.

In some examples, the weight compositional ratio of the carrier material to the therapeutic composition of one or more bioactive agents may be within a range of about 1:5 to 3:2.

Non-limiting examples of polymeric materials that can compose the carrier material include polyesters, polylactide, polyglycolide, poly(ε-caprolactone), polydioxanone, poly(hydroxyalkanoates), poly(L-lactide-co-D-lactide), poly(L-lactide-co-D,L-lactide), poly(D-lactide-co-D,L-lactide), poly(lactide-co-glycolide) (including 70:30 to 99:1 PLA-co-PGA, such as 85:15 PLA-co-PGA), poly(lactide-co-ε-caprolactone) (including 70:30 to 99:1 PLA-co-PCL, such as 90:10 PLA-co-PCL), poly(glycolide-co-ε-caprolactone), poly(lactide-co-dioxanone), poly(glycolide-co-dioxanone), poly(lactide-co-trimethylene carbonate), poly(glycolide-co-trimethylene carbonate), poly(lactide-co-ethylene carbonate), and copolymers and combinations thereof, wherein lactide includes L-lactide, D-lactide and D,L-lactide. The polymeric material may comprise a material selected from a group of non-degradable polymeric materials consisting of polyacrylates, polymethacrylates, poly(n-butyl methacrylate), poly(hydroxyethylmethacrylate), polyamides, nylons, nylon 12, Dacron, Polyethylene terephthalate, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate), and copolymers and combinations thereof.

Non-limiting examples of biodegradable polymeric materials that can compose the body of the device, a layer of the body, or a coating include polyesters, poly(α-hydroxyacids), polylactide, polyglycolide, poly(ε-caprolactone), polydioxanone, poly(hydroxyalkanoates), poly(hydroxypropionates), poly(3-hydroxypropionate), poly(hydroxybutyrates), poly(3-hydroxybutyrate), poly(4-hydroxybutyrate), poly(hydroxypentanoates), poly(3-hydroxypentanoate), poly(hydroxyvalerates), poly(3-hydroxyvalerate), poly(4-hydroxyvalerate), poly(hydroxyoctanoates), poly(3-hydroxyoctanoate), polysalicylate/polysalicylic acid, polycarbonates, poly(trimethylene carbonate), poly(ethylene carbonate), poly(propylene carbonate), tyrosine-derived polycarbonates, L-tyrosine-derived polycarbonates, polyiminocarbonates, poly(DTH iminocarbonate), poly(bisphenol A iminocarbonate), poly(amino acids), poly(ethyl glutamate), poly(propylene fumarate), polyanhydrides, polyorthoesters, poly(DETOSU-1,6HD), poly(DETOSU-t-CDM), polyurethanes, polyphosphazenes, polyamides, nylons, nylon 12, polyoxyethylated castor oil, poly(ethylene glycol), polyethylene oxide (PEO), polyvinylpyrrolidone, poly(L-lactide-co-D-lactide), ethylene-vinyl acetate, poly(L-lactide-co-D,L-lactide), poly(D-lactide-co-D,L-lactide), poly(lactide-co-glycolide) (including 70:30 to 99:1 PLA-co-PGA, such as 85:15 PLA-co-PGA), poly(lactide-co-F-caprolactone) (including 70:30 to 99:1 PLA-co-PCL, such as 90:10 PLA-co-PCL), poly(glycolide-co-F-caprolactone), poly(lactide-co-dioxanone), poly(glycolide-co-dioxanone), poly(lactide-co-trimethylene carbonate), poly(glycolide-co-trimethylene carbonate), poly(lactide-co-ethylene carbonate), poly(glycolide-co-ethylene carbonate), poly(lactide-co-propylene carbonate), poly(glycolide-co-propylene carbonate), poly(lactide-co-2-methyl-2-carboxyl-propylene carbonate), poly(glycolide-co-2-methyl-2-carboxyl-propylene carbonate), poly(lactide-co-hydroxybutyrate), poly(lactide-co-3-hydroxybutyrate), poly(lactide-co-4-hydroxybutyrate), poly(glycolide-co-hydroxybutyrate), poly(glycolide-co-3-hydroxybutyrate), poly(glycolide-co-4-hydroxybutyrate), poly(lactide-co-hydroxyvalerate), poly(lactide-co-3-hydroxyvalerate), poly(lactide-co-4-hydroxyvalerate), poly(glycolide-co-hydroxyvalerate), poly(glycolide-co-3-hydroxyvalerate), poly(glycolide-co-4-hydroxyvalerate), poly(3-hydroxybutyrate-co-4-hydroxybutyrate), poly(hydroxybutyrate-co-hydroxyvalerate), poly(3-hydroxybutyrate-co-3-hydroxyvalerate), poly(3-hydroxybutyrate-co-4-hydroxyvalerate), poly(4-hydroxybutyrate-co-3-hydroxyvalerate), poly(4-hydroxybutyrate-co-4-hydroxyvalerate), poly(F-caprolactone-co-fumarate), poly(F-caprolactone-co-propylene fumarate), poly(ester-co-ether), poly(lactide-co-ethylene glycol), poly(glycolide-co-ethylene glycol), poly(F-caprolactone-co-ethylene glycol), poly(ester-co-amide), poly(DETOSU-1,6HD-co-DETOSU-t-CDM), poly(lactide-co-cellulose ester), poly(lactide-co-cellulose acetate), poly(lactide-co-cellulose butyrate), poly(lactide-co-cellulose acetate butyrate), poly(lactide-co-cellulose propionate), poly(glycolide-co-cellulose ester), poly(glycolide-co-cellulose acetate), poly(glycolide-co-cellulose butyrate), poly(glycolide-co-cellulose acetate butyrate), poly(glycolide-co-cellulose propionate), poly(lactide-co-glycolide-co-F-caprolactone), poly(lactide-co-glycolide-co-trimethylene carbonate), poly(lactide-co-F-caprolactone-co-trimethylene carbonate), poly(glycolide-co-F-caprolactone-co-trimethylene carbonate), poly(3-hydroxybutyrate-co-3-hydroxyvalerate-co-4-hydroxybutyrate), poly(3-hydroxybutyrate-co-4-hydroxyvalerate-co-4-hydroxybutyrate), collagen, casein, polysaccharides, cellulose, cellulose esters, cellulose acetate, cellulose butyrate, cellulose acetate butyrate, cellulose propionate, chitin, chitosan, dextran, starch, modified starch, and copolymers and combinations thereof, wherein lactide includes L-lactide, D-lactide and D,L-lactide.

Examples of non-degradable polymeric materials that can compose the body of the device, a layer of the body, or a coating include without limitation polyacrylates, polymethacrylates, poly(n-butyl methacrylate), poly(hydroxyethylmethacrylate), poly(styrene-b-isobutylene-b-styrene), phosphorylcholine polymer, poly(ethylene-co-vinyl acetate), poly(n-butyl methacrylate), blend of thermoplastic Silicone-Polycarbonate-urethane with poly n-butyl methacrylate, poly(vinylidene-co-hexafluoropropylene), Blend of polyvinylpyrrolidone, poly(hexylmethacrylate)-co-polyvinylpyrrolidone-co-poly vinyl acetate, and poly(n-butyl methacrylate)-co-poly(vinyl acetate), Poly(styrene-butylene styrene), poly(tyrosine-derived polycarbonate), polyamides, nylons, nylon 12, poly(ethylene glycol), polyethylene oxide (PEO), polydimethylsiloxane, polyvinylpyrrolidone, ethylene-vinyl acetate, phosphorylcholine-containing polymers, poly(2-methacryloyloxyethylphosphorylcholine), poly(2-methacryloyloxyethylphosphorylcholine-co-butyl methacrylate), polyvinylpyridine block with poly methyl methacrylate (PMMA), poly N-(2-Hydroxypropyl) methacrylamide, Polyethylenimine (PEI), dextran, dextrin, chitosans, poly(L-lysine), and poly(aspartamides), polyamides, Polyethylene glycol (PEG), Silicones, poly(anhydride), poly ortho esters, polystyrene-b-polyvinylpyridine, poly(styrene)-poly(butadiene)-poly(vinyl pyridine), poly(styrene-poly(methacrylic acid), poly(styrene)-poly(ethylene oxide), poly(vinyl pyridine)-poly(butadiene)-poly(vinyl pyridine), and poly(styrene)-poly(vinyl pyridine)-poly(ethylene oxide) and copolymers and/or combinations thereof.

Non-limiting examples of corrodible metals and metal alloys that can compose the body of the device, a layer of the body, or a coating include cast ductile irons (e.g., 80-55-06 grade cast ductile iron), corrodible steels (e.g., AISI 1010 steel, AISI 1015 steel, AISI 1430 steel, AISI 5140 steel and AISI 8620 steel), melt-fusible metal alloys, bismuth-tin alloys (e.g., 40% bismuth-60% tin and 58% bismuth-42% tin), bismuth-tin-indium alloys, magnesium, magnesium alloys, tungsten alloys, zinc alloys, shape-memory metal alloys, and superelastic metal alloys. Examples of non-corrodible metals and metal alloys that can compose the body of the device, a layer of the body, or a coating include without limitation stainless steels (e.g., 316L stainless steel), cobalt-chromium alloys (e.g., L-605 and MP35N cobalt-chromium alloys), gold, molybdenum-rhenium alloys, nickel-titanium alloys, palladium, platinum, platinum-iridium alloys, tantalum, and alloys thereof.

In some examples, the device is coated. The coating layer may comprise a therapeutic agent and an additive. In some examples, the coating layer overlying an exterior surface of the exterior surface of the medical device consists essentially of the therapeutic agent and the additive.

In some examples, the additive is selected from PEG (polyethylene glycol), polyalkylene oxide, e.g., polyethylene oxide, polypropylene oxide, or a copolymer thereof (e.g., a polyethylene oxide-polypropylene oxide-polyethylene oxide copolymers), polyphenylene oxide, copolymers of PEG and polyalkylene oxide, poly(methoxyethyl methacrylate benzoate), poly (a methacryloyloxy one phosphatidylcholine), perfluorinated polyether, dextran or poly vinylpyrrolidone, poly (ethylene-vinyl acetate), polypeptides, water soluble surfactants, water soluble vitamins, and proteins, PEG fatty esters and alcohols, glycerol fatty esters, sorbitan fatty esters, PEGylation (PEG-drug conjugation), PEG glyceryl fatty esters, PEG sorbitan fatty esters, sugar fatty esters, PEG sugar esters, vitamins and derivatives, amino acids, multi amino acids and derivatives, peptides, polypeptides, oligomers, copolymers, block polymers, proteins, albumin, quaternary ammonium salts such as but not limited to benzalkonium chloride, benzethonium chloride, docecyl trimethyl ammonium bromide, sodium docecylsulfates, dialkyl methylbenzyl ammonium chloride, dialkylesters of sodium sulfonsuccinic acid, organic acids, salts and anhydrides and combinations thereof.

In one example, the device is drug coated balloon. The balloon can optionally adopt carrier excipient to coat to facilitate drug transfer to the vessel wall and control release rate. A variety of carrier excipients are not limited to contrast agent (i.e. iopromide), urea, dextrane, shellac, shelloic acid, keratosis (a naturally derived protein), Plasticizer (i.e. butyryl-tri-hexyl citrate, acetyl tributyl citrate, citrate ester, glycerol, other organic ester), hydrophilic space, Polyvinylpyrrolidone (PVP) and its hydrogels, Surfactants, Non-ionic surfactant Polysorbate/sorbitol (i.e. Tween20, Tween60 or Tween80), nordihydroguaiaretic acid (NDGA), hydrophibic excipient such as phospholipid, amphiphilic polymer such as Poly(ethylene glycol) (i.e PEG 8000), poly(ethylene oxide) (PEO) (molecular weight range from 100,000 to 10,000,000), Polyethylenimine (PEI) or polyaziridine linear or branched, amphiphilic block co-polymers composed of poly(ethylene oxide) (PEO) as the hydrophilic block and poly(ether)s, poly(amino acid)s), hydrophobic polymer space, biodegradable polymers such as Poly DL lactide-co-glycolide, Poly L Lactide-co-caprolactone, durable polymers, individually or combinations thereof.

In one example, several anticoagulants delivered locally were tested in-vivo in an animal model including Heparin, Rivaroxaban (factor Xa inhibitor), and Argatroban (factor IIa inhibitor). It was an unexpected result that only Rivaroxaban was shown to inhibit fibrin formation at 7 days.

In another example, two formulations of Rivaroxaban were tested in a local delivery in-vivo animal model, wherein one formulation comprised a faster release dose release within 7 days versus control within 7 days. It was unexpected result that the composition comprising faster release dose released within 7 days was more effective than control, within 7 days. The composition comprising faster dose inhibited fibrin more effectively through 28 days compared to 7 days with the composition comprising a slower release dose.

A surprising finding was that composition comprising the fast release of Rivaroxaban in combination with m-TOR inhibitor released locally was more effective at inhibiting fibrin at 7 days and 28 days as compared to control while a slower release formulation of Rivaroxaban in combination with m-TOR inhibitor was less effective at inhibiting fibrin formation at 28 days from implant.

A surprising finding was that a composition comprising Rivaroxaban released locally in combination with m-TOR inhibitor inhibits fibrin formation after injury. Many attempts using heparin, and other anticoagulants have failed to show such effects when combined with m-TOR inhibitors.

A surprising finding was that a composition comprising Rivaroxaban released locally in combination with Argatroban inhibited fibrin formation after injury.

A surprising finding was that a composition comprising Rivaroxaban released locally in combination with Argatroban inhibited smooth muscle cell proliferation after injury.

A surprising finding was that a composition comprising Rivaroxaban released locally in combination with Argatroban and an m-TOR inhibitor further inhibited smooth muscle cell proliferation after injury.

In one example, a device for use in a body lumen is configured to release locally a composition comprising factor Xa inhibitor, preferably Rivaroxaban, more preferably Apixaban, wherein said device is configured to release at least 89 μg, preferably at least 150 μg (micro-grams) of said factor Xa inhibitor, within 3 hours, within 12 hours, within 1 day, within 3 days, or within 7 days from time of injury.

In another example, a device for use in a body lumen is configured to release locally a composition comprising factor Xa inhibitor, preferably Rivaroxaban, more preferably Apixaban, wherein said device is configured to release at least 6.36 μg per millimeter of device length, preferably release at least 10.7 μg per millimeter of device length, of said factor Xa inhibitor within 3 hours, within 12 hours, within 1 day, within 3 days, or within 7 days from time of injury.

In another example, a device for use in a body lumen is configured to release locally a composition comprising factor Xa inhibitor, preferably Rivaroxaban, more preferably Apixaban, wherein said device from time of injury to 3 hours, 12 hours, 1 day, 3 days, or to 7 days is configured to release 89 μg or more or 6.36 μg or more/mm of device length of said drug, preferably configured to release 150 μg or more or 10.7 μg/mm of device length or more of said drug.

It was surprisingly found extended release formulation comprising a factor IIa inhibitor and/or a factor Xa inhibitor, inhibited one or more of clot formation, SMC proliferation, inflammation, and injury, wherein the extended release of the one or more drugs extended beyond 7 days, extended beyond 14 days, extended beyond 21 days, extended beyond 28 days, or extended beyond 3 months.

In another example, a device for use in a body lumen is configured to release locally a factor Xa inhibitor, preferably Rivaroxaban, more preferably Apixaban, wherein said device from time of injury to 3 hours, 12 hours, 1 day, 3 days, or to 7 days is configured to release from 89 μg to 150 μg of said drug, or release from 6.36 μg/mm of device length to 10.7 μg/mm of device length of said drug.

In another example, a device for use in a body lumen is configured to release locally a factor Xa inhibitor, preferably Rivaroxaban, more preferably Apixaban, wherein said device from time of injury to 3 hours, 12 hours, 1 day, 3 days, or to 7 days is configured to release from 89 μg to 150 μg or more of said drug, or release from 6.36 μg/mm of device length to 10.7 μg or more/mm of device length of said drug.

In another example, a device for use in a body lumen is configured to release locally a factor Xa inhibitor, preferably Rivaroxaban, more preferably Apixaban, wherein said device from time of injury to 3 hours, 12 hours, 1 day, 3 days, or to 7 days is configured to release from 89 μg or more to 150 μg of said drug, or release from 6.36 μg or more/mm of device length to 10.7 μg/mm of device length of said drug.

In another example, a device for use in a body lumen is configured to release locally a factor Xa inhibitor, preferably Rivaroxaban, more preferably Apixaban, wherein said device from time of injury to 3 hours, 12 hours, 1 day, 3 days, or to 7 days is configured to release from 89 μg or more to 500 μg of said drug, or release from 6.36 μg or more/mm of device length to 40 μg/mm of device length of said drug.

In another example, a device for use in a body lumen is configured to release locally a factor Xa inhibitor, preferably Rivaroxaban, more preferably Apixaban, wherein said device from time of injury to 28 days is configured to release at least 92 μg of said drug, preferably release at least 150 μg of said drug, more preferably release at least 200 μg of said drug, most preferably release at least 250 μg of said drug.

In another example, a device for use in a body lumen is configured to release locally a factor Xa inhibitor, preferably Rivaroxaban, more preferably Apixaban, wherein said device from time of injury to 28 days is configured to release at least 6.6 μg or more/mm of device length, preferably release at least 10.7 μg/mm of device length of said drug, more preferably release at least 14.3 μg/mm of device length of said drug, most preferably release at least 17.86 μg/mm of device length of said drug.

In another example, a device for use in a body lumen is configured to release locally a factor Xa inhibitor, preferably Rivaroxaban, more preferably Apixaban, wherein said device from time of injury to 28 days is configured to release from 92 μg to 300 μg.

In another example, a device for use in a body lumen is configured to release locally a factor Xa inhibitor, preferably Rivaroxaban, more preferably Apixaban, wherein said device from time of injury to 28 days is configured to release from 92 μg or more to 500 μg of said drug, or release from 6.6 μg or more/mm of device length to 40 μg/mm of device length of said drug.

In another example, a device for use in a body lumen is configured to release locally a factor Xa inhibitor, preferably Rivaroxaban, more preferably Apixaban, wherein the tissue concentration in the device segment (stented segment) by or at 3 hours ranges from 3.9 ng/mg of tissue to 200 ng/mg of tissue, preferably ranges from 3.9 ng/mg of tissue to 150 ng/mg of tissue. In other examples, the tissue concentration at 3 hours is at least 3.9 ng/mg of tissue, preferably at least 25 ng/mg of tissue, more preferably at least 50 ng/mg of tissue, and most preferred at least 75 ng/mg of tissue.

In another example, a device for use in a body lumen is configured to release locally a factor Xa inhibitor, preferably Rivaroxaban, more preferably Apixaban, wherein the tissue concentration in the device segment (stented segment) by or at 3 hours, 12 hours, 1 day, 3 days, or 7 days ranges from 3.9 ng/mg of tissue to 200 ng/mg of tissue, preferably ranges from 3.9 ng/mg of tissue to 150 ng/mg of tissue. In other examples, the tissue concentration at 3 hours, 12 hours, 1 day, 3 days, or at 7 days have tissue concentration of at least 3.9 ng/mg of tissue, preferably at least 25 ng/mg of tissue, more preferably at least 50 ng/mg of tissue, and most preferred at least 75 ng/mg of tissue.

In another example, a device for use in a body lumen is configured to release locally a factor Xa inhibitor, preferably Rivaroxaban, more preferably Apixaban, wherein the tissue concentration in the device segment (stented segment) by or at 28 days range from 1.69 ng/mg of tissue to 10 ng/mg of tissue, preferably ranges from 3.9 ng/mg of tissue to 5 ng/mg of tissue. In other examples, the tissue concentration at 28 days have tissue concentration of at least 1.69 ng/mg of tissue, preferably at least 3.6 ng/mg of tissue, more preferably at least 3.9 ng/mg of tissue, and most preferred at least 5 ng/mg of tissue.

In another example of any of the examples, a timepoint such as 3 hours, 1 day, 7 days, or 28 days refer to one of from time of injury, from time of release of drug, from time of implant releasing device, or from time of end procedure.

In another example of any of the example, a device is configured to release locally a factor Xa inhibitor to one or more of injured tissue segment, tissue segment adjacent to the device, adjacent tissue segment to the injured tissue segment, ±5 mm adjacent tissue to the injured tissue segment, 5 mm proximal adjacent tissue to the injured tissue segment, 5 mm distal adjacent tissue to the injured tissue segment, the device surface, to a body lumen wall, to a body lumen, to the abluminal surface of the device, to the luminal surface of the device.

In another example of any of the examples, from time of injury comprises one or more of time from injury by device releasing drug, time from injury by another device before device releasing drug, time from injury by another device 5, 10, 15, or 30 minutes before device releasing.

In some examples, the device for use in a body lumen wherein said device is configured to release one or more of factor Xa inhibitor wherein the dose ranges from 100 micrograms to 1000 micrograms, preferably ranging from 150 micrograms to 500 micrograms, more preferably ranging from 150 micrograms to 300 micrograms.

In another example, a device for use in a body lumen is configured to release locally a factor Xa inhibitor, preferably Rivaroxaban, more preferably Apixaban, wherein said device is configured to release one or more factor Xa inhibitors wherein the drug dose ranges from 7.14 μg/mm of device length to 71 μg/mm of device length, preferably ranges from 10.71 μg/mm of device length to 35.7 μg/mm of device length, more preferably ranges from 10.71 μg/mm of device length to 21.4 μg/mm of device length.

In some examples, the device releasing factor Xa, preferably releasing Rivaroxaban, more preferably releasing Apixaban, wherein the device is configured to release said drug at a rate ranging from 88.9% to 99.7% from time of injury to 3 hours, 12 hours, 1 day, 3 days, or 7 days.

In some examples, the device releasing factor Xa, preferably releasing Rivaroxaban, more preferably releasing Apixaban, wherein the device is configured to release said drug at a rate ranging from 92% to 99.7% from time of injury to 28 days.

In some examples, the device releasing factor Xa, preferably releasing Rivaroxaban, more preferably releasing Apixaban, wherein the device is configured to release said drug at a rate ranging from 92% to 100% from time of injury to 28 days.

In another example the factor Xa inhibitor drug release rate composed of release of 100% by 28 days, preferably ranging from 90% to 100%, more preferably ranging from 95% to 100% by 28 days.

In another example, a device for use in a body lumen comprising a factor Xa inhibitor drug, wherein the drug is Rivaroxaban, preferably Apixaban, and wherein the device has a drug dose and wherein the drug is released at a rate ranging from 50% to 90%, preferably ranging from 55% to 85%, more preferably ranging from 60% to 80% of the drug dose within 3 hours, 12 hours, or 3 days from time of injury.

In another example, a device for use in a body lumen comprising a factor Xa inhibitor drug, wherein the drug is Rivaroxaban, preferably Apixaban, and wherein the device has a drug dose and wherein the drug is released at a rate ranging from 70% to 100%, preferably ranging from 80% to 99%, more preferably ranging from 85% to 99% of the drug dose within 3 hours, 12 hours, or 3 days from time of injury.

In another example, a device for use in a body lumen comprising a factor Xa inhibitor drug, wherein the drug is Rivaroxaban, preferably Apixaban, and wherein the device has a drug dose and wherein the drug is released at a rate ranging from 88% to 100%, preferably ranging from 92% to 100%, more preferably ranging from 85% to 99% of the drug dose within 7 days from time of injury.

In another example, a device for use in a body lumen comprising a factor Xa inhibitor drug, wherein the drug is Rivaroxaban, preferably Apixaban, and wherein the device has a drug dose and wherein the drug is released at a rate ranging from 12.86 μg to 200 μg, preferably ranging from 15 μg to 150 μg, more preferably ranging from 20 μg to 150 μg within an hour, within 3 hours, within 12 hours, within 3 days, or within 7 days from time of injury.

In some examples, a device for use in a body lumen wherein said device is configured to release one or more of factor Xa inhibitors within 28 day or more from time of injury wherein said release within 28 days ranges from 100 to 1000 micrograms, preferably ranges from 150 to 600 micrograms, more preferably ranges from 150 to 300 micrograms.

In another example, a device for use in a body lumen wherein said device is configured to release beyond 28 days one or more of factor Xa inhibitors, preferably Rivaroxaban, more preferably Apixaban, wherein said release beyond 28 days from time of injury ranges from 0.1 micrograms to micrograms, preferably ranges from 1 microgram to 25 micrograms, more preferably ranges from 1 microgram to 5 microgram.

In another example, a factor Xa inhibitor, preferably Rivaroxaban, more preferably Apixaban released locally from a device after injury wherein said drug inhibits fibrin formation, thrombin formation, and/or clot formation.

In another example, a factor Xa inhibitor, preferably Rivaroxaban, more preferably Apixaban released locally from a device after injury wherein said drug inhibits fibrin formation thereby inhibiting clot formation.

In another example, a factor Xa inhibitor, preferably Rivaroxaban, more preferably Apixaban in combination with Argatroban released from a device locally after injury wherein said drug combination inhibits smooth muscle cell proliferation.

In another example, a factor Xa inhibitor, preferably Rivaroxaban, more preferably Apixaban in combination with Argatroban released locally from a device after injury wherein said drug combination inhibits fibrin formation thereby inhibiting clot formation.

In another example, a factor Xa inhibitor, preferably Rivaroxaban, more preferably Apixaban in combination with Argatroban released locally from a device after injury wherein said drug combination inhibits fibrin formation or clot formation.

In another example, a factor Xa inhibitor, preferably Rivaroxaban, more preferably Apixaban released from a device locally at a dose of at least 150 μg within 7 days from implant (or from vessel injury) to inhibit fibrin formation, or to inhibit clot formation.

In another example, a factor Xa inhibitor, preferably Rivaroxaban, more preferably Apixaban released locally from a device after injury wherein said drug is released at a dose of at least 1.8 μg/mm² within 7 days from vessel injury to inhibit clot formation or fibrin formation.

In another example, a factor Xa inhibitor, preferably Rivaroxaban, more preferably Apixaban released locally from a device after injury wherein said device releases said drug at a dose of at least 10.7 μg/mm of stent length within 7 days from vessel injury inhibiting clot formation or fibrin formation.

In another example, a factor Xa inhibitor, preferably Rivaroxaban, more preferably Apixaban released locally from a device after injury wherein the drug dose of at least 150 μg, or of at least 1.8 μg/mm², or at least 10.7 μg/mm of device length, are released from the device at a release rate of about 99.6% within 7 days from time of injury to inhibit fibrin formation or clot formation.

In another example, a factor Xa inhibitor, preferably Rivaroxaban, more preferably Apixaban released locally from a device after injury wherein the drug is released at a release rate of at least 70.9% when combined with Argatroban at a release rate of at least 96.9% within 7 days from time of injury to inhibit fibrin formation or clot formation.

In another example, a factor Xa inhibitor, preferably Rivaroxaban, more preferably Apixaban released locally from a device after injury wherein said drug is released from said device at a dose of at least 100 μg, or at a dose of at least 1.2 μg/mm², or at a dose of at least 7.14 μg/mm of stent length, and at a release rate of at least 70.9% within 7 days when combined with Argatroban released from a stent at a dose of at least 100 μg, or at a dose of at least 1.2 μg/mm², or at a dose of at least 7.14 μg/mm of stent length, and at a release rate of at least 96.9% within 7 days from device implantation to inhibit clot formation.

In another example, a factor Xa inhibitor, preferably Rivaroxaban, more preferably Apixaban released locally from a balloon catheter wherein said drug is released from said device at a dose of at least 500 μg, or at a dose of at least 10 μg/mm², or at a dose of at least 10 μg/mm of balloon length, within 10 seconds to 5 minutes after expansion of the balloon to inhibit clot formation.

In another example, a factor Xa inhibitor, preferably Rivaroxaban, more preferably Apixaban released locally from a balloon catheter to inhibit clot formation after vessel injury.

In another example, a factor Xa inhibitor, preferably Rivaroxaban, more preferably Apixaban released locally in combination with Argatroban from a balloon catheter to inhibit clot formation after vessel injury.

In another example, a factor Xa inhibitor, preferably Rivaroxaban, more preferably Apixaban released locally in combination with Argatroban from a balloon catheter to inhibit smooth muscle proliferation after vessel injury.

In another example, a factor Xa inhibitor, preferably Rivaroxaban, more preferably Apixaban released locally in combination with Argatroban and an m-TOR inhibitor from a balloon catheter to inhibit smooth muscle proliferation after vessel injury and/or clot formation.

In another example, a factor Xa inhibitor, preferably Rivaroxaban, more preferably Apixaban released locally in combination with Argatroban from an implant to inhibit fibrin or clot formation.

In another example, a factor Xa inhibitor, preferably Rivaroxaban, more preferably Apixaban released locally in combination with Argatroban from an implant to inhibit fibrin, clot formation, and/or smooth muscle cell proliferation.

In another example, a factor Xa inhibitor, preferably Rivaroxaban, more preferably Apixaban released locally by an implant to inhibit clot formation.

In one example, a device delivery one or more drugs locally, wherein locally comprises delivering said one or more drugs to one or more of site specific location, to a vessel wall, adjacent to a vessel wall, in a body lumen, to a body organ, within a body organ, to the device surface in a body lumen, to a tissue, or to an injured tissue.

In another example, a factor Xa inhibitor, preferably Rivaroxaban, more preferably Apixaban is released locally in combination with an m-TOR inhibitor to inhibit fibrin formation or clot formation, or to inhibit fibrin formation or clot formation through 7 days, or to inhibit fibrin formation or clot formation through 28 days.

In another example, a factor Xa inhibitor, preferably Rivaroxaban, more preferably Apixaban is released locally from a device releasing said drug in combination with an m-TOR inhibitor at a dose of at least 88.9 μg, or a dose of at least 1.2 μg/mm², or a dose of at least 7.14 μg/mm of device length, within 7 days from vessel injury (or from implantation) to inhibit fibrin or clot formation.

In another example, a factor Xa inhibitor, preferably Rivaroxaban, more preferably Apixaban is released locally in combination with an m-TOR inhibitor from a stent at a rate of at least 92.5% within 28 days and a dose of at least 92.5 μg, or a dose of at least 1.1 μg/mm², or a dose of at least 6.6 μg/mm of stent length, within said 28 days after vessel injury (or from implantation) to inhibit clot formation.

In another example, a factor Xa inhibitor, preferably Rivaroxaban, more preferably Apixaban is released locally in combination with an m-TOR inhibitor is released at a rate of at least 88.9% within 7 days and a dose of at least 88.9 μg, or a dose of at least 1.2 μg/mm², or a dose of at least 7.14 μg/mm of stent length, was released within said 7 days from vessel injury (or from implantation), and at a release rate of at least 92.5% within 28 days and a dose of at least 92.5 μg, or a dose of at least 1.1 μg/mm², or a dose of at least 6.6 μg/mm of stent length, is released within said 28 days from vessel injury (or from implantation) to inhibit clot formation.

In another example, a factor Xa inhibitor, preferably Rivaroxaban, more preferably Apixaban is released locally in combination with an m-TOR inhibitor from a stent at a rate of at least 68.1 μg, or at rate of 0.84 μg/mm², or at a rate of at least 4.86 μg/mm of stent length, within 7 days after implantation to inhibit clot formation.

In another example, a factor Xa inhibitor, preferably Rivaroxaban, more preferably Apixaban is released locally in combination with an m-TOR inhibitor wherein tissue concentration of the factor Xa ranges from at least 3.96 ng/mg of tissue adjacent to the stented segment to at least 15 ng/mg of tissue adjacent to the stented segment, within or at 7 days, or within or 28 days from implant (or tissue injury)

In one example, Argatroban in combination with Rivaroxaban or Apixaban are configured to be released from a device locally in a body lumen wherein said drugs have the same or different dose and wherein the Argatroban is configured to be released at a rate ranging from 70% to 99% within 3 hours, 3 days, or within 7 days, preferably configured to be released at a rate ranging from 80% to 99% within said 3 hours, 3 days, or within 7 day period, while the Rivaroxaban or Apixaban are configured to be released at a rate ranging from 50% to 99% within 3 hours, 3 days, or within 7 day period, preferably released at a rate ranging from 60% to 99% within 3 hours, 3 days, or within 7 days. In another example, Argatroban in combination with Rivaroxaban or Apixaban are configured to be released from a device locally in a body lumen wherein said drugs each have a dose ranging from 50 μg to 500 μg, or each has a dose ranging from 1.1 μg/mm² to 10 μg/mm², or each has a dose ranging from 3 μg/mm to 30 μg/mm of stent length, and wherein the Argatroban is configured to be released at a rate ranging from 70% to 99% within 3 hours, 3 days, or within 7 days, preferably configured to be released at a rate ranging from 80% to 99% within said 3 hours, 3 days, or within 7 day period, while the Rivaroxaban or Apixaban are configured to be released at a rate ranging from 50% to 99% within 3 hours, 3 days, or within 7 day period, preferably released at a rate ranging from 60% to 99% within 3 hours, 3 days, or within 7 days. The two drugs may be configured to release at same or similar release rate or different release rates, the two agents may have the same dose or different dose. In another example, a third antiproliferative drug is configured to be released from the device in combination with Argatroban and Rivaroxaban or Apixaban, at similar dose and release rate or different dose and release rate. In a specific example, the anti-proliferative drug is Sirolimus or its analogs (including deuterated analog), metabolites, or salts.

In one example, a device delivery one or more drugs locally, wherein locally comprises delivery of said one or more drugs to one or more of site specific location, adjacent to a vessel wall, to a vessel wall, in a body lumen, to the device surface in a body lumen, to a tissue, to an injured tissue, wherein the local concentration of the one or more dugs maybe higher than in the systemic concentration of the one or more drugs.

In a preferred example, a device releasing factor Xa inhibitor in a body lumen wherein said device inhibits fibrin formation thereby inhibiting clot formation.

In another unexpected finding that the combination of factor Xa inhibitor Apixaban and Argatroban combination was shown to enhance the SMC proliferation inhibition when released together with m-TOR inhibitor, Sirolimus. Further finding showed Apixaban or Rivaroxaban and Argatroban combination had synergistic effects, anti-fibrin formation, or anti clot formation effects that was better than either alone.

In an unexpected finding, composition comprising of a factor Xa inhibitor Apixaban, factor IIa inhibitor Argatroban, and the M-Tor inhibitor Sirolimus exhibited more efficacy at inhibiting one or more of the following at 28 days and/or 90 day time points: cell proliferation, inflammation, injury, fibrin formation inhibition, clot formation, and fibrin dissolution acceleration.

The composition comprising a combination of factor Xa inhibitor Apixaban, a factor II inhibitor Argatroban and an anti-proliferative (M-tor) were surprisingly more effective than an anti-proliferative (M-tor) alone.

It was surprisingly found extended release formulation comprising a factor IIa inhibitor and/or a factor Xa inhibitor, inhibited one or more of clot formation, SMC proliferation, inflammation, and injury, wherein the extended release of the one or more drugs extended beyond 7 days, extended beyond 14 days, extended beyond 21 days, extended beyond 28 days, or extended beyond 3 months.

In another example, a device for use in a body lumen wherein said device is configured to release locally an effective dose of factor Xa inhibitor, preferably Rivaroxaban, more preferably Apixaban, wherein said dose is sufficient to inhibit one or more of thrombin formation, fibrin formation, and clot formation. In another example the device is configured to release in addition to the factor Xa inhibitor, release a factor IIa inhibitor, preferably Argatroban, wherein said dose are sufficient to inhibit one or more of thrombin formation, fibrin formation, and clot formation. In another example the device is configured to release in addition to the factor Xa inhibitor, release a factor IIa inhibitor, preferably Argatroban, wherein said dose are sufficient to inhibit one or more of thrombin formation, fibrin formation, clot formation, and smooth muscle cell proliferation. In another example the device is configured to release in addition to the factor Xa inhibitor, release a factor IIa inhibitor, preferably Argatroban, and in addition release of an anti-proliferative, preferably Sirolimus, analogs (including deuterated analogs), metabolites, or salts, wherein said dose are sufficient to inhibit one or more of thrombin formation, fibrin formation, clot formation, and smooth muscle cell proliferation.

In one example, a device comprising a stent wherein said stent being expandable from a crimped configuration to an expanded configuration, wherein said stent comprises one or more expandable circumferential rings wherein adjacent rings are joined (or connected by one or more links), wherein said one or more rings comprise struts joined by crown. In one example, the stent is balloon expandable. In another example, the stent is self-expandable. In yet another example, the stent is non degradable. In another example, the stent is degradable. In yet another example, the stent is metallic, polymeric, or a hybrid of both. In yet another example, the stent is formed from a shape memory alloy such as nitinol. In another example, the stent is formed from a tubular body, from a sheet, from 3D printing, or a bent wire. In another example, the stent has a helical back bone. In one example, the stent is coated with one or more coating. In another example, the coating comprises one or more polymeric material. In yet another example, the polymeric material is coated as a matrix formed by mixing said one or more factor Xa inhibitors drugs, the said polymeric material, and one or more solvents, and spraying said mixture onto one or more stent surfaces, preferably spraying said mixture onto all stent surfaces. In yet some examples, the mixture is sprayed onto one or more surfaces such as abluminal surface of the stent or luminal surface of the stent. In yet another example, a polymeric material is coated as a top layer or coat wherein the drug is coated onto the stent first and then a top layer or coat is coated on top of said drug to control release of said drug. Alternatively, or in addition, a top layer or coat may be coated on top of a drug/polymer matrix to control release of the drug. In yet another example, the drug is coated directly onto one or more surfaces of the stent. In another example, the drug is contained in stent material. In yet another example, the drug is contained in a reservoir on or in the stent. In yet another example, the drug and/or coating are applied to the stent by spraying, dipping, printing, or other methods known in the art. In one example the one or more factor Xa inhibitors are released in combination with one or more factor IIa inhibitor agents. In another example the one or more factor Xa inhibitors are released in combination with one or more factor IIa inhibitor agents and in combination with one or more antiproliferative agents.

In another example, a device comprising a catheter wherein said catheter comprises an expandable member at a distal segment of the catheter, wherein said expandable member outer surface comprises one or more factor Xa inhibitor drugs. In one example, the one or more drugs are contained in a drug polymer matrix. In another example, the one or more drugs are contained under a top layer or coat. In yet another example, the one or more drugs are contained within a polymer, a microsphere, a nanosphere, a carrier, an excipient, a hydrogel, or other. In yet another example, the drug is contained inside the expandable member and is released through holes or other means through the expandable member. In a preferred example, the expandable member is an expandable balloon. The one or more drugs in this example are released by or more means comprising friction when the expandable member is expanded against a vessel wall or tissue, diffusion gradient, creation of a reservoir at the vessel wall or tissue site, release of the drug, or other. In one example the one or more factor Xa inhibitors are released in combination with one or more factor IIa inhibitor agents. In another example the one or more factor Xa inhibitors are released in combination with one or more factor IIa inhibitor agents, and in combination with one or more antiproliferative agents.

In another example, a device comprising a catheter wherein said catheter comprises holes in the distal segment of the catheter, preferably on the abluminal surface of the catheter, wherein one or more factor Xa inhibitor drugs are released or injected through said holes. In another example, the one or more factor Xa inhibitors are released or injected through the distal end of the catheter. In yet another example, the catheter comprises two or more expandable members to prevent the one or more drugs from escaping into the systemic circulation, and wherein the one or more agents are released or injected in the space between said two or more expandable members. In one example the one or more factor Xa inhibitors are released in combination with one or more factor IIa inhibitor agents. In another example the one or more factor Xa inhibitors are released in combination with one or more factor IIa inhibitor agents, and in combination with one or more antiproliferative agents.

In another example, a device comprising an implant configured to be implanted in a body lumen wherein said body lumen comprises one or more of a vessel, duct, foramen, heart, heart valve, atrium, ventricle, aorta, or other, wherein said implant is configured to release one or more factor Xa inhibitors, and optionally in combination with one or more factor IIa inhibitors. The one or more drugs are coated onto the device surface, within or onto a sleeve covering one or more of one surface, all surfaces, part of the devices or all of the device surfaces, or in a reservoir on or in the device. In one example the one or more factor Xa inhibitors are released in combination with one or more factor IIa inhibitor agents. In another example the one or more factor Xa inhibitors are released in combination with one or more factor IIa inhibitor agents, and in combination with one or more antiproliferative agents.

In another example, a factor IIa inhibitor, preferably Argatroban, released locally from a device, wherein said agent is released at a rate, concentrations, dose, duration, as any of the examples given in this application. In a preferred example, Argatroban is released from a device over a period ranging from 28 days to 1 year, preferably over a period ranging from 90 days to one year. In yet another preferred example, Argatroban is released from a device wherein Argatroban is contained in a therapeutic composition and wherein the therapeutic composition comprises a first fast release rate and a second slower release rate.

In yet another example, a factor Xa inhibitor, preferably Apixaban or Rivaroxaban, released locally from a device, wherein said agent is released at a rate, concentrations, dose, duration, as any of the examples given in this application. In a preferred example, Argatroban is released from a device over a period ranging from 28 days to 1 year, preferably over a period ranging from 90 days to one year.

In yet another preferred example, Argatroban is released from a device wherein Argatroban is contained in a therapeutic composition and wherein the therapeutic composition comprises a first fast release rate and a second slower release rate.

The present disclosure is described in relation to treating pulmonary disease or conditions (e.g., inflammatory pulmonary disease) in a patient via pulmonary delivery of a therapeutic composition. However, one of skill in the art will appreciate that this is not intended to be limiting and the devices and methods disclosed herein may be used in other anatomical areas and in other therapeutic procedures.

As used herein, the term coagulation comprises one or more of thrombin formation, fibrin formation, platelet activation, platelet aggregation, and/or thrombus/clot formation. Coagulation typically arises in response to a body part injury and/or to a foreign body such as a device, and/or an infection from a virus or bacteria, and/or caused by irritants.

As used herein, the term anti-coagulant refers to an agent that inhibits one or more of thrombin formation, fibrin formation, platelet activation (typically indirectly), platelet aggregation, thrombus (clot) formation, thrombin dissolution, fibrin dissolution, or thrombus dissolution, thereby inhibiting one or more of blockage of a lumen or vessel partially or fully, formation of clot, and/or adverse clinical events.

The terms anti-thrombin, thrombin inhibiter, and thrombin formation inhibitor are used interchangeably herein. Also, the terms anti-fibrin, fibrin inhibitor, and fibrin formation inhibitor are used interchangeably herein.

The terms therapeutically active substance and pharmaceutical agent are used interchangeably herein and refer to any bioactive agent. It will be understood by one of ordinary skill in the art that the devices and methods described herein may be used in combination with one or more additional bioactive agents. Such substances and/or agents optionally include anti-coagulants, anti-mitotic agents, anti-proliferative agents, cytostatic agents, anti-migratory agents, anti-fibrotic agents, immunomodulators, immunosuppressants, anti-inflammatory agents, anti-ischemia agents, anti-hypertensive agents, vasodilators, anti-hyperlipidemia agents, anti-diabetic agents, metformin, anti-cancer agents, anti-tumor agents, anti-angiogenic agents, angiogenic agents, anti-chemokine agents, healing-promoting agents, anti-viral agents, anti-bacterial agents, anti-fungal agents, steroids, interferons, and combinations thereof. It is understood that a bioactive agent may exert more than one biological effect.

As used herein, a direct factor Xa inhibitor refers to a direct, selective inhibitor of factor Xa that acts directly on factor Xa without using antithrombin as a mediator. The term “direct factor Xa inhibitor” is used herein interchangeably with the term “factor Xa inhibitor” or “anti-factor Xa”. Direct factor Xa inhibitors inhibit thrombin formation and/or fibrin formation, thereby inhibiting clot formation. Direct factor Xa inhibitors include, but are not limited to, Apixaban, betrixaban, edoxaban, otamixaban, razaxaban, Rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), or 2-[(7-carbamimidoylnaphthalen-2-yl)methyl-[4-(1-ethanimidoylpiperidin-4-yl)oxyphenyl]sulfamoyl]acetic acid (YM-466 or YM-60828), and eribaxaban (PD 0348292), or 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), (S)-3-(7-carbamimidoylnaphthalen-2-yl)-2-(4-(((S)-1-(1-iminoethyl)pyrrolidin-3-yl)oxy)phenyl)propanoic acid hydrochloride pentahydrate (DX9065a), letaxaban (TAK-442), GW813893, JTV-803, KFA-144, DPC-423, RPR-209685, MCM-09, and antistasin, and salt, isomer, solvate, analog, derivative, metabolite, or prodrugs thereof. Preferred direct Xa inhibitors include Apixaban and Rivaroxaban.

As used herein, a direct factor IIa inhibitor refers to a direct, selective inhibitor of factor IIa (also referred to herein as thrombin) which acts directly on factor IIa/thrombin. The term “direct factor IIa inhibitor” is used herein interchangeably with the term “factor IIa inhibitor” or “anti-factor IIa”. Direct factor IIa inhibitors inhibit thrombin formation and/or fibrin formation, thereby inhibiting clot formation. Direct thrombin/factor IIa inhibitors include, but are not limited to, Argatroban, dabigatran, ximelagatran, melagatran, efegatran, inogatran, atecegatran metoxil (AZD-0837), hirudin, hirudin analogs, bivalirudin, desirudin, and lepirudin, and salt, isomer, solvate, analog, derivative, metabolite, or prodrugs thereof. Preferred direct factor IIa inhibitors include Argatroban.

As used herein, an anti-fibrotic agent refers to an agent which acts to reduce or eliminate tissue fibrosis. Anti-fibrotic agents include, but are not limited to nintedanib, pirfenidone, and salt, isomer, solvate, analog, derivative, metabolite, or prodrugs thereof.

Described herein are devices, compositions, and methods for inhibiting clot formation, fibrin, fibrosis, and/or inflammation of pulmonary disease or condition in a patient. A therapeutic composition may be provided as described herein. A therapeutically effective dose of the therapeutic composition may be delivered to the site of the disease or condition in the patient's lung(s). The therapeutically effective dose of the therapeutic composition may be effective to suppress or prevent initiation, progression, or relapses of disease, including the progression of established disease. In some examples, the pulmonary disease or condition may be caused by a viral infection, bacterial infection, smoke inhalation, chemical exposure, genetic mutation, injury, smog and other air pollutions, inhalation of environmental and occupational pollutants, work-related lung diseases, hypersensitivity pneumonitis, and combinations thereof. For example, the pulmonary disease or condition may be pneumonia, bronchitis, emphysema, asthma, lung cancer, pulmonary edema, pulmonary embolism, pulmonary fibrosis, other diseases that involve connective lungs tissue, such as sarcoidosis and granulomatosis with polyangiitis, chronic obstructive pulmonary disease, bronchiectasis, acute respiratory distress syndrome (ARDS), SARS, or COVID-19. In some examples, the patient may be diagnosed as having the pulmonary disease prior to delivering the therapeutically effective dose of the therapeutic composition.

Inflammation, fibrosis, and/or clot formation of the lung tissue caused by the pulmonary disease or condition may lead to fluid build-up, fibrosis, and/or fibrin formation in the lungs, which can damage tissue in the lungs. Lung tissues include, but are not limited to, the alveolar sac where blood oxygenation takes place. Fibrin/clot formation, fibrosis, and/or inflammation in the lungs and airway ventilation can reduce surfactant activity in the lungs, thereby limiting lung function, and can lead to other morbidities and mortality.

In some examples, the therapeutic composition is locally delivered to the lungs. In some examples, the therapeutic composition is delivered to lung tissue including the lower part of the lung and/or the alveolar tissue. The therapeutic composition may be locally delivered to the lungs by inhalation, ventilation, instillation, ultrasounic delivery, vibration, injection, or the like. The therapeutic composition may be delivered to the lungs by any method or device for pulmonary delivery. For example, the therapeutic composition may be delivered with an inhaler (e.g., metered dose inhalers or dry powder inhalers), a ventilator, a nebulizer (e.g., jet nebulizers, ultrasonic nebulizers, or vibrating mesh nebulizers), syringe, catheter, or the like.

In some examples, the therapeutic composition may be delivered to the lungs by one or more methods or devices. For example, a first portion (e.g., one or more bioactive agents) of the therapeutic composition may be delivered to the lungs with a first device (e.g., inhaler) and a second portion (e.g., one or more additional pharmaceutical agents) of the therapeutic composition may be delivered to the lungs with a second device (e.g., inhaler) before or after delivery of the first portion of the therapeutic composition. Alternatively, all components (e.g., all bioactive agents) of the therapeutic composition may be delivered with the same device (e.g., inhaler). It will be understood by one of ordinary skill in the art that the therapeutic composition may comprise one or many components which may be delivered simultaneously or sequentially in any order or combination as desired with any combination of methods or devices desired.

In some examples, local delivery of the therapeutic composition may be preferable to systemic delivery of the therapeutic composition in at least some instances. For example, local delivery of the therapeutic composition may reduce off-target effects by reducing systemic concentrations and/or increase the tissue concentration of the therapeutic composition at the target site compared to what can be achieved with systemic delivery safety maximums.

In some examples, the therapeutic composition may comprise one or more of a pharmaceutically acceptable carrier, a propellant, a blowing agent, an excipient, a surfactant, a binding agent, an adjuvant agent, a flavoring agent or taste masking agent, a coloring agent, an emulsifying agent, a stabilizing agent, an isotonic agent, and targeting co-molecules.

In some examples, the therapeutic composition may be atomized, nebulized, aerosolized, pressurized, micronized, nanosized, in the form of a dry powder, or combinations thereof.

In some examples, the therapeutic composition may comprise or be co-administered with one or more additional bioactive agents which address or treat the underlying disease or infection in order to improve therapeutic outcome.

While many of the examples described herein depict the therapeutic composition being atomized, nebulized, aerosolized, micronized, in the form of a dry powder, or combinations thereof for local delivery, it will be understood by one of ordinary skill in the art that local delivery may be achieved through any other means desired. For examples, the therapeutic composition may be coated, dipped, printed, deposited, painted, brushed, loaded, or otherwise disposed on one or more surfaces of a device for local delivery. In some examples, the therapeutic composition may be locally delivered via direct injection using a device (e.g., a catheter, a needle, etc.) comprising or coupled to a drug reservoir.

It is an objective of this application to show that pulmonary delivery of the direct factor Xa inhibitor and/or additional therapeutically active substance (e.g., an anti-coagulant such as a direct factor IIa inhibitor), and optionally in combination with one or more additional pharmaceutical agent, inhibits one or more of inflammation, thrombin formation, fibrin formation, clot formation, smooth muscle proliferation, fibrosis, viral replication, bacterial replication, and/or adverse clinical events.

It is an objective of this application to show that pulmonary delivery of a pharmaceutical agent to inhibits one or more of inflammation, fibrosis, viral replication, bacterial replication, and/or adverse clinical events.

Described herein are devices and methods for locally delivering a therapeutic composition to a patient. The therapeutic composition includes one or more anti-coagulant agents which inhibit thrombin, fibrin, and/or thrombus formation or promote thrombin, fibrin, and/or thrombus dissolution. In preferred examples, the therapeutic composition includes a direct Xa inhibitor. In another preferred example, the therapeutic composition includes a direct Xa inhibitor and/or a direct IIa inhibitor. In another preferred example, one or more additional pharmaceutical agents may be added to the therapeutic composition of the direct Xa inhibitor and/or the direct IIa inhibitor. The additional pharmaceutical agents may, for example, include one or more anti-fibrotic agents, metformin, steroids, interferons, or combinations thereof.

In some examples, the pharmaceutical agents may, for example, include one or more anti-fibrotic agents, metformin, metformin hydroxychloride (HCl), steroids, interferons, or combinations thereof.

In some examples, the pharmaceutical agent may, for example, include one or more anti-viral/anti-diabetic agents such as metformin or its salt metformin HCl. Metformin was originally introduced as an anti-influenza drug and was found to have glucose-lowering side effects, making it effective as both an anti-viral and an anti-diabetic agent. Metformin activates AMP-activated protein kinase (AMPK) which, among other things, phosphorylates and upregulates angiotensin-converting enzyme 2 (ACE2) and inhibits the mTOR signaling cascade. The anti-viral activity of metformin may, in at least some instances, be related to these activities. For example, SARS-CoV-2, which causes COVID-19, uses ACE2 as its receptor and phosphorylation thereof could prevent viral binding and infection. Additionally, once SARS-CoV-2 is inside the cell, ACE2 is downregulated, resulting in an imbalance in the renin-angiotensin-aldosterone system (RAS) which promotes pro-inflammatory and pro-fibrotic activity in the lungs. ACE2 upregulation by metformin could potentially reverse or prevent these effects. Additionally, the PI3K/AKT/mTOR pathway plays a major role in influenza and MERS CoV infection and inhibition of mTOR/AKT by metformin could reduce pathogenesis. Similarly, mTOR inhibition could be effective at preventing or reducing pathogenesis of SARS-CoV-2.

In some examples, the therapeutic composition comprises a direct factor Xa inhibitor, a additional therapeutically active substance (e.g., an anti-coagulant), and/or an additional pharmaceutical agent (e.g., an anti-fibrotic agent, an anti-viral agent, an anti-diabetic agent, and/or an anti-proliferative agent).

In some examples, the therapeutic composition comprises a direct factor Xa inhibitor, a direct factor IIa inhibitor, and/or an anti-fibrotic agent. In some examples, the therapeutic composition may comprise a direct factor Xa inhibitor, a direct factor IIa inhibitor, and an anti-fibrotic agent. In some examples, the therapeutic composition may comprise a direct factor Xa inhibitor and a direct factor IIa inhibitor but no anti-fibrotic agent. In some examples, the therapeutic composition may comprise a direct factor Xa inhibitor and an anti-fibrotic agent but no direct factor IIa inhibitor. In some examples, the therapeutic composition may comprise a direct factor Xa inhibitor but no direct factor IIa inhibitor or anti-fibrotic agent.

In some examples, the direct factor Xa inhibitor, direct factor IIa inhibitor, and/or anti-fibrotic agent may be delivered simultaneously or sequentially in any order or combination as desired with any combination of methods or devices desired. For example, the same inhaler may be used to deliver the direct factor Xa inhibitor, the direct factor IIa inhibitor, and the anti-fibrotic agent. Alternatively, a first inhaler may be used to deliver the direct factor Xa inhibitor and a second inhaler may be used to deliver the direct factor IIa inhibitor and the anti-fibrotic agent. Alternatively, a first inhaler may be used to deliver the direct factor Xa inhibitor and the direct factor IIa inhibitor and a second inhaler may be used to deliver the anti-fibrotic agent. Alternatively, each agent may be delivered by its own dedicated inhaler.

In some examples, the therapeutic composition comprises a direct factor Xa inhibitor, a direct factor IIa inhibitor, and/or an anti-viral/anti-diabetic agent. In some examples, the therapeutic composition may comprise a direct factor Xa inhibitor, a direct factor IIa inhibitor, and anti-viral/anti-diabetic agent. In some examples, the therapeutic composition may comprise a direct factor Xa inhibitor and a direct factor IIa inhibitor but no anti-viral/anti-diabetic agent. In some examples, the therapeutic composition may comprise a direct factor Xa inhibitor and an anti-viral/anti-diabetic agent but no direct factor IIa inhibitor. In some examples, the therapeutic composition may comprise a direct factor Xa inhibitor but no direct factor IIa inhibitor or anti-viral/anti-diabetic agent.

In some examples, the direct factor Xa inhibitor, direct factor IIa inhibitor, and/or anti-viral/anti-diabetic agent may be delivered simultaneously or sequentially in any order or combination as desired with any combination of methods or devices desired. For example, the same inhaler may be used to deliver the direct factor Xa inhibitor, the direct factor IIa inhibitor, and the anti-viral/anti-diabetic agent. Alternatively, a first inhaler may be used to deliver the direct factor Xa inhibitor and a second inhaler may be used to deliver the direct factor IIa inhibitor and the anti-viral/anti-diabetic agent. Alternatively, a first inhaler may be used to deliver the direct factor Xa inhibitor and the direct factor IIa inhibitor and a second inhaler may be used to deliver the anti-viral/anti-diabetic agent. Alternatively, each agent may be delivered by its own dedicated inhaler.

In some examples, the therapeutic composition comprises a direct factor Xa inhibitor, a direct factor IIa inhibitor, and/or an anti-proliferative agent. In some examples, the therapeutic composition may comprise a direct factor Xa inhibitor, a direct factor IIa inhibitor, and an anti-proliferative agent. In some examples, the therapeutic composition may comprise a direct factor Xa inhibitor and a direct factor IIa inhibitor but no anti-proliferative agent. In some examples, the therapeutic composition may comprise a direct factor Xa inhibitor and an anti-proliferative agent but no direct factor IIa inhibitor. In some examples, the therapeutic composition may comprise a direct factor Xa inhibitor but no direct factor IIa inhibitor or anti-proliferative agent.

In some examples, the direct factor Xa inhibitor, direct factor IIa inhibitor, and/or anti-proliferative agent may be delivered simultaneously or sequentially in any order or combination as desired with any combination of methods or devices desired. For example, the same inhaler may be used to deliver the direct factor Xa inhibitor, the direct factor IIa inhibitor, and the anti-proliferative agent. Alternatively, a first inhaler may be used to deliver the direct factor Xa inhibitor and a second inhaler may be used to deliver the direct factor IIa inhibitor and the anti-proliferative agent. Alternatively, a first inhaler may be used to deliver the direct factor Xa inhibitor and the direct factor IIa inhibitor and a second inhaler may be used to deliver the anti-proliferative agent. Alternatively, each agent may be delivered by its own dedicated inhaler.

When the therapeutic composition comprises a direct factor Xa inhibitor and a direct factor IIa inhibitor, the weight compositional ratio of the direct factor Xa inhibitor to the direct factor IIa inhibitor in the therapeutic composition may be within a range of about 3:1 to about 1:3. For example, the weight compositional ratio of the direct factor Xa inhibitor to the direct factor IIa inhibitor in the therapeutic composition may be about 1:1.

In some examples, the direct factor Xa inhibitor may have an inhibition potency for factor Xa ranging from about 0.001 nM to about 50 nM.

In some examples, the direct factor IIa inhibitor may have an inhibition potency for factor IIa ranging from about 0.001 nM to about 100 nM.

In some examples, the direct factor IIa inhibitor may comprise Argatroban, or a salt, isomer, solvate, analog (including deuterated analog), derivative, metabolite, or prodrugs thereof.

In some examples, the direct factor Xa inhibitor may comprise Apixaban, or a salt, isomer, solvate, analog (including deuterated analog), derivative, metabolite, or prodrugs thereof.

In some examples, the direct factor Xa inhibitor may comprise Rivaroxaban, or a salt, isomer, solvate, analog (including deuterated analog), derivative, metabolite, or prodrugs thereof.

In some examples, the anti-fibrotic agent may comprise nintedanib, or a salt, isomer, solvate, analog (including deuterated analog), derivative, metabolite, or prodrugs thereof.

In some examples, the anti-fibrotic agent may comprise pirfenidone, or a salt, isomer, solvate, analog (including deuterated analog), derivative, metabolite, or prodrugs thereof.

In some examples, the anti-viral/anti-diabetic agent may comprise metformin, or a salt, isomer, solvate, analog (including deuterated analog), derivative, metabolite, or prodrugs thereof.

In some examples, the anti-proliferative agent may comprise Sirolimus, or a salt, isomer, solvate, analog (including deuterated analog), derivative, metabolite, or prodrugs thereof.

In some examples, the anti-proliferative agent may comprise novolimus, or a salt, isomer, solvate, analog (including deuterated analog), derivative, metabolite, or prodrugs thereof.

In some examples, the direct factor Xa inhibitor may comprise Apixaban and the direct factor IIa inhibitor may comprise Argatroban.

In some examples, the direct factor Xa inhibitor may comprise Rivaroxaban and the direct factor IIa inhibitor may comprise Argatroban.

In some examples, the direct factor Xa inhibitor may comprise Apixaban, the direct factor IIa inhibitor may comprise Argatroban, and the anti-fibrotic agent may comprise nintedanib.

In some examples, the direct factor Xa inhibitor may comprise Apixaban, the direct factor IIa inhibitor may comprise Argatroban, and the anti-fibrotic agent may comprise pirfenidone.

In some examples, the direct factor Xa inhibitor may comprise Rivaroxaban, the direct factor IIa inhibitor may comprise Argatroban, and the anti-fibrotic agent may comprise nintedanib.

In some examples, the direct factor Xa inhibitor may comprise Rivaroxaban, the direct factor IIa inhibitor may comprise Argatroban, and the anti-fibrotic agent may comprise pirfenidone.

In some examples, the direct factor Xa inhibitor may comprise Apixaban, the direct factor IIa inhibitor may comprise Argatroban, and the anti-diabetic agent may comprise metformin or its salt.

In some examples, the direct factor Xa inhibitor may comprise Rivaroxaban, the direct factor IIa inhibitor may comprise Argatroban, and the anti-diabetic agent may comprise metformin or its salt.

In some examples, the direct factor Xa inhibitor may comprise Apixaban, the direct factor IIa inhibitor may comprise Argatroban, and the anti-proliferative agent may comprise Sirolimus or its salt.

In some examples, the direct factor Xa inhibitor may comprise Apixaban, the direct factor IIa inhibitor may comprise Argatroban, and the anti-proliferative agent may comprise novolimus or its salt.

In some examples, the direct factor Xa inhibitor may comprise Rivaroxaban, the direct factor IIa inhibitor may comprise Argatroban, and the anti-proliferative agent may comprise Sirolimus or its salt.

In some examples, the direct factor Xa inhibitor may comprise Rivaroxaban, the direct factor IIa inhibitor may comprise Argatroban, and the anti-proliferative agent may comprise novolimus or its salt.

In some examples, the direct factor Xa inhibitor is present in the therapeutic composition at a concentration within a range of about 0.1 nM to about 1,000,000 nM. In some examples, the direct factor Xa inhibitor may present in the therapeutic composition at a concentration within a range of about 1 nM to about 1,000,000 nM. In some examples, the direct factor Xa inhibitor may present in the therapeutic composition at a concentration within a range of about 10 nM to about 1,000,000 nM. In some examples, the direct factor Xa inhibitor may present in the therapeutic composition at a concentration within a range of about 100 nM to about 1,000,000 nM.

In some examples, the direct factor IIa inhibitor is present in the therapeutic composition at a concentration within a range of about 0.1 nM to about 1,000,000 nM. In some examples, the direct factor IIa inhibitor may present in the therapeutic composition at a concentration within a range of about 1 nM to about 1,000,000 nM. In some examples, the direct factor IIa inhibitor may present in the therapeutic composition at a concentration within a range of about 10 nM to about 1,000,000 nM. In some examples, the direct factor IIa inhibitor may present in the therapeutic composition at a concentration within a range of about 100 nM to about 1,000,000 nM.

In some examples, the anti-fibrotic agent comprises pirfenidone and is present in the therapeutic composition at a concentration within a range of about 1,000 nM to about 10,000,000 nM. In some examples, the anti-fibrotic agent may present in the therapeutic composition at a concentration within a range of about 10,000 nM to about 10,000,000 nM. In some examples, the anti-fibrotic agent may present in the therapeutic composition at a concentration within a range of about 100,000 nM to about 10,000,000 nM.

In some examples, the anti-fibrotic agent comprises nintedanib and is present in the therapeutic composition at a concentration within a range of about 0.1 nM to about 1,000,000 nM. In some examples, the anti-fibrotic agent may present in the therapeutic composition at a concentration within a range of about 1 nM to about 1,000,000 nM. In some examples, the anti-fibrotic agent may present in the therapeutic composition at a concentration within a range of about 10 nM to about 1,000,000 nM. In some examples, the anti-fibrotic agent may present in the therapeutic composition at a concentration within a range of about 100 nM to about 1,000,000 nM.

In some examples, the anti-viral/anti-diabetic agent comprises metformin or its salt and is present in the therapeutic composition at a concentration within a range of about 1,000 nM to about 100,000,000 nM. In some examples, the anti-viral/anti-diabetic agent may present in the therapeutic composition at a concentration within a range of about 100,000 nM to about 100,000,000 nM. In some examples, the anti-viral/anti-diabetic agent may present in the therapeutic composition at a concentration within a range of about 1,000,000 nM to about 100,000,000 nM.

In some examples, the direct factor Xa inhibitor is present in the therapeutic composition at a concentration effective to achieve a tissue concentration of the direct factor Xa inhibitor of about 0.1 ng/mg tissue to about 10 ng/mg tissue when a therapeutically effective dose of the therapeutic composition is delivered to the tissue. In some examples, the therapeutically effective dose may be sufficient to generate a tissue concentration of the direct factor Xa inhibitor of about 0.2 ng/mg tissue to about 10 ng/mg tissue. In some examples, the therapeutically effective dose may be sufficient to generate a tissue concentration of the direct factor Xa inhibitor of about 0.5 ng/mg tissue to about 5 ng/mg tissue. In some examples, the therapeutically effective dose may be sufficient to generate a tissue concentration of the direct factor Xa inhibitor of about 1 ng/mg tissue to about 5 ng/mg tissue.

In some examples, the direct factor IIa inhibitor is present in the therapeutic composition at a concentration effective to achieve a tissue concentration of the direct factor IIa inhibitor of about 0.1 ng/mg tissue to about 10 ng/mg tissue when a therapeutically effective dose of the therapeutic composition is delivered to the tissue. In some examples, the therapeutically effective dose may be sufficient to generate a tissue concentration of the direct factor IIa inhibitor of about 0.2 ng/mg tissue to about 10 ng/mg tissue. In some examples, the therapeutically effective dose may be sufficient to generate a tissue concentration of the direct factor IIa inhibitor of about 0.5 ng/mg tissue to about 5 ng/mg tissue. In some examples, the therapeutically effective dose may be sufficient to generate a tissue concentration of the direct factor IIa inhibitor of about 1 ng/mg tissue to about 5 ng/mg tissue.

In some examples, the anti-fibrotic agent comprises nintedanib and is present in the therapeutic composition at a concentration effective to achieve a tissue concentration of the anti-fibrotic agent of about 0.03 ng/mg tissue to about 5 ng/mg tissue when a therapeutically effective dose of the therapeutic composition is delivered to the tissue. In some examples, the therapeutically effective dose may be sufficient to generate a tissue concentration of the anti-fibrotic agent of about 0.05 ng/mg tissue to about 5 ng/mg tissue. In some examples, the therapeutically effective dose may be sufficient to generate a tissue concentration of the anti-fibrotic agent of about 0.1 ng/mg tissue to about 5 ng/mg tissue.

In some examples, the anti-fibrotic agent comprises pirfenidone and is present in the therapeutic composition at a concentration effective to achieve a tissue concentration of the anti-fibrotic agent of about 5 ng/mg tissue to about 30 ng/mg tissue when a therapeutically effective dose of the therapeutic composition is delivered to the tissue. In some examples, the therapeutically effective dose may be sufficient to generate a tissue concentration of the anti-fibrotic agent of about 7 ng/mg tissue to about 30 ng/mg tissue. In some examples, the therapeutically effective dose may be sufficient to generate a tissue concentration of the anti-fibrotic agent of about 10 ng/mg tissue to about 30 ng/mg tissue.

In some examples, the anti-viral/anti-diabetic agent comprises metformin or its salt and is present in the therapeutic composition at a concentration effective to achieve a tissue concentration of the anti-viral/anti-diabetic agent of about 1 ng/mg tissue to about 20 ng/mg tissue when a therapeutically effective dose of the therapeutic composition is delivered to the tissue. In some examples, the therapeutically effective dose may be sufficient to generate a tissue concentration of the anti-viral/anti-diabetic agent of about 1.5 ng/mg tissue to about 10 ng/mg tissue. In some examples, the therapeutically effective dose may be sufficient to generate a tissue concentration of the anti-viral/anti-diabetic agent of about 2 ng/mg tissue to about 5 ng/mg tissue.

In some examples, a therapeutically effective dose of the therapeutic composition may be delivered to the target tissue to generate a therapeutically effective concentration in the target tissue. The therapeutic composition may be formulated such that the therapeutically effective concentration may be substantially higher than a systemic blood concentration of each agent in order to treat effectively the disease or condition of the lung. Preferably, the tissue concentration is at least about 1 times a median maximum serum concentration (Cₘₐₓ) blood concentration, at least 1.5 times, 2 times, or 5 times higher than the systemic Cₘₐₓ blood concentration or about 10 times higher than the systemic dose.

In some examples, a therapeutically effective concentration of the therapeutic composition may be delivered to the target tissue. The therapeutic composition may be formulated such that the therapeutically effective concentration may be substantially higher than a systemic therapeutic blood concentration of each agent, preferably higher than a systemic therapeutic blood Cₘₐₓ dose of each agent, while the agent blood Cₘₐₓ (from agent diffusing into the systemic circulation) remains under systemic dose of the agent blood Cₘₐₓ. For example, tissue concentration of the agent Apixaban in the lung tissue (the alveolar sac tissue) may range from 0.1 ng/mg tissue while the blood concentration in the systemic circulation remains below 100 ng/ml of blood.

For example, typical blood concentrations for systemically-delivered Apixaban ranges from about 10 ng/ml to about 40 ng/ml in the blood and the Cₘₐₓ depending on the doses ranges from about 50 ng/ml to about 100 ng/ml (sometimes up to about 200 ng/ml). If we assume density of the tissue (being mostly water) is about 1 gm/ml, these ranges can be converted from ng/ml to ng/mg, with 10 ng/ml being equivalent to about 0.01 ng/mg tissue and 200 ng/ml being equivalent to about 0.2 ng/mg tissue, which means Cₘₐₓ is at or below 0.2 ng/mg of tissue.

In some examples, the therapeutically effective dose in the lung tissue may be sufficient to generate a blood concentration of the direct factor Xa inhibitor which is less than about 200 ng/ml, 100 ng/ml, 50 ng/ml, 40 ng/ml, or 10 ng/ml.

In some examples, the therapeutically effective dose may be sufficient to generate a blood concentration of the direct factor Xa inhibitor which is smaller than a median maximum serum concentration (Cₘₐₓ) of the direct factor Xa inhibitor generated by systemic delivery of the direct factor Xa inhibitor to achieve the same tissue concentration at the site of the inflammatory, fibrotic, and/or clot formation pulmonary disease or condition. In some examples, the therapeutically effective dose may be sufficient to generate a blood concentration of the direct factor Xa inhibitor which does not exceed a median maximum serum concentration (Cₘₐₓ) of the direct factor Xa inhibitor generated by systemic delivery of the direct factor Xa inhibitor to achieve the same tissue concentration at the site of the inflammatory, fibrotic, and/or clot formation pulmonary disease or condition for more than about 6 hours to about 12 hours. In some examples, the systemic delivery comprises a single oral dose, a daily oral dose, or a smallest oral dose of the direct factor Xa inhibitor. In some examples, the Cₘₐₓ is measured using one of plasma blood, serum blood, or whole blood. In other examples, the median Cₘₐₓ is 80 ng/ml, or 123 ng/ml, or 171 ng/ml, or 321 ng/ml, or 480 ng/ml of blood.

In some examples, the therapeutic composition is formulated to release a dose of the direct factor Xa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor Xa inhibitor generated by systemic delivery of the direct factor Xa inhibitor to achieve the same tissue concentration at the injury site. In other examples, the therapeutic composition is formulated to release a dose of the direct factor Xa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor Xa inhibitor generated by systemic delivery of the direct factor Xa inhibitor when taking one or more oral dose of said factor Xa inhibitor. In some examples, the systemic delivery comprises a single oral dose, a daily oral dose, or a smallest oral dose of the direct factor Xa inhibitor. In some examples, the (AUC (0-24) or AUC (0-∞)) is measured using one of plasma blood, serum blood, or whole blood. In other examples, the median (AUC (0-24) or AUC (0-∞)) is 724 ng·h/ml, or1437 ng·h/ml, or 2000 ng·h/ml, or 4000 ng·h/ml.

In some examples, the therapeutically effective dose in lung tissue may be sufficient to generate a blood concentration of the direct factor IIa inhibitor which is less than about 200 ng/ml, 100 ng/ml, 50 ng/ml, 40 ng/ml, or 10 ng/ml.

In some examples, the therapeutically effective dose may be sufficient to generate a blood concentration of the direct factor IIa inhibitor which is smaller than a median maximum serum concentration (Cₘₐₓ) of the direct factor IIa inhibitor generated by systemic delivery of the direct factor IIa inhibitor to achieve the same tissue concentration at the site of the inflammatory, fibrotic, and/or clot formation pulmonary disease or condition. In some examples, the therapeutically effective dose may be sufficient to generate a blood concentration of the direct factor IIa inhibitor which does not exceed a median maximum serum concentration (Cₘₐₓ) of the direct factor IIa inhibitor generated by systemic delivery of the direct factor IIa inhibitor to achieve the same tissue concentration at the site of the inflammatory, fibrotic, and/or clot formation pulmonary disease or condition for more than about 6 hours to about 12 hours. In some examples, the systemic delivery comprises a single oral dose, a daily oral dose, or a smallest oral dose of the direct factor IIa inhibitor. In some examples, the systemic delivery comprises a single oral dose, a daily oral dose, or a smallest oral dose of the direct factor IIa inhibitor. In some examples, the Cₘₐₓ is measured using one of plasma blood, serum blood, or whole blood. In other examples, the median Cₘₐₓ is 80 ng/ml, or 123 ng/ml, or 171 ng/ml, or 321 ng/ml, or 480 ng/ml of blood.

In some examples, the therapeutic composition is formulated to release a dose of the direct factor IIa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor IIa inhibitor generated by systemic delivery of the direct factor IIa inhibitor to achieve the same tissue concentration at the injury site. In other examples, the therapeutic composition is formulated to release a dose of the direct factor IIa inhibitor sufficient to generate a plasma drug level area under the curve (AUC (0-24) or AUC (0-∞)) in ng·h/ml which is smaller than a median (AUC (0-24) or AUC (0-∞)) in ng·h/ml of the direct factor IIa inhibitor generated by systemic delivery of the direct factor IIa inhibitor when taking one or more oral dose of said factor IIa inhibitor. In some examples, the systemic delivery comprises a single oral dose, a daily oral dose, or a smallest oral dose of the direct factor IIa inhibitor. In some examples, the (AUC (0-24) or AUC (0-∞)) is measured using one of plasma blood, serum blood, or whole blood. In other examples, the median (AUC (0-24) or AUC (0-∞)) is 724 ng·h/ml, or 1437 ng·h/ml, or 2000 ng·h/ml, or 4000 ng·h/ml.

In some examples, the therapeutically effective dose in lung tissue may be sufficient to generate a blood concentration of the anti-fibrotic agent comprising nintedanib which is less than about 100 ng/ml, 50 ng/ml, 40 ng/ml, or 30 ng/ml.

In some examples, the therapeutically effective dose may be sufficient to generate a blood concentration of nintedanib which is smaller than a median maximum serum concentration (Cₘₐₓ) of nintedanib generated by systemic delivery of nintedanib to achieve the same tissue concentration at the site of the inflammatory, fibrotic, and/or clot formation pulmonary disease or condition. In some examples, the therapeutically effective dose may be sufficient to generate a blood concentration of nintedanib which does not exceed a median maximum serum concentration (Cₘₐₓ) of nintedanib generated by systemic delivery of nintedanib to achieve the same tissue concentration at the site of the inflammatory, fibrotic, and/or clot formation pulmonary disease or condition for more than about 6 hours to about 12 hours. In some examples, the systemic delivery comprises a single oral dose, a daily oral dose, or a smallest oral dose of nintedanib. In some examples, the systemic delivery comprises a single oral dose, a daily oral dose, or a smallest oral dose of nintedanib. In some examples, the Cₘₐₓ is measured using one of plasma blood, serum blood, or whole blood. In other examples, the median Cₘₐₓ is 31.8 ng/ml or 43.2 ng/ml of blood.

In some examples, the therapeutic composition is formulated to release a dose of nintedanib sufficient to generate a plasma drug level area under the curve (AUC (0-24) or AUC (0-∞)) in ng·h/ml which is smaller than a median (AUC (0-24) or AUC (0-∞)) in ng·h/ml of nintedanib generated by systemic delivery of nintedanib to achieve the same tissue concentration at the injury site. In other examples, the therapeutic composition is formulated to release a dose of nintedanib sufficient to generate a plasma drug level area under the curve (AUC (0-24) or AUC (0-∞)) in ng·h/ml which is smaller than a median (AUC (0-24) or AUC (0-∞)) in ng·h/ml of nintedanib generated by systemic delivery of nintedanib when taking one or more oral dose of nintedanib. In some examples, the systemic delivery comprises a single oral dose, a daily oral dose, or a smallest oral dose of nintedanib. In some examples, the (AUC (0-24) or AUC (0-∞)) is measured using one of plasma blood, serum blood, or whole blood. In other examples, the median (AUC (0-24) or AUC (0-∞)) is 266 ng·h/ml.

In some examples, the therapeutically effective dose in lung tissue may be sufficient to generate a blood concentration of the anti-fibrotic agent comprising pirfenidone which is less than about 20000 ng/ml, 12000 ng/ml, 7000 ng/ml, or 6500 ng/ml.

In some examples, the therapeutically effective dose may be sufficient to generate a blood concentration of pirfenidone which is smaller than a median maximum serum concentration (Cₘₐₓ) of pirfenidone generated by systemic delivery of pirfenidone to achieve the same tissue concentration at the site of the inflammatory, fibrotic, and/or clot formation pulmonary disease or condition. In some examples, the therapeutically effective dose may be sufficient to generate a blood concentration of pirfenidone which does not exceed a median maximum serum concentration (Cₘₐₓ) of pirfenidone generated by systemic delivery of pirfenidone to achieve the same tissue concentration at the site of the inflammatory, fibrotic, and/or clot formation pulmonary disease or condition for more than about 6 hours to about 12 hours. In some examples, the systemic delivery comprises a single oral dose, a daily oral dose, or a smallest oral dose of pirfenidone. In some examples, the systemic delivery comprises a single oral dose, a daily oral dose, or a smallest oral dose of pirfenidone. In some examples, the Cₘₐₓ is measured using one of plasma blood, serum blood, or whole blood. In other examples, the median Cₘₐₓ is 6560 ng/ml, or 7640 ng/ml, or 12300 ng/ml, or 12500 ng/ml, or 12600 ng/ml, or 19800 ng/ml of blood.

In some examples, the therapeutic composition is formulated to release a dose of pirfenidone sufficient to generate a plasma drug level area under the curve (AUC (0-24) or AUC (0-∞)) in ng·h/ml which is smaller than a median (AUC (0-24) or AUC (0-∞)) in ng·h/ml of pirfenidone generated by systemic delivery of pirfenidone to achieve the same tissue concentration at the injury site. In other examples, the therapeutic composition is formulated to release a dose of pirfenidone sufficient to generate a plasma drug level area under the curve (AUC (0-24) or AUC (0-∞)) in ng·h/ml which is smaller than a median (AUC (0-24) or AUC (0-∞)) in ng·h/ml of pirfenidone generated by systemic delivery of pirfenidone when taking one or more oral dose of pirfenidone. In some examples, the systemic delivery comprises a single oral dose, a daily oral dose, or a smallest oral dose of pirfenidone. In some examples, the (AUC (0-24) or AUC (0-∞)) is measured using one of plasma blood, serum blood, or whole blood. In other examples, the median (AUC (0-24) or AUC (0-∞)) is 39800 ng·h/ml, or 40900 ng·h/ml, or 49400 ng·h/ml, or 49700 ng·h/ml, or 55900 ng·h/ml, or 92900 ng·h/ml.

In some examples, the therapeutically effective dose in lung tissue may be sufficient to generate a blood concentration of the anti-viral/anti-diabetic agent comprising metformin which is less than about 1300 ng/ml, 1000 ng/ml, or 800 ng/ml.

In some examples, the therapeutically effective dose may be sufficient to generate a blood concentration of metformin which is smaller than a median maximum serum concentration (Cₘₐₓ) of metformin generated by systemic delivery of metformin to achieve the same tissue concentration at the site of the inflammatory, fibrotic, and/or clot formation pulmonary disease or condition. In some examples, the therapeutically effective dose may be sufficient to generate a blood concentration of metformin which does not exceed a median maximum serum concentration (Cₘₐₓ) of metformin generated by systemic delivery of metformin to achieve the same tissue concentration at the site of the inflammatory, fibrotic, and/or clot formation pulmonary disease or condition for more than about 6 hours to about 12 hours. In some examples, the systemic delivery comprises a single oral dose, a daily oral dose, or a smallest oral dose of metformin. In some examples, the systemic delivery comprises a single oral dose, a daily oral dose, or a smallest oral dose of metformin. In some examples, the Cₘₐₓ is measured using one of plasma blood, serum blood, or whole blood. In other examples, the median Cₘₐₓ is 811.9 ng/ml, or 959.1 ng/ml, or 1301.4 ng/ml of blood.

In some examples, the therapeutic composition is formulated to release a dose of metformin sufficient to generate a plasma drug level area under the curve (AUC (0-24) or AUC (0-∞)) in ng·h/ml which is smaller than a median (AUC (0-24) or AUC (0-∞)) in ng·h/ml of metformin generated by systemic delivery of metformin to achieve the same tissue concentration at the injury site. In other examples, the therapeutic composition is formulated to release a dose of metformin sufficient to generate a plasma drug level area under the curve (AUC (0-24) or AUC (0-∞)) in ng·h/ml which is smaller than a median (AUC (0-24) or AUC (0-∞)) in ng·h/ml of metformin generated by systemic delivery of pirfenidone when taking one or more oral dose of metformin. In some examples, the systemic delivery comprises a single oral dose, a daily oral dose, or a smallest oral dose of metformin. In some examples, the (AUC (0-24) or AUC (0-∞)) is measured using one of plasma blood, serum blood, or whole blood. In other examples, the median (AUC (0-24) or AUC (0-∞)) is 14182 ng·h/ml, or 15260 ng·h/ml, or 15342 ng·h/ml.

In some examples, measurements of blood or tissue described herein comprise one or more of mammalian blood or tissue, porcine blood or tissue, human blood or tissue, rabbit blood or tissue, rat blood or tissue, mouse blood or tissue, or the like.

In some examples, measurements of dose described herein are of human and adjustment to dose to account for total blood and/or fluid in other animal species compared to human may be necessary to equate to human dose.

In some examples, one or more agents in the therapeutic composition may have low solubility. For example, one or more of the direct factor Xa inhibitor, the direct factor IIa inhibitor, the anti-fibrotic agent, the anti-viral/anti-diabetic agent, etc. may have low solubility.

Delivering low solubility compounds to humans by alternative routes often requires the bioactive agent to be formulated in water-based aqueous solutions. Subcutaneous injections and delivery to the lung by inhalation often require aqueous based formulations for the delivery devices, such as syringes and nebulizers. Many low solubility compounds like rapamycin, novolimus, Argatroban, Rivaroxaban, Apixaban (direct factor Xa inhibiting compounds) are formulated in tablets as solids to be taken orally and delivered to patients by the gastrointestinal route.

To be delivered by alternative routes these compounds typically require much higher concentrations of the agent to be solubilized to achieve the correct dosing targets. Therefore, novel formulation techniques that blend in novel proportions of various solubility enhancing agents is required. Excipients such as cyclodextrins, hydroxypropylmethylcellulose (HPMC), polyethylene glycol (PEG) polyvinylpyrrolidone (PVP) can increase poorly soluble drugs 10 to 1000 times normal. This includes blending in the correct proportion of salts and buffering agents such as sodium chloride, citric acid, acetic acid, sodium bicarbonate, sodium phosphate, tromethamine, and other buffering agents, to achieve tonicity similar to human physiology. The excipients are also limited to a group that are generally regarded as safe (GRAS) and must be compatible with safe delivery to the lung.

In the following detailed description, reference is made to the accompanying figures, which form a part hereof. In the figures, similar symbols typically identify similar components, unless context dictates otherwise. The illustrative examples described in the detailed description, figures, and claims are not meant to be limiting. Other examples may be utilized, and other changes may be made, or combined in whole or in part, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain examples and examples are disclosed below, inventive subject matter extends beyond the specifically disclosed examples to other alternative examples and/or uses, and to modifications and equivalents thereof. Thus, the scope of the claims appended hereto is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples, however, the order of description should not be construed to imply that these operations are order dependent. Additionally, the structures, systems, and/or devices described herein may be embodied as integrated components or as separate components.

For purposes of comparing various examples, certain aspects and advantages of these examples are described. Not necessarily all such aspects or advantages are achieved by any particular embodiment. Thus, for example, various examples may be carried out in a manner that achieves or optimizes one advantage or group of advantages as taught herein without necessarily achieving other aspects or advantages as may also be taught or suggested herein.

Every embodiment of the present disclosure may optionally be combined with any one or more of the other examples described herein. Every patent literature, and every non-patent literature, cited herein is incorporated herein by reference in its entirety.

### EXPERIMENTAL EXAMPLES

### Example 1: Preparation of Anticoagulant (Rivaroxaban, Agratroban, and Dalteparin) Eluting Stents

Poly(n-butyl methacrylate) polymer was dissolved into dichloromethane (tetrahydrofuran (THF) was used for Dalteparin) at room temperature and vortexed until the polymer had uniformly dissolved/dispersed. Rivaroxaban was dissolved into dichloromethane at room temperature and vortexed until the drug was uniformly dissolved/dispersed. Argatroban (and Argatroban in combination with Rivaroxaban) was dissolved in Methanol and dichloromethane and vortexed at room temperature until the drug was uniformly dispersed/dissolved. Dalteparin was dissolved in water and THE until fully dissolved.

Each polymer solution and each drug solution were combined together (Rivaroxaban to poly(n-butyl methacrylate) by weight ratio was 6:1), (Argatroban to poly(n-butyl methacrylate) by weight ratio was 3:4), (Dalteparin to poly(n-butyl methacrylate) by weight ratio was 2:3), and (Rivaroxaban in combination with Argatroban to Poly (n-butyl methacrylate) weight ratio was 3:2:2) according to the target drug dose of 150 μg for each drug (and 100 μg each for the Rivaroxaban and Argatroban combination).

A microprocessor-controlled ultrasonic sprayer was used to coat each of the stents' 14 mm length uniformly with each of the drug/polymer matrix solutions. After coating, the stents were placed in a vacuum chamber to remove the solvent. The stents were then mounted on balloon catheters and crimped. The catheters were then inserted in coils and packaged. The pouches were sterilized. The bare metal control stents were the same as the other stents without a drug or polymer coating.

### Example 2: In Vivo Testing of Drug Eluting Stent with Different Drugs

The drug eluting stent systems containing different anticoagulants prepared as described in Example 1 were evaluated at 3 hours, 6 hours, 1 day, 3 days, 6 days, 7 days, or 28 days following implantation in a porcine coronary artery model.

The porcine model was chosen as this model has been used extensively for stent and angioplasty studies resulting in a large volume of data on the pulmonary response properties and its correlation to human pulmonary response (Schwartz et al, Circulation. 2002; 106:1867 1873). The animals were housed and cared for in accordance with the Guide for the Care and Use of Laboratory Animals as established by the National Research Council.

After induction of anesthesia, the left or right femoral artery was accessed using standard techniques and an arterial sheath was introduced and advanced into the artery. Vessel angiography was performed under fluoroscopic guidance, a 7 Fr. guide catheter was inserted through the sheath and advanced to the appropriate location where intracoronary nitroglycerin was administered. An appropriate implantation segment of coronary artery was randomly selected and a 0.014″ guidewire inserted. Quantitative Coronary Angiography (QCA) was performed to document the results. The appropriately-sized stent was advanced to the deployment site. The balloon was inflated at a steady rate to a pressure sufficient to achieve a balloon to artery ratio of approximately 1.1 to 1.0 but less than 1.2:1.

Follow up angiography was performed at the designated timepoint for each of the animals. Late lumen loss (LLL) can be expressed as:

LLL=Post-stent minimum lumen diameter−Final minimum lumen diameter

The LLL is an indicator of the amount smooth muscle cell (SMC) proliferation or inhibition. It is used to measure efficacy between drugs for SMC proliferation inhibition. The smaller the LLL, the better the efficacy of the drug.

Stented portions of coronary arteries were embedded in methyl methacrylate (MMA), then divided into a target of at least three blocks of approximately similar lengths for histology evaluation. Quantitative histopathological evaluation of stented artery sections was then performed and scored as indicated. For Fibrin formation, scores ranged from 0 to 3, with a score of 0 indicating absent or rare minimal spotting around struts of the stent, a score of 1 indicating the presence of fibrin in small amounts localized only around the struts, a score of 2 indicating the moderately abundant or denser presence of fibrin around and extending beyond the struts, and a score of 3 indicating the presence of abundant and dense fibrin and/or bridging of the fibrin between the sruts. The mean score was calculated and reported. The mean of each section was then averaged to provide a mean fibrin score per stent. The smaller the fibrin score, the better efficacy.

The percentage and/or amount of each anticoagulant drug for or by each time point indicated were analyzed for each stent from the different devices in the example and the average drug tissue concentration reported.

Tissue concentrations and the amount of drug released from the stents were measured using stents implanted in porcine arteries for the drugs as indicated. The arteries at the designated time point were excised and a length of stented artery spanning from 5 mm proximal to the stented segment to 5 mm distal to the stented segment was cut. The stented artery was cut longitudinally with surgical scissors. The stents were separated from the tissue. The tissue content of each drug was analyzed using liquid chromatography mass spectroscopy (LCMS) and reported as a mean for each of the timepoint indicated. For drug remaining on each stent, each drug was extracted from the stent, measured using HPLC, and reported as a mean for each of the timepoint indicated as drug released or drug remaining on a stent (where drug remaining is equal to 1000 minus the percentage of drug released).

TABLE 1
Histopathology Scores, Quantitative Coronary Angiography data and PK data of Rivaroxaban, Argatroban,
and Dalteparin (low molecular weight heparin) released from 14 mm stents at day 7.

| | | Cumulative | | | | | Diameter |
|---|---|---|---|---|---|---|---|
| Stent coating | | Percent | Tissue | | | | Stenosis |
| information | Fibrin | Release of | concentration | | | | from |
| (n = 3 for | score at | drug by | at day 7 | LLL at | | | Coronary |
| each arm) | day 7 | day 7 | (ng/mg) | day 7 | Injury | Inflammation | (%) |
| 150 μg Rivaroxaban & 25 μg | 0.72 ± 0.12 | 99.6% | 3.9 ± 0.6 | 0.31 ± 0.20 | 0.03 ± 0.04 | 1.15 ± 0.34 | 11.4 ± 2.5 |
| Poly(n-butyl methacrylate) | | | | | | | |
| matrix coated stent | | | | | | | |
| 150 μg Argatroban & 200 μg | 1.25 ± 0.87 | 47.7% | 5.7 ± 1.8 | 0.34 ± 0.20 | 0.08 ± 0.08 | 0.81 ± 0.46 | 2.4 ± 2.3 |
| Poly(n-butyl methacrylate) | | | | | | | |
| matrix coated stent | | | | | | | |
| 100 μg Argatroban and | 0.80 ± 0.39 | 70.9% for | 48.1 ± 43.6 for | 0.04 ± 0.06 | 0.06 ± 0.07 | 1.11 ± 0.35 | 0.5 ± 0.9 |
| 100 μg Rivaroxaban in 150 | | Rivaroxaban | Rivaroxaban | | | | |
| μg Poly(n-butyl | | 96.5% for | 8.9 ± 6.3 for | | | | |
| methacrylate) matrix coated | | Argatroban | Argatroban | | | | |
| stent | | | | | | | |
| 150 μg Dalteparin(low | 1.50 ± 0.82 | 98.3% | Not tested | 0.23 ± 0.20 | 0.05 ± 0.06 | 0.65 ± 0.30 | 3.7 ± 5.2 |
| molecular weight heparin) in | | | | | | | |
| 225 μg Poly(n-butyl | | | | | | | |
| methacrylate) matrix coated | | | | | | | |
| stent | | | | | | | |
| Bare metal control stent | 1.02 ± 0.35 | N/A | N/A | 0.17 ± 0.22 | 0.05 ± 0.08 | 0.77 ± 0.45 | 3.3 ± 1.7 |
| (BMS) | | | | | | | |

As shown in Table 1, Rivaroxaban composition released from stents was more effective at inhibiting fibrin formation compared to bare metal control stents at 7 days, while Argatroban composition released from stents or Dalteparin composition released from stents were not more effective at inhibiting fibrin formation compared to bare metal control stents at 7 days.

As shown in Table 1, Rivaroxaban, Argatroban, or Dalteparin compositions released from stents as single agents had larger LLLs compared to control and thus were not more effective at inhibiting smooth muscle cell proliferation compared to bare metal control stents at 7 days.

As shown in Table 1, the combination of Rivaroxaban and Argatroban composition released from stents had a smaller LLL compared to control and thus was more effective at inhibiting smooth muscle cell proliferation compared to bare metal control stents at 7 days. Furthermore, the combination of Rivaroxaban and Argatroban composition released from stents was more effective at inhibiting fibrin formation compared to bare metal control stents.

As shown in Table 1, Rivaroxaban composition comprising fast released from stents at a dose of about 150 μg within 7 days from implant (or from vessel injury) was more effective at inhibiting fibrin formation at or within 7 days.

As shown in Table 1, Rivaroxaban composition comprising fast released from stents at a dose of about 1.8 μg/mm2 within 7 days from implant (or from vessel injury) was more effective at inhibiting fibrin formation at or within 7 days.

As shown in Table 1, Rivaroxaban composition comprising fast released from stents at a dose of about 10.7 μg/mm of stent length within 7 days from implant (or from vessel injury) was more effective at inhibiting fibrin formation.

As shown in Table 1, Rivaroxaban composition comprising a dose of about 150 μg, and/or of about 1.8 μg/mm², and/or of about 10.7 μg/mm of device length, released from a stents device at a release rate comprising of about 99.5% within 7 days from implant (or from time of injury) was more effective at inhibiting fibrin formation.

As shown in Table 1, Rivaroxaban composition released from stents at a release rate comprising of about 70.9% within 7 days when combined with Argatroban composition at a release rate comprising of about 96.9% within 7 days from implant (or from time of injury) was more effective at inhibiting fibrin formation.

Table 1 shows Rivaroxaban composition released from a stent at a dose of about 100 μg, or at a dose comprising of about 1.2 μg/mm², and/or at a dose of about 7.14 μg/mm of stent length, at a release rate comprising of about 70.9% within 7 days when combined with Argatroban composition released from a stent at a dose comprising of about 100 μg, and/or at a dose of about 1.2 μg/mm², and/or at a dose of about 7.14 μg/mm of stent length, at a release rate comprising of about 96.5% within 7 days from implant (or from time of injury) was more effective at inhibiting fibrin formation.

### Example 3: Preparation of Rivaroxaban and m-TOR Inhibitor Releasing Stent

Base coat of Novolimus (m-TOR inhibitor) and Poly (n-butyl methacrylate) matrix: Poly(n-butyl methacrylate) polymer was dissolved into dichloromethane at room temperature and vortex until the polymer had uniformly dissolved/dispersed. Novolimus was placed in another vial and dissolved in dichloromethane at room temperature until uniformly dissolved or dispersed, The polymer solution and drug solutions were mixed together and coated as a matrix (the drug to polymer weight ratio was 2:3 by weight).

Top layer or coat of Rivaroxaban and poly (n-butyl methacrylate) matrix: Poly(n-butyl methacrylate) polymer was dissolved in dichloromethane at room temperature and vortex until the polymer had uniformly dissolved/dispersed. Rivaroxaban was dissolved into dichloromethane at room temperature and vortex until the drug was uniformly dissolved/dispersed. Each polymer solution and each drug solutions were mixed together as a matrix (Rivaroxaban to poly(n-butyl methacrylate) by weight ratio was 6:1 for the Rivaroxaban fast formulation without m-TOR). Rivaroxaban to poly (n-butyl methacrylate) ratio was 4:1 for the fast release formulation with m-TOR base coat matrix, and 2:1 for the slow release formulation with m-TOR base coat matrix according to the target drug dose of 100 μg Rivaroxaban and 25 μg poly(n-butyl methacrylate) for fast release formulation, and 100 μg Rivaroxaban and 50 μg poly(n-butyl methacrylate) for the slow release formulation.

A microprocessor-controlled ultrasonic sprayer was used to coat each of the stents' 14 mm length uniformly with each of the drug/polymer matrix solution with the base coat matrix first, placing the stents in vacuum chamber to remove the solvent, followed by the top layer or coat matrix. The stents were placed in a vacuum chamber again to remove the solvents. The stents were then mounted on balloon catheters and crimped. The catheters were then inserted in coils and packaged. The pouches were sterilized. The Novolimus (m-TOR inhibitor) stents controls (DES) consisted of only the base coat drug/polymer matrix, without the top layer or coat drug/polymer matrix, otherwise being the same as the other stents. The bare metal control stents (BMS) were the same as the other stents without a drug or polymer coating.

TABLE 2
Histopathology Scores, Quantitative Coronary Angiography data and PK of Rivaroxaban releasing 14 mm stents at day 7 and at day 28.

| | Stent coating | | | | | | | |
|---|---|---|---|---|---|---|---|---|
| | information | | [symbol] | | | | | |
| | n = 5 for | | percent | Tissue | | | Inflam- | |
| Time | each arm for | [symbol] | release | [symbol] | [symbol] | Injury | mation | Diameter |
| point | each time point | [symbol] | of drug | [symbol] | [symbol] | score | score | [symbol] |
| Day 7 | 25 μg Poly(n-butyl methacrylate) & | 0.79 ± 0.14 | 99.7% | 3.95 ± 1.57 | 0.26 ± 0.24 | 0.07 ± 0.05 | 1.19 ± 0.26 | 8.5 ± 5.2 |
| (n = 5) | 150 μg Rivaroxaban matrix | | | | | | | |
| | 66 μg Novolimus (m-TOR inhibitor) and | 1.16 ± 0.60 | 88.9% | 6.18 ± 5.37 | 0.21 ± 0.26 | 0.17 ± 0.09 | 1.32 ± 0.22 | 5.5 ± 5.9 |
| | 100 μg Poly(n-butyl methacrylate) matrix | | | | | | | |
| | as a base coat then 100 μg Rivaroxaban | | | | | | | |
| | and 25 μg Poly(n-butyl methacrylate) | | | | | | | |
| | matrix as a top coat | | | | | | | |
| | 66 μg Novolimus (m-TOR inhibitor) and | 1.18 ± 0.53 | 68.1% | 8.56 ± 3.56 | 0.23 ± 0.22 | 0.26 ± 0.19 | 1.48 ± 0.13 | 4.1 ± 3.6 |
| | 100 μg Poly(n-butyl methacrylate) matrix | | | | | | | |
| | as a base coat then 100 μg Rivaroxaban | | | | | | | |
| | and 50 μg Poly(n-butyl methacrylate) | | | | | | | |
| | matrix as a top coat | | | | | | | |
| | 66 μg Novolimus (m-TOR inhibitor) and | 1.51 ± 0.71 | — | — | 0.13 ± 0.15 | 0.20 ± 0.13 | 1.32 ± 0.51 | 9.0 ± 7.3 |
| | 100 μg Poly(n-butyl methacrylate) matrix | | | | | | | |
| | (DES) control | | | | | | | |
| | Bare metal control (BMS) | 1.35 ± 0.41 | — | — | 0.05 ± 0.09 | 0.13 ± 0.07 | 1.22 ± 0.30 | 5.4 ± 3.4 |
| Day 28 | 25 μg Poly(n-butyl methacrylate) & | 0.07 ± 0.05 | 100% | 3.93 ± 2.43 | 0.62 ± 0.21 | 0.25 ± 0.16 | 0.73 ± 0.24 | 22.7 ± 8.7 |
| (n = 5) | 150 μg Rivaroxaban matrix | | | | | | | |
| | 66 μg Novolimus (m-TOR inhibitor) and | 1.06 ± 0.46 | 92.5% | 14.61 ± 17.68 | 0.88 ± 0.96 | 0.68 ± 0.57 | 1.04 ± 0.70 | 38.9 ± 36.0 |
| | 100 μg Poly(n-butyl methacrylate) matrix | | | | | | | |
| | as a base coat then 100 μg Rivaroxaban | | | | | | | |
| | and 25 μg Poly(n-butyl methacrylate) | | | | | | | |
| | matrix as a topcoat | | | | | | | |
| | 66 μg Novolimus (m-TOR inhibitor) and | 1.49 ± 0.37 | 72.7% | 15.23 ± 8.12 | 0.84 ± 0.73 | 0.53 ± 0.58 | 0.72 ± 0.86 | 22.4 ± 20.8 |
| | 100 μg Poly(n-butyl methacrylate) matrix | | | | | | | |
| | as a base coat then 100 μg Rivaroxaban | | | | | | | |
| | and 50 μg Poly(n-butyl methacrylate) | | | | | | | |
| | matrix as a topcoat | | | | | | | |
| | 66 μg-Novolimus (m-TOR inhibitor) and | 1.37 ± 0.44 | — | — | 0.88 ± 0.21 | 0.55 ± 0.33 | 0.79 ± 0.80 | 24.8 ± 13.9 |
| | 100 μg Poly(n-butyl methacrylate) matrix | | | | | | | |
| | (DES) control | | | | | | | |

[symbol]

indicates data missing or illegible when filed

As shown in Table 2, Rivaroxaban composition comprising fast released formulation from a stent was more effective at inhibiting fibrin formation compared to control at 7 days and/or at 28 days when it was released at a faster rate formulation.

As shown in Table 2, Rivaroxaban composition released from a stent was more effective at inhibiting fibrin formation compared to control at 7 days and/or at 28 days when it was released at a rate comprising of about 88.900 within 7 days and/or when a dose of about 88.9 μg, and/or a dose of about 1.2 μg/mm², and/or a dose of about 7.14 μg/mm of stent length, was released within 7 days from vessel injury (or from implantation).

As shown in Table 2, Rivaroxaban formulation released from a stent was more effective at inhibiting fibrin formation compared to control at 7 days and/or at 28 days when it was released at a rate comprising of about 92.5% within 28 days and/or when a dose of about 92.5 μg, and/or a dose of about 1.1 μg/mm², or a dose of about 6.6 μg/mm of stent length, was released at or within 28 days from vessel injury (or from implantation).

As shown in Table 2, Rivaroxaban composition released from a stent was more effective at inhibiting fibrin formation compared to control at 7 days and/or at 28 days when it was released at a rate comprising of 88.9% within 7 days and/or when a dose of 88.9 μg, and/or a dose of 1.2 μg/mm², and/or a dose of 7.14 μg/mm of stent length, was released within 7 days after vessel injury (or from implantation), and/or at a rate comprising of about 92.5% within 28 days and/or when a dose of about 92.5 μg, and/or a dose of about 1.1 μg/mm², and/or a dose of about 6.6 μg/mm of stent length, was released within 28 days after vessel injury (or from implantation).

As shown in Table 2, Rivaroxaban composition released in combination with an m-TOR inhibitor from a stent was more effective at inhibiting fibrin formation compared to control at 7 days and/or at 28 days when it was released in a faster formulation rate in accordance with the experiment.

As shown in Table 2, Rivaroxaban composition released in combination with an m-TOR formulation inhibitor from a stent was more effective at inhibiting fibrin formation compared to control at 7 days and/or at 28 days when Rivaroxaban composition was released at a rate comprising of about 88.9 μg, or a dose of about 1.2 μg/mm², or a dose of about 7.14 μg/mm of stent length, within 7 days, and/or released at a rate comprising of about 92.5 μg, and/or a dose of about 1.1 μg/mm², and/or a dose of about 6.6 μg/mm of stent length, within 28 days.

As shown in Table 2, Rivaroxaban composition released in combination with an m-TOR inhibitor formulation from a stent was more effective at inhibiting fibrin formation compared to control at 7 days when Rivaroxaban composition was released at a rate comprising of about 68.1 μg, or at rate comprising of 0.84 μg/mm², and/or at a rate comprising of about 4.86 μg/mm of stent length, within 7 days.

As shown in Table 2, Rivaroxaban tissue concentration ranges from at least 3.96 ng/mg of tissue adjacent to the stented segment to at least 15 ng/mg of tissue adjacent to the stented segment, within or at 7 days, or within or 28 days from implant (or tissue injury)

It was reported that Rivaroxaban IC₅₀ for factor Xa inhibition to be about 21 nM or 0.0092 ng/mg. As shown in Table 2, the tissue concentration for Rivaroxaban was at least 426 times Rivaroxaban IC 50 for factor Xa inhibition.

TABLE 3
Tissue concentration of Rivaroxaban and Argatroban at 7 days show multiple folds higher
(or times higher) than IC50 for Anti-factor Xa/IIa and antiplatelet for the respective drugs.

| | Rivaroxaban* | | Argatroban** | |
|---|---|---|---|---|
| | Tissue | Tissue | Tissue | Tissue |
| | concentration | concentration | concentration | concentration |
| | at day 7 in folds | at day 7 in folds | at day 7 in folds | at day 7 in folds |
| Stent coated | higher than | higher than | higher than | higher than |
| with combination of | IC50 of anti- | IC50 of anti- | IC50 of anti- | IC50 of anti- |
| Rivaroxaban and Argatroban | Factor Xa | platelet | Factor IIa | platelet |
| 100 μg Argatroban and | 5253 | 354 | 835 | 1223 |
| 100 μg Rivaroxaban in | | | | |
| Poly(n-butyl | | | | |
| methacrylate) matrix | | | | |

*Rivaroxaban IC50 for Anti-Factor Xa is 21 nM or 0.00916 ng/mg
*Rivaroxaban IC50 for Tissue factor generated antiplatelet is 312 nM or 0.136 ng/mg
**Argatroban IC50 for Anti-Factor IIa is 21 nM or 0.0107 ng/mg
**Argatroban IC50 for Tissue factor generated antiplatelet is 79 nM or 0.04 ng/mg

Table 3 shows the tissue PK data for Rivaroxaban and Argatroban at or by or within 7 days from implants of stented vessels. It shows Rivaroxaban and Argatroban has therapeutic tissue concentrations in the tissue segment up to 7 days. Table 3 is Rivaroxaban and Argatroban concentration (ng/mg) in the tissue of treated area of the implanted device fold higher than IC₅₀ for anti-Factor Xa or Anti-Factor IIa and anti-platelet. It shows that Rivaroxaban and Argatroban in tissue concentrations have several order of magnitudes, has from 2 to 4 orders of magnitude of tissue concentration for each of the drugs compared to their IC₅₀, in the treated tissue segments up to 7 days, therefore inhibiting or enhancing dissolution of one or more of cell proliferation, fibrin formation, or clot formation on the device surfaces, the stented segment tissue, and/or the tissue adjacent to the stented segment.

### Example 4: Preparation of Anticoagulant1/Anticoagulant2/mTOR Eluting Stents

Poly(L-lactide acid-co-glycolic acid) polymer was dissolved into dichloromethane at room temperature and vortex until the polymer had uniformly dissolved/dispersed. Sirolimus and anticoagulants (Apixaban or Rivaroxaban and Argatroban) were placed in a vial and dissolved in dichloromethane or dichloromethane/Methanol at room temperature and vortex until all the drug was uniformly dissolved/dispersed.

Each polymer solution and each drug (or combined drugs) solutions were combined together (SS7 arm anticoagulant (Apixaban to Argatroban was 1:1) to poly(L-lactide acid-co-glycolic acid) matrix by weight ratio was 3:1 as a base coat and Siroliums to poly(L-lactide acid-co-glycolic acid) matrix by weight ratio was 2:3 and coated as a top coat), (SS9 arm Siroliums and anticoagulant Apixaban and Argatroban was (1:1:1) to poly(L-lactide acid-co-glycolic acid) by weight ratio was 5:2 on matrix), (SS15 arm Sirolimus and Apixaban and Argatroban were combined in a ratio of (1:1:1) with poly(L-lactide acid-co-glycolic acid) by weight ratio which was (1:2)(by weight of 23 μg Sirolimus, 23 μg Apixaban and 23 μg Argatroban combined with 138 μg poly(L-lactide acid-co-glycolic acid)) and mixed together, and coated as a base coat (drug/polymer matrix as a base coat). In addition, Sirolimus and Apixaban and Argatroban were combined in the ratio of (3:4:4) with poly(L-lactide acid-co-glycolic acid) by weight ratio which was (5:3) and coated as a top layer or coat (drug/polymer matrix top layer or coat), (by weight of 71 μg Sirolimus, 94 μg Apixaban and 94 μg Argatroban and combined with 155 μg poly(L-lactide acid-co-glycolic acid) and coated as a top layer or coat, for cumulative total target drug dose of 117 μg for each anticoagulant and 94 μg for Sirolimus for a 14 mm stent length, (Slider II Arm1 (SS16) Sirolimus and Rivaroxaban and Argatroban were combined together in the ratio of (1:1:1) and were combined with poly(L-lactide acid-co-glycolic acid) by weight ratio which was (1:2) and coated as a base coat (drug/polymer matrix as base coat). In addition, Sirolimus and Rivaroxaban and Argatroban were combined in the ratio of (3:4:4) and combined with poly(L-lactide acid-co-glycolic acid) by weight ratio was (5:3) and coated as a top layer or coat (drug/polymer matrix as top layer or coat), (by weight of 23 μg Sirolimus, 23 μg Rivaroxaban and 23 μg Argatroban and 138 μg poly(L-lactide acid-co-glycolic acid) mixed together and coated as base coat; and by weight of 71 μg Sirolimus, 94 μg Rivaroxaban and 94 μg Argatroban and 155 μg poly(L-lactide acid-co-glycolic acid) mix together in a matrix and coated as top layer or coat, for a total target drug dose of 117 μg for each anticoagulant and 94 μg for Sirolimus for a 14 mm stent length, (Slider II Arm2 (SS17) Sirolimus and Rivaroxaban and Argatroban were combined in a ratio of (4:1:1) and combined with poly(L-lactide acid-co-glycolic acid) by weight ratio which was (1:1) and coated as a base coat (drug/polymer matrix as base coat). In addition, Rivaroxaban and Argatroban were combined in a ratio of (1:1) and combined with poly(L-lactide acid-co-glycolic acid) by weight ratio which was (5:3) and coated as a top layer or coat on the stent (drug/polymer matrix as a top layer or coat), (by weight of 94 μg Sirolimus, 23 μg Rivaroxaban and 23 μg Argatroban and 140 μg poly(L-lactide acid-co-glycolic acid) mixed together and coated as base coat; and by weight of 94 μg Rivaroxaban and 94 μg Argatroban and 113 μg poly(L-lactide acid-co-glycolic acid) were mixed together and coated as top layer or coat, for a total target drug dose of 117 μg for each anticoagulant and 94 μg for Sirolimus for a 14 mm stent length. The preceding doses for SS7, SS9, SS15, SS16, and SS17 were for 14 mm stent lengths. Drug and polymer doses are adjusted accordingly for each stent length. Control was 14 mm stent length eluting 65 μg Novolimus (m-TOR inhibitor). A microprocessor controlled ultrasonic sprayer was used to coat each of the stents' 14 mm length uniformly with each of the drug/polymer matrix solution. After coating, the stents were placed in a 70° C. oven for about 2 hours to remove the solvent. The stents were then mounted on balloon catheters and crimped. The catheters were then inserted in coils and packaged. The pouches were sterilized.

The following tables 4A-4K describe results from in vivo testing for the following arms of SS7, SS9, SS15, SS16, and SS17 from example 4.

TABLE 4A
In-vivo cumulative percent drug release profile of Rapamycin,
Apixaban/Rivaroxaban and Argatroban in stented segments.

| Sample | Sample | Time | | | | | | | |
|---|---|---|---|---|---|---|---|---|---|
| Matrix | Size | period | 1 H | 3 H | 24 H | 6 D | 7 D | 28 D | 90 D |
| SS7 Argatroban/ | n = 1 | Apixaban, % | N/A | 30 | 91 | 98 | 99 | 99 | N/A |
| Apixaban/ | | Argatroban, % | N/A | 91 | 99 | 98 | 99 | 99 | N/A |
| Sirolimus | | Sirolimus, % | N/A | 50 | 70 | 69 | 73 | 77 | N/A |
| SS9 Argatroban/ | n = 1 | Apixaban, % | N/A | 68 | 97 | 98 | 98 | 99 | N/A |
| Apixaban/ | | Argatroban, % | N/A | 79 | 98 | 98 | 98 | 99 | N/A |
| Sirolimus | | Sirolimus, % | N/A | 60 | 91 | 93 | 94 | 97 | N/A |
| SS15 Argatroban/ | n = 5 | Apixaban, % | 49 | 61 | 77 | N/A | 80 | 84(n = 3) | 87(n = 3) |
| Apixaban/ | | Argatroban, % | 51 | 63 | 77 | N/A | 80 | 84(n = 3) | 86(n = 3) |
| Sirolimus | | Sirolimus, % | 44 | 55 | 71 | N/A | 81 | 90(n = 3) | 97(n = 3) |
| SS16 Argatroban/ | n = 5 | Rivaroxaban, % | 36 | 47 | 83 | N/A | 86 | 89 | N/A |
| Rivaroxaban/ | | Argatroban, % | 35 | 49 | 82 | N/A | 85 | 87 | N/A |
| Sirolimus | | Sirolimus, % | 29 | 44 | 73 | N/A | 87 | 94 | N/A |
| SS17 Argatroban/ | n = 5 | Rivaroxaban, % | 65 | 71 | 86 | N/A | 92 | 94 | N/A |
| Rivaroxaban/ | | Argatroban, % | 78 | 80 | 86 | N/A | 91 | 93 | N/A |
| Sirolimus | | Sirolimus, % | 8 | 18 | 63 | N/A | 77 | 87 | N/A |

N/A: Not available

Table 4A: SS7 and SS9 provide a therapeutic composition where about 90% of the factor Xa and factor IIa inhibitors are released from the stent within 24 hours. It also shows that these agents are released substantially completely within about 28 days.

Table 4A: SS15, SS16, and SS17 provide therapeutic compositions where each composition providing a bolus drug release from time of injury and/or implant, and an extended drug release from time of injury and/or implant for each of Apixaban, Rivaroxaban, and Argatroban.

Table 4A: SS15 provides a therapeutic composition providing a bolus drug release phase (or formulation) from time of injury and/or implant and an extended drug release phase (or formulation) from time of injury and/or implant for the combination of Apixaban and Argatroban, wherein the bolus drug release occurs within an hour, within 3 hours, or within 24 hours, from time of injury and/or implant; and the extended drug release extends beyond 7 day, extends beyond 28 days, or extends beyond 90 days from time of injury and/or implant.

Table 4A: SS15 provides a therapeutic composition a bolus drug release phase (or formulation) from time of injury and/or implant and an extended drug release phase (or formulation) from time of injury and/or implant for the combination of Apixaban, Argatroban, and Sirolimus, wherein the bolus drug release occurs within an hour to within 24 hours from time of injury and/or implant and the extended drug release extends beyond 7 day, extends beyond 28 days, or extends beyond 90 days from time of injury and/or implant.

Table 4A: SS15 provides a therapeutic composition providing a bolus drug release phase (or formulation) from time of injury and/or implant and an extended drug release phase (or formulation) from time of injury and/or implant for the combination of Apixaban and Argatroban, wherein the bolus drug release occurs within an hour from time of injury and/or implant and wherein Apixaban bolus release is about 49% within an hour and wherein Argatroban bolus release is about 51% within an hour and the extended drug release of each of the drugs is about 80% within 7 days, about 84% within 28 days, and about 86% within 90 days from time of injury and/or implant. In this arm, the drugs are released or commence release substantially about the same time.

Table 4A: SS16 and SS17 provide therapeutic composition providing a bolus drug release phase (or formulation) from time of injury and/or implant and an extended drug release phase (or formulation) from time of injury and/or implant for the combination of Rivaroxaban and Argatroban, wherein the bolus drug release occurs within an hour to within 24 hours from time of injury and/or implant and the extended drug release extends beyond 7 day, or extends beyond 28 days from time of injury and/or implant.

Table 4A: SS16 provides a therapeutic composition providing a bolus drug release phase (or formulation) from time of injury and/or implant and an extended drug release phase (formulation) from time of injury and/or implant for the combination of Rivaroxaban, Argatroban, and Sirolimus, wherein the bolus drug release occurs within an hour to within 24 hours from time of injury and/or implant and the extended drug release extends beyond 7 day, or extends beyond 28 days from time of injury and/or implant. In this arm, the drugs are released or commence release substantially about the same time.

Table 4A: SS16 and SS17 provide therapeutic compositions providing a bolus drug release phase (or formulation) from time of injury and/or implant and an extended drug release phase (or formulation) from time of injury and/or implant for the combination of Rivaroxaban and Argatroban, wherein the bolus drug release occurs within an hour to within 24 hours from time of injury and/or implant and wherein Rivaroxaban bolus release ranges from 36% to 68% within an hour and wherein Argatroban bolus release ranges from 35% to 78% within an hour and the extended drug release of each of the drugs ranges from 85% to 92% for Rivaroxaban within 7 days, 86%-91% for Argatroban within 7 days, ranges from 89%-94% within 28 days for Rivaroxaban and 87%-93% for Argatroban from time of injury and/or implant to within 28 days.

Table 4A: SS16 and SS17 formulations each has one formulation providing a bolus drug release and another formulation providing an extended drug release for the combination of Rivaroxaban and Argatroban, wherein the bolus drug release occurs within an hour to within 24 hours of injury or implantation and the extended drug release extends beyond 7 day, or extends beyond 28 days.

Table 4A: SS16 and SS17 shows multiple formulations providing a bolus drug release formulation and an extended drug release formulation for the combination of each of Rivaroxaban and Argatroban, wherein the extended release extends beyond 7 day, or extends beyond 28 days.

Table 4A: SS15, SS16, and SS17 Provides therapeutic compositions comprising two drugs/polymer formulations each, wherein each formulation contains at least two drugs: a factor Xa inhibitor and a factor IIA inhibitor. A third drug being an M-tor inhibitor is present in each of the formulations except in SS17 where it is present in only one formulation (base formulation) configured to delay the release of M-tor in SS17 providing a smaller bolus within the first hour for M-tor. All formulations provide an extended release of the drugs beyond 7 days, or beyond 28 days. Arm SS17 factor IIa inhibitor and factor Xa inhibitor commence release prior to the anti-proliferative which was intended/configured to delay commence of its release compared to the other two drugs.

TABLE 4B
Tissue drug concentration (ng/mg) of Apixaban, Rivaroxaban, Argatroban and Rapamycin
in the stented segment tissue at the indicated time points following implantation.

| Sample | Sample | Time | | | | | | | |
|---|---|---|---|---|---|---|---|---|---|
| Matrix | Size | period | 1 H | 3 H | 24 H | 6 D | 7 D | 28 D | 90 D |
| SS7 Argatroban/ | n = 1 | | | | | | | | |
| Apixaban/ | | Apixaban | N/A | 102.9 | 16.5 | 0.07 | 0.03 | 0.12 | N/A |
| Sirolimus | | Argatroban | N/A | 54 | 0.75 | 0.09 | 0.05 | 0.15 | N/A |
| | | Sirolimus | N/A | 7.34 | 4.46 | 1.38 | 0.73 | 1.79 | N/A |
| SS9 Argatroban/ | n = 1 | Apixaban | N/A | 91.8 | 4.17 | 0.28 | 2.65 | 1.69 | N/A |
| Apixaban/ | | Argatroban | N/A | 123.1 | 0.18 | 0.33 | 2.54 | 1.76 | N/A |
| Sirolimus | | Sirolimus | N/A | 40.57 | 0.93 | 1.61 | 3.24 | 2.48 | N/A |
| SS15 Argatroban/ | n = 5 | Apixaban | 66.94 ± 27.33 | 25.31 ± 11.21 | 13.25 ± 10.17 | NA | 1.15 ± 0.52 | 1.28 ± | 3.05 ± |
| Apixaban/ | | | | | | | | 0.47(n = 3) | 1.77(n = 3) |
| Sirolimus | | Argatroban | 71.37 ± 31.32 | 27.65 ± 15.00 | 15.64 ± 12.08 | NA | 1.41 ± 0.69 | 1.69 ± | 3.74 ± |
| | | | | | | | | 0.64(n = 3) | 1.89(n = 3) |
| | | Sirolimus | 43.22 ± 14.73 | 23.37 ± 6.88 | 29.17 ± 18.65 | NA | 1.54 ± 0.35 | 1.67 ± | 1.28 ± |
| | | | | | | | | 0.22(n = 3) | 0.07(n = 3) |
| SS16 Argatroban/ | n = 5 | Rivaroxaban | 48.75 ± 25.52 | 21.48 ± 5.80 | 3.67 ± 5.59 | N/A | 0.31 ± 0.24 | 0.34 ± 0.27 | N/A |
| Rivaroxaban/ | | Argatroban | 61.87 ± 24.60 | 32.81 ± 10.96 | 3.80 ± 4.87 | N/A | 0.42 ± 0.32 | 0.52 ± 0.37 | N/A |
| Sirolimus | | Sirolimus | 45.10 ± 14.77 | 38.30 ± 9.29 | 9.18 ± 5.69 | N/A | 1.46 ± 0.38 | 0.94 ± 0.19 | N/A |
| SS17 Argatroban/ | n = 5 | Rivaroxaban | 38.31 ± 16.08 | 26.23 ± 23.50 | 1.31 ± 0.28 | N/A | 1.07 ± 1.88 | 0.52 ± 0.63 | N/A |
| Rivaroxaban/ | | Argatroban | 11.80 ± 2.69 | 8.06 ± 3.48 | 1.19 ± 0.46 | N/A | 1.35 ± 2.41 | 0.67 ± 0.87 | N/A |
| Sirolimus | | Sirolimus | 21.34 ± 7.51 | 27.32 ± 6.86 | 10.85 ± 3.55 | N/A | 3.80 ± 4.86 | 1.73 ± 1.52 | N/A |

Table 4B shows drug concentration in tissue adjacent to the stented segment for each of the drugs: Apixaban of about 67 ng/mg within one hour, of about 25 ng/mg tissue within 3 hours, of about 1.15 ng/mg tissue within 7 days, 1.28 ng/mg tissue within 28 days, and of about 3 ng/mg tissue within 90 days from time of injury and/or implant; Rivaroxaban of about 38 ng/mg, or of about 49 ng/mg within one hour, of about 21 ng/mg, or of about 26 ng/mg tissue within 3 hours, of about 0.3 ng/mg, or of about 1.1 ng/mg tissue within 7 days, of about 0.34 ng/mg, or of about 0.52 ng/mg tissue within 28 days, from time of injury and/or implant; Argatroban of about 12 ng/mg, of about 62 ng/mg tissue, or of about 71 ng/mg tissue within 1 hour, of about 8 ng/mg tissue, of about 33 ng/mg tissue, or of about 27 ng/mg tissue within 3 hours, of about 0.42 ng/mg tissue, of about 1.35 ng/mg tissue, or of about 1.41 ng/mg tissue within 7 days, of about 0.52 ng/mg tissue, of about 0.67 ng/mg tissue, or of about 1.69 ng/mg tissue within 28 days, and of about 3.74 ng/mg tissue within 90 days from time of injury and/or implant; and Sirolimus of about 21 ng/mg tissue, of about 45 ng/mg tissue, or of about 43 ng/mg tissue within one hour, of about 27 ng/mg tissue, or about 38 ng/mg tissue, or of about 24 ng/mg tissue within 3 hours, of about 1.46 ng/mg tissue, of about 3.8 ng/mg tissue, or of about 1.54 ng/mg tissue within 7 days, of about 0.94 ng/mg tissue, of about 1.73 ng/mg tissue, or of about 1.67 ng/mg tissue within 28 days, and of about 1.28 ng/mg tissue within 90 days, from time of tissue injury and/or implant.

TABLE 4C
In Vivo drug remaining on stent (μg) and average cumulative percentage releases (%) of Apixaban,
Rivaroxaban, Argatroban and Sirolimus in the stent at the indicated time points following implantation.

| | | Sample matrix* (n = 5) | | | | | | | | |
|---|---|---|---|---|---|---|---|---|---|---|
| | | SS15 Argatroban/Apixaban/ | | | SS16 Argatroban/Rivaroxaban/ | | | SS17 Argatroban/Rivaroxaban/ | | |
| | | Sirolimus in base coat | | | Sirolimus in base coat | | | Sirolimus in base coat | | |
| | | and in topcoat | | | and in topcoat | | | and in topcoat | | |
| | | Drug remaining on stent, | | | | | | | | |
| | | μg & percentage released (%) | | | | | | | | |
| Time (hrs) | | Apixaban | Argatroban | Sirolimus | Rivaroxaban | Argatroban | Sirolimus | Rivaroxaban | Argatroban | Sirolimus |
| 0 | | 119 | 123 | 96 | 120 | 119 | 90 | 121 | 121 | 96 |
| 1 | H | 61 ± 4.8 | 60 ± 5.5 | 54 ± 2.8 | 76 ± 7.1 | 77 ± 59.2 | 63 ± 4.7 | 43 ± 3.5 | 26 ± 0.8 | 88 ± 1.5 |
| | | (49%) | (51%) | (44%) | (36%) | (35%) | (29%) | (65%) | (78%) | (8%) |
| 3 | H | 46 ± 5.2 | 46 ± 5.1 | 43 ± 3.9 | 64 ± 9.5 | 61 ± 13.9 | 50 ± 6.1 | 34 ± 5.1 | 24 ± 1.9 | 79 ± 3.5 |
| | | (61%) | (63%) | (55%) | (47%) | (49%) | (44%) | (71%) | (80%) | (18%) |
| 24 | H | 27 ± 3.6 | 28 ± 1.4 | 28 ± 0.8 | 21 ± 4.8 | 21 ± 1.6 | 24 ± 7.1 | 17 ± 5.9 | 17 ± 3.8 | 36 ± 7.3 |
| | | (77%) | (77%) | (71%) | (83%) | (82%) | (73%) | (86%) | (86%) | (63%) |
| 7 | D | 24 ± 1.4 | 25 ± 1.0 | 18 ± 0.3 | 16 ± 2.5 | 18 ± 1.7 | 12 ± 1.2 | 9 ± 0.3 | 11 ± 0.4 | 22 ± 0.5 |
| | | (80%) | (80%) | (81%) | (86%) | (85%) | (87%) | (92%) | (91%) | (77%) |
| 28 | D | 19 ± 0.9 | 20 ± 6.1 | 9 ± 0.3 | 14 ± 1.0 | 15 ± 0.8 | 5 ± 0.4 | 7 ± 0.4 | 9 ± 0.7 | 13 ± 0.7 |
| | | (84%) | (84%) | (90%) | (89%) | (87%) | (94%) | (94%) | (93%) | (87%) |
| 90 | D | 16 ± 1.6 | 17 ± 0.6 | 3 ± 0.1 | N/A | N/A | N/A | N/A | N/A | N/A |
| | | (87%) | (86%) | (97%) | | | | | | |

N/A: Not available
*SS15 28 D and 90 D (n = 3)

TABLE 4D
In Vivo drug concentration (ng/mg) in the tissue within 5 mm proximal and 5
mm distal (tissue adjacent to the stented segment) to the stented segment.

| Sample | | | | | | | | | |
|---|---|---|---|---|---|---|---|---|---|
| Matrix | | | | | | | | | |
| (Sample | | 1 Hour | | 3 Hour | | 1 Day | | 6 Day | |
| Size) | Drug | Proximal | Distal | Proximal | Distal | Proximal | Distal | Proximal | Distal |
| SS15 | Apixaban, | 7.86 ± 3.01 | 3.53 ± 1.95 | 5.15 ± 1.59 | 6.77 ± 2.22 | 0.14 ± 0.08 | 0.36 ± 0.29 | N/A | N/A |
| (n = 5 | ng/mg | | | | | | | | |
| except | Argatroban | 8.57 ± 3.66 | 3.84 ± 2.42 | 5.43 ± 2.67 | 6.46 ± 3.14 | 0.05 ± 0.03 | 0.17 ± 0.09 | N/A | N/A |
| 28 D & 90 D | ng/mg | | | | | | | | |
| n = 3) | Sirolimus | 2.82 ± 1.15 | 1.66 ± 0.78 | 3.52 ± 0.31 | 3.51 ± 0.61 | 0.12 ± 0.11 | 3.18 ± 3.01 | N/A | N/A |
| | ng/mg | | | | | | | | |
| SS16 | Rivaroxaban, | 2.03 ± 1.14 | 6.51 ± 2.45 | 1.72 ± 0.53 | 2.16 ± 0.83 | 0.09 ± 0.03 | 0.09 ± 0.02 | N/A | N/A |
| (Slider | ng/mg | | | | | | | | |
| II Arm 1 | Apixaban, | 3.74 ± 1.93 | 7.87 ± 3.04 | 2.48 ± 0.36 | 2.63 ± 0.87 | 0.07 ± 0.03 | 0.09 ± 0.03 | N/A | N/A |
| (n = 5)) | ng/mg | | | | | | | | |
| except | Sirolimus, | 2.36 ± 1.22 | 5.25 ± 1.62 | 2.62 ± 0.82 | 3.78 ± 0.81 | 0.18 ± 0.06 | 1.10 ± 0.44 | N/A | N/A |
| 3 H n = 4, | ng/mg | | | | | | | | |
| 28 d n = 5) | | | | | | | | | |
| SS17 | Rivaroxaban, | 1.32 ± 0.70 | 2.61 ± 1.38 | 3.00 ± 1.50 | 2.72 ± 1.66 | 0.09 ± 0.05 | 0.07 ± 0.02 | N/A | N/A |
| (Slider | ng/mg | | | | | | | | |
| II Arm2 | Apixaban | 0.65 ± 0.23 | 0.91 ± 0.51 | 0.68 ± 0.28 | 0.07 ± 0.52 | 0.04 ± 0.01 | 0.05 ± 0.01 | N/A | N/A |
| (n = 5 | Sirolimus, | 1.39 ± 0.83 | 1.31 ± 0.83 | 2.49 ± 1.02 | 3.95 ± 3.09 | 0.26 ± 0.27 | 0.85 ± 0.48 | N/A | N/A |
| except | ng/mg | | | | | | | | |
| 3 H n = 4)), | | | | | | | | | |
| 28 d n = 6) | | | | | | | | | |

| Sample | | | | | | | |
|---|---|---|---|---|---|---|---|
| Matrix | | | | | | | |
| (Sample | | 7 Day | | 28 Day | | 90 Day | |
| Size) | Drug | Proximal | Distal | Proximal | Distal | Proximal | Distal |
| SS15 | Apixaban, | 0.01 ± 0.01 | 0.01 ± 0.01 | 0.04 | 0.02 | 0.0003 ± 0.0002 | 0.0002 ± 0.0001 |
| (n = 5 | ng/mg | | | (n = 1) | (n = 1) | (n = 2) | (n = 3) |
| except | | | | BQL | BQL | BOL | |
| 28 D & 90 D | | | | (n = 2) | (n = 2) | (n = 1) | |
| n = 3) | Argatroban | 0.01 ± 0.01 | 0.01 ± 0.003 | 0.04 | 0.02 | 0.008 | 0.0006 ± 0.0004 |
| | ng/mg | | | (n = 1) | (n = 1) | (n = 1) | (n = 2) |
| | | | | BQL | BQL | BQL | BQL |
| | | | | (n = 2) | (n = 2) | (n = 2) | (n = 1) |
| | Sirolimus | 0.02 ± 0.01 | 0.27 ± 0.11 | 0.02 ± 0.01 | 0.14 ± 0.04 | 0.01 ± 0.03 | 0.02 ± 0.01 |
| | ng/mg | | | | | | |
| SS16 | Rivaroxaban, | BQL | BQL | 0.02 | 0.02 | N/A | N/A |
| (Slider | ng/mg | | | (n = 1); | (n = 1); | | |
| II Arm 1 | | | | BQL | BQL | | |
| (n = 5)) | | | | (n = 5) | (n = 5) | | |
| except | Apixaban, | BQL | BQL | BQL | BQL | N/A | N/A |
| 3 H n = 4, | ng/mg | | | | | | |
| 28 d n = 5) | Sirolimus, | 0.04 ± 0.03 | 0.55 ± 0.22 | 0.05 ± 0.05 | 0.11 ± 0.05 | N/A | N/A |
| | ng/mg | | | (n = 4); | | | |
| | | | | BQL | | | |
| | | | | (n = 2) | | | |
| SS17 | Rivaroxaban, | 0.01 | 0.01 | 0.02 ± 0.001 | BQL | N/A | N/A |
| (Slider | ng/mg | (n = 1); | (n = 1); | (n = 2); | | | |
| II Arm2 | | BQL | BQL | BQL | | | |
| (n = 5 | | (n = 4) | (n = 4) | (n = 4) | | | |
| except | Apixaban | BQL | BQL | BQL | BQL | N/A | N/A |
| 3 H n = 4)), | Sirolimus, | 0.01 | 0.34 ± 0.21 | 0.03 ± 0.02 | 0.13 ± 0.06 | N/A | N/A |
| 28 d n = 6) | ng/mg | (n = 3); | | (n = 4); | | | |
| | | BQL | | BQL | | | |
| | | (n = 2) | | (n = 2) | | | |

BQL: Below Quantification Limit
N/A: Not Available

TABLE 4E
In Vivo Apixaban concentration (ng/mg) in the stented segment tissue and adjacent
segments of 5 mm proximal and 5 mm distal to the implanted device.

| | | | | | | | |
|---|---|---|---|---|---|---|---|
| Stent | | | | | | | |
| (Sample | | | | | | | |
| Size) | 1 hour | 3 hours | 1 day | 6 days | 7 days | 28 days | 90 days |

Apixaban in treated area tissue content, ng/mg

| | | | | | | | |
|---|---|---|---|---|---|---|---|
| SS15 | 66.94 ± 27.33 | 25.31 ± 11.21 | 13.25 ± 10.17 | N/A | 1.15 ± 0.52 | 1.28 ± 0.47 | 3.05 ± 1.77 |
| (n = 5) | | | | | | (n = 3) | (n = 3) |

Apixaban in Proximal tissue content, ng/mg

| | | | | | | | |
|---|---|---|---|---|---|---|---|
| SS15 | 7.86 ± 3.01 | 5.15 ± 1.59 | 0.14 ± 0.08 | N/A | 0.01 ± 0.01 | 0.04 (n = 1) | 0.0003 ± |
| (n = 5) | | | | | | BQL (n = 2) | 0.0002 (n = 2) |
| | | | | | | | BQL (n = 1) |

Apixaban in Distal tissue content, ng/mg

| | | | | | | | |
|---|---|---|---|---|---|---|---|
| SS15 | 3.53 ± 1.95 | 6.77 ± 2.22 | 0.36 ± 0.29 | N/A | 0.01 ± 0.01 | 0.02 (n = 1) | 0.0002 ± 0.0001 |
| (n = 5) | | | | | | BQL (n = 2) | (n = 3) |

BQL: Below Quantification Limit

TABLE 4F
In Vivo Rivaroxaban concentration (ng/mg) in the stented segment tissue and adjacent segments
of 5 mm proximal and 5 mm distal to the implanted device.
Rivaroxaban in treated area tissue content, ng/mg

| Stent (Sample Size) | 1 hour | 3 hours | 1 day | 7 days | 28 days |
|---|---|---|---|---|---|
| SS16: Slider II Arm1 (n = 5 | 48.75 ± 25.52 | 21.48 ± 5.80 | 3.67 ± 5.59 | 0.31 ± 0.24 | 0.34 ± 0.27 |
| except 3 H n = 4, 28 d n = 6) | | | | | |
| SS17: Slider II Arm2 (n = 5 | 38.31 ± 16.08 | 26.23 ± 3.50 | 1.31 ± 0.28 | 1.07 ± 1.88 | 0.52 ± 0.63 |
| except 3 H n = 4, 28 d n = 6) | | | | | |

Rivaroxaban in Proximal tissue content, ng/mg

| Stent (Sample Size) | 1 hour | 3 hours | 1 day | 7 days | 28 days |
|---|---|---|---|---|---|
| SS16: Slider II Arm1 (n = 5 | 2.63 ± 1.14 | 1.72 ± 0.53 | 0.09 ± 0.03 | BQL | 0.02 (n = 1); |
| except 3 H n = 4, 28 d n = 6) | | | | | BQL(n = 5) |
| SS17: Slider II Arm2 (n = 5 | 1.52 ± 0.70 | 3.00 ± 1.50 | 0.09 ± 0.05 | 0.01(n = 1); | 0.02 ± 0.001 |
| except 3 H n = 4, 28 d n = 6) | | | | BQL(n = 4) | (n = 2); BQL(n = 4) |

Rivaroxaban in Distal tissue content, ng/mg

| Stent (Sample Size) | 1 hour | 3 hours | 1 day | 7 days | 28 days |
|---|---|---|---|---|---|
| SS16: Slider II Arm1 (n = 5 | 6.51 ± 2.45 | 2.16 ± 0.83 | 0.09 ± 0.02 | BQL | 0.02(n = 1); |
| except 3 H n = 4, 28 d n = 6) | | | | | BQL(n = 5) |
| SS17: Slider II Arm2 (n = 5 | 2.61 ± 1.38 | 2.72 ± 1.66 | 0.07 ± 0.02 | 0.01(n = 1); | BQL |
| except 3 H n = 4, 28 d n = 6) | | | | BQL (n = 4) | |

BQL: Below Quantification Limit
N/A: Not Available

TABLE 4G
In Vivo Argatroban concentration (ng/mg) in the stented segment tissue and adjacent
tissue segment of 5 mm proximal and 5 mm distal to the implanted device.

| | | | | | | | |
|---|---|---|---|---|---|---|---|
| Stent | | | | | | | |
| (Sample | | | | | | | |
| Size) | 1 hour | 3 hours | 1 day | 6 days | 7 days | 28 days | 90 days |

Argatroban in treated tissue content, ng/mg

| | | | | | | | |
|---|---|---|---|---|---|---|---|
| SS7 | N/A | 54.00 | 0.75 | 0.09 | 0.05 | 0.15 | N/A |
| (n = 1) | | | | | | | |
| SS9 | N/A | 123.10 | 0.18 | 0.33 | 2.54 | 1.76 | N/A |
| (n = 1) | | | | | | | |
| SS15 | 71.37 ± 31.32 | 27.65 ± 15.00 | 15.64 ± 12.08 | N/A | 1.41 ± 0.69 | 1.69 ± 0.64 | 3.74 ± 1.89 |
| (n = 5) | | | | | | (n = 3) | (n = 3) |
| SS16: Slider | 61.87 ± 24.60 | 32.81 ± 10.96 | 3.80 ± 4.87 | N/A | 0.42 ± 0.32 | 0.52 ± 0.37 | N/A |
| II Arm1 | | | | | | | |
| (n = 5 except | | | | | | | |
| 3 H n = 4, | | | | | | | |
| 28 d n = 6) | | | | | | | |
| SS17: Slider | 11.80 ± 2.69 | 8.06 ± 3.48 | 1.19 ± 0.46 | N/A | 1.35 ± 2.41 | 0.67 ± 0.87 | N/A |
| II Arm2 | | | | | | | |
| (n = 5 except | | | | | | | |
| 3 H n = 4, | | | | | | | |
| 28 d n = 6) | | | | | | | |

Argatroban in Proximal tissue content, ng/mg

| | | | | | | | |
|---|---|---|---|---|---|---|---|
| SS7 | N/A | 13.39 | BQL | BQL | BQL | BQL | N/A |
| (n = 1) | | | | | | | |
| SS9 | N/A | 1.27 | BQL | BQL | BQL | BQL | N/A |
| (n = 1) | | | | | | | |
| SS15 | 8.57 ± 3.66 | 5.43 ± 2.67 | 0.05 ± 0.03 | N/A | 0.01 ± 0.01 | 0.04 (n = 1) | 0.0008 (n = 1) |
| (n = 5) | | | | | | BQL (n = 2) | BQL (n = 2) |
| SS16: Slider | 3.74 ± 1.93 | 2.48 ± 0.36 | 0.07 ± 0.03 | N/A | BQL | BQL | N/A |
| II Arm1 | | | | | | | |
| (n = 5 except | | | | | | | |
| 3 H n = 4, | | | | | | | |
| 28 d n = 6) | | | | | | | |
| SS17: Slider | 0.65 ± 0.23 | 0.68 ± 0.28 | 0.04 ± 0.01 | N/A | BQL | BQL | N/A |
| II Arm2 | | | | | | | |
| (n = 5 except | | | | | | | |
| 3 H n = 4, | | | | | | | |
| 28 d n = 6) | | | | | | | |

Argatroban in Distal tissue content, ng/mg

| | | | | | | | |
|---|---|---|---|---|---|---|---|
| SS7 (n = 1) | N/A | 5.20 | BQL | BQL | BQL | BQL | N/A |
| SS9 (n = 1) | N/A | 11.33 | BQL | N/A | BQL | BQL | N/A |
| SS15 (n = 5) | 3.84 ± 2.42 | 6.46 ± 3.14 | 0.17 ± 0.09 | N/A | 0.01 ± 0.003 | 0.02 (n = 1) | 0.0006 ± 0.0004 |
| | | | | | | BQL (n = 2) | (n = 2) |
| | | | | | | | BQL (n = 1) |
| SS16: Slider | 7.87 ± 3.04 | 2.63 ± 0.87 | 0.09 ± 0.03 | N/A | BQL | BQL | N/A |
| II Arm1 | | | | | | | |
| (n = 5 except | | | | | | | |
| 3 H n = 4, | | | | | | | |
| 28 d n = 6) | | | | | | | |
| SS17: Slider | 0.91 ± 0.51 | 0.87 ± 0.52 | 0.03 ± 0.01 | N/A | BQL | BQL | N/A |
| II Arm2 | | | | | | | |
| (n = 5 except | | | | | | | |
| 3 H n = 4, | | | | | | | |
| 28 d n = 6) | | | | | | | |

BQL: Below Quantification Limit
N/A: Not available

TABLE 4H
In Vivo Rapamycin concentration (ng/mg) in the tissue of treated segment (stented segment)
and adjacent tissue segment of 5 mm proximal and 5 mm distal to the implanted device.

| | | | | | | | |
|---|---|---|---|---|---|---|---|
| Stent | | | | | | | |
| (Sample | | | | | | | |
| Size) | 1 hour | 3 hours | 1 day | 6 days | 7 days | 28 days | 90 days |

Sirolimus in treated tissue content, ng/mg

| | | | | | | | |
|---|---|---|---|---|---|---|---|
| SS7 | N/A | 7.34 | 4.46 | 1.38 | 0.73 | 1.79 | N/A |
| (n = 1) | | | | | | | |
| SS9 | N/A | 40.57 | 0.93 | 1.61 | 3.24 | 2.48 | N/A |
| (n = 1) | | | | | | | |
| SS15 | 43.22 ± 14.73 | 23.37 ± 6.88 | 29.17 ± 18.65 | N/A | 1.54 ± 0.35 | 1.67 ± 0.22 | 1.28 ± 0.07 |
| (n = 5) | | | | | | (n = 3) | (n = 3) |
| SS16: Slider | 45.10 ± 14.77 | 38.30 ± 9.29 | 9.18 ± 5.69 | N/A | 1.46 ± 0.38 | 0.94 ± 0.19 | N/A |
| II Arm1 | | | | | | | |
| (n = 5 | | | | | | | |
| except | | | | | | | |
| 3 H n = 4, | | | | | | | |
| 28 d n = 6) | | | | | | | |
| SS17: Slider | 21.34 ± 7.51 | 27.32 ± 6.86 | 10.85 ± 3.55 | N/A | 3.80 ± 4.86 | 1.73 ± 1.52 | N/A |
| II Arm2 | | | | | | | |
| (n = 5 | | | | | | | |
| except | | | | | | | |
| 3 H n = 4, | | | | | | | |
| 28 d n = 6) | | | | | | | |

Sirolimus in Proximal tissue content, ng/mg

| | | | | | | | |
|---|---|---|---|---|---|---|---|
| SS7 | N/A | 3.21 | BQL | 0.14 | BQL | 0.01 | N/A |
| (n = 1) | | | | | | | |
| SS9 | N/A | 0.38 | 0.58 | BQL | 0.15 | 0.1 | N/A |
| (n = 1) | | | | | | | |
| SS15 | 2.82 ± 1.15 | 3.52 ± 0.51 | 0.12 ± 0.11 | N/A | 0.02 ± 0.01 | 0.02 ± 0.01 | 0.01 ± 0.01 |
| (n = 5) | | | | | | (n = 3) | (n = 3) |
| SS16: Slider | 2.30 ± 1.22 | 2.62 ± 0.82 | 0.18 ± 0.06 | N/A | 0.04 ± 0.03 | 0.05 ± 0.05 | N/A |
| II Arm1 | | | | | | (n = 4); | |
| (n = 5 | | | | | | BQL (n = 2) | |
| except | | | | | | | |
| 3 H n = 4, | | | | | | | |
| 28 d n = 6) | | | | | | | |
| SS17: Slider | 1.39 ± 0.83 | 2.49 ± 1.02 | 0.26 ± 0.27 | N/A | 0.01 (n = 3); | 0.03 ± 0.02 | N/A |
| II Arm2 | | | | | BQL (n = 2) | (n = 4); | |
| (n = 5 | | | | | | BQL (n = 2) | |
| except | | | | | | | |
| 3 H n = 4, | | | | | | | |
| 28 d n = 6) | | | | | | | |

Sirolimus in Distal tissue content, ng/mg

| | | | | | | | |
|---|---|---|---|---|---|---|---|
| SS7 (n = 1) | N/A | 1.86 | 0.99 | 0.69 | 0.46 | 0.14 | N/A |
| SS9 (n = 1) | N/A | 6.56 | 1.88 | 0.39 | 0.61 | 0.21 | N/A |
| SS15 (n = 5) | 1.66 ± 0.78 | 3.81 ± 0.61 | 3.18 ± 3.01 | N/A | 0.27 ± 0.11 | 0.14 ± 0.04 | 0.02 ± 0.01 |
| | | | | | | (n = 3) | (n = 3) |
| SS16: Slider | 5.25 ± 1.62 | 3.78 ± 0.81 | 1.10 ± 0.44 | N/A | 0.55 ± 0.22 | 0.11 ± 0.05 | N/A |
| II Arm1 | | | | | | | |
| (n = 5 | | | | | | | |
| except | | | | | | | |
| 3 H n = 4, | | | | | | | |
| 28 d n = 6) | | | | | | | |
| SS17: Slider | 1.81 ± 0.83 | 3.95 ± 3.09 | 0.86 ± 0.48 | N/A | 0.34 ± 0.21 | 0.13 ± 0.06 | N/A |
| II Arm2 | | | | | | | |
| (n = 5 | | | | | | | |
| except | | | | | | | |
| 3 H n = 4, | | | | | | | |
| 28 d n = 6) | | | | | | | |

BQL: Below Quantification Limit
N/A: Not available

TABLE 4I
Tissue concentration of Rivaroxaban, Argatroban and Sirolimus number of orders of magnitude higher than
IC50 for Anti-factor Xa/IIa or anti- cell proliferation for the respective drugs in the tissue of treated
segment and adjacent tissue segment of 5 mm proximal and 5 mm distal to the implanted device for SS15.

| Ratio of | | | | | | | | | |
|---|---|---|---|---|---|---|---|---|---|
| Tissue | | | | | | | | | |
| content | 1 Hour | | | 3 Hours | | | 1 Day | | |
| to IC50 | Proximal | Treated | Distal | Proximal | Treated | Distal | Proximal | Treated | Distal |
| Apixaban | 215142 | 1821895 | 95316 | 141612 | 688998 | 185185 | 3813 | 362200 | 9804 |
| Argatroban | 806 | 6693 | 356 | 506 | 2587 | 609 | 5 | 1462 | 16 |
| Sirolimus | 28000 | 432000 | 17000 | 35000 | 234000 | 38000 | 1200 | 292000 | 31800 |

| Ratio of | | | | | | | | | |
|---|---|---|---|---|---|---|---|---|---|
| Tissue | | | | | | | | | |
| content | 7 Days | | | 28 dyas | | | 3 month | | |
| to IC50 | Proximal | Treated | Distal | Proximal | Treated | Distal | Proximal | Treated | Distal |
| Apixaban | 272 | 31318 | 272 | 1089 | 34858 | 545 | 8 | 83061 | 5 |
| Argatroban | 1 | 132 | 1 | 4 | 158 | 2 | 0 | 351 | 0 |
| Sirolimus | 200 | 15400 | 2700 | 200 | 16700 | 1400 | 100 | 12800 | 200 |

*IC50 of Apixaban for anti-Xa 0.08 nM or 0.00004 ng/mg
*IC50 of Argatroban for anti-IIa 21 nM or 0.01 ng/mg
*IC50 of Sirolimus for cell proliferation 0.1 nM or 0.0001 ng/mg

TABLE 4J
Tissue concentration of Rivaroxaban, Argatroban and Sirolimus number of orders of magnitude higher than IC50
for Anti-factor Xa/IIa or anti- cell proliferation for the respective drugs in the tissue of treated segment
and adjacent tissue segment of 5 mm proximal and 5 mm distal to the implanted device for Slider II Arm1.

| Ratio of | | | | | | | | | |
|---|---|---|---|---|---|---|---|---|---|
| Tissue | | | | | | | | | |
| content | 1 Hour | | | 3 Hours | | | 1 Day | | |
| to IC50 | Proximal | Treated | Distal | Proximal | Treated | Distal | Proximal | Treated | Distal |
| Rivaroxaban | 286 | 5299 | 708 | 187 | 2335 | 235 | 10 | 399 | 10 |
| Argatroban | 351 | 5800 | 738 | 232 | 3076 | 247 | 7 | 356 | 8 |
| Rapamycin | 23000 | 451000 | 52500 | 26200 | 383000 | 37800 | 1800 | 91800 | 11000 |

| Ratio of | | | | | | |
|---|---|---|---|---|---|---|
| Tissue | | | | | | |
| content | 7 Days | | | 28 days | | |
| to IC50 | Proximal | Treated | Distal | Proximal | Treated | Distal |
| Rivaroxaban | N/A | 34 | N/A | 2 | 37 | 2 |
| Argatroban | N/A | 39 | N/A | N/A | 49 | N/A |
| Rapamycin | 400 | 14600 | 5500 | 500 | 9400 | 1100 |

*IC50 of Rivaroxaban for anti-Xa 21 nM or 0.0092 ng/mg
*IC50 of Argatroban for anti-IIa 21 nM or 0.01 ng/mg
*IC50 of Sirolimus for cell proliferation 0.1 nM or 0.0001 ng/mg

TABLE 4K
Tissue concentration of Rivaroxaban, Argatroban and Sirolimus number of orders of
magnitude higher than IC50 for Anti-factor Xa/IIa or anti- cell proliferation for
the respective drugs in the tissue of treated segment and adjacent tissue segment
of 5 mm proximal and 5 mm distal to the implanted device for Slider II Arm2.

| Ratio of | | | | | | | | | |
|---|---|---|---|---|---|---|---|---|---|
| Tissue | | | | | | | | | |
| content | 1 Hour | | | 3 Hours | | | 1 Day | | |
| to IC50 | Proximal | Treated | Distal | Proximal | Treated | Distal | Proximal | Treated | Distal |
| Rivaroxaban | 165 | 4164 | 284 | 326 | 2851 | 296 | 10 | 142 | 8 |
| Argatroban | 61 | 1106 | 85 | 64 | 756 | 82 | 4 | 112 | 3 |
| Rapamycin | 13900 | 213400 | 18100 | 24900 | 273200 | 39500 | 2600 | 108500 | 8600 |

| Ratio of | | | | | | |
|---|---|---|---|---|---|---|
| Tissue | | | | | | |
| content | 7 Days | | | 28 days | | |
| to IC50 | Proximal | Treated | Distal | Proximal | Treated | Distal |
| Rivaroxaban | 1 | 116 | 1 | 2 | 57 | N/A |
| Argatroban | N/A | 127 | N/A | N/A | 63 | N/A |
| Rapamycin | 100 | 38000 | 3400 | 300 | 17300 | 1300 |

IC50 of Rivaroxaban for anti-Xa 21 nM or 0.0092 ng/mg
IC50 of Argatroban for anti-IIa 21 nM or 0.01 ng/mg
IC50 of Sirolimus for cell proliferation 0.1 nM or 0.0001 ng/mg

Tables 4D-4K show that all three drugs Apixaban, Argatroban, and rapamycin maintain therapeutic tissue concentrations in the tissue segment up to 28 days, up to 90 days or longer, and furthermore achieve therapeutic tissue concentration in the adjacent tissue segment (±5 mm from the tissue segment such as Proximal and distal) at 1 hour, 3 hours and at/or up to 1 day. This can be important to inhibit thrombus formation in the stented segment, the device surface, and in the tissue adjacent to the stented segment as in many cases such tissue is injured by balloon deployment or stent edges.

Taking Apixaban IC50 for factor Xa inhibition to be about 0.08 nM or 0.00004 ng/mg; Rivaroxaban IC50 for factor Xa inhibition to be about 21 nM or 0.0092 ng/mg; Argatroban IC50 for factor IIa inhibition to be about 21 nM or 0.01 ng/mg; Sirolimus IC50 for cell proliferation to be about 0.1 nM or 0.0001 ng/mg. Table 4I-Table 4K are the tissue concentration of Apixaban or Rivaroxaban, Argatroban and Sirolimus are several order of magnitude higher (or times higher) than IC50 for Anti-factor Xa, anti-IIa, or anti-cell proliferation for the respective drugs in the tissue of treated segment and adjacent tissue segment of 5 mm proximal and 5 mm distal to the implanted device. It shows that Apixaban, Rivaroxaban, Argatroban and/or Sirolimus in tissue concentrations have one or more order of magnitudes higher concentration at the times specified, has from 1 to 6 orders of magnitude of tissue concentration for each of the drugs compared to their IC50, in the treated tissue segments up to 28 days, or up to 90 days.

### Example 5: In Vivo Animal Study of Anticoagulant1/Anticoagulant2/mTOR Eluting Stents (Scaffolds)

The test drug eluting stent systems containing anticoagulants were prepared as described in Example 4 and were evaluated at 28 days and 90 days following implantation in a porcine coronary artery. The control device was the Novolimus (m-TOR) eluting DESyne X2 stent.

The porcine artery was chosen as this model has been used extensively for stent and angioplasty studies resulting in a large volume of data on the pulmonary response properties and its correlation to human pulmonary response (Schwartz et al, Circulation. 2002; 106:1867 1873). The animals were housed and cared for in accordance with the Guide for the Care and Use of Laboratory Animals as established by the National Research Council.

All animals were pretreated with aspirin (325 mg) and Clopidogrel (75 mg) per oral dose beginning at least 3 days prior to the intervention and continuing for the duration of the study. After induction of anesthesia, the left or right femoral artery was accessed using standard techniques and an arterial sheath was introduced and advanced into the artery. Vessel angiography was performed under fluoroscopic guidance, a 7 Fr. guide catheter was inserted through the sheath and advanced to the appropriate location where intracoronary nitroglycerin was administered. An appropriate implantation segment of coronary artery was randomly selected and a 0.014″ guidewire inserted. Quantitative Coronary Angiography (QCA) was performed to document the results. The appropriately sized stent (3.0×14 mm or 3.5×14 mm) was advanced to the deployment site. The balloon was inflated at a steady rate to a pressure sufficient to achieve a balloon to artery ratio of approximately 1.1 to 1.0 but less than 1:2:1. Pressure was maintained for approximately 10 seconds before the balloon was deflated. Each pig was implanted with 3 test devices and one control device in the coronary arteries. Each time point a whole blood was drawn from animals for blood drug concentration test.

Follow up angiography imaging was performed at the designated endpoint for each of the animals. Quantitative coronary angiographic analysis was performed and the average percent diameter stenosis values and late lumen loss for the test arms and control DESyne X2 for the 28 days and 3-month time points are shown in Table 5A.

Upon completion of follow-up angiography imaging, the animals were euthanized. The hearts were harvested from each animal. Any myocardial lesions or unusual observations were reported. The coronary arteries were perfused with 10% buffered formalin at 100 to 120 mm Hg with the animal's ear tag until processed for histology.

Stented portions of coronary arteries were embedded in methyl methacrylate (MMA), then divided into a target of three blocks of approximately similar length (about 4 mm), identified as proximal, mid and distal segments. From three blocks, 3 to 5 cuts were made for histology evaluation.

Quantitative histopathological evaluation of stented artery sections was then performed and scored as indicated. The mean of each section was recorded and then averaged to provide a mean score per stent for the different parameters (Table 5A). The smaller the score, the better the efficacy.

Fibrin (strut-by-strut)

0=absent, or rare minimal spotting around struts
1=fibrin in small amounts, localized only around struts
2=fibrin moderately abundant or denser, extending beyond struts
3=abundant, dense fibrin, bridging between struts
Each strut in the section was scored; the mean fibrin score for each section was calculated and reported. The mean of the section means was calculated and reported, providing a mean fibrin score per stent.

Injury based on Schwartz et al. J Am Coll Cardiol 1992: 19:267-274. (strut-by-strut):

0=IEL intact
1=IEL lacerated
2=media completely lacerated
3=EEL lacerated
Each strut in the section as scored and the mean injury score for each section was calculated and reported. The mean of the section means was calculated and reported, providing a mean injury score per stent.

Inflammation (strut-by-strut)

0=no or very few (≤3) inflammatory cells around strut
1=few (˜4-10) inflammatory cells around strut
2=many (>10) inflammatory cells around strut, can extend into but does not efface surrounding tissue
3=many (>10) inflammatory cells, effacing surrounding tissue
Each strut in the section was scored and the mean inflammation score for each section was calculated and reported. The mean of the section means was calculated and reported, providing a mean inflammation score per device.

TABLE 5A
Histopathology Scores and Quantitative Coronary Angiography data Apixaban/Rivaroxaban,
Argatroban and Sirolimus releasing 14 mm stents at 7 days, 28 days and 3 month

| | | | | | | Diameter | |
|---|---|---|---|---|---|---|---|
| Time Point | | Device | Injury | Inflammation | Fibrin | Stenosis % | LLL, mm |
| 7 | Day | SS7 | 0.15 | 1.52 | 0.62 | N/A | N/A |
| | | (n = 1) | | | | | |
| | | SS9 | 0.61 | 1.66 | 0.44 | N/A | N/A |
| | | (n = 1) | | | | | |
| 28 | Day | SS7 | 0.21 | 0.49 | 0.94 | 16.7 | 0.71 |
| | | (n = 1) | | | | | |
| | | SS9 | 0.36 | 0.32 | 0.64 | 21.3 | 0.76 |
| | | (n = 1) | | | | | |
| | | SS15 | 0.38 ± 0.34 | 0.62 ± 0.12 | 1.67 ± 0.38 | 19.8 ± 4.1 | 0.45 ± 0.15 |
| | | (n = 3) | | | | | |
| | | DESyne X2 | 1.34 | 1.83 | 1.87 | 51.6 | 1.14 |
| | | (n = 1) | | | | | |
| | | SS16 (Slider | 0.91 ± 0.95 | 1.52 ± 1.03 | 1.90 ± 0.26 | 19.4 ± 13.8 | 0.72 ± 0.32 |
| | | II Arm1, | | | | | |
| | | n = 6) | | | | | |
| | | DESyne X2 | 1.17 ± 1.60 | 1.97 ± 1.46 | 1.32 ± 1.11 | 36.6 ± 27.3 | 0.99 ± 0.48 |
| | | (n = 2) | | | | | |
| | | SS17 (Slider | 1.01 ± 0.47 | 1.46 ± 0.43 | 1.88 ± 0.48 | 33.1 ± 12.8 | 0.86 ± 0.34 |
| | | II Arm2, | | | | | |
| | | n = 6) | | | | | |
| | | DESyne X2 | 0.87 ± 0.74 | 1.59 ± 0.95 | 1.93 ± 0.31 | 21.9 ± 11.6 | 0.71 ± 0.62 |
| | | (n = 2) | | | | | |
| 3 | Month | SS15 | 0.15 ± 0.18 | 0.52 ± 0.19 | 0.08 ± 0.06 | 18.2 ± 9.3 | 0.25 ± 0.29 |
| | | (n = 3) | | | | | |
| | | DESyne X2 | 0.43 | 0.59 | 0.76 | 22.3 | 0.52 |
| | | (n = 1) | | | | | |

LLL is an indicator of the amount cell proliferation or inhibition potency. It is used to measure efficacy between drugs for proliferation inhibition in mammalian arteries. The smaller the LLL, the better the efficacy of the drug.

As shown in Table 5A, SS15 composition providing the combination of Sirolimus, Apixaban and Argatroban released from stents had a smaller LLL compared to control which only had m-TOR inhibitor (Novolimus) and thus was unexpectedly more effective at inhibiting smooth muscle cell proliferation compared to Novolimus releasing stents at 28 days, and at 90 days. This was an unexpected finding for the test SS15 stents in comparison to the control DESyne X2 stents at the 28-day time point and/or at 90 days.

In an unexpected finding, SS15 stents composition eluting Apixaban, Argatroban, and the M-Tor inhibitor rapamycin exhibited more efficacy at inhibiting one or more of the following at 28 days and/or 90 day time points: cell proliferation, inflammation, injury, fibrin formation inhibition, and fibrin dissolution acceleration.

The LLL is an indicator of the amount cell proliferation or inhibition potency. It is used to measure efficacy between drugs for proliferation inhibition in mammalian arteries. The smaller the LLL, the better the efficacy of the drug.

As shown in Table 5A, SS16 shows the combination of Sirolimus, Rivaroxaban and Argatroban released from stents had a smaller LLL compared to control which only had m-TOR inhibitor (Novolimus) and thus was unexpectedly more effective at inhibiting smooth muscle cell proliferation compared to Novolimus releasing stents at 28 days.

As shown in Table 5A, SS17 composition configured to delay the release and tissue concentration of rapamycin within the first 1 hour and/or within the first 3 hours by incorporating rapamycin in the base coating shows the combination of Sirolimus, and/or lower tissue concentration of Rivaroxaban and Argatroban within at least the first hour showed less inhibition of SMC proliferation at 28 days.

TABLE 5B
Whole Blood PK Results of Apixaban/Argatroban/Sirolimus Eluting Stents from SS15 (from
study with SS15; target dose Apixaban/Argatroban/Sirolimus = 117/117/94; n = 5)

| | | Apixaban (ng/mL) | | | | | Argatroban (ng/mL) | | | | | Sirolimus (ng/mL) | | | | |
|---|---|---|---|---|---|---|---|---|---|---|---|---|---|---|---|---|
| Time Points | | 1 H | 3 H | 1 D | 7 D | 28 D | 1 H | 3 H | 1 D | 7 D | 28 D | 1 H | 3 H | 1 D | 7 D | 28 D |
| Pre- | | BQL | BQL | BQL | BQL | BQL | BQL | BQL | BQL | BQL | BQL | BQL | BQL | BQL | BQL | BQL |
| implant | | | | | | | | | | | | | | | | |
| Post 1^{st} | | 0.441 | 0.71 | 4.02 | 0.16 | 0.30 | 1.36 | 2.02 | 4.44 | 0.58 | 0.66 | 0.70 | 0.59 | 2.52 | 0.69 | 0.32 |
| implant | | | | | | | | | | | | | | | | |
| Post 2^{nd} | | 0.95 | 1.17 | 3.51 | 0.77 | 0.44 | 3.16 | 4.35 | 5.74 | 4.41 | 1.83 | 1.90 | 1.94 | 4.16 | 2.13 | 1.06 |
| implant | | | | | | | | | | | | | | | | |
| Post 3^{rd} | | 1.8 | 2.18 | 4.29 | 1.31 | 0.91 | 6.71 | 7.59 | 9.26 | 7.85 | 3.72 | 3.30 | 3.74 | 7.08 | 3.28 | 2.49 |
| implant | | | | | | | | | | | | | | | | |
| Post 4^{th} | | 2.56 | 2.91 | 5.01 | 1.72 | 1.65 | 10.2 | 11.0 | 11.4 | 10.9 | 7.33 | 4.80 | 5.22 | 8.06 | 4.17 | 4.73 |
| implant | | | | | | | | | | | | | | | | |
| Post 5^{th} | | 3.27 | 3.55 | 5.84 | 2.12 | 2.45 | 13.4 | 13.3 | 14.5 | 12.8 | 10.3 | 6.71 | 7.13 | 9.98 | 4.75 | 5.91 |
| implant | | | | | | | | | | | | | | | | |
| 15 | min. | 3.91 | 4.60 | — | — | — | 17.8 | 21.7 | — | — | — | 8.77 | 11.3 | — | — | — |
| 30 | min. | 4.78 | 5.20 | 8.69 | — | — | 22.7 | 24.6 | 22.6 | — | — | 11.2 | 12.3 | 17.2 | — | — |
| 45 | min. | 6.29 | — | — | — | — | 28.9 | — | — | — | — | 13.4 | — | — | — | — |
| 60 min. | | 6.32 | 6.28 | 9.91 | — | — | 28.2 | 29.0 | 24.4 | — | — | 12.8 | 12.7 | 21.3 | — | — |
| (1 hr) | | | | | | | | | | | | | | | | |
| 90 | min. | — | 7.26 | — | — | — | — | 31.1 | — | — | — | — | 13.7 | — | — | — |
| 120 min. | | — | 7.76 | 12.7 | — | — | — | 28.5 | 20.7 | — | — | — | 12.8 | 21.3 | — | — |
| (2 hr) | | | | | | | | | | | | | | | | |
| 150 | min. | — | 7.38 | — | — | — | — | 28.9 | — | — | — | — | 12.4 | — | — | — |
| 180 min. | | — | 6.89 | 12.8 | — | — | — | 20.4 | 16.9 | — | — | — | 9.32 | 14.0 | — | — |
| (3 hr) | | | | | | | | | | | | | | | | |
| 4 | hr | — | — | 13.3 | — | — | — | — | 12.9 | — | — | — | — | 12.4 | — | — |
| 5 | hr | — | — | 14.7 | — | — | — | — | 9.25 | — | — | — | — | 11.0 | — | — |
| 6 | hr | — | — | 13.3 | — | — | — | — | 7.02 | — | — | — | — | 10.5 | — | — |

| | | | | | | | | | | | | | | | |
|---|---|---|---|---|---|---|---|---|---|---|---|---|---|---|---|
| 24 hr | — | — | 1.51 | | — | — | — | 0.383 | — | — | — | — | 2.65 | — | — |
| (Day 1) | | | | | | | | | | | | | | | |
| Day 7 | — | — | — | BQL | — | — | — | — | BQL | — | — | — | — | 0.384 | — |
| Day 28 | — | — | — | — | BQL | — | — | — | — | BQL | — | — | — | — | BQL |

Note.
Blood volume in porcine model is about 40%-50% of adult human. Thus drug concentrations in human would typically be lower than the figures shown in table 5B.

TABLE 5C
Whole Blood PK Results of Rivaroxaban/Argatroban/Sirolimus
Eluting Stents from SS16 (Rivaroxaban Arm1; target dose
Rivaroxaban/Argatroban/Sirolimus = 117/117/94;
n = 5 except D 28 n = 6)

| | | Rivaroxaban (ng/mL) | | | | Argatroban (ng/mL) | | | | Sirolimus (ng/mL) | | | |
|---|---|---|---|---|---|---|---|---|---|---|---|---|---|
| Time Points | | 1 H | 1 D | 7 D | 28 D | 1 H | 1 D | 7 D | 28 D | 1 H | 1 D | 7 D | 28 D |
| Pre-implant | | BQL | BQL | BQL | BQL | BQL | BQL | BQL | BQL | BQL | BQL | BQL | BQL |
| Post 1^{st} | | 0.72 | 1.14 | 1.07 | 0.69 | 1.96 | 2.94 | 2.22 | 1.89 | 0.52 | 0.69 | 0.66 | 0.48 |
| implant | | | | | | | | | | | | | |
| Post 2^{nd} | | 1.34 | 2.16 | 2.14 | 1.44 | 5.05 | 6.92 | 5.91 | 4.76 | 1.56 | 1.47 | 1.75 | 1.29 |
| implant | | | | | | | | | | | | | |
| Post 3^{rd} | | 2.10 | 2.57 | 2.94 | 2.17 | 10.3 | 10.1 | 10.2 | 8.22 | 3.53 | 2.22 | 3.35 | 2.32 |
| implant | | | | | | | | | | | | | |
| Post 4^{th} | | 2.67 | 3.36 | 3.52 | 2.80 | 15.0 | 17.2 | 13.6 | 11.69 | 4.69 | 3.41 | 4.69 | 3.59 |
| implant | | | | | | | | | | | | | |
| Post 5^{th} | | 2.92 | 4.32 | 3.95 | 3.33 | 18.9 | 19.9 | 19.1 | 14.42 | 6.15 | 5.37 | 7.24 | 5.08 |
| implant | | | | | | | | | | | | | |
| Post 6^{th} | | — | — | — | 4.32 | — | — | — | 19.05 | — | — | — | 6.58 |
| implant | | | | | | | | | | | | | |
| 15 | min. | 3.32 | 4.05 | — | — | 23.5 | 22.8 | — | — | 8.38 | 6.92 | — | — |
| 30 | min. | 3.34 | 3.76 | — | — | 27.5 | 27.0 | — | — | 10.1 | 8.56 | — | — |
| 45 | min. | 3.68 | 3.47 | — | — | 31.9 | 31.5 | — | — | 10.9 | 9.93 | — | — |
| 60 min. | | 4.08 | 2.61 | — | — | 31.1 | 31.5 | — | — | 11.2 | 10.7 | — | — |
| (1 hr) | | | | | | | | | | | | | |
| 90 | min. | — | 2.43 | — | — | — | 30.8 | — | — | — | 12.3 | — | — |
| 120 min. | | — | 2.30 | — | — | — | 26.6 | — | — | — | 12.1 | — | — |
| (2 hr) | | | | | | | | | | | | | |
| 150 | min. | — | 2.08 | — | — | — | 26.6 | — | — | — | 11.7 | — | — |
| 180 min. | | — | 1.95 | — | — | — | 25.1 | — | — | — | 10.3 | — | — |
| (3 hr) | | | | | | | | | | | | | |
| 4 | hr | — | 1.23 | — | — | — | 18.4 | — | — | — | 7.62 | — | — |
| 5 | hr | — | 1.11 | — | — | — | 13.5 | — | — | — | 6.89 | — | — |
| 6 | hr | — | 1.03 | — | — | — | 11.0 | — | — | — | 6.09 | — | — |

| | | | | | | | | | | | | |
|---|---|---|---|---|---|---|---|---|---|---|---|---|
| 24 hr | — | BQL | — | — | — | 1.92 | — | — | — | 2.14 | — | — |
| (Day 1) | | | | | | | | | | | | |
| Day 7 | — | — | BQL | — | — | — | BQL | — | — | — | 0.44 | — |
| Day 28 | — | — | — | BQL | — | — | — | BQL | — | — | — | BQL |

Note.
Blood volume in porcine model is about 40%-50% of adult human. Thus drug concentrations in human would typically be lower than the figures shown in table 5C.

TABLE 5D
Whole Blood PK Results of Rivaroxaban/Argatroban/Sirolimus Eluting Stents from
SS17 Rivaroxaban/Argatroban/Sirolimus = 117/117/94; n = 5 except D 28 n = 6)

| | | Rivaroxaban (ng/mL) | | | | Argatroban (ng/mL) | | | | Sirolimus (ng/mL) | | | |
|---|---|---|---|---|---|---|---|---|---|---|---|---|---|
| Time Points | | 1 H | 1 D | 7 D | 28 D | 1 H | 1 D | 7 D | 28 D | 1 H | 1 D | 7 D | 28 D |
| Pre- | | BQL | BQL | BQL | BQL | BQL | BQL | BQL | BQL | BQL | BQL | BQL | BQL |
| implant | | | | | | | | | | | | | |
| Post 1^{st} | | 3.77 | 3.62 | 3.18 | 3.20 | 11.7 | 11.1 | 11.0 | 14.05 | BQL | 0.11 | BQL | BQL |
| implant | | | | | | | | | | | | | |
| Post 2^{nd} | | 5.27 | 6.16 | 5.90 | 5.79 | 20.5 | 24.7 | 25.9 | 32.44 | 0.17 | 0.36 | BQL | 0.06 |
| implant | | | | | | | | | | | | | |
| Post 3^{rd} | | 9.27 | 11.4 | 8.41 | 8.66 | 41.3 | 45.2 | 44.3 | 52.20 | 0.28 | 0.44 | 0.22 | 0.21 |
| implant | | | | | | | | | | | | | |
| Post 4^{th} | | 10.9 | 16.7 | 10.8 | 9.02 | 54.4 | 59.6 | 63.2 | 65.75 | 0.47 | 0.91 | 0.77 | 0.46 |
| implant | | | | | | | | | | | | | |
| Post 5^{th} | | 12.5 | 22.3 | 12.0 | 10.70 | 65.7 | 70.3 | 73.6 | 79.38 | 0.98 | 1.38 | 1.08 | 0.69 |
| implant | | | | | | | | | | | | | |
| Post 6^{th} | | — | — | — | 13.42 | — | — | — | 96.18 | — | — | — | 1.20 |
| implant | | | | | | | | | | | | | |
| 15 | min. | 10.8 | 24.0 | — | — | 73.4 | 67.2 | — | — | 2.11 | 2.88 | — | — |
| 30 | min. | 8.44 | 20.1 | — | — | 51.9 | 51.7 | — | — | 2.79 | 4.65 | — | — |
| 45 | min. | 7.82 | 19.5 | — | — | 42.4 | 46.5 | — | — | 3.96 | 5.35 | — | — |
| 60 min. | | 7.54 | 18.5 | — | — | 38.3 | 44.2 | — | — | 4.32 | 6.97 | — | — |
| (1 hr) | | | | | | | | | | | | | |
| 90 | min. | — | 15.0 | — | — | — | 32.9 | — | — | — | 8.08 | — | — |
| 120 min. | | — | 11.6 | — | — | — | 25.8 | — | — | — | 8.12 | — | — |
| (2 hr) | | | | | | | | | | | | | |
| 150 | min. | — | 11.3 | — | — | — | 20.5 | — | — | — | 7.97 | — | — |
| 180 min. | | — | 8.39 | — | — | — | 16.3 | — | — | — | 7.10 | — | — |
| (3 hr) | | | | | | | | | | | | | |
| 4 | hr | — | 3.74 | — | — | — | 9.30 | — | — | — | 5.63 | — | — |
| 5 | hr | — | 3.90 | — | — | — | 6.78 | — | — | — | 6.80 | — | — |
| 6 | hr | — | 3.10 | — | — | — | 5.40 | — | — | — | 6.30 | — | — |

| | | | | | | | | | | | | |
|---|---|---|---|---|---|---|---|---|---|---|---|---|
| 24 hr | — | 0.16 | — | — | — | 0.48 | — | — | — | 2.92 | — | — |
| (Day 1) | | | | | | | | | | | | |
| Day 7 | — | | BQL | — | — | — | 0.36 | — | — | — | 0.49 | — |
| Day 28 | — | | — | BQL | — | — | — | BQL | — | — | — | 0.66 |

Note.
Blood volume in porcine model is about 40%-50% of adult human. Thus drug concentrations in human would typically be lower than the figures shown in table 5D.

Tables 5B, 5C, and 5D show although local (tissue adjacent to the device) concentrations of Apixaban, Rivaroxaban, and Argatroban reached therapeutic levels, the systemic blood concentrations for each of the drugs were below one or more of the following to achieve systemic therapeutic concentrations: systemic Cmax, Systemic Cmean, Systemic Ctrough. These tables also show that each of these agents reached BQL levels within one of the following: 1 day, 7 days, 28 days, or 90 days

### Example 6: Anti-Proliferative Activity of Apixaban, Argatroban and Rapamycin Combination

Anti-proliferative activity of Apixaban, Argatroban, and Rapamycin was tested in Human Aortic SMC (HAoSMC, ATCC, PCS-100-012). Cell proliferation assay was done in 96-well format. Low passage cells were trypsinized and seeded in 96 well plates at a density of ˜4000 cells/well. The cells are allowed to attach overnight in a CO₂ incubator. Next day, the medium was removed and replaced with fresh complete medium containing various concentrations of the test compounds. The final concentration of vehicle (DMSO) in the test medium was 0.1%. After adding test compounds, the cells were incubated for 72 hours. Following this period, the medium was removed and then added fresh medium (100 μl) containing CellTiter Aqueous (1× concentration final) to the wells and incubated for 2 hours in the CO2 incubator. At the end of incubation measured fluorescence with a plate-reader. Controlled incubations with untreated cells and blank incubations containing only medium were included and tested similarly. Based on the cell viability assay the percentage inhibition of the cell proliferation was determined at the different concentrations of the drug tested.

The cell proliferation assay was performed with different concentrations of Apixaban and Argatroban when combined with Rapamycin. Following the cell proliferation assay as indicated earlier, the percent cell proliferation inhibition was determined, and the assay results plotted to determine the IC50.

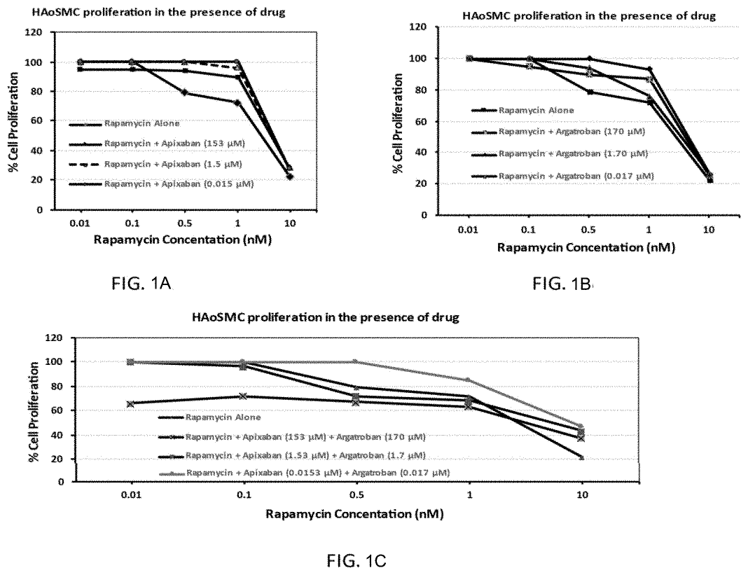

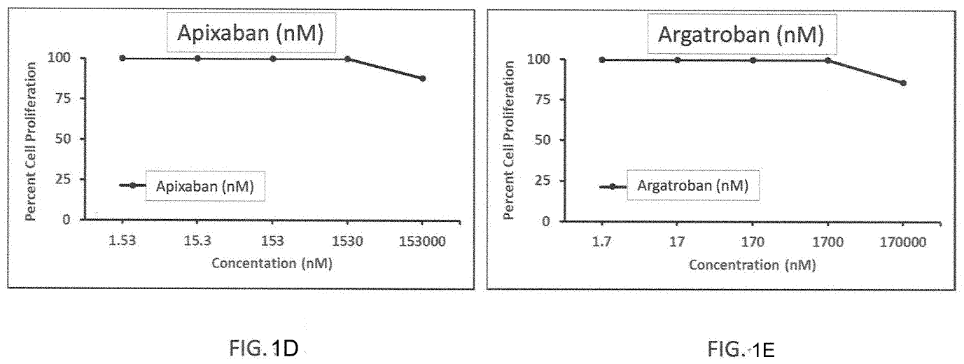

FIGS. 1A-1C show HAoSMC proliferation inhibition in the presence of different drug combinations. The data shows the combination of Apixaban, Argatroban surprisingly and unexpectedly enhanced the anti-proliferative effects of rapamycin on smooth muscle cell proliferation as measured by cell proliferation test when Apixaban and Argatroban were combined with rapamycin, i.e the combination of Apixaban, Argatroban, and rapamycin were more potent than rapamycin alone at inhibiting SMS proliferation.

FIGS. 1D and 1E show HAoSMC proliferation in presence of different concentrations of Apixaban or Argatroban. In order to determine if Apixaban or Argatroban independently had inhibitory effect on the proliferation of HAoSMC, a proliferation assay in the presence of either of these two drugs at different concentrations were tested as described earlier. Various concentration of Apixaban alone or Argatroban alone had small to no inhibition of HAoSMC proliferation was observed as shown in FIGS. 1D-lE.

### Example 7: Activated Clotting Time (ACT) Evaluation of Apixaban, Argatroban or a Combination of Apixaban and Argatroban

The activated clotting time (ACT) evaluation of anticoagulants was performed in Calcium-reconstituted sheep blood and recorded employing the Hemochron® Response device.

The ACT measurements were made in citrated sheep blood. 1.9 ml of citrated sheep blood was added to a test tube containing an activator (Hemochron@Celite@ ACT tubes, Lot F8FTE026 from Accriva Diagnostics, Inc.). A target amount of drug solution was then added into the test tube. The test tube was gently swirled so that the blood and drug was well-mixed. 0.1 ml of 0.3M calcium chloride was then added. The tube was gently shaken before being inserted into the Hemochron Response detector. The ACT read out was recorded and reported. The ACT of the control blood in the absence of any drug as first determined to establish a baseline. Then ACT was determined in the presence of different concentrations of the drug as a single component. Selected drug combinations, were then tested to evaluate for potential synergy in action between the two drugs.

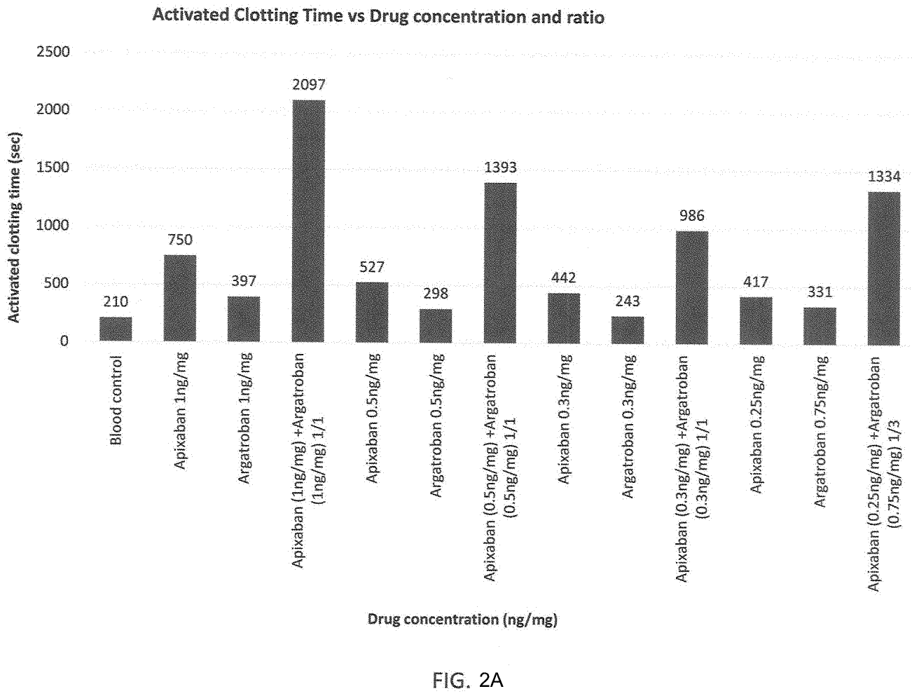

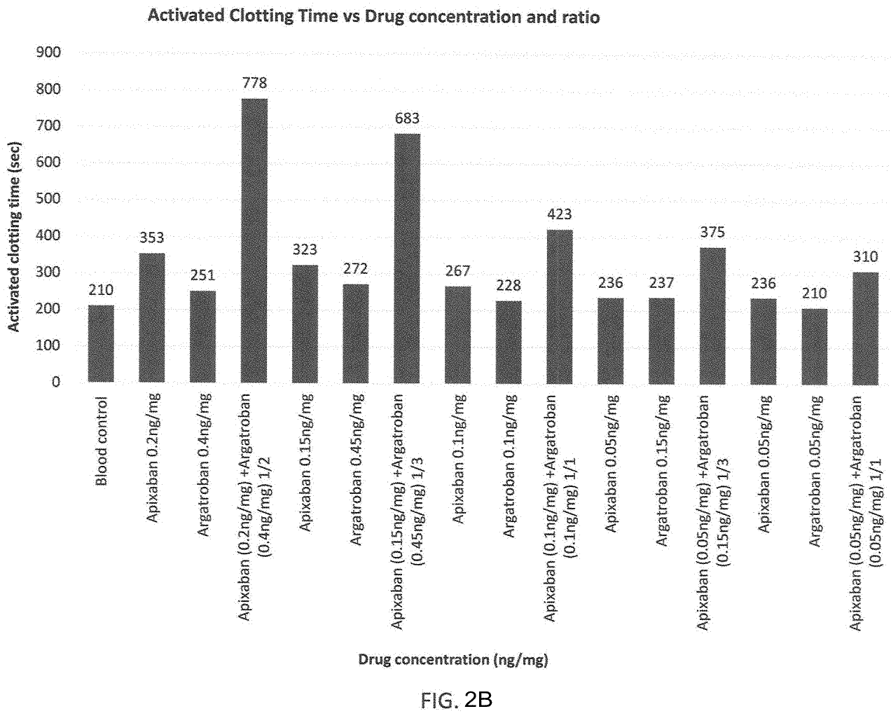

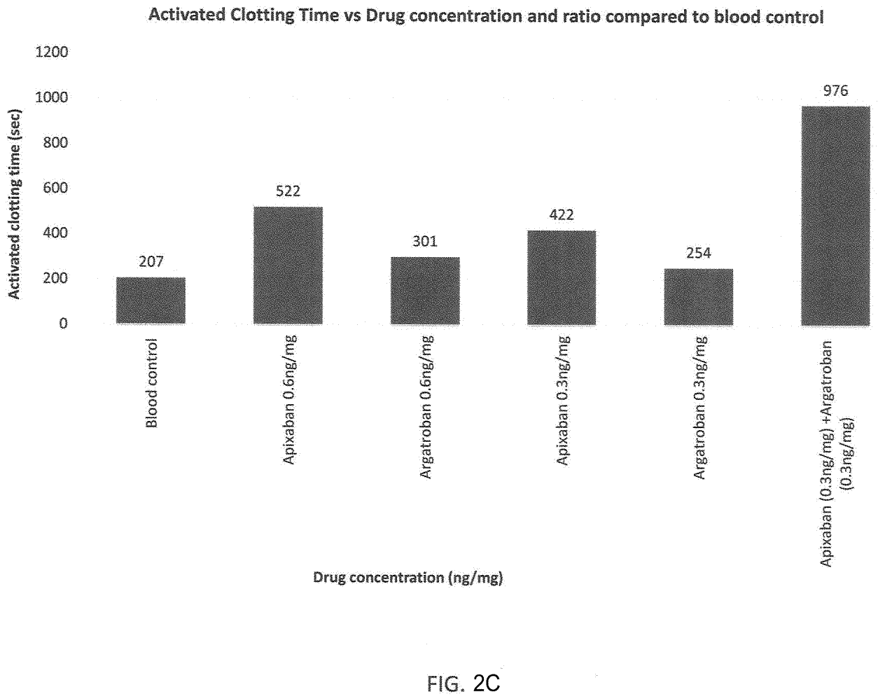

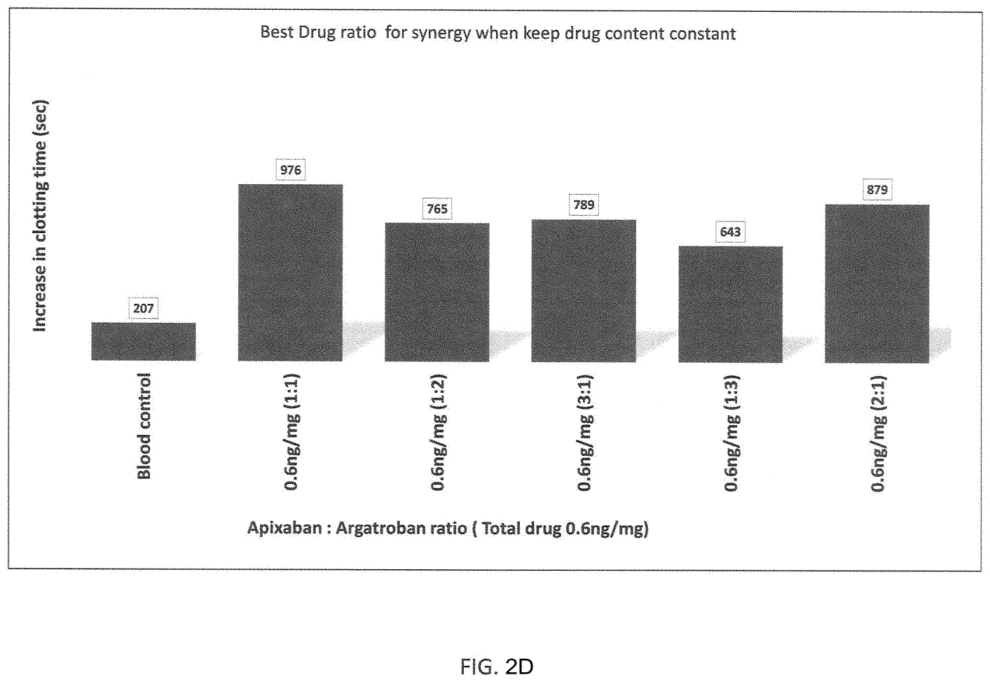

As shown in FIGS. 2A-2D, the clotting time was observed to be significantly extended or increased at a higher drug combination concentration. The Apixaban/Argatroban combination achieved ACT levels that were higher than the sum of the individual ACT values, indicating a synergistic effect between these drug combinations. This may be particularly important when delivering these drugs locally (adjacent to injured tissue) to inhibit clot formation. The figures are presented in ng/mg wherein the density of blood and tissue are approximately the same.

It was found, unexpectedly, that the combination drug concentrations of 0.025 ng/mg for each Apixaban and Argatroban drug extended the ACT by a larger time (as shown in FIG. 2B).

It was found, unexpectedly, that the combination drug concentrations of 0.3 ng/mg for each drug (0.6 ng/mg total) extended the ACT by a larger time (i.e., was more effective) than the ACT for each individual drug at 0.6 ng/mg concentration (ACT of 976 for the combination versus 522 for Apxaban versus 301 for Argatroban as shown in FIG. 2C).

FIG. 2C further shows, unexpectedly, that the combination drug concentrations of 0.3 ng/mg for each drug (0.6 ng/mg total) extended the ACT by a larger time (i.e., was more effective) than the ACT for the sum of each individual drug ACT at 0.3 ng/mg or at 0.6 ng/mg concentration. (ACT of 976 for the combination versus 676 (for the sum of individual drugs having 0.3 ng/mg concentrations).

It is important to note that drug tissue concentrations for factor Xa inhibitors like Rivaroxiban or Apixaban alone or in combination with factor IIa Argatrban to have sufficient tissue concentrations in the stented tissue segment and in the adjacent tissue segment to have therapeutic levels for each drug to be larger than 0.02 ng/mg, larger than 0.1 ng/mg, preferably larger than 0.2 ng/mg of tissue, preferably 0.3 ng/mg of tissue, more preferably larger than 1 ng/mg of tissue at or within 3 hours after implantation, or at or within 1 day after implantation, to inhibit clot formation.

It was also shown that the combination of Apixaban and Argatroban with m-TOR inhibitor inhibited thrombus formation in a shunt model (e.g., as in Example 8).

### Example 8: Ex Vivo Testing of Drug Eluting Stent Compared with 2 Anticoagulants and mTOR Eluting Stents

The thrombogenicity of a drug eluting stent system with two anticoagulant Apixaban and Argatroban in combination with rapamycin at two different loading drug doses was evaluated at 1 hour in an arteriovenous ex vivo shunt in a porcine model wherein the devices were deployed in a blood compatible polymeric tubing.

The control stents were 16-o-demethyl rapamycin m-TOR inhibitor (Novolimus) drug eluting coronary stent (DESyne, Elixir) and m-TOR inhibitor Zotarolimus eluting coronary stent (Resolute, Medtronic, USA).

The test arm for this experiment were SS9, SS9*, and SS10* and were manufactured as follows: Each polymer solution and each drug solutions were combined together ((Sirolimus and anticoagulant Apixaban and Argatroban was 1:1:1) to poly(L-lactide acid-co-glycolic acid) by weight ratio was 5:2 matrix) according to the target drug dose of 235 μg for each anticoagulant and 94 μg for Sirolimus for SS9, SS9* test arm was about ⅓ of each of the drugs dose as follows: Sirolimus and anticoagulant Apixaban and Argatroban was 1:1:1) to poly(L-lactide acid-co-glycolic acid) by weight ratio was 5:2 on matrix) according to the target drug dose of 78.3 μg for each anticoagulant and 31.3 μg for Sirolimus, and SS10* arm was Sirolimus and anticoagulant Apixaban and Argatroban was 1:1:3) to poly(L-lactide acid-co-glycolic acid) by weight ratio was 5:2 matrix) according to the target drug dose of 39.2 μg for Apixaban and 117.5 μg for Argatroban and 31.3 μg for Sirolimus.

A microprocessor controlled ultrasonic sprayer was used to coat each of the stents 14 mm length uniformly with each of the drug/polymer matrix solution. After coating, the stents were placed in a 70° C. oven for about 2 hours to remove the solvent. The stents were then mounted on balloon catheters and crimped. The catheters were then inserted in coils and packaged. The pouches were sterilized.

The ex-vivo shunt model to evaluate thrombogenicity has been extensively employed to evaluate the biocompatibility of different drug eluting stents (Waksman et al. Circ Cardiovasc Interv. 2017; 10:e004762, Otsuka et al. J Am Coll Cardiol Intv 2015; 8:1248-60, Lipinski et al. EuroInterv 2018; Jaa-369 2018) The animals were housed and cared for in accordance with the Guide for the Care and Use of Laboratory Animals as established by the National Research Council.

The two pigs that were employed in this study did not receive any aspirin or clopidogel pretreatment. Further all procedures were performed in the absence of any anticoagulant including heparin. After induction of anesthesia, an arterial bypass shunt from the femoral artery to the femoral vein was created. Blood flow was established through the shunt. Flow rates through the shunt was continuously monitored during the procedure with a flow probe that was placed on the shunt tubing proximal to the arterial flow.

In the first pig three control devices were deployed in the first shunt and the blood flow through the shunt was performed for a period of 1 hour. Following perfusion, the shunt tubing containing the stents was rinsed with saline and then fixed in situ with 10% buffered formalin in order to capture the thrombus, if any, that are deposited on the stent surface.

Similar procedure with 3 shunts with only one stent SS9* or SS9 in each shunt was tested with a perfusion time of 1 hour for each of the shunts.

In the second pig three control devices were deployed in the first shunt and the blood flow through the shunt was performed for a period of 1 hour. Following perfusion, the shunt tubing containing the stents was rinsed with saline and then fixed in situ with 10% buffered formalin in order to capture the thrombus, if any, that are deposited on the stent surface.

Similar procedure with 2 shunts with only one stent SS10* in each shunt was tested with a perfusion time of 1 hour for each of the shunts.

Promptly following perfusion in each of the shunts, the tubing containing the stents was gently rinsed with saline under gravity flow and then fixed in situ with 10% buffered formalin in order to anchor the thrombus, if any, that are deposited on the stent surface.

The stents were then removed from the tubing and bisected longitudinally. Low magnification photographs of the luminal side of two halves of the control and test stents were recorded.

The two halves of the stents were then processed for scanning electron microscopy (SEM) so as to examine the thrombus on the luminal side of the stent. Low (15×) and high (200×) magnification images of the stent surface were captured to evaluate the extent of thrombus deposition on the luminal surface of the stent.

Significant number of thrombus was observed on the luminal surface (inner surface) of the control DES stents as seen on the low and high magnification SEM images whereas there was little or no thrombus deposits on the test stents with combinations of Apixaban and Argatroban and m-TOR. The number of thrombus deposits on the control and test stents as evaluated from SEM images are shown in Table 6. The data shows that the combinations of Apixaban and Argatroban and m-TOR inhibited thrombus formation in the shunt model better than control.

Table 6 shows several therapeutic compositions of factor Xa inhibitor, factor IIa, and M-tor inhibitor releasing stents had less thrombus (clot formation) compared to M-Tor inhibitor alone releasing stents.

The composition comprising a combination of factor Xa inhibitor, a factor II inhibitor and an anti-proliferative were surprisingly more effective than the anti-proliferative alone.

TABLE 6
Thrombus deposits on the control and test
stents as evaluated from SEM images

| | | | Number of |
|---|---|---|---|
| | | | Thrombus |
| Animal # | Control/Test DES | Device | deposits |
| 1 | Control | DESyne-1 | 18 |
| | | DESyne-2 | 40 |
| | | Resolute | 14 |
| | Test—Apixaban:Argatroban(1:1) | SS9* | 4 |
| | | SS9* | 0 |
| | | SS9 | 3 |
| 2 | Control | DESyne-1 | 17 |
| | | DESyne-2 | 17 |
| | | Resolute | 15 |
| | Tes—Apixaban:Argatroban(1:3) | SS10* | 2 |
| | | SS10* | 2 |

### Example 9 Preparation of Anticoagulant or mTOR Inhibitors Coated Balloon

Rivaroxaban, Apixaban, Novolimus, or Rapamycin were dissolved into dichloromethane at room temperature and vortex until the drug was uniformly dissolved/dispersed; Ethylene Vinyl Acetate/Polyvinylpyrrolidone (MW=1.3M) was dissolved into 6.2500 methanol in dichloromethane at room temperature and vortex until the polymer had uniformly dissolved/dispersed (1000% Dichloromethane was used for Polyethylene oxide (MW=8M)).

Each polymer solution and each drug solutions were combined together (Rivaroxaban to Ethylene vinyl acetate/Polyvinylpyrrolidone (MW=1.3M) by weight ratio was 2:1:1 for SV300), (Apixaban to Polyethylene oxide (MW=8M)/Polyvinylpyrrolidone (MW=1.3M)/Butylated hydroxytoluene by weight ratio was 2/1/1/0.01 for VGR), (Novolimus to Ethylene vinyl acetate/Polyvinylpyrrolidone (MW=1.3M) by weight ratio was 2:1:1 NEV250 and NEV200), and (Rapamycin to Ethylene vinyl acetate/Polyvinylpyrrolidone (MW=1.3M) by weight ratio was 2:1:1 for REV200) according to the target drug dose of 300 μg for Rivaroxaban in SV300, 800 μg for Apixaban in VGR, 250 μg for Novoliums in NEV250, 200 μg for Novoliums in NEV200 and 200 μg for Rapamycin in REV200.

A microprocessor controlled ultrasonic sprayer was used to coat each of the balloon 14 mm or 18 mm length uniformly (as shown in Table 7) with each of the drug/polymer matrix solutions. Balloons were inflated prior to coating and held by a rotating fixture. A rotational motor rotated the catheter and balloon 360 degrees while a mandrel and a clamp securely held the catheter tail in place and rotate. The coating parameter was adjusted to ideal coating texture and the morphology and the profile of the interface between drug and balloon surface. After coating, the balloons were placed in a vacuum chamber to remove the solvent. The balloons were then tri-folded before putting on the protective sheath. The balloon catheters were then inserted in coils and packaged. The pouches were sterilized.

### Example 10: In Vivo Pharmacokinetics of Drug Eluting Balloon with Anticoagulant

The pharmacokinetics of the drug eluting balloon systems with anticoagulant of Example 9 were evaluated in porcine coronary/internal thoracic arteries in the non-diseased porcine coronary artery model. The balloon (e.g., the balloon of a balloon-catheter of a stent-delivery system) was advanced to a site to be treated (e.g., an occluded or weakened section of a blood vessel to be opened up or supported by a stent) in the body of a subject and inflated to a desired inflation diameter. Before, during, and/or after inflation of the balloon, the balloon released the therapeutic composition to, into, or at the treatment site, or to, into, or at an area adjacent thereto, by any suitable mechanism (e.g., concentration gradient, diffusion, pressure, or mechanical force, or a combination thereof). Safety of the device was evaluated at the 7- and 28-day time points following treatment in the coronary arteries and the tissue pharmacokinetics were evaluated following treatment in the coronary/thoracic arteries at the 7 and 28 day time points. Following treatment with the 14 mm length test drug coated balloon or the control plain balloon, an 18 mm length drug eluting balloon was deployed over the balloon treated segment of the coronary/thoracic artery. The tissue concentration is shown in ng/mg tissue.

The coated balloons were evaluated for drug delivery efficiency in an animal study. Drug transfer of the coated balloons into arterial segments were evaluated using harvested pig arteries. The arterial wall was separated after animal study. The arterial walls were then stored in an individual labeled vial. All samples were kept on dry ice until stored in the −80° C. freezer. All samples were then frozen to 70° C. prior to being analyzed. The tissue was extracted with Acetonitrile/methanol for Rivaroxaban, Apixaban, Novolimus and Rapamycin. The tissue content of Rivaroxaban, Apixaban, Novolimus and Rapamycin from the different drug coated balloon were analyzed using liquid chromatography mass spectroscopy (LCMS) with corresponding reference standards.

TABLE 7
Coated Balloon tissue concentration in vivo by different time period.

| Balloon | | | | Time | Tissue |
|---|---|---|---|---|---|
| type | Balloon Coating information | Artery type | Drug | period | concentration |
| SV300 | 300 μg Rivaroxaban in 150 μg | Coronary | Rivaroxaban | 15 min | 1.23 ± 0.37 |
| 18 mm | Ethylene vinyl acetate and | Artery | | | (n = 4) |
| Balloon | 150 μg Polyvinylpyrrolidone | Superficial | Rivaroxaban | 15 min | 40.58 ± 64.71 |
| | (MW = 1.3M) matrix | Femoral | | | (n = 4) |
| | | Artery (SFA) | | | |
| VGR | 800 μg Apixaban in 400 μg | Coronary | Apixaban | Acute | 60.09 ± 80.49 |
| 14 mm | Polyethylene oxide (MW = 8M), | Artery | | | (n = 2) |
| Balloon | 400 μg Polyvinylpyrrolidone | | | ID | 0.08 ± 0.08 (n = 3) |
| | (MW = 1.3M) and 4 μg Butylated | | | | |
| | hydroxytoluene matrix | | | 7D | 0.0001 ± 0.0001 |
| | | | | | (n = 3) |
| | | | | 28D | 0.003 ± 0.005 |
| | | | | | (n = 3) |
| NEV250 | 250 μg Novolimus in 125 μg | Coronary | Novolimus | 6H | 6.52 ± 3.68 (n = 3) |
| 18 mm | Ethylene vinyl acetate and | Artery | | 3D | 3.77 ± 1.62 (n = 3) |
| Balloon | 125 μg Polyvinylpyrrolidone | | | 7D | 1.47 ± 0.37 (n = 3) |
| | (MW = 1.3M) matrix | Superficial | Novolimus | 6H | 5.94 ± 3.66 (n = 4) |
| | | Femoral | | 3D | 4.4 ± 1.95(n = 4) |
| | | Artery (SFA) | | 7D | 1.16 ± 0.23 (n = 4) |
| NEV200 | 200 μg Novolimus in 100 μg | Coronary | Novolimus | 7D | 0.60 ± 0.64(n = 7) |
| 14 mm | Ethylene vinyl acetate and 100 | Artery | | | |
| Balloon | μg Polyvinylpyrrolidone | | | | |
| REV200 | 200 μg Rapamycin in 100 μg | Coronary | Rapamycin | 7D | 1.45 ± 1.23(n = 8) |
| 14 mm | Ethylene vinyl acetate and 100 | Artery | | | |
| Balloon | μg Polyvinylpyrrolidone | | | | |

As shown in Table 7, Rivaroxaban tissue concentration of tissue adjacent to the balloon treated segment ranges from at least 1.23 ng/mg within or at 15 min in coronary artery tissue to at least 40.58 ng/mg in Superficial Femoral Artery (SFA) tissue, within or at 15 minutes; Apixaban tissue concentration of tissue adjacent to the balloon treated segment in coronary artery tissue ranges from at least 60.09 ng/mg at acute to at least 0.08 ng/mg within or at 1 day to at least 0.0001 ng/mg within or at 7 days to at least 0.003 ng/mg within or at 28 days; Novolimus tissue concentration of tissue adjacent to the balloon treated segment in coronary artery tissue ranges from at least 6.52 ng/mg within or at 6 hours to at least 3.77 ng/mg within or at 3 day to at least 1.47 ng/mg within or at 7 days; Novolimus tissue concentration of tissue adjacent to the balloon treated segment in Superficial Femoral Artery (SFA) tissue ranges from at least 5.94 ng/mg within or at 6 hours to at least 4.4 ng/mg within or at 3 day to at least 1.16 ng/mg within or at 7 days; Rapamycin tissue concentration of tissue adjacent to the balloon treated segment in coronary artery tissue ranges from at least 1.45 ng/mg within or at 7 days. Rivaroxaban, Apixaban, Novolimus or Rapamycin released locally sufficiently from a coated balloon catheter to inhibit smooth muscle proliferation after vessel injury at sufficient tissue concentration up to 7 days.

### Example 11: Preparation of Anticoagulant Eluting Valve Implant or Part of the Implant not Covered by a Sleeve with Carrier

A valve or valve repair implant or part of the implant comprising the valve is coated with a coating containing anticoagulant Apixaban or Rivaroxaban and Argatroban.

Poly (L-lactide acid-co-glycolic acid) polymer is dissolved into dichloromethane at room temperature and vortex until the polymer had uniformly dissolved/dispersed. Anticoagulant (Apixaban or Rivaroxaban & Argatroban) are placed in a vial and dissolved in dichloromethane/Methanol at room temperature and vortex until all the drug was uniformly dissolved/dispersed.

Each polymer solution and each drug solutions are combined together (anticoagulant (Apixaban or Rivaroxaban & Argatroban with weight ratio 1 to 1) to poly (L-lactide acid-co-glycolic acid) by weight ratio was 3:1) according to the target drug dose.

The valve, and/or valve repair implant, and/or at least part of the implant comprising the valve optionally undergo surface treatment if the surface is not porous (i.e. plasma treatment or other surface friction treatment).

A microprocessor controlled ultrasonic sprayer was used to coat the valve of the drug containing carrier solution to the entire surface of the implant or part of the surface. After coating, the implant is placed in a 70° C. oven for about 2 hours to remove the residue solvent. The transcatheter valve or valve repair implant is then mounted on the delivery catheter. The catheters is then inserted in coils and packaged. The pouches were sterilized.

### Example 12: Preparation of Anticoagulant Eluting Valve Implant or Part of the Implant Covered by a Sleeve with Carrier

A valve or valve repair implant or part of the implant covered by a sleeve can have a polymer coating containing anticoagulant Apixaban or Rivaroxaban, Argatroban or a combination of both on top, part of, or adjacent to the sleeve made from ePTFE, Dacron, knitted or weaved fabric, or those known in the art.

The sleeve is infused with a polymer coating in a solvent solution with anticoagulant Apixaban or Rivaroxaban, Argatroban or a combination of both drugs and drug solution into the said sleeve and said solvent is allowed to evaporate leaving either polymer coating or drug in the pores of the sleeve. The sleeve is placed prior to being attached to the valve or valve repair implant or after it has been attached to the implant.

Poly (L-lactide acid-co-glycolic acid) polymer is dissolved into dichloromethane at room temperature and vortex until the polymer is uniformly dissolved/dispersed. Anticoagulant (Apixaban or Rivaroxaban & Argatroban) is placed in a vial and dissolved in dichloromethane/Methanol at room temperature and vortex until all the drug is uniformly dissolved/dispersed.

Each polymer solution and each drug solutions is combined together (anticoagulant (Apixaban or Rivaroxaban and Argatroban with weight ratio 1 to 1) to poly (L-lactide acid-co-glycolic acid) by weight ratio was 3:1) according to the target drug dose.

The sleeve optionally undergoes surface treatment if the surface is not porous (i.e. plasma treatment or other friction surface treatment). After surface treatment, the coating is spray coated or dip coated. The coating can be inside the sleeve or dipped onto the sleeve or coated on the sleeve.

When spray coated, a microprocessor controlled ultrasonic sprayer is used to coat the sleeve containing drug/excipient solution to the entire surface of the implant. After coating, the sleeve is placed in a 70° C. oven for about 2 hours to remove the residue solvent. The sleeve is then attached to the valve or valve repair implant if not prior to being attached to the implant. The valve or valve repair implant with sleeve attached is then mounted on the delivery catheter. The transcatheter valve or valve repair device is then inserted in a coil and packaged. The pouches were sterilized.

### Example 13: Preparation of Drug Eluting Stent Having Anticoagulant Argatroban Crosslinked with Poly N-(2-Hydroxypropyl) Methacrylamide by Ester Linker

Example 13 includes methods for applying chemically crosslinked polymers and anticoagulant onto stent. The reactive polymer and anti-coagulant can be reacted and purified before making the coating solution or can be mixed together with initiator, then coated one layer for slow release of anticoagulant.

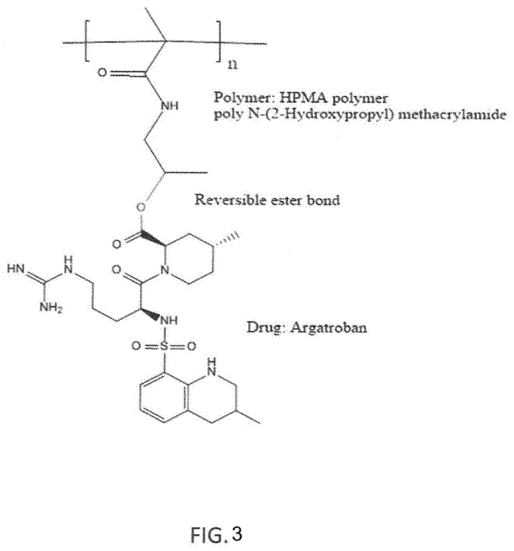

Anticoagulant (Apixaban or Rivaroxaban, Argatroban, Rivaroxaban etc.) is conjugated with biocompatible polymers via a reversible covalent bond, which can slowly release anticoagulant in a controlled manner. For example, Argatroban is linked to poly N-(2-Hydroxypropyl) methacrylamide by a reversible ester bond as show in FIG. 3. When this ester bond is hydrolyzed, the drug Argatroban is released.

Argatroban reacted with poly N-(2-Hydroxypropyl) methacrylamide is dissolved in THF. This polymer solution is air sprayed onto a stainless-steel coronary stent by a microprocessor controlled ultrasonic sprayer until a target weight achieved. After coating, the stents is placed in a 70° C. oven for about 2 hours to remove the solvent. The stents are then mounted on balloon catheters and crimped. The catheters are then inserted in coils and packaged. The pouches are sterilized.

In Vivo, when Argatroban-poly N-(2-Hydroxypropyl) methacrylamide ester bond is hydrolyzed, the drug Argatroban will be released.

### Example 14: Preparation of Drug Eluting Stent Having Anticoagulant Argatroban Crosslinked with PAMAM-OH Dendrimer by Ester Linker

PAMAM-OH dendrimer (generation 2) and Argatroban (1 to 1.2 mole ratio) are dissolved in THF, 4-Dimethylaminopyridine (DMAP) 1% was added as catalyst. The reaction mixture stirred overnight followed by purification. The purified Argatroban-PAMAM-OH dendrimer is dissolved in Tetrahydrofuran (THF). This polymer solution is air sprayed onto a stainless-steel coronary stent by a microprocessor controlled ultrasonic sprayer until a target weight achieved. After coating, the stents are placed in a 70° C. oven for about 2 hours to remove the solvent. The stents are then mounted on balloon catheters and crimped. The catheters are then inserted in coils and packaged. The pouches were sterilized.

In Vivo, when Argatroban crosslinked with PAMAM-OH dendrimer ester bond is hydrolyzed, the drug Argatroban will be released.

### Example 15: Preparation of Drug Eluting Stent Having Anticoagulant and Polymer Microsphere

Anticoagulant (Apixaban, Argatroban, Rivaroxaban etc.) are embodied within biocompatible materials (such as polymers, metals, ceramics, natural plant and/or animal materials). The polymers can be selected from polyesters (poly lactic acid, poly glycolic acid, Polyurethanes), Poly methyl methacrylate (PMMA), poly N-(2-Hydroxypropyl) methacrylamide, Polyethylenimine (PEI), dextran, dextrin, chitosans, poly(L-lysine), and poly(aspartamides), polyethylene, polypropylene, polyamides, Polyethylene glycol (PEG), Polytetrafluoroethylene (PTFE), Silicones, poly(anhydride), poly ortho esters etc. Anticoagulant (Apixaban, Argatroban, Rivaroxaban etc.) with polymers to form drug-polymer nano particles, microsphere, polymeric micelles as the polymer drug delivery systems, which can have high drug loading capacity in the hydrophobic core especially for hydrophobic drugs, and rapid cellular uptake facilitated by their nano-size characteristics.

0.5 mL of poly(D,L-lactide) dichloromethane solution (0.5% w/v) and anticoagulant (Rivaroxaban, Apixaban or Argatroban) dichloromethane solution (0.5% w/v) are slowly added dropwise to polyvinyl alcohol water solution (5% w/w) with magnetic stirring at 1000-1500 rpm. These dispersions are continue stirred for 2 hours at 40° C. and 200 rpm until the microspheres are very small (ie less than 1 m in diameter) to form colloids and therefore the suspension does not settle under gravity. This polymer-anticoagulant suspension is dip coated multiple times to the stent surface until target drug weight achieved. The stent is air dried first then the stents are placed in a 70° C. oven for about 4 hours to remove the solvent. The stents are then mounted on balloon catheters and crimped. The catheters are then inserted in coils and packaged. The pouches were sterilized.

### Example 16: Preparation and Use of Anticoagulant-Impregnated Balloon

A balloon made of a biodegradable or non-degradable polymeric material (e.g., a nylon) and having openings (e.g., pores, holes, etc.) in the body and/or at the surface of the balloon is immersed in a mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator, e.g. tPA) and a solvent, and optionally another kind of bioactive agent (e.g., an anti-proliferative agent, such as rapamycin or a derivative thereof, or an anti-inflammatory agent). The balloon can optionally have one or more coatings comprising a fibrin/thrombus formation-inhibiting or fibrin/thrombus dissolution-promoting agent and/or another kind of bioactive agent and can optionally have a coating comprising no bioactive agent.

The balloon (e.g., the balloon of a balloon-catheter of a stent-delivery system) is advanced to a site to be treated (e.g., an occluded or weakened section of a blood vessel to be opened up or supported by a stent) in the body of a subject and is inflated to a desired inflation diameter. Before, during and/or after inflation of the balloon, the balloon releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 17: Preparation and Use of Anticoagulant-Coated Catheter

A first mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator, e.g. tPA) and a solvent, and optionally a biodegradable or non-degradable polymeric material and/or another kind of bioactive agent (e.g., an anti-proliferative agent, such as rapamycin or a derivative thereof, or an anti-inflammatory agent), is applied (e.g., by spraying or dipping) to a catheter made of a biodegradable or non-degradable polymeric material (e.g., a nylon or a polyether block amide, such as PEBAX®) to form a first coating on the catheter. The first coating can cover any surfaces (e.g., the exterior surface, any other surfaces or all surfaces) of the catheter.

Optionally, a second mixture containing another kind of bioactive agent (e.g., an anti-proliferative agent, such as rapamycin or a derivative thereof, or an anti-inflammatory agent) and a solvent, and optionally a biodegradable or non-degradable polymeric material and/or a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator, e.g. tPA), is applied to the catheter (e.g., by spraying or dipping) to form a second coating on the catheter. The optional second coating can cover any surfaces (e.g., the exterior surface, any other surfaces or all surfaces) of the catheter. The first mixture and the optional second mixture can be applied to the catheter in any order.

Optionally, a third mixture containing a biodegradable or non-degradable polymeric material and a solvent is applied to the catheter (e.g., by spraying or dipping) to form a third coating over the first coating and the optional second coating. The optional third coating can be, e.g., a top layer or coat or a diffusion barrier that controls release of the fibrin/thrombus formation-inhibiting or fibrin/thrombus dissolution-promoting agent and the optional other kind of bioactive agent from the first coating and the optional second coating. The optional third coating can cover the exterior surface or any other surfaces, or all surfaces, of the catheter.

The coated catheter is optionally heated to stabilize the coating(s) and is placed in a container (e.g., a pouch) and sterilized (e.g., by exposure to e-beam radiation).

The catheter (e.g., the catheter of a stent-delivery system) is advanced to a site to be treated (e.g., an occluded or weakened section of a blood vessel to be opened up or supported by a stent) in the body of a subject. Before, while and/or after the catheter is positioned at the treatment site or at an area adjacent thereto, the catheter releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 18: Preparation and Use of Anticoagulant-Impregnated Catheter

A catheter (e.g., an infusion catheter) made of a biodegradable or non-degradable polymeric material (e.g., a nylon or a polyether block amide, such as PEBAX®) and having openings (e.g., pores, holes, etc.) in the body and/or at the surface of the catheter is immersed in a mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator) and a solvent, and optionally another kind of bioactive agent (e.g., an anti-proliferative agent, such as rapamycin or a derivative thereof, or an anti-inflammatory agent). The catheter can optionally have one or more coatings comprising a fibrin/thrombus formation-inhibiting or fibrin/thrombus dissolution-promoting agent and/or another kind of bioactive agent, and can optionally have a coating comprising no bioactive agent.

The catheter (e.g., the catheter of a stent-delivery system) is advanced to a site to be treated (e.g., an occluded or weakened section of a blood vessel to be opened up or supported by a stent) in the body of a subject. Before, while and/or after the catheter is positioned at the treatment site or at an area adjacent thereto, the catheter releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 19: Preparation and Use of Anticoagulant-Coated Balloon-Catheter

A first mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator) and a solvent, and optionally a biodegradable or non-degradable polymeric material and/or another kind of bioactive agent (e.g., an anti-proliferative agent, such as rapamycin or a derivative thereof, or an anti-inflammatory agent), is applied (e.g., by spraying or dipping) to the balloon portion and/or the catheter portion of a balloon-catheter made of a biodegradable or non-degradable polymeric material (e.g., a nylon) to form a first coating on the balloon portion and/or the catheter portion. The first coating can cover any surfaces (e.g., the exterior surface, any other surfaces or all surfaces) of the balloon and/or the catheter.

Optionally, a second mixture containing another type of bioactive agent (e.g., an anti-proliferative agent, such as rapamycin or a derivative thereof, or an anti-inflammatory agent) and a solvent, and optionally a biodegradable or non-degradable polymeric material and/or a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator), is applied to the balloon portion and/or the catheter portion (e.g., by spraying or dipping) to form a second coating on the balloon portion and/or the catheter portion. The optional second coating can cover any surfaces (e.g., the exterior surface, any other surfaces or all surfaces) of the balloon and/or the catheter. The first mixture and the optional second mixture can be applied to the balloon portion and/or the catheter portion in any order.

Optionally, a third mixture containing a biodegradable or non-degradable polymeric material and a solvent is applied to the balloon portion and/or the catheter portion (e.g., by spraying or dipping) to form a third coating over the first coating and the optional second coating. The optional third coating can be, e.g., a top layer or coat or a diffusion barrier that controls release of the fibrin/thrombus formation-inhibiting or fibrin/thrombus dissolution-promoting agent and the optional other kind of bioactive agent from the first coating and the optional second coating. The optional third coating can cover the exterior surface or any other surfaces, or all surfaces, of the balloon and/or the catheter.

The coated balloon-catheter is optionally heated to stabilize the coating(s) and is placed in a container (e.g., a pouch) and sterilized (e.g., by exposure to e-beam radiation).

The balloon-catheter (e.g., the balloon-catheter of a stent-delivery system) is advanced to a site to be treated (e.g., an occluded or weakened section of a blood vessel to be opened up or supported by a stent) in the body of a subject, and the balloon is inflated to a desired inflation diameter. Before, during and/or after inflation of the balloon, the balloon portion and/or the catheter portion of the balloon-catheter releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 20: Preparation and Use of Anticoagulant-Impregnated Balloon-Catheter

The balloon portion and/or the catheter portion of a balloon-catheter (e.g., a weeping catheter) made of a biodegradable or non-degradable polymeric material (e.g., a nylon) and having openings (e.g., pores, holes, etc.) in the body and/or at the surface of the balloon and/or the catheter is immersed in a mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator) and a solvent, and optionally another type of bioactive agent (e.g., an anti-proliferative agent, such as rapamycin or a derivative thereof, or an anti-inflammatory agent). The balloon portion and/or the catheter portion of the balloon-catheter can optionally have one or more coatings comprising a fibrin/thrombus formation-inhibiting or fibrin/thrombus dissolution-promoting agent and/or another kind of bioactive agent, and can optionally have a coating comprising no bioactive agent.

The balloon-catheter (e.g., the balloon-catheter of a stent-delivery system) is advanced to a site to be treated (e.g., an occluded or weakened section of a blood vessel to be opened up or supported by a stent) in the body of a subject, and the balloon is inflated to a desired inflation diameter. Before, during and/or after inflation of the balloon, the balloon portion and/or the catheter portion of the balloon-catheter releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 21: Use of Anticoagulant-Delivering Infusion Catheter

An infusion catheter contains one or more lumens for delivering one or more drugs. The infusion catheter (e.g., the catheter of a stent-delivery system) is advanced to a site to be treated (e.g., an occluded or weakened section of a blood vessel to be opened up or supported by a stent) in the body of a subject. Before, while and/or after the catheter is positioned at the treatment site or at an area adjacent thereto, a first mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator) and a solvent (e.g., saline), and optionally another kind of bioactive agent (e.g., an anti-proliferative agent, such as rapamycin or a derivative thereof, or an anti-inflammatory agent), is injected through one or more drug-delivering lumens of the catheter. Before, while and/or after the catheter is positioned at the treatment site or at an area adjacent thereto, a second mixture containing another kind of bioactive agent (e.g., an anti-proliferative agent or an anti-inflammatory agent) and a solvent (e.g., saline), and optionally a fibrin/thrombus formation-inhibiting or fibrin/thrombus dissolution-promoting agent, optionally is injected through one or more drug-delivering lumens of the catheter. The catheter delivers the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto.

### Example 22: Preparation and Use of Anticoagulant-Coated Surgical Instrument

A surgical instrument (e.g., a cutting instrument, such as a knife) made of a biodegradable or non-degradable metal or metal alloy (e.g., stainless steel) optionally undergoes surface treatment (e.g., microblasting) to improve adhesion of a coating (e.g., a polymeric coating) to a metal surface.

A first mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator) and a solvent, and optionally a biodegradable or non-degradable polymeric material and/or another kind of bioactive agent (e.g., an anti-proliferative agent, such as rapamycin or a derivative thereof, or an anti-inflammatory agent), is applied to the surgical instrument (e.g., by dipping or spraying) to form a first coating on the surgical instrument. The first coating can cover the exterior surface, any other surfaces or all surfaces of the surgical instrument.

Optionally, a second mixture containing another kind of bioactive agent (e.g., an anti-proliferative agent, such as rapamycin or a derivative thereof, or an anti-inflammatory agent) and a solvent, and optionally a biodegradable or non-degradable polymeric material and/or a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator), is applied to the surgical instrument (e.g., by dipping or spraying) to form a second coating on the surgical instrument. The optional second coating can cover the exterior surface, any other surfaces or all surfaces of the surgical instrument. The first mixture and the optional second mixture can be applied to the surgical instrument in any order.

Optionally, a third mixture containing a biodegradable or non-degradable polymeric material and a solvent is applied to the surgical instrument (e.g., by dipping or spraying) to form a third coating over the first coating and the optional second coating. The optional third coating can be, e.g., a top layer or coat or a diffusion barrier that controls release of the fibrin/thrombus formation-inhibiting or fibrin/thrombus dissolution-promoting agent and the optional other kind of bioactive agent from the first coating and the optional second coating. The optional third coating can cover the exterior surface, any other surfaces or all surfaces of the surgical instrument.

The coated surgical instrument is optionally heated to stabilize the coating(s) and is placed in a container (e.g., a pouch) and sterilized (e.g., by exposure to e-beam radiation).

The surgical instrument (e.g., a cutting instrument, such as a knife) is advanced to a site in the body of a subject undergoing a surgery or intervention (e.g., a tissue to be cut or treated). Before, while and/or after the surgical instrument is positioned at the treatment site or at an area adjacent thereto, or before, while and/or after the surgical instrument contacts the tissue to be treated, the surgical instrument releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 23: Preparation and Use of Anticoagulant-Impregnated Surgical Instrument

A surgical instrument made of a biodegradable or non-degradable metal or metal alloy (e.g., stainless steel) and having openings (e.g., pores, holes, etc.) in the body and/or at the surface of the surgical instrument is immersed in a mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator) and a solvent, and optionally another kind of bioactive agent (e.g., an anti-proliferative agent, such as rapamycin or a derivative thereof, or an anti-inflammatory agent). The surgical instrument can optionally undergo surface treatment, can optionally have one or more coatings comprising a fibrin/thrombus formation-inhibiting or fibrin/thrombus dissolution-promoting agent and/or another kind of bioactive agent, and can optionally have a coating comprising no bioactive agent.

The surgical instrument (e.g., a cutting instrument, such as a knife) is advanced to a site in the body of a subject undergoing a surgery or intervention (e.g., a tissue to be cut or treated). Before, while and/or after the surgical instrument is positioned at the treatment site or at an area adjacent thereto, or before, while and/or after the surgical instrument contacts the tissue to be treated, the surgical instrument releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 24: Use of Anticoagulant-Delivering Infusion Surgical Instrument

An infusion surgical instrument [e.g., a cutting instrument (e.g., a knife) or an injection device (e.g., a needle)] contains one or more lumens for delivering one or more drugs. The surgical instrument is advanced to a site in or on the body of a subject undergoing a surgery or intervention (e.g., a tissue to be cut or treated). Before, while and/or after the surgical instrument is positioned at the treatment site or at an area adjacent thereto, or before, while and/or after the surgical instrument contacts the tissue to be treated, a first mixture containing a fibrin/thrombus formation-inhibiting or fibrin/thrombus dissolution-promoting agent (e.g., an anticoagulant, such as Rivaroxaban or a derivative thereof, or a fibrinolytic or thrombolytic agent, such as a plasminogen activator) and a solvent (e.g., saline), and optionally another kind of bioactive agent (e.g., an anti-proliferative agent, such as rapamycin or a derivative thereof, or an anti-inflammatory agent), is injected through one or more drug-delivering lumens of the surgical instrument. Before, while and/or after the surgical instrument is positioned at the treatment site or at an area adjacent thereto, or before, while and/or after the surgical instrument contacts the tissue to be treated, a second mixture containing another kind of bioactive agent (e.g., an anti-proliferative agent or an anti-inflammatory agent) and a solvent (e.g., saline), and optionally a fibrin/thrombus formation-inhibiting or fibrin/thrombus dissolution-promoting agent, optionally is injected through one or more drug-delivering lumens of the surgical instrument. The surgical instrument delivers the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto.

### Example 25 Preparation of mTOR Inhibitors and/or Anticoagulant Coated Balloon with Excipient

The balloon can optionally adopt carrier excipient to coat to facilitate drug transfer to the vessel wall and control release rate. A variety of carrier excipients and techniques can be used. The selected excipient could be contrast agent (i.e. iopromide), urea, dextrane, shellac, shelloic acid, keratosis (a naturally derived protein), Plasticizer (i.e. butyryl-tri-hexyl citrate, acetyl tributyl citrate, citrate ester, glycerol, other organic ester), hydrophilic space, Polyvinylpyrrolidone (PVP) and its hydrogels, Surfactants, Non-ionic surfactant Polysorbate/sorbitol (i.e. Tween20, Tween60 or Tween80), nordihydroguaiaretic acid (NDGA), hydrophibic excipient such as phospholipid, amphiphilic polymer such as Poly(ethylene glycol) (i.e PEG 8000), poly(ethylene oxide) (PEO) (molecular weight range from 100,000 to 10,000,000), Polyethylenimine (PEI) or polyaziridine linear or branched, amphiphilic block co-polymers composed of poly(ethylene oxide) (PEO) as the hydrophilic block and poly(ether)s, poly(amino acid)s), hydrophobic polymer space, biodegradable polymers such as Poly DL lactide-co-glycolide, Poly L Lactide-co-caprolactone, durable polymers, individually or combinations thereof.

A balloon made of a biodegradable or non-degradable polymeric material (e.g., a nylon) and having openings (e.g., pores, holes, etc.) in the body and/or at the surface of the balloon was used. The drug was selected from a mixture containing a mTOR inhibitors/Anticoagulant1/Anticoagulant2. The mTOR inhibitors was selected from Sirolimus, Novolimus, temsirolimus, zotarolimus and everolimus etc. The anticoagulants were selected from Apixaban, Argatroban, Rivaroxaban or a derivative thereof, and/or a fibrinolytic or thrombolytic agent, such as a plasminogen activator) individually or combinations thereof. For example, Siroliums and Anticoagulant1/Anticoagulant2 (Apixaban and Argatroban) were placed in a vial and dissolved in dichloromethane or dichloromethane/Methanol combination at 2 to 10 mg/ml. The carrier excipient was dissolved in a proper solvent. The solution and drug solutions were combined at a target ratio of 3 to 1, 1 to 1, 2 to 1, or 1 to 3 ratios according to the target drug loading. Further dilution with dichloromethane was conducted if needed. Optionally anti-solvent was used to control the coating morphology of particles and drug release rate.

Optionally the balloon can undergo physical surface treatment before coated such that the surface has microspores, micro-holes, or chemical surface treatment before coated such that the balloon materials have photo-link or other chemical function group that can be reacted under UV or other techniques to easy coating. After surface treatment, the coating can be spray coat or dip coat or use other coating techniques (i.e. 3D printer).

The coating can optionally cover any surfaces (e.g., the exterior surface, any other surfaces or all surfaces) of the balloon. The coating solution can be homogeneous or non-homogeneous such as suspension or emulsions.

The coated balloon can optionally combine multi-strategies (e.g., electrospinning, plasma treatment, Layer-by-Layer Self-Assembly or a combination thereof) to form finely controlling structural, mechanical, and surface properties. With tuned coating techniques and solvent removal technique, the produces powder particles can be optionally homogenous, porous, and uniform in size and shape. The morphology of particles could be micro-crystalline, nanoparticles, Nano-encapsulated to provide release rate control.

When spray coat, a microprocessor controlled ultrasonic sprayer was used to apply the drug containing drug solution to cover any surface of a balloon. A mandrel was placed through catheter tips and underneath an ultrasonic spray nozzle (Micromist System with Ultrasonic Atomizing Nozzle Sprayer, Sono-Tek, N.Y.), which was rotating at 80 rpm and move longitudinally at a rate of 0.050 inches/minutes. The coating parameter can optionally adjusted to ideal coating texture and the morphology and the profile of the interface between drug and balloon surface.

After coating, the balloon was placed in a vacuum chamber to remove the residue solvent. Optionally the coated balloon can be tri-folded to protect coated drug with a folded and/or wrapped balloon thereon to a pre-annealing step to induce a fold/wrap memory in the resulting pre-annealed balloon and/or coated balloon has a protector which need to peel off before use.

The balloon catheter was then inserted in a coil and packaged. The pouch was sterilized by Ethylene oxide or E-beam. The pouch was further packaged in a foil pouch with oxygen scavengers and nitrogen purge and vacuum sealed.

The balloon (e.g., the balloon of a balloon-catheter of a balloon-delivery system) was advanced to a site to be treated (e.g., an occluded or weakened section of a blood vessel to be opened up or supported by a balloon) in the body of a subject and was inflated to a desired inflation diameter. Before, during and/or after inflation of the balloon, the balloon releases the fibrin/thrombus formation-inhibiting or fibrin/thrombus dissolution-promoting agent and the optional other kind of bioactive agent (e.g., an anti-proliferative agent or an anti-inflammatory agent) to, into or at the treatment site, or to, into or at an area adjacent thereto, by any suitable mechanism (e.g., concentration gradient, diffusion, pressure or mechanical force, or a combination thereof).

### Example 26: Preparation of Drug Coated Balloon Having Anticoagulant and Polymer Microsphere

Anticoagulant (Apixaban, Argatroban, Rivaroxaban etc.) are embodied within biocompatible materials (such as polymers, metals, ceramics, albumin, liposome, natural plant and/or animal materials). The polymers can be selected from polyesters (poly lactic acid, poly glycolic acid, poly lactic acid-co-glycolic acid, poly lactic acid-co-caprolactone, poly ethylene glycol-block-poly caprolactone, Polyurethanes etc.), Poly methyl methacrylate (PMMA), poly N-(2-Hydroxypropyl) methacrylamide, Polyethylenimine (PEI), dextran, dextrin, chitosans, poly(L-lysine), and poly(aspartamides), polyethylene, polypropylene, polyamides, Polyethylene glycol (PEG), Silicones, poly(anhydride), poly ortho esters etc. Anticoagulant (Apixaban, Argatroban, Rivaroxaban etc.) with polymers to form drug-polymer nano particles, microsphere, polymeric micelles as the polymer drug delivery systems, which can have high drug loading capacity in the hydrophobic core especially for hydrophobic drugs, and rapid cellular uptake facilitated by their micro/nano-size characteristics.

0.5 mL of poly(D,L-lactide) dichloromethane solution (0.5% w/v) and anticoagulant (Rivaroxaban, Apixaban or Argatroban) dichloromethane solution (0.5% w/v) are slowly added dropwise to polyvinyl alcohol water solution (5% w/w) with magnetic stirring at 1000-1500 rpm. These dispersions are continue stirred for 2 hours at 40° C. and 200 rpm until the microspheres are very small (ie less than 1 m in diameter) to form colloids and therefore the suspension does not settle under gravity. This polymer-anticoagulant suspension is dip coated multiple times to the balloon surface until target drug weight achieved. After coating, the balloons were air dried first then were placed in a vacuum chamber to remove the solvent. The balloons were then tri-folded before putting on the protective sheath. The balloon catheters were then inserted in coils and packaged. The pouches were sterilized.

### Example 27: Preparation of Drug Coated Balloon Having Anticoagulant and Polymer Self-Assembly Hollow Nanoparticles

Anticoagulant (Apixaban, Argatroban, Rivaroxaban etc.) are monodispersed within hollow polymer nanoparticle. The polymers with molecular range from 100K to 10K (optimally from 40 k to 20K) can be selected from block degradable polymers of poly lactic acid, poly glycolic acid, poly lactic acid-co-glycolic acid, poly lactic acid-co-caprolactone, poly ethylene glycol-block-poly caprolactone etc., or block non-degradable polymers selected from Polyvinylpyridine block with Poly methyl methacrylate (PMMA), poly N-(2-Hydroxypropyl) methacrylamide, Polyethylenimine (PEI), dextran, dextrin, chitosans, poly(L-lysine), and poly(aspartamides), polyamides, Polyethylene glycol (PEG), Silicones, poly(anhydride), poly ortho esters, polystyrene-b-polyvinylpyridine, poly(isoprene)-b-poly(vinyl pyridine), poly(vinyl pyridine)-b-poly(styrene)-b-poly(vinyl pyridine), poly(styrene)-b-polyvinyl pyridine)-b-poly(styrene), poly(styrene-b-poly(acrylic acid), poly(styrene)-b-poly(butadiene)-b-poly(vinyl pyridine), poly(styrene-b-poly(methacrylic acid), poly(styrene)-b-poly(ethylene oxide), poly(butadiene)-b-poly(acrylic acid), poly(butadiene)-b-poly(ethylene oxide), poly(vinyl pyridine)-b-poly(butadiene)-b-poly(vinyl pyridine), poly(ethylene)-b-poly(ethylene oxide), and poly(styrene)-b-poly(vinyl pyridine)-b-poly(ethylene oxide) etc. Anticoagulant (Apixaban, Argatroban, Rivaroxaban etc.) with polymers to form drug-polymer nano particles as the polymer drug delivery systems, which can have high drug loading capacity in the hydrophobic core especially for hydrophobic drugs, and rapid cellular uptake facilitated by their nano-size characteristics.

Poly(ethylene glycol)-block-poly(4-vinyl pyridine) or poly(styrene)-block-poly(4-vinyl pyridine) or other poly(4-vinyl pyridine) block polymers is dissolved in DMF or 1,4-dioxane to prepare a solution of 5 mg/ml; this solution is added to solutions containing varied amount of Azo compounds in the same solvent (the monomer molar ratio of 4-vinyl pyridine: Azo compounds from 1:0.2 to 1:2). Azo compounds are selected from Metanil Yellow, Orange II sodium salt,2,2′-Dihydroxyazobenzene,2-(4-Hydroxyphenylazo) benzoic acid,5-[(2-Carboxyphenyl) azo]-2-hydroxybenzoic acid, Olsalazine, 5-[(4-aminophenyl)azo]-2-hydroxy-Benzoic acid as hydrogen bonding agent for self-assembly. After stirring and reflux overnight, the self-assembly nanoparticles were collected by centrifuging. Using ethanol wash to remove hydrogen bonding agent results in monodisperse hollow nanoparticles with tunable hollow cavity size and internal surface reactivity. The resulting nanoparticles are redispersed in chloroform and mixed with anticoagulant (Rivaroxaban, Apixaban or Argatroban) solution in the same solvent; the balloon can be coated with this solution by dip- or spin-coating method to the balloon surface until target drug weight achieved with anticoagulant (Rivaroxaban, Apixaban or Argatroban) is hydrogen bonding with this hollow nanoparticle polymers.

After coating, the balloons were air dried first then were placed in a vacuum chamber to remove the solvent. The balloons were then tri-folded before putting on the protective sheath. The balloon catheters were then inserted in coils and packaged. The pouches were sterilized.

### Example 28: Preparation of Drug Coated Balloon Having Colocalized Synergized Delivery of m-TOR and Paclitaxel Self-Assembly Hollow Nanoparticles

m-TOR (Sirolimus, biolimus, everolimus, myolimus, novolimus, ridaforolimus, temsirolimus, zotarolimus, or salts, isomers, solvates, analogs, derivatives, metabolites etc.) and paclitaxel are embodied within hollow polymer nanoparticles. The polymers with molecular range from 100K to 10K (optimally from 40 k to 20K) can be selected from block degradable polymers of poly lactic acid, poly glycolic acid, poly lactic acid-co-glycolic acid, poly lactic acid-co-caprolactone, poly ethylene glycol-block-poly caprolactone etc., or block non-degradable polymers selected from Polyvinylpyridine block with Poly methyl methacrylate (PMMA), poly N-(2-Hydroxypropyl) methacrylamide, Polyethylenimine (PEI), dextran, dextrin, chitosans, poly(L-lysine), and poly(aspartamides), polyamides, Polyethylene glycol (PEG), Silicones, poly(anhydride), poly ortho esters, polystyrene-b-polyvinylpyridine, poly(isoprene)-b-poly(vinyl pyridine), poly(vinyl pyridine)-b-poly(styrene)-b-poly(vinyl pyridine), poly(styrene)-b-polyvinyl pyridine)-b-poly(styrene), poly(styrene-b-poly(acrylic acid), poly(styrene)-b-poly(butadiene)-b-poly(vinyl pyridine), poly(styrene-b-poly(methacrylic acid), poly(styrene)-b-poly(ethylene oxide), poly(butadiene)-b-poly(acrylic acid), poly(butadiene)-b-poly(ethylene oxide), poly(vinyl pyridine)-b-poly(butadiene)-b-poly(vinyl pyridine), poly(ethylene)-b-poly(ethylene oxide), and poly(styrene)-b-poly(vinyl pyridine)-b-poly(ethylene oxide). M-TOR with paclitaxel and polymers to form drug-polymer nano particles as the polymer drug delivery systems, which can have high drug loading capacity in the hydrophobic core especially for hydrophobic drugs, and rapid cellular uptake facilitated by their nano-size characteristics.

Poly(ethylene glycol)-block-poly(4-vinyl pyridine) or poly(styrene)-block-poly(4-vinyl pyridine) or other poly(4-vinyl pyridine) block polymers is dissolved in DMF or 1,4-dioxane to prepare a solution of 5 mg/ml; this solution is added to solutions containing varied amount of Azo compounds in the same solvent (the monomer molar ratio of 4-vinyl pyridine: Azo compounds from 1:0.2 to 1:2). Azo compounds are selected from Metanil Yellow, Orange II sodium salt, 2,2′-Dihydroxyazobenzene,2-(4-Hydroxyphenylazo) benzoic acid,5-[(2-Carboxyphenyl) azo]-2-hydroxybenzoic acid, Olsalazine, 5-[(4-aminophenyl) azo]-2-hydroxy-Benzoic acid as hydrogen bonding agent. After stirring and reflux overnight, the nanoparticles were collected by centrifuging. Using ethanol wash to remove hydrogen bonding agent results in self-assembly monodisperse hollow nanoparticles with tunable hollow cavity size and internal surface reactivity. The resulting nanoparticles are redispersed in chloroform and mixed with m-TOR and paclitaxel (ranging from 3:1 to 1:3 by weight) solution in the same solvent; the balloon can be coated with this solution by dip- or spin-coating method to the balloon surface until target drug weight achieved with m-TOR and paclitaxel are hydrogen bonding with this self-assembly hollow nanoparticle.

After coating, the balloons were air dried first then were placed in a vacuum chamber to remove the solvent. The balloons were then tri-folded before putting on the protective sheath. The balloon catheters were then inserted in coils and packaged. The pouches were sterilized.

### Example 29: Preparation of Anticoagulant and/or mTOR Coated Sleeve in a Covered Stent

A stent covered by a sleeve can have a polymer coating containing anticoagulant and/or a combination with mTOR on top, part of, and/or adjacent to the sleeve made from polymer selected from non-degradable polymers such as polytetrafluoroethylene, fluorinated ethylene propylene, Dacron, polyethylene terephthalate, polyurethanes, polycarbonate, polypropylene, Pebax, polyethylene and biological polymers such as modified cellulose, collagen, fibrin, and elastin, and biodegradable polymer such as poly(alpha-hydroxy acid), poly-L-lactide (PLLA), poly-D-lactide (PDLA), polyglycolide (PGA), polydioxanone, polycaprolactone (PCL), polygluconate, polylactic acid-polyethylene oxide copolymers, poly(hydroxybutyrate), polyanhydride, polyphosphoester, or poly(aminoacides), knitted or weaved fabric material or film material which have been previously cast by brushing, dipping, electrospun or electrospray technique or other means onto the metallic bare or polymer stent, or those known in the art, which coated with anticoagulant and/or mTOR. The sleeve surface can be porous or non-porous.

The sleeve can be infused with a polymer coating in a solvent solution with anticoagulant such as Apixaban, Rivaroxaban, Argatroban or a combination with mTOR such as rapamycin, everolimus, biolimus, temsirolimus, ridaforolimus, zotarolimus, myolimus and novolimus drug solution into the said sleeve and said solvent can evaporate leaving either polymer coating or drug in the pores of the sleeve. This process also applicable to stent graft.

Poly (L-lactide acid-co-glycolic acid) polymer is dissolved into dichloromethane at room temperature and vortex until the polymer is uniformly dissolved/dispersed. Anticoagulant (Apixaban or Rivaroxaban & Argatroban) is placed in a vial and dissolved in dichloromethane/Methanol at room temperature and vortex until all the drug is uniformly dissolved/dispersed.

Each polymer solution and each drug solutions is combined anticoagulant (Apixaban or Rivaroxaban) & Argatroban and/or mTOR with weight ratio 1 to 1 or other ratio) to poly (L-lactide acid-co-glycolic acid) by weight ratio was 3:1 or other ratio according to the target drug dose.

The sleeve can optionally undergo surface treatment if the surface is not porous (i.e. plasma treatment or other friction surface treatment). After surface treatment, the coating could be spray coated or dip coated. The coating can be inside the sleeve or dipped onto the sleeve or coated on the sleeve.

When spray coated, a microprocessor controlled ultrasonic sprayer is used to coat the sleeve containing drug/excipient solution to the entire surface of the implant. After coating, the sleeve is placed in a 70° C. oven for about 2 hours to remove the residue solvent. The covered stent is then mounted on the delivery catheter and then is inserted in a coil and packaged. The pouches were sterilized.

### Example 30: Formulation Composition Development of Inhalation for Pulmonary Delivery Contains Direct Factor Xa Inhibitor Apixaban and/or Direct Factor IIa Inhibitor Argatroban

An inhalation solution for pulmonary delivery of Apixaban and Argatroban may effectively treat and reduce severe lung inflammation. The combination of the 2 drugs may create unusual synergy or efficacy. The feasibility of Apixaban and Argatroban combined into one water-based drug solution was evaluated and the solutions compatibility with hospital nebulizers was evaluated. Additionally, the physical and chemical stability of the formulation was evaluated.

The Apixaban and Argatroban can be made into individual inhalation solutions and dosed separately or combined in one solution and administered simultaneously. For lung inflammation the target tissue of the drugs is the overall lung combined with the microcapillaries of the alveoli. This requires reevaluating the effective therapeutic dose for the lung based on what is known about current oral and intravenous dosing regimens and specific indications.

The current dosing of Argatroban targets delivery to the entire body systemically for the indication of stent placement surgery and is given by intravenous delivery of isotonic solution at 1.0 mg/mL. A typical human dose for a 60 kg person is a bolus infusion for 5 minutes of 21 mg, with a 15 mg dose infused for 10 minutes at 1.5 mg/minute. Therefore, approximately 36 mg of Argatroban is given in 10-15 minutes.

The current dosing for Apixaban targets the entire body systemically to reduce the risk of stroke but is given orally. The recommended dose is 5 mg tablets given twice per day for a total of 10.0 mg per 60 kg person per day.

Several challenges arise to determine the feasibility and effective dose for the lung inflammation indication. The first problem is the drugs are both considered low solubility compounds and to deliver to the lung via water-based nebulizers in a timely manner is difficult. For this product concept the drug delivery should be less than 30 minutes but this requires very concentrated drug formulations, which previously have not been achieved. The second challenge is maximizing the resident time for the drug to stay in the target tissue (lung) to treat the inflammation.

Conceptually the dose of drug can be substantially reduced since the lungs tissue will initially receive the entire dose and therefore have therapeutically higher relevant dose (ng/mg) in the lung tissue versus the entire body. Inhaled drugs are substantially absorbed systemically into the plasma when delivered to the lung but at a much lower efficiency. The proposed dose range delivered to the lung is approximately 1.0 mg of Apxiban 3 times a day, and 10.0 mg of Argatroban 3 times a day for much improved systemic safety profile. Table 7A is the typical systematic dose of Anticoagulants

TABLE 7A
Typical systematic dose of Anticoagulants

| Drug/Delivery | Dose per day (mg) | Total per day (mg) |
|---|---|---|
| Apixaban Pill/Oral | 5.0 mg × 2 | 10.0 |
| Argatroban Sol/IV | 36 mg in 15 minutes | 125 |
| Apixiban Inhaled | 1.0 mg × 3 | 3.0 |
| Argatroban Inhaled | 10.0 mg × 3 | 30.0 |

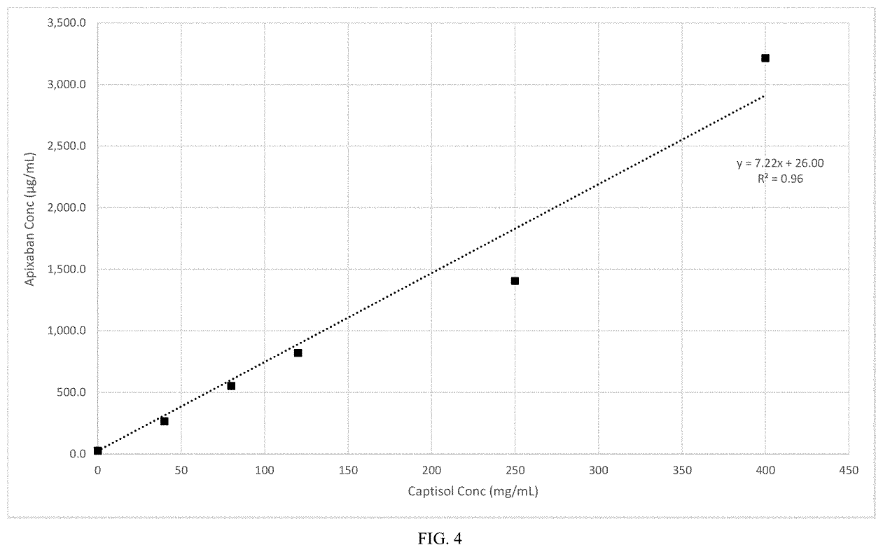

A formulation with a high effective dose and maintain physical compatibility with a drug delivery device required solubility studies with a variety of excipients. Cyclodextrins proved to be the most versatile solubilizing agents and required low amounts of the sugar to maintain device functionality without sacrificing the solubility of the drugs. The most useful solubilizer was Captisol (sulfobutylether β-cyclodextrin). FIG. 4 shows the solubility of Apixaban as a function of Captisol concentration.

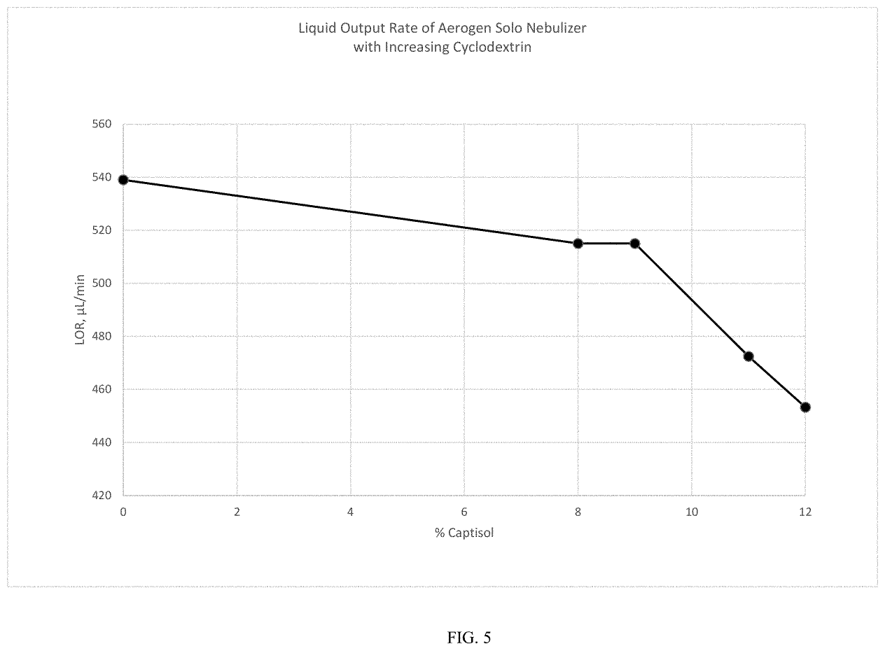

Useful formulations can be in the range of 4-10% Captisol (40-100 mg/mL) which allows 100-1000 ug/ml of Apixaban drug to be delivered through an Aerogen Solo or hospital type nebulizer. Above these range limits of Captisol concentration, the liquid output rate (LOR) of the nebulizer devices begin to have reduced liquid output rate of the aqueous based formulation. Therefore, the invention is creating a successful product configuration to balance between counter variables. The widest range of functionality with Captisol and the standard hospital nebulizers such as aerogen solo and Aero Eclipse jet nebulizers is 0% to 25% Captisol. FIG. 5 was the Liquid Output Rate of Aerogen Solo Nebulizer vs percentage of Cyclodextrin. Above 25% Captisol concentration, the nebulizers have severely reduced liquid output rates that increase dosing times to several hours. FIG. 5 was the Liquid Output Rate of Aerogen Solo Nebulizer vs percentage of Cyclodextrin.

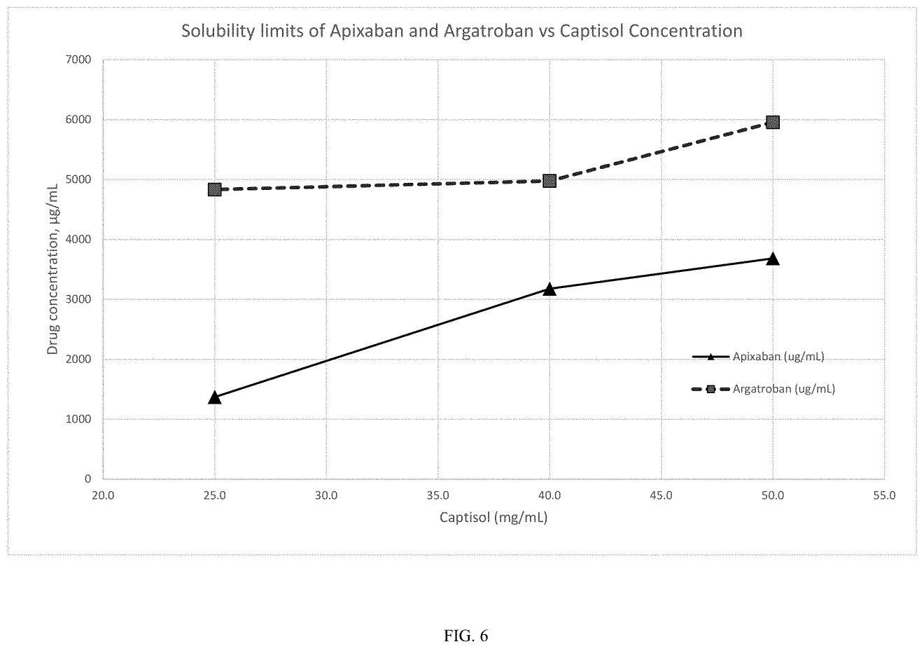

The solubility of Argatroban has higher solubility of ˜3 mg/ml in water versus Apixaban ˜28 ug/mL in water. Therefore, creating a formulation with 0.200 mg/ml of Apixaban combined with 2.00 mg/mL in the 8% concentration range of Captisol was achieved, providing a 10:1 drug ratio of Argatroban: Apixaban in formulation compatible with a nebulizer. The combination formulation was tested for upper limits of drug solubility at very high concentrations of Captisol, even though the formulations have very low liquid output rates in the device. FIG. 6 shows the solubility limits of Argatroban and Apixaban in Captisol.

A rapid-acting drug delivery formulation or a sustained release drug delivery formulation delivered via the pulmonary route comprising a direct factor Xa inhibitor and/or a direct factor IIa inhibitor can be achieved by adjusting the formulation such that drug released from lungs into blood or tissue within 5 minutes or last to up to hours. The formulation contains a direct factor Xa inhibitor Apixaban and a direct factor IIa inhibitor Argatroban which can be released at either the same time or a different time.

The formulation could also comprise a direct factor Xa inhibitor Apixaban and a direct factor IIa inhibitor Argatroban combined with Azelastine and Hydroxychloroquine in Captisol®/citrate buffer and the drugs can release either same time or different time.

A fast-acting formulation of Apixiban and Argatroban for inhalation can be created. A dry powder formulation containing Argatroban and Apixaban at a 10:1 ratio with a bulking agent can be spray dried. Apixaban has a fast permeation through the lung with a Tmax of approximately 5-10 minutes. Argatroban appears to have Tmax of approximately 60 minutes. The rate of systemically absorbed Argatroban could be increased by using a penetration enhancer in the dry powder formulation containing both compounds without modifying the Apixaban absorption profile substantially thus creating a fast-acting combination anticoagulant.

The composition is formulated for local/regional delivery by inhalation directly delivered to the lungs to create the local high concentration that cannot be obtained by typical systemic delivery route such as oral tablets, intravenous, intramuscular, and subcutaneous injection and reduced the side effect of high systemic drug concentrations.

The composition is also formulated for systemic delivery by inhalation as a method for a rapid acting systemic delivery by incorporating penetration enhancers to local/regional delivery formulation to reach the capillary bed and thus a pathway for a rapid systemic delivery, thus bypassing the local deposition in lung tissue.

### Example 31: Preparation of 2.5 mg/ml Direct Factor Xa Inhibitor Apixaban for Use in a Nebulizer Device

Prepare a solution of direct factor Xa inhibitor drug Apixaban at 2.5 mg/mL suitable for use in a nebulizer device using solubility enhancers. Using a 10 mL volumetric flask add 125 mg of USP grade hydroxypropyl beta cyclodextrin. Add 8.0 mL of USP water for injection. Seal the flask and gently agitate until all the dextrin is dissolved and the solution is clear. Accurately weigh 25.0 mg of direct factor Xa inhibitor drug Apixaban and add it to the volumetric flask. Accurately weigh 50 mg of USP grade sodium citrate dihydrate and add it to the flask. Cover the flask. Using a sonicating water bath at 35° C. sonicate the solution for 10 minutes or until the solution is clear, and the cyclodextrin has complexed the drug. Measure the pH of the solution and adjust to pH 7.0 using 10 μL aliquots of 100 mM citric acid (prepared pH adjuster solution). Bring the final volume of the flask to 10.0 mL mark using water for injection. Filter the solution through a 0.2 μm Durapore membrane. Transfer 2.0 mL to a nebulizer device and measure the liquid output rate to confirm at least 300 μL per minute output rate from the device.

### Example 32: Preparation of 5.0 mg/ml Direct Factor Xa Inhibitor Apixaban for Use in a Nebulizer Device

Prepare a solution of direct factor Xa inhibitor drug Apixaban at 5.0 mg/mL suitable for use in a nebulizer device using solubility enhancers. Using a 10 mL volumetric flask add 175 mg of USP grade hydroxypropyl beta cyclodextrin. Add 100 mg of USP polyvinylpyrrolidone. Add 8.0 mL of USP water for injection. Seal the flask and gently agitate until all the dextrin is dissolved and the solution is clear. Accurately weigh 50.0 mg of direct factor Xa inhibitor drug Apixaban and add it to the volumetric flask. Accurately weigh 50 mg of USP grade sodium citrate dihydrate and add it to the flask. Cover the flask. Using a sonicating water bath at 35° C. sonicate the solution for 10 minutes or until the solution is clear and the cyclodextrin has complexed the drug. Measure the pH of the solution and adjust to pH 7.0 using 10 μL aliquots of 100 mM citric acid (prepared pH adjuster solution). Bring the final volume of the flask to the 10.0 mL mark using water for injection. Filter the solution through a 0.2 μm Durapore membrane. Transfer 2.0 mL to a nebulizer device and measure the liquid output rate to confirm at least 300 μL per minute output rate from the device.

### Example 33: Preparation of Direct Factor Xa Inhibitor Apixaban for Use in a Dry Powder Inhaler

Prepare a spray dried powder formulation 25% by weight of direct factor Xa inhibitor drug Apixaban. Particle size of the powder is targeted to be 3 to 10 um median mass aerodynamic diameter (MMAD). Using a spray nozzle attached to a Buchi spray dryer. Prepare a 10% (V/V) ethanol (or dimethyl sulfoxide (DMSO)) in a water cosolvent solution by adding 100 mL of USP grade ethanol to 900 mL USP grade water for injection and stir well. Pour a 100 mL aliquot into a bottle. To the 100 mL of cosolvent solution prepare a solution at 1.6% solids (16 mg/mL). Accurately weigh 400 mg of direct factor Xa inhibitor drug Apixaban and add it to the cosolvent solution. Accurately weigh 600 mg of USP grade mannitol and add it to the solution. Accurately weigh 600 mg of USP grade leucine and added to the solution. Stir the solution for 15 minutes until clear. Put the solution onto the spray dryer and collect the fine particles in the glass bottle collection system. After measuring the weight of the glass bottle (tare weight), measure the spray drying recovery yield compared to 1600 mg of starting solids. Expect approximately 80% recovery approximately 1200 mg of powdered formulation. Accurately weigh 15 mg of the powder into five hydroxypropyl methylcellulose (HPMC) capsules. Put the capsules into a capsule based inhaler and measure the average emitted dose and particle size of the powder. Each 15 mg capsule (3.75 mg of drug) will deliver approximately 80% emitted dose which delivers 3.0 mg of direct factor Xa inhibitor to the lung.

### Example 34: Preparation of Metformin for Use in a Nebulizer Device

Prepare a solution of Metformin HCl drug at 60 mg/mL suitable for use in a nebulizer device using water. Using a 10 mL volumetric flask add 600 mg of USP grade metformin HCl. Add 14.8 mg of USP grade sodium citrate dihydrate. Add 8.0 mL of USP water for injection. Seal the flask and gently agitate until all the drug and buffer are dissolved and the solution is clear. Measure the pH of the solution and adjust to pH 7.0 using 1.0M NaOH. Bring the final volume of the flask to 10.0 mL mark using water for injection. Filter the solution through a 0.2 μm Durapore membrane. Transfer 2.0 mL to a nebulizer device and measure the liquid output rate to confirm at least 300 μL per minute output rate from the device.

### Example 35: Preparation of Anti-Fibrotic Ag-Ent (Nintedanib or Perfenidone) for Use in a Dry Powder Inhaler

Prepare a 10% formulation of Nintedanib or Perfenidone blended with 90% lactose carrier. Using a 1″ micromaster sanitary jet mill, add 100 g of Nintedanib or Perfenidone. Run the mill for approximately 60 minutes to produce crystalline Nintedanib or Perfenidone microparticles in the 3 to 5 μm range. Add 900 g of lactose carrier particles to a dry powder mixing machine particle blender. Add the 100 g of Nintedanib or Perfenidone to the blending hopper and mix for approximately 60 minutes. Fill 30 mg of powder into large gelatin capsules. Using a dry powder inhaler such as a TurboSpin, puncture the capsules and measure the emitted dose which is approximately 80% of the 30 mg. The amount of drug delivered to the lung is approximately 2.4 mg.

### Example 36: Preparation of 2.5 mg/ml Direct Factor Ha Inhibitor Argatroban for Use in a Nebulizer Device

Prepare a solution of direct factor IIa inhibitor drug Argatroban at 2.5 mg/mL suitable for use in a nebulizer device using solubility enhancers. Using a 10 mL volumetric flask add 125 mg of USP grade hydroxypropyl beta cyclodextrin. Add 8.0 mL of USP water for injection. Seal the flask and gently agitate until all the dextrin is dissolved and the solution is clear. Accurately weigh 25.0 mg of direct factor IIa inhibitor drug Argatroban and add it to the volumetric flask. Accurately weigh 50 mg of USP grade sodium citrate dihydrate and add it to the flask. Cover the flask. Using a sonicating water bath at 35° C. sonicate the solution for 10 minutes or until the solution is clear, and the cyclodextrin has complexed the drug. Measure the pH of the solution and adjust to pH 7.0 using 10 μL aliquots of 100 mM citric acid (prepared pH adjuster solution). Bring the final volume of the flask to 10.0 mL mark using water for injection. Filter the solution through a 0.2 μm Durapore membrane. Transfer 2.0 mL to a nebulizer device and measure the liquid output rate to confirm at least 300 μL per minute output rate from the device.

### Example 37: Preparation of 5.0 mg/ml Direct Factor Ha Inhibitor Argatroban for Use in a Nebulizer Device

Prepare a solution of direct factor IIa inhibitor drug Argatroban at 5.0 mg/mL suitable for use in a nebulizer device using solubility enhancers. Using a 10 mL volumetric flask add 175 mg of USP grade hydroxypropyl beta cyclodextrin. Add 100 mg of USP polyvinylpyrrolidone. Add 8.0 mL of USP water for injection. Seal the flask and gently agitate until all the dextrin is dissolved and the solution is clear. Accurately weigh 50.0 mg of direct factor IIa inhibitor drug Argatroban and add it to the volumetric flask. Accurately weigh 50 mg of USP grade sodium citrate dihydrate and add it to the flask. Cover the flask. Using a sonicating water bath at 35° C. sonicate the solution for 10 minutes or until the solution is clear and the cyclodextrin has complexed the drug. Measure the pH of the solution and adjust to pH 7.0 using 10 μL aliquots of 100 mM citric acid (prepared pH adjuster solution). Bring the final volume of the flask to the 10.0 mL mark using water for injection. Filter the solution through a 0.2 μm Durapore membrane. Transfer 2.0 mL to a nebulizer device and measure the liquid output rate to confirm at least 300 μL per minute output rate from the device.

### Example 38: Preparation of Direct Factor Ha Inhibitor Argatroban for Use in a Dry Powder Inhaler

Prepare a spray dried powder formulation 25% by weight of direct factor IIa inhibitor drug Argatroban. Particle size of the powder is targeted to be 3 to 10 um median mass aerodynamic diameter (MMAD). Using a spray nozzle attached to a Buchi spray dryer. Prepare a 10% (V/V) ethanol (or dimethyl sulfoxide (DMSO)) in a water cosolvent solution by adding 100 mL of USP grade ethanol to 900 mL USP grade water for injection and stir well. Pour a 100 mL aliquot into a bottle. To the 100 mL of cosolvent solution prepare a solution at 1.6% solids (16 mg/mL). Accurately weigh 400 mg of direct factor IIa inhibitor drug Argatroban and add it to the cosolvent solution. Accurately weigh 600 mg of USP grade mannitol and add it to the solution. Accurately weigh 600 mg of USP grade leucine and added to the solution. Stir the solution for 15 minutes until clear. Put the solution onto the spray dryer and collect the fine particles in the glass bottle collection system. After measuring the weight of the glass bottle (tare weight). Measure the spray drying recovery yield compared to 1600 mg of starting solids. Expect approximately 80% recovery approximately 1200 mg of powdered formulation. Accurately weigh 15 mg of the powder into five hydroxypropyl methylcellulose (HPMC) capsules. Put the capsules into a capsule based inhaler and measure the average emitted dose and particle size of the powder. Each 15 mg capsule (3.75 mg of drug) will deliver approximately 80% emitted dose which delivers 3.0 mg of direct factor IIa inhibitor to the lung.

### Example 39: Preparation of 2.5 mg/ml Direct Factor Ha Inhibitor Argatroban and 2.5 mg/ml Direct Factor Xa Inhibitor Apixaban (Direct Factor Ha Inhibitor to Direct Factor Xa Inhibitor Ratio is 1 to 1) for Use in a Nebulizer Device

Prepare a solution of drug at 2.5 mg/mL direct factor IIa Argatroban and 2.5 mg/ml direct factor Xa inhibitor Apixaban suitable for use in a nebulizer device using solubility enhancers. Using a 10 mL volumetric flask add 125 mg of USP grade hydroxypropyl beta cyclodextrin. Add 8.0 mL of USP water for injection. Seal the flask and gently agitate until all the dextrin is dissolved and the solution is clear. Accurately weigh 25.0 mg of direct factor IIa inhibitor drug Argatroban and 25.0 mg direct factor Xa inhibitor Apixaban add both drugs to the volumetric flask. Accurately weigh 50 mg of USP grade sodium citrate dihydrate and add it to the flask. Cover the flask. Using a sonicating water bath at 35° C. sonicate the solution for 10 minutes or until the solution is clear, and the cyclodextrin has complexed the drug. Measure the pH of the solution and adjust to pH 7.0 using 10 μL aliquots of 100 mM citric acid (prepared pH adjuster solution). Bring the final volume of the flask to 10.0 mL mark using water for injection. Filter the solution through a 0.2 μm Durapore membrane. Transfer 2.0 mL to a nebulizer device and measure the liquid output rate to confirm at least 300 μL per minute output rate from the device.

### Example 40: Preparation of 5.0 mg/ml Direct Factor Ha Inhibitor Argatroban and 5.0 mg/ml Direct Factor Xa Inhibitor Apixaban (Direct Factor Ha Inhibitor to Direct Factor Xa Inhibitor Ratio is 1 to 1) for Use in a Nebulizer Device

Prepare a solution of drug at 5.0 mg/mL direct factor IIa Argatroban and 5.0 mg/ml direct factor Xa inhibitor Apixaban suitable for use in a nebulizer device using solubility enhancers. Using a 10 mL volumetric flask add 175 mg of USP grade hydroxypropyl beta cyclodextrin. Add 100 mg of USP polyvinylpyrrolidone. Add 8.0 mL of USP water for injection. Seal the flask and gently agitate until all the dextrin is dissolved and the solution is clear. Accurately weigh 50.0 mg of direct factor IIa inhibitor drug Argatroban and 50.0 mg direct factor Xa inhibitor Apixaban and add both of drug to the volumetric flask. Accurately weigh 50 mg of USP grade sodium citrate dihydrate and add it to the flask. Cover the flask. Using a sonicating water bath at 35° C. sonicate the solution for 10 minutes or until the solution is clear and the cyclodextrin has complexed the drug. Measure the pH of the solution and adjust to pH 7.0 using 10 μL aliquots of 100 mM citric acid (prepared pH adjuster solution). Bring the final volume of the flask to the 10.0 mL mark using water for injection. Filter the solution through a 0.2 μm Durapore membrane. Transfer 2.0 mL to a nebulizer device and measure the liquid output rate to confirm at least 300 μL per minute output rate from the device.

### Example 41: Preparation of 3.0 mg/ml Direct Factor Ha Inhibitor Argatroban and 1.0 m/Ml Direct Factor Xa Inhibitor Apixaban (Direct Factor IIa Inhibitor to Direct Factor Xa Inhibitor Ratio is 3 to 1) for Use in a Nebulizer Device

Prepare a solution of drug at 3.0 mg/mL direct factor IIa Argatroban and 1.0 mg/ml direct factor Xa inhibitor Apixaban suitable for use in a nebulizer device using solubility enhancers. Using a 10 mL volumetric flask add 125 mg of USP grade hydroxypropyl beta cyclodextrin. Add 8.0 mL of USP water for injection. Seal the flask and gently agitate until all the dextrin is dissolved and the solution is clear. Accurately weigh 30.0 mg of direct factor IIa inhibitor drug Argatroban and 10.0 mg direct factor Xa inhibitor Apixaban, and add both drugs to the volumetric flask. Accurately weigh 50 mg of USP grade sodium citrate dihydrate and add it to the flask. Cover the flask. Using a sonicating water bath at 35° C. sonicate the solution for 10 minutes or until the solution is clear, and the cyclodextrin has complexed the drug. Measure the pH of the solution and adjust to pH 7.0 using 10 μL aliquots of 100 mM citric acid (prepared pH adjuster solution). Bring the final volume of the flask to 10.0 mL mark using water for injection. Filter the solution through a 0.2 μm Durapore membrane. Transfer 2.0 mL to a nebulizer device and measure the liquid output rate to confirm at least 300 μL per minute output rate from the device.

### Example 42: Preparation of Direct Factor Xa Inhibitor Apixaban Combined with Capitsol in Citrate Formulation for Application in an Aqueous Aerosol Device for Inhalation Therapy for Lungs

The procedure described below was to prepare a solution of low water-soluble direct factor Xa inhibitor drug Apixaban at saturated concentration suitable for use in a nebulizer device using solubility enhancers especially Capitsol (sulfobutylether-beta-cyclodextrin) in citrate buffer per the formulation as shown in Table 8. Apixaban has a very low solubility in aqueous buffers at about 28 μg/mL. Therefore, the main challenge is to formulate a high enough concentration to minimize the patient dosing time to the 40 minute time frame.

Capitsol was prepared at 40 mg/mL and was dissolved in 15 mM Sodium Citrate in a 25.0 mL volumetric flask. The solution was sonicated and mixed until clear and the measured pH was 7.7. This solution labeled as 4% Captisol® Solution.

6.0 mg of Apixaban was weighed into a separate vial and 20.0 mL of 4% Captisol solution prepared above was added. This 20 mL of Apixaban solution was sonicated with heat at 40° C. for approximately 30 minutes to maximally dissolve the Apixaban. The solution contained excess undissolved Apixaban crystals. This “saturated Apixaban solution prepared above (about 0.3 mg/ml)” was cooled and filtered to create a clear solution of Apixaban in 4% Captisol. The cooled solution was filtered, and pH was measured at 7.2±0.5. The concentration of Apixaban was measured by High-performance liquid chromatography (HPLC) at 265 ug/mL. The liquid output rate test was performed on an aqueous aerosol device for inhalation therapy. This strategy of dissolving excess Apixaban crystals and filtering was employed for higher concentrations (8% and 12%) of Captisol to create maximally saturated Apixaban solutions in Captisol.

Nebulization is the conversion of bulk liquids into micro-droplets suitable for inhalation into the lungs. The effectiveness of a nebulizer therapy is determined by how much of the drug bypasses the throat and deposits in the lung. The deposition location is determined by the size and velocity of the micro-droplets containing drug. These two parameters are influenced by several factors such as formulation, as well as the physics of the delivery devices in the atomization process which can affect the droplet size and velocity. Table 8 shows results of Apixaban content in droplet, pH and liquid output rate in a nebulizer for different drug formulation.

TABLE 8
Apixaban content in droplet, pH and liquid output rate of direct factor
Xa inhibitor Apixaban combined with Capitsol in citrate for application
in an aqueous aerosol device for inhalation therapy for lungs.

| | Apixaban | | Liquid |
|---|---|---|---|
| | Concentration | | output |
| | by HPLC, | | rate, |
| Sample description | μg/mL | pH | μL/min |
| Saturated Apixaban, 0% Captisol | 26 | 8.2 | 547 |
| 0.45% Saline | | | |
| Saturated Apixaban, 4% Captisol, | 265 | 7.8 | 603 |
| 15 mM Sodium Citrate | | | |
| Saturated Apixaban, 4% Captisol, 0.2% | 293 | 7.4 | 617 |
| Polyvinylpyrrolidone 15 mM Sodium Citrate | | | |
| Saturated Apixaban, 8% Captisol, | 553 | 7.5 | 614 |
| 15 mM Sodium Citrate | | | |
| Saturated Apixaban, 12% Captisol, | 821 | 7.5 | 579 |
| 15 mM Sodium Citrate | | | |

The ideal formulation for Nebulizer indication anticoagulants in the lungs should have high drug content in formulation to minimize dosing time. The formulation and device should provide a pH between 5.0-9.0, a particle size distribution below 10 μm (optimally between 2 μm to 6 μm) for efficient deep lung delivery. The liquid output rate should support short dosing times however more important is maintaining particle size distribution and emitted dose within an acceptable range. This product can perform with liquid output rates between 200 μL/min to 600 μL/min in standard hospital nebulizers. Table 8 shows the formulation of saturated Apixaban with 12% Captisol in 15 mM Sodium Citrate gave high Apixaban concentration (821 μg/mL) and relative high liquid output rate (579 μL/min) in a pH 7.5 close to physiological pH 7.4. With decreased Captisol content (from 12% to 8% to 4%) resulted a high output rate more than 600 μL/min (from 579 μL/min to 614 μL/min to 603 μL/min); However, a lower Apixaban content in droplet (from 821 μg/mL to 553 μg/mL to 265 μg/mL). All the formulation gave Apixaban concentration in droplet about 10 times to 32 times higher (from 265 μg/mL to 821 μg/mL) compared to Apixaban alone in aqueous buffer (about 26 μg/mL).

### Example 43: Preparation of Direct Factor Ha Inhibitor Argatroban in Citrate and Saline Formulation for Application in an Aqueous Aerosol Device for Inhalation Therapy for Lungs

The procedure described below was to prepare a solution of low water-soluble direct factor IIa inhibitor Argatroban for use in a nebulizer device in citrate and Saline per the formulation as shown in Table 9. Argatroban has a low solubility in aqueous buffers at about 3.0 mg/mL. Therefore, the main challenge is to formulate a high enough concentration to minimize the patient dosing time to the 40 minute time frame.

Argatroban formulations at 3 different concentration levels were prepared in Table 9. The drug was weighed into a vial and dissolved in 15 mM Sodium Citrate and 0.45% saline. The solution was sonicated with heat at 40° C. for approximately 30 minutes to maximally dissolve the Argatroban. The solution was cooled and filtered to create clear solutions of Argatroban in 15 mM Sodium Citrate and 0.45% saline. The cooled solution was filtered, and pH was measured at 6.0±0.5. The concentration of Argatroban was measured by High-performance liquid chromatography (HPLC). The liquid output rate test was performed on an aqueous aerosol device (Aerogen solo) for inhalation therapy (Table 9).

TABLE 9
Argatroban content, pH and liquid output rate of direct factor
IIa inhibitor Argatroban in citrate for application in an aqueous
aerosol device (Aerogen Solo) for inhalation therapy for lungs.

| | Argatroban | | Liquid |
|---|---|---|---|
| | Concentration | | output |
| | by HPLC, | | rate, |
| Sample description | μg/mL | pH | μL/min |
| 750 μg/mL Argatroban, 15 mM | 646 | 6.1 | 540 |
| Sodium Citrate, 0.45% Saline | | | |
| 1000 μg/mL Argatroban, 15 mM | 873 | 5.9 | 536 |
| Sodium Citrate, 0.45% Saline | | | |
| 1500 μg/mL Argatroban, 15 mM | 1340 | 6.1 | 498 |
| Sodium Citrate, 0.45% Saline | | | |

Table 9 shows that increased Argatroban concentration in the formulation (from 646 μg/mL to 1340 μg/mL) causes a slight reduction in liquid output rate from 540 to 500 μL/min liquid output rate. Therefore, very high drug loading may reduce device performance and product performance.

### Example 44: Preparation of Direct Factor Xa Inhibitor Apixaban and Direct Factor IIa Inhibitor Argatroban Combined with Capitsol/PVP in Citrate Formulation for Application in an Aqueous Aerosol Device for Inhalation Therapy for Lungs

The procedure described below was to prepare a solution of low water-soluble direct factor Xa inhibitor Apixaban and direct factor IIa inhibitor Argatroban formulation suitable for use in a nebulizer device using solubility enhancers combined with Capitsol/PVP in citrate per the formulation as shown in Table 10.

Capitsol/PVP calculated by formulation in Table 10 was dissolved in 15 mM Sodium Citrate in a 25.0 mL volumetric flask and the solution was sonicated and mixed until clear. This solution labeled as Captisol/PVP Solution. Certain amount of Apixaban and Argatroban calculated by formulation in Table 10 were weighed into a separate vial and 20.0 mL of Captisol/PVP solution prepared above was added. This 20 mL of Apixaban and Argatroban solution was sonicated with heat at 40° C. for approximately 30 minutes to maximally dissolve the anticoagulants. This solution was cooled and filtered to create a clear solution of Apixaban and Argatroban in Captisol Solution. The cooled solution was filtered, and pH was measured. The concentrations of the anticoagulants were measured by High-performance liquid chromatography (HPLC). The liquid output rate test was performed on an aqueous aerosol device for inhalation therapy. Such an aerosol device was used for nebulizing a drug for inhalation by a patient whereby the nebulized drug is administered to the patient through deposition in his lungs. The effectiveness of Apixaban and Argatroban formulation was examined by drug content and liquid output rate in a nebulizer. Table 10 shows results of Apixaban and Argatroban content, pH and liquid output rate in a nebulizer for different drug formulation.

TABLE 10
Drug content, pH and liquid output rate of direct factor Xa inhibitor Apixaban and direct factor
IIa inhibitor Argatroban combined with Capitsol/ Polyvinylpyrrolidone (PVP) in citrate
formulation for application in an aqueous aerosol device for inhalation therapy for lungs

| | Apixaban | Argatroban | | Liquid |
|---|---|---|---|---|
| | Concentration | Concentration | | output |
| | by HPLC, | by HPLC, | | rate, |
| Sample description | μg/mL | μg/mL | pH | μL/min |
| 150 μg/mL Apixaban, 750 μg/mL Argatroban, 7% Captisol, 10 mM sodium citrate, | 119 | 752 | 7.3 | 422 |
| 0.15% Polyvinylpyrrolidone | | | | |
| 150 μg/mL Apixaban, 750 μg/mL Argatroban, 8% Captisol, 10 mM sodium citrate, | 156 | 790 | 7.2 | 404 |
| 0.15% Polyvinylpyrrolidone | | | | |
| 175 μg/mL Apixaban, 875 μg/mL Argatroban, 8% Captisol, 10 mM sodium citrate, | 151 | 890 | 7.2 | 415 |
| 0.15% Polyvinylpyrrolidone | | | | |
| 200 μg/mL Apixaban, 1000 μg/mL Argatroban, 10% Captisol, 10 mM sodium citrate, | 190 | 992 | 7.2 | 421 |
| 0.15% Polyvinylpyrrolidone | | | | |
| 160 μg/mL Apixaban, 750 μg/mL Argatroban, 8% Captisol, 15 mM sodium citrate | 140 | 772 | 7.6 | 514 |
| 160 μg/mL Apixaban, 750 μg/mL Argatroban, 9% Captisol, 15 mM sodium citrate | 125 | 776 | 7.7 | 519 |
| 175 μg/mL Apixaban, 750 μg/mL Argatroban, 10% Captisol, 15 mM sodium citrate | 184 | 819 | 7.7 | 484 |
| 200 μg/mL Apixaban, 1000 μg/mL Argatroban, 11% Captisol, 15 mM sodium citrate | 198 | 1006 | 7.4 | 417 |
| 300 μg/mL Apixaban, 750 μg/mL Argatroban, 15 mM sodium citrate, 11% Captisol | 305 | 741 | 7.5 | 480 |
| 300 μg/mL Apixaban, 1000 μg/mL Argatroban, 15 mM sodium citrate, 11% Captisol | 312 | 992 | 7.5 | 472 |
| 300 μg/mL Apixaban, 1000 μg/mL Argatroban, 15 mM sodium citrate, 12% Captisol | 281 | 977 | 7.5 | 453 |
| 200 μg/mL Apixaban, 1000 μg/mL Argatroban, 11% Captisol, 15 mM Sodium Citrate | 198 | 1006 | 7.4 | 417 |
| 200 μg/mL Apixaban, 1600 μg/mL Argatroban, 15 mM Sodium Citrate, 7% Captisol | 202 | 1569 | 7.5 | 255 |
| 200 μg/mL Apixaban, 1800 μg/mL Argatroban, 15 mM Sodium Citrate, 7% Captisol | 212 | 1783 | 7.5 | 254 |
| 200 μg/mL Apixaban, 2000 μg/mL Argatroban, 15 mM Sodium Citrate, 7% Captisol | 226 | 1963 | 7.7 | 229 |
| 300 μg/mL Apixaban, 750 μg/mL Argatroban, 15 mM Sodium Citrate, 11% Captisol | 305 | 741 | 7.5 | 480 |
| 300 μg/mL Apixaban, 1000 μg/mL Argatroban, 15 mM Sodium Citrate, 11% Captisol | 312 | 992 | 7.5 | 472 |
| 300 μg/mL Apixaban, 1000 μg/mL Argatroban, 15 mM Sodium Citrate, 12% Captisol | 281 | 977 | 7.5 | 453 |
| 400 μg/mL Apixaban, 2000 μg/mL Argatroban, 16.5 mM Sodium Citrate, | 390 | 1978 | 6.0 | 275 |
| 11.5% Captisol | | | | |
| 450 μg/mL Apixaban, 2250 μg/mL Argatroban, 16.5 mM Sodium Citrate, | 428 | 2188 | 6.0 | 249 |
| 11.5% Captisol | | | | |

Table 10 shows the nebulizer used formulation with direct factor Xa inhibitor Apixaban and direct factor IIa inhibitor Argatroban combined with Capitsol/PVP in citrate delivered drug amount in droplet corresponding to their loaded drug amount with pH close to human physiological pH 7.4 and liquid output rate within an acceptable range between 250 μg/mL to 519 μg/mL. All the formulations gave Apixaban concentration in droplet about 5 times to 16 times higher (from 119 μg/mL to 428 μg/mL) compared to Apixaban alone in aqueous buffer (about 26 μg/mL).

### Example 45: Nebulized Aerosol Testing of Direct Factor Xa Inhibitor Apixaban and Direct Factor IIa Inhibitor Argatroban Combined with Capitsol in Citrate Formulation EM-AM220 for Application in an Aqueous Aerosol Device for Inhalation Therapy for Lungs

An inhalation solution of Apixaban and Argatroban could effectively treat and reduce severe lung inflammation. The lead nebulizer is a piezo driven vibrating mesh device. The Aerogen solo and Ultra mouthpiece are designed to create 5 μm droplets, ideal for deep lung delivery of water-based drug solutions. The Aerogen device is generally capable of delivering from 1 mL to 20 mL of dosing solution in 2 minutes to 60 minutes. To design the formulation, we chose an approximate adult lung dose of 1.0 mg of Apixaban and 10 mg of Argatroban. Due to multiple points of inefficiency in nebulizers, the lung dose is about 50% of the dose placed in the nebulizer reservoir.

Nebulization is a method of administering drugs by converting the solution into aerosols. Most aerosolized particles for therapeutic purposes are in the range of 2 μm 5 μm and diffusion is the predominant mechanism for lung deposition. The optimal technique for aerosolization is important to achieve distal airway and alveolar deposition. The deposition characteristics of droplets in the alveolar region, airways, mouth, and throat and other parts of the body depend heavily on particle size. The particle size and velocity of the droplets are affected by different factors including the breathing mode of the patient, the nebulizer system, the geometry of the nozzle, and the aerosol properties. To be effective, aerosol droplets generated in nebulizers must present a certain size distribution which allow them to pass the oropharynx and be distributed in the bronchial airways. To assess the quality of inhalation aerosol, the reliable measurement of droplet size distribution was studied.

The formulation of EM-AM220 was prepared as below. Added 100 mL water and 29.41 g of trisodium citrate dihydrate into a flask and stirred until clear to make 1.0 M trisodium citrate dihydrate. In a 100 mL clean dry class A volumetric flask, added 7392 mg of Captisol and 90 mL of water into the flask and sonicated until clear, then added 1.5 mL of 1.0 M citrate buffer to the flask with sonication and stirring. Brought to final volume by adding water to the calibration mark (100.0 mL) and this was 7% Captisol/15 mM citrate buffer solution.

In a separate 10.0 mL clean dry class A volumetric flask, added 2.004 mg of Apixaban and 20.73 mg of Argatroban then added 10.0 mL of the 7% Captisol/15 mM citrate buffer solution to the calibration mark with the cap and sonicated with heat about 30° C. to 40° C. for 30 minutes or until the solution is clear. This is formulation EM-AM220. The pH was 7.2±0.5. The final solution was filtered through a sterilized 25 mm 0.22 um Durapore syringe filter using a 50 mL syringe. The drug concentrations targets were verified by High-performance liquid chromatography (HPLC) methods and results were summarized in Table 13.

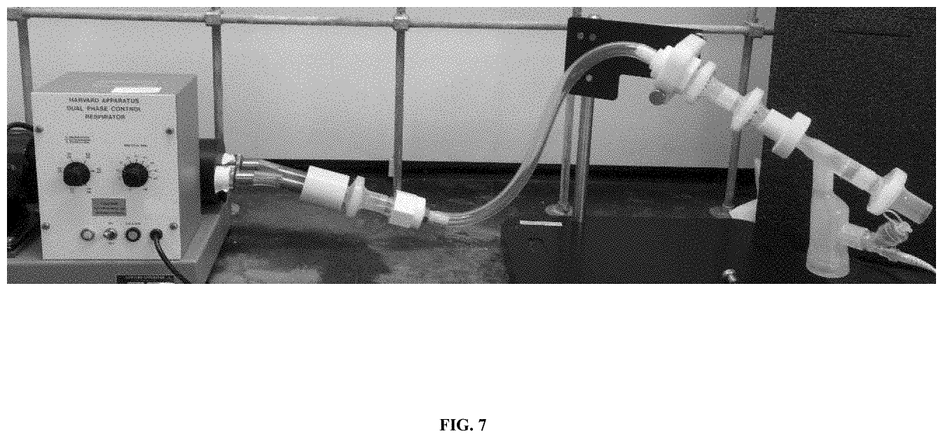

The formulation solution was nebulized by the Aerogen Solo (coupled with the Pro-X controller and the Ultra-Chamber). Droplet size distribution by laser diffraction and nebulizer output rate by gravimetric analysis were used for formulation and device testing and results are summarized in Table 11 below. USP1601 guidance was followed to measure delivered dose and collected by tidal volume breath simulator until dose completion of 10.0 mL (1 delivered dose=(2×5 mL) charged to the nebulizer reservoir), and the results are summarized in Table 5 below. The Respirgard filters were replaced every 4 minutes during aerosol dose collection. Each dose sample was collected and numbered. Each collection filter was capped at both ends and stored in individual bitran bags to prevent evaporating for assay. Aerosol samples were extracted from the collection filter then quantitated by High-performance liquid chromatography (HPLC) analysis and results are summarized in Table 13 below. FIG. 7 shows the setup used when performing delivered dose (DD) testing of nebulizer.

TABLE 11
Comparison of the droplet size distribution with 1-minute constant
inhalation flow rate at 15 L/min of three nebulizers tested (n = 2).

| | | | | | | Geometric | Fine | Fine |
|---|---|---|---|---|---|---|---|---|
| | | Charge | | | | Standard | Particle | Particle |
| Formulation | Device | Volume | Dv10* | Dv50* | Dv90* | Deviation | Fraction <3.3 μm | Fraction <5.0 μm |
| ID | ID | (mL) | (μm) | (μm) | (μm) | (μm) | (%) | (%) |
| EM-AM220 | C200530-0366 | 2 | 1.97 ± 0.01 | 4.52 ± 0.06 | 9.85 ± 0.21 | 1.81 ± 0.01 | 31.5 ± 0.7 | 56.5 ± 0.7 |
| | C200530-0501 | 2 | 2.05 ± 0.18 | 4.44 ± 0.40 | 9.18 ± 0.71 | 1.71 ± 0.01 | 31.0 ± 6.4 | 58.0 ± 5.7 |
| | C200530-0643 | 2 | 2.09 ± 0.01 | 4.63 ± 0.04 | 9.95 ± 0.34 | 1.75 ± 0.02 | 30.0 ± 0.0 | 54.5 ± 0.7 |

*Dv10: 10th percentile of the cumulative droplet/particle size distribution by volume (mass).
*Dv50: Median diameter of the cumulative droplet/particle size distribution by volume (mass).
*Dv90: 90th percentile of the cumulative droplet/particle size distribution by volume (mass).

Table 12 shows the value of the Dv50 by laser analysis is about 4.4 μm to 4.6 μm volume diameter. Dv50 can be used to estimate the MMAD (Mass Median Aerodynamic Diameter) or particle size distribution of an aerosol. Aerosols with a MMAD of less than 5.0 μm are considered ideal for deep lung drug delivery.

TABLE 11
Comparison of the droplet size distribution with 1-minute constant
inhalation flow rate at 15 L/min of three nebulizers tested (n = 2).

| | | | | | | | Fine | Fine |
|---|---|---|---|---|---|---|---|---|
| | | | | | | Geometric | Particle | Particle |
| | | Charge | | | | Standard | Fraction | Fraction |
| Formulation | | Volume | Dv10* | Dv50* | Dv90* | Deviation | <3.3 μm | <5.0 μm |
| ID | Device ID | (mL) | (μm) | (μm) | (μm) | (μm) | (%) | (%) |
| EM-AM220 | C200530-0366 | 2 | 1.97 ± 0.01 | 4.52 ± 0.06 | 9.85 ± 0.21 | 1.81 ± 0.01 | 31.5 ± 0.7 | 56.5 ± 0.7 |
| | C200530-0501 | 2 | 2.05 ± 0.18 | 4.44 ± 0.40 | 9.18 ± 0.71 | 1.71 ± 0.01 | 31.0 ± 6.4 | 58.0 ± 5.7 |
| | C200530-0643 | 2 | 2.09 ± 0.01 | 4.63 ± 0.04 | 9.95 ± 0.34 | 1.75 ± 0.02 | 30.0 ± 0.0 | 54.5 ± 0.7 |

*Dv10: 10th percentile of the cumulative droplet/particle size distribution by volume (mass).
*Dv50: Median diameter of the cumulative droplet/particle size distribution by volume (mass).
*Dv90: 90th percentile of the cumulative droplet/particle size distribution by volume (mass).

Table 12 shows that 99% of loaded drug solution was nebulized from the device. The (Delivered Dose) DD is an in vitro measurement of the amount of drug delivered to a patient's lung. The DD of nebulizers typically ranges from 45 to 85% of the amount of drug nebulized or aerosolized. The test shows the DD of the formulation was ˜60%. The nebulization time for this test ranged between 250 to 290 μl/min, which means the dose can be delivered in approximately 40 minutes. This meets the criteria that the formulation and Aerogen solo device combination is capable of delivering a volume from 1 mL to 20 mL of dosing solution in 2 minutes to 60 minutes.

TABLE 13
Nebulizer Delivered dose results by High-performance liquid chromatography (HPLC) analysis (n = 2).

| | | | | | | Apixaban | Argatroban |
|---|---|---|---|---|---|---|---|
| | | Theoretical | Apixaban | Theoretical | Argatroban | Recovery | Recovery |
| | | Apixaban | Found | Argatroban | Found | (% of | (% of |
| Formulation | Nebulizer | dose | dose | dose | dose | Theoretical | Theoretical |
| ID | ID | (μg) | (μg) | (μg) | (μg) | Dose)* | Dose)* |
| EM-AM220 | C200530-0366 | 2016 | 1213 ± 23 | 19775 | 12595 ± 244 | 60.0 ± 1.4 | 64.0 ± 1.4 |
| | C200530-0501 | 2016 | 1131 ± 36 | 19775 | 11762 ± 371 | 56.0 ± 2.1 | 59.0 ± 2.1 |
| | C200530-0643 | 2016 | 1171 ± 26 | 19775 | 12149 ± 254 | 58.0 ± 1.4 | 61.5 ± 0.7 |

*Theoretical dose based on 10 mL charge volume

Results from Table 13 shows that the average delivered dose was about 1,200 μg Apixaban and 12,000 μg Argatroban, which was about 60% of total aerosolized drug mass.

### Example 47: In Vivo Study of Direct Factor Xa Inhibitor Apixaban and Direct Factor IIa Inhibitor Argatroban Combined with Capitsol in Citrate Formulation EM-AM15 for Application in an Aqueous Aerosol Device for Inhalation Therapy for Lungs

The formulation of EM-AM15 was prepared as below. Added 100 mL water and 29.41 g of trisodium citrate dihydrate into a flask and stirred until clear to make 1.0 M trisodium citrate dihydrate. In a 200 mL clean dry class A volumetric flask, added 11554.62 mg of Captisol and 190 mL of water into the flask and sonicated until clear, then added 1.5 mL of 1.0 M citrate buffer to the flask with sonication and stirring. Brought to final volume by adding water to the calibration mark (200.0 mL) and this was 11% Captisol/15 mM citrate buffer.

In a separate 50.0 mL clean dry class A volumetric flask, added 10.02 mg of Apixaban and 51.81 mg of Argatroban then added 45.0 mL of the 11% Captisol/15 mM citrate buffer solution to the calibration mark with the cap and sonicated with heat about 30° C. to 40° C. for about 30 minutes or until the solution is clear. This is formulation EM-AM15. The pH confirmed was 7.4. The final solution was filtered through sterilized 25 mm 0.22 um Durapore syringe filters using a 50 mL syringe. Verified the drug concentrations by High-performance liquid chromatography (HPLC) methods.

The in vivo study was conducted to investigate the whole blood and bronchoalveolar lavage (BAL) pharmacodynamics and lung pathology following a single intra-tracheal instillation of anti-coagulation Formulation EM-AM15 contains direct factor Xa inhibitor Apixaban and direct factor IIa inhibitor Argatroban combined with Capitsol in citrate to male Sprague-Dawley rats and evaluated at 5 minutes, 1 hour, 2 hours, 3.5 hours, 5 hours, and 6 hours post-dose intervals.

At their assigned time-point, rats were anesthetized and subjected to exsanguination via an abdominal vessel and gross necropsy. Whole blood (two ˜1.5 ml aliquots, K2EDTA) was collected. The whole blood was stored frozen at −60° C. or lower until shipped overnight with dry ice for further evaluation.

The lungs were removed and divided into left and right lung. Each half of the lung was weighed and placed in separate vials (for Pharmacokinetics analysis), snap frozen in liquid nitrogen and stored frozen at −60° C. or lower until shipped overnight on dry ice for further evaluation.

The in-vivo study showed that intra-tracheal administration of EM-AM15 at 250 μL/kg was well tolerated by all animals. There were no adverse clinical observations observed. All samples were collected on time.

Table 14 is the results of drug content in tissue and whole blood of in vivo Pharmacokinetics study after intra-tracheal administration of EM-AM15 at 250 μL/kg in animals. The formulation EM-AM15 gave 75 μg of Argatroban and 15 μg of Apixaban after 75 μl of intra-tracheal administration of EM-AM15 for a 0.3 kg animal.

TABLE 14
In vivo Pharmacokinetics results of drug content in tissue
and whole blood of after intra-tracheal administration
of EM-AM15 at 250 μL/kg in animals*(n = 6).

| | Argatroban ( 75 μg dose) | | Apixaban ( 15 μg dose) | |
|---|---|---|---|---|
| | Lung Tissue, | Blood, | Lung Tissue, | Blood, |
| Time | ng/mg | ng/ml | ng/mg | ng/ml |
| 5 min | 32.1 ± 9.3 | 18.59 ± 15.01 | 0.75 ± 0.60 | 45.36 ± 14.71 |
| 1 H | 8.0 ± 5.1 | 23.42 ± 11.44 | 0.02 ± 0.00 | 8.82 ± 2.49 |
| 2 H | 3.0 ± 1.4 | 2.64 ± 0.58 | 0.01 ± 0.00 | 2.49 ± 0.80 |
| 3.5 H | 1.2 ± 0.2 | BQL | 0.01 ± 0.00 | 1.19 |
| 5 H | 0.4 ± 0.3 | BQL | BOL | BQL |
| 6 H | 0.1 ± 0.2 | BQL | BOL | BQL |

*Animal weighing 0.3 kg received 75 μl of the formulation

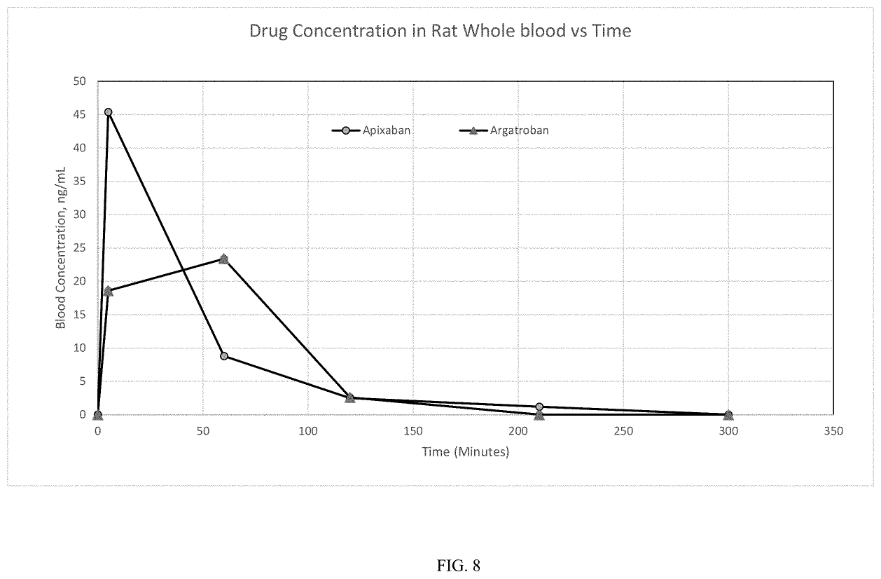

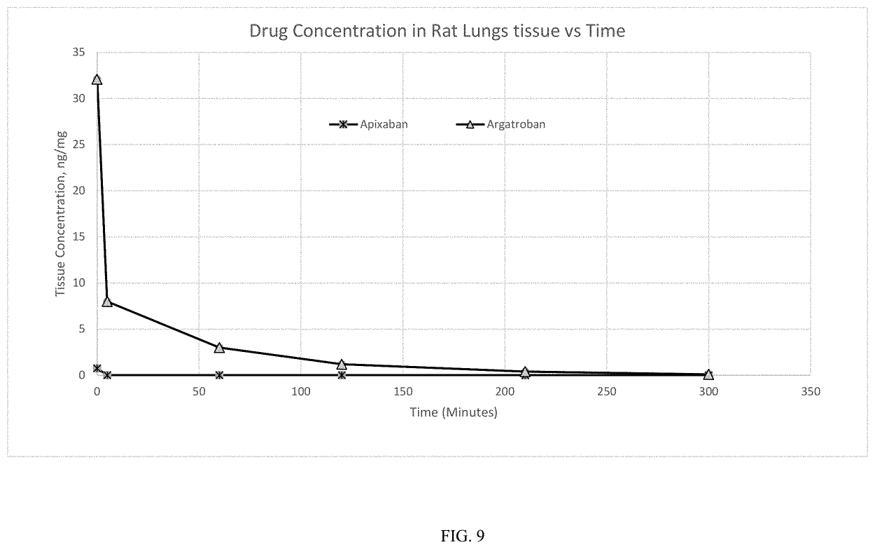

FIG. 8 is the drug concentration in rat whole blood vs time and FIG. 9 is the drug concentration in rat lungs tissue vs time. Table 14 and FIGS. 8 and 9 show that with 75 μg of Argatroban and 15 μg of Apixaban after 75 μl of intra-tracheal administration of EM-AM15 for a 0.3 kg animal, the systemic drug in whole blood lasted up to 2 hours for Argatroban and up to 3.5 hours for Apixaban; the drug content in lung tissue lasted more than 6 hours for Argatroban and up to 3.5 hours for Apixaban.

### Example 48: In-Vivo Study of Direct Factor Xa Inhibitor Apixaban and Direct Factor Ha Inhibitor Argatroban Combined with Azelastine and Hydroxychloroquine in Capitsol/Citrate Buffer Formulation EM-AMAQ15 for Application in an Aqueous Aerosol Device for Inhalation Therapy for Lungs

The formulation of EM-AMAQ15 was prepared as below. Added 100 mL water and 29.41 g of trisodium citrate dihydrate into a flask and stirred until clear to make 1.0 M trisodium citrate dihydrate. In a 200 mL clean dry class A volumetric flask, added 14705.88 mg of Captisol and 190 mL of water into the flask and sonicated until clear, then added 3.0 mL of 1.0 M citrate buffer to the flask with sonication and stirring. Brought to final volume by adding water to the calibration mark (200.0 mL) and this was the 7% Captisol/15 mM citrate buffer solution.

In a separate 100.0 mL clean dry class A volumetric flask, added 10.0 mg of Azelastine hydrochloride, 10.02 mg of Apixaban, 103.62 mg of Argatroban and 150.30 mg of Hydroxychloroquine sulfate then added 90.0 mL of the 7% Captisol/15 mM citrate buffer solution to the calibration mark with the cap and sonicated with heat about 30° C. to 40° C. for about 30 minutes or until the solution is clear. This is formulation EM-AMAQ15. The pH confirmed was 7.2±0.5. The final solution was filtered through sterilized 25 mm 0.22 um Durapore syringe filters using a 50 mL syringe. Verified the drug concentrations by High-performance liquid chromatography (HPLC) methods.

The in vivo study was undertaken to evaluate the effects of an intra-tracheal instillation of EM-AMAQ15 at 375 μL/kg in a rat model following a single intra-tracheal instillation of anti-coagulation Formulation EM-AMAQ15 contains direct factor Xa inhibitor Apixaban, direct factor IIa inhibitor Argatroban combined with Azelastine and Hydroxychloroquine in Capitsol/citrate buffer to male Sprague-Dawley rats and evaluated at 5 minutes, 30 minutes, 1 hour, 2 hours, 3.5 hours and 5 hours post-dose intervals.

Whole blood (two ˜100 μL aliquots, K2EDTA) was collected from the catheter of conscious rats for systemic pharmacokinetics (PK) at the time-points. Whole blood samples were stored at −60° C. or lower until shipped overnight on dry ice for further evaluation.

The lungs from animals were removed and divided into left and right lung at the time-points listed in the table above. Each half of the lung was weighed and placed in separate vials (for Pharmacokinetics analysis), snap frozen in liquid nitrogen and stored frozen at −60° C. or lower until shipped overnight on dry ice to the sponsor for further evaluation.

The in-vivo study showed that intra-tracheal administration of EM-AMAQ15 at 375 μL/kg was well tolerated by all animals. There were no adverse clinical observations observed. All samples were collected on time.

Table 15 is the results of drug content in tissue and whole blood of in vivo Pharmacokinetics study after intra-tracheal administration of EM-AMAQ15 at 375 μL/kg in animals. The formulation EM-AMAQ15 gave 112.5 μg of Argatroban, 11.25 μg of Apixaban, 168.5 μg of Hydroxychloroquine and 11.25 μg of Azelastine after 112.5 μl of intra-tracheal administration of EM-AMAQ15 for a 0.3 kg animal.

TABLE 15
In vivo Pharmacokinetics results of drug content in tissue and whole blood of after
intratracheal administration of EM-AMAQ15 at 375 μL/kg in animals* (n = 6).

| | | Argatroban | | Apixaban | | Hydroxychloroquine | | Azelastine | |
|---|---|---|---|---|---|---|---|---|---|
| | | (112.5 μg dose) | | (11.25 μg dose) | | (168.5 μg dose) | | (11.25 μg dose) | |
| | | Lung | | Lung | | Lung | | Lung | |
| | | Tissue, | Blood, | Tissue, | Blood, | Tissue, | Blood, | Tissue, | Blood, |
| Time | | ng/mg | ng/ml | ng/mg | ng/ml | ng/mg | ng/ml | ng/mg | ng/ml |
| 5 | min | 34.31 ± 16.61 | 28.97 ± 10.94 | 0.33 ± 0.15 | 37.62 ± 7.70 | 40.07 ± 20.61 | 137.86 ± 49.79 | 0.59 ± 0.15 | 6.98 ± 2.17 |
| 30 | min | 49.23 ± 18.08 | 30.18 ± 10.20 | 0.03 ± 0.01 | 17.34 ± 4.19 | 23.39 ± 6.40 | 76.94 ± 31.53 | 0.25 ± 0.10 | 6.08 ± 6.62 |
| 1 | H | 15.76 ± 7.71 | 41.80 ± 14.30 | 0.01 ± 0.00 | 6.23 ± 1.90 | 13.25 ± 3.74 | 52.13 ± 18.74 | 0.10 ± 0.01 | 2.21 ± 0.51 |
| 2 | H | 4.45 ± 2.48 | 6.28 ± 4.35 | BQL | 2.10 ± 0.37 | 10.60 ± 6.93 | 23.77 ± 13.38 | 0.04 ± 0.01 | 1.24 ± 0.29 |
| 3.5 | H | 1.29 ± 0.31 | 1.54 ± 0.89 | BQL | BQL | 2.07 ± 2.96 | 13.96 ± 9.11 | 0.01 ± 0.00 | BQL |
| 5 | H | 0.55 ± 0.27 | BQL | BQL | BQL | 4.99 ± 2.01 | 17.25 ± 3.43 | 0.01 ± 0.01 | BQL |

*Animal weighing 0.3 kg received 112.5 μl of the formulation

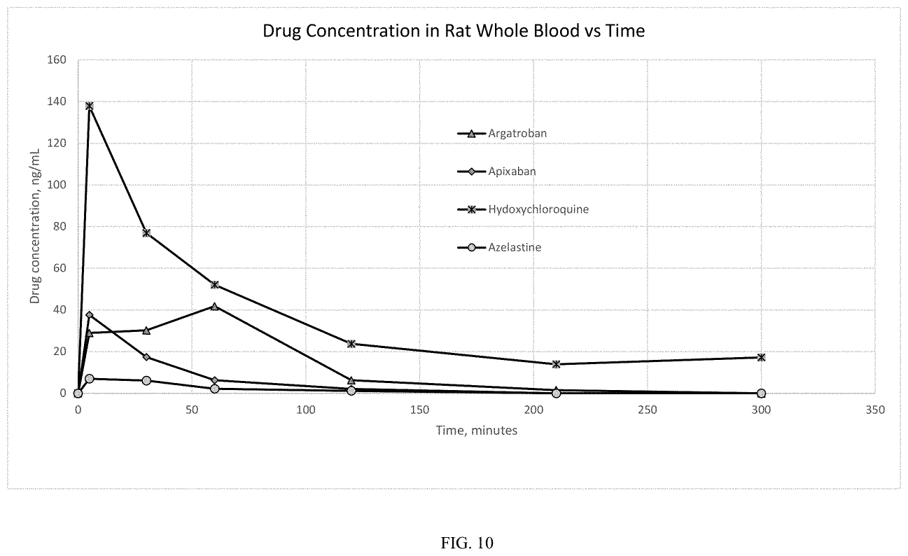

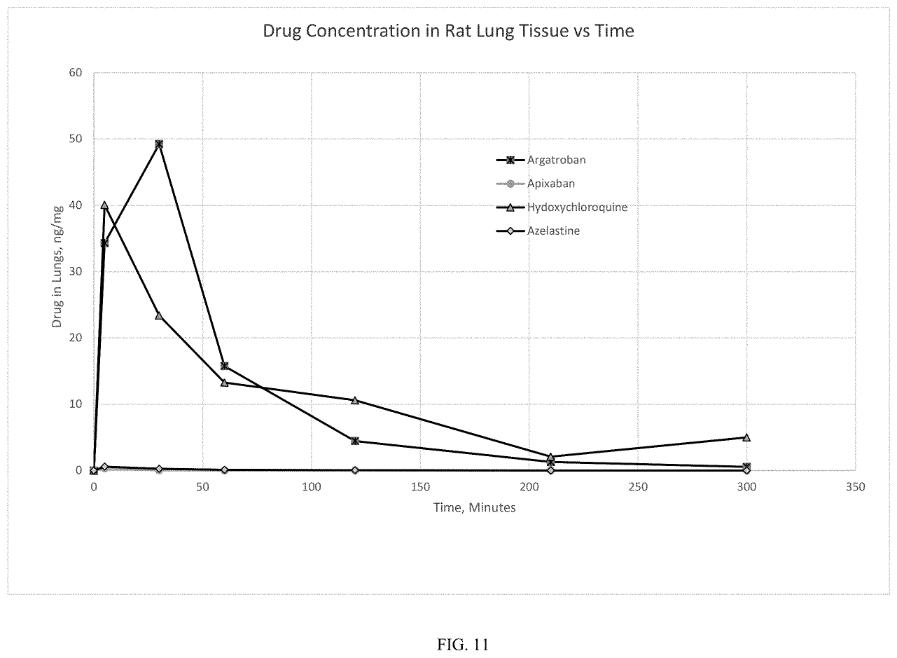

FIG. 10 the drug concentration in rat whole blood vs time and FIG. 11 is the drug concentration in rat lungs tissue vs time. Table 15 and FIGS. 10 and 11 shows that with 112.5 μg of Argatroban, 11.25 μg of Apixaban, 168.5 μg of Hydroxychloroquine and 11.25 μg of Azelastine after 112.5 μl of intra-tracheal administration of EM-AMAQ15 for a 0.3 kg animal, the systemic drug in whole blood lasted up to 3.5 hours for Argatroban and up to 2 hours for Apixaban, lasted more than 5 hours for Hydroxychloroquine and lasted up to 2 hours for Azelastine; the drug content in lungs tissue lasted more than 5 hours for Argatroban and up to 1 hours for Apixaban, lasted more than 5 hours for Hydroxychloroquine and lasted more than 5 hours for Azelastine.

### Example 49: Preparation of Direct Factor Xa Inhibitor Apixaban Capsule Formulation for Dry Powder Inhalation

Dry powder inhalers provide substantially improved lung deposition, faster delivery, and more convenient administration compared to the nebulized formulation. Capsule based dry powder inhalers can easily delivery 1 mg to 20 mg of drug in a single dose. Typical capsules can deliver 5 mg to 100 mg of formulated powder to the deep lung per inhalation. Solvent spray drying can use several different organic solvents to solubilize low solubility compounds to produce inhalation powders. Several different organic solvents can be used to solubilize water insoluble drugs for spray drying techniques. These different systems can create dry powders with different degrees of crystallinity or amorphous phase of the final drug. The degree of crystallinity can affect the Pharmacokinetics profile of the inhaled drug by slowing dissolution kinetics.

Preparation of dry powder inhalation formulation for Capsule containing a direct factor Xa inhibitor Apixaban was spray dried using 70% Pharmaceutical Grade Dimethyl sulfoxide (DMSO) and 30% HPLC grade water. The dry powder contains 5% Apixaban. A 40 mg dose of powder contains 2.0 mg of Apixaban. A 325 mL of solution was prepared by weighing the following components into an Erlenmeyer flask. 93.75 mg of Apixaban, 1500 mg of trehalose, and finally 281.25 mg of leucine with a total of 1875 mg of solids was weighed. 227.5 mL of Dimethyl sulfoxide and 97.5 mL water were added to the mark of Erlenmeyer flask and the solution was stirred for 15 minutes until clear. A solvent spray drying system was used to spray the powder into a collector. The yield was 60% of a fine powder suitable for inhalation. The particle size was determined to be approximately 4.5 um median mass aerodynamic diameter (MMAD). 40 mg of the powder was filled into HPMC capsules and the emitted dose was measured to be 75% using Plastiape RS01 inhaler. Therefore, one capsule would deliver 1.5 mg of Apixaban to the patient's lung.

### Example 50: Preparation of a Direct Factor Ha Inhibitor Argatroban Capsule Formulation for Dry Powder Inhalation

Preparation of dry powder inhalation formulation for Capsule containing a direct factor IIa inhibitor Argatroban was spray dried using 70% Pharmaceutical Grade Dimethyl sulfoxide (DMSO) and 30% HPLC grade water. The dry powder contains 25% Argatroban by weight. A 40 mg dose of powder contains 0 mg of Argatroban. A 375 mL of solution was prepared by weighing the following components into an Erlenmeyer flask. 468.75 mg of Argatroban, 1218.75 mg of trehalose, and finally 187.5 mg of leucine with a total of 1875 mg of solids was weighed. 262.5 mL of Dimethyl sulfoxide and 112.5 mL water were added to the mark of Erlenmeyer flask and the solution was stirred for 15 minutes until clear. A solvent spray drying system was used to spray the powder into a collector. The yield was 60% of a fine powder suitable for inhalation. The particle size was determined to be approximately 4.5 um median mass aerodynamic diameter (MMAD). 40 mg of the powder was filled into HPMC capsules and the emitted dose was measured to be 75% using Plastiape RS01 inhaler. Therefore, one capsule would deliver 7.5 mg of Argatroban to the patient's lung.

### Example 51: Preparation of 50% Direct Factor Ha Inhibitor Argatroban and 5% Direct Factor Xa Inhibitor Apixaban Capsule Formulation for Dry Powder Inhalation

Dry powder inhalers provide substantially improved lung deposition, faster delivery, and more convenient administration compared to the nebulized formulation. Capsule based dry powder inhalers can easily delivery 1 mg to 20 mg of drug in a single dose. Typical capsules can deliver 5 mg to 100 mg of formulated powder to the deep lung per inhalation. Solvent spray drying can use several different organic solvents to solubilize low solubility compounds to produce inhalation powders. Several different organic solvents can be used to solubilize water insoluble drugs for spray drying techniques. These different systems can create dry powders with different degrees of crystallinity or amorphous phase of the final drug. The degree of crystallinity can affect the Pharmacokinetics profile of the inhaled drug by slowing dissolution kinetics. Additionally, 2 or 3 drugs can be combined into a combination formulation and spray dried into a single delivery platform.

Preparation of dry powder inhalation formulation for Capsule containing 50% direct factor IIa inhibitor Argatroban and 5% direct factor Xa inhibitor Apixaban was spray dried using 70% Pharmaceutical Grade Dimethyl sulfoxide (DMSO) and 30% HPLC grade water. The dry powder contains 5% Apixaban and 50% Argatroban by weight. A 40 mg dose of powder contains 2.0 mg of Apixaban and 20 mg of Argatroban. A 325 mL of solution was prepared by weighing the following components into an Erlenmeyer flask. 93.75 mg of Apixaban, 937.5 mg of Argatroban, 656.25 mg of trehalose, and finally 187.5 mg of leucine with a total of 1875 mg of solids was weighed. 227.5 mL of Dimethyl sulfoxide and 97.5 mL water were added to the mark of Erlenmeyer flask and the solution was stirred for 15 minutes until clear. A solvent spray drying system was used to spray the powder into a collector. The yield was 60% of a fine powder suitable for inhalation. The particle size was determined to be approximately 4.5 um median mass aerodynamic diameter (MMAD). 40 mg of the powder was filled into HPMC capsules, and the emitted dose was measured to be 75% using Plastiape RS01 inhaler. Therefore, one capsule would deliver 1.5 mg of Apixaban and 15.0 mg of Argatroban to the patient's lung.

### Example 52: Preparation of Direct Factor Ha Inhibitor Argatroban and Direct Factor Xa Inhibitor Apixaban Microsphere Inhalation Formulation

Polylactic-co-glycolic acid (PLGA) microspheres can be spray dried in a solvent spray drying system as a dry powder. Apixaban and Argatroban can be incorporated into the PLGA spheres at 5% to 60% drug loading. The PLGA spray drying system can manufacture stable dry powders with particle sizes of 3 to 6 μm median mass aerodynamic diameter (MMAD), ideal for deep lung delivery with capsule based dry powder inhalers. PLGA microspheres offer the potential of sustained release drug delivery to treat the specific target tissue.

### Example 53: Preparation of 25% Direct Factor Xa Inhibitor Apixaban with Lactose Formulation for Jet Mill Inhalation

Low solubility crystalline drugs can be micronized with a jet mill to a uniform particle size of approximately 1 μm. The milled drug particles can then be blended at a various weight percent with the 30 μm to 90 μm lactose particles. A 25% drug blending with lactose creates 2.5 mg of drug delivered per 10 mg of powder. The powder and drug can be delivered to the deep lung upon patient inhalation.

Jet mill inhalation formulation can be prepared with 25% direct factor Xa inhibitor Apixaban and Lactose. Using a 1″ micromaster sanitary jet mill, add 100 g of Apixaban. Run the mill for approximately 60 minutes to produce crystalline Apixaban microparticles in the 2 to 4 μm range. Add 800 g of lactose carrier particles to a dry powder Turbula® T2F particle blender. Add 250 g of Apixaban to the blending hopper and mix for approximately 30-60 minutes. After mixing is complete fill 10.0 mg of powder into gelatin capsules. Using a dry powder inhaler such as a turbospin, puncture the capsules and measure the emitted dose which is approximately 80% of the 10 mg or 8.0 mg of powder is inhaled. Since the formulation is 25% drug, the amount of Apixaban delivered to the lung is approximately 2.0 mg of Apixaban (25% of the 8.0 mg powder).

### Example 54: Preparation of 25% Direct Factor IIa Inhibitor Argatroban with Lactose Formulation for Jet Mill Inhalation

Jet mill inhalation formulation can be prepared with 25% direct factor IIa inhibitor Argatroban and Lactose. The milled drug particles can then be blended at various weight percent with the 30 μm to 90 μm lactose particles. A 25% drug blending with lactose creates 2.5 mg of drug delivered per 10 mg of powder. The powder and drug can be delivered to the deep lung upon patient inhalation. The procedure is as below.

Using a 1″ micromaster sanitary jet mill, add 250 g of Argatroban. Run the mill for approximately 60 minutes to produce crystalline Argatroban microparticles in the 2 to 4 μm range. Add 750 g of lactose carrier particles to a dry powder Turbula® T2F particle blender. Add 250 g of Argatroban to the blending hopper and mix for approximately 30-60 minutes. After mixing is complete, fill 40.0 mg of powder into gelatin capsules. Using a dry powder inhaler such as a turbospin, puncture the capsule and measure the emitted dose which is approximately 80% of the 40 mg or 32.0 mg of powder is inhaled. Since the formulation is 25% drug, the amount of Argatroban delivered to the lung is approximately 8.0 mg of Argatroban (25% of the 32.0 mg of powder).

### Example 55: Preparation of Direct Factor Ha Inhibitor Argatroban and Direct Factor Xa Inhibitor Apixaban with Lactose Formulation for Jet Mill Inhalation

Jet mill inhalation formulation can be prepared with a 25% formulation of Argatroban combined with Apixaban in a 10:1 mass ratio, blended with 75% lactose carrier. For a combination drug therapy, a patient can inhale 1 capsule with a specific dose of Apixaban and inhale a second capsule with a specific dose of Argatroban. It is possible to blend the 2 drugs in combination with the carrier lactose so both drugs can be inhaled in one dose. The powder and drug can be delivered to the deep lung upon patient inhalation. The procedure is as below. Using a 1″ micromaster sanitary jet mill, add 100 g of Argatroban and 10 g of Apixaban. Run the mill for approximately 60 minutes to produce crystalline Argatroban and Apixaban microparticles in the 2 to 4 μm range. Add 750 g of lactose carrier particles to a dry powder Turbula® T2F particle blender. Add 250 g of combination 10:1 Argatroban and Apixaban to the blending hopper and mix for approximately 30-60 minutes. After mixing is complete fill 40.0 mg of powder into gelatin capsules. Using a dry powder inhaler such as a turbospin, puncture the capsules and measure the emitted dose which is approximately 80% of the 40 mg or 32.0 mg of powder is inhaled. Since the formulation is 1% Apixaban, the amount of Apixaban delivered to the lung is approximately 0.32 mg of Apixaban (1% of the 32.0 mg of powder). Since the formulation is 10% Argatroban, the amount of Argatroban delivered to the lung is approximately 3.2 mg of Argatroban (10% of the 32.0 mg of powder).

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the present disclosure. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for inhibiting blood clotting in a patient's lung alveoli, the method comprising:
selecting a patient suffering from or at risk of suffering from blood clotting and/or fibrin formation in the patient's lung alveoli;
providing a therapeutic composition comprising each of a direct factor IIa inhibitor and a direct factor Xa inhibitor; and
delivering the therapeutic composition to the patient's lung alveoli at a dose sufficient to inhibit clot formation and/or fibrin formation therein.

2. The method of claim 1, delivering the dose of the therapeutic composition comprises one or more of inhalation, nebulization, ventilation, instillation, ultrasound dispersion, and injection.

3. The method of claim 1, wherein the patient is selected based upon blood clotting and/or fibrin formation caused by viral infection, bacterial infection, smoke inhalation, chemical exposure, genetic mutation, injury, smog and other air pollutions, inhalation of pollutants, work-related lung diseases, hypersensitivity pneumonitis, or a risk thereof.

4. The method of any one of claim 1, wherein the patient is selected based upon blood clotting and/or fibrin formation caused by pneumonia, bronchitis, emphysema, asthma, pulmonary fibrosis, lung cancer, pulmonary edema, pulmonary embolism, sarcoidosis and granulomatosis with polyangiitis, chronic obstructive pulmonary disease, bronchiectasis, acute respiratory distress syndrome (ARDS), severe acute respiratory syndrome (SARS), and COVID-19.

5. The method of any one of claim 1, wherein the direct factor Xa inhibitor is selected from the group consisting of apixaban, betrixaban, edoxaban, otamixaban, razaxaban, rivaroxaban, (r)-n-(2-(4-(1-methylpiperidin-4-yl)piperazin-1-yl)-2-oxo-1-phenylethyl)-1h-indole-6-carboxamide (LY-517717), daraxaban (YM-150), 2-[(7-carbamimidoylnaphthalen-2-yl)methyl-[4-(1-ethanimidoylpiperidin-4-yl)oxyphenyl]sulfamoyl]acetic acid (YM-466 or YM-60828), eribaxaban (PD 0348292), 2-(5-carbamimidoyl-2-hydroxy-phenyl) 4-[5-(2,6-dimethyl-piperidin-1-yl)-pentyl]-3-oxo-3, 4-dihydro-quinoxaline-6-carboxylic acid (PD0313052), (S)-3-(7-carbamimidoylnaphthalen-2-yl)-2-(44(S)-1-(1-iminoethyl)pyrrolidin-3-yl)oxy)phenyl)propanoic acid hydrochloride pentahydrate (DX9065a), letaxaban (TAK-442), GW813893, JTV-803, KFA-144, DPC-423, RPR-209685, MCM-09, or antistasin.

6. The method of claim 5, wherein the direct factor Xa inhibitor comprises rivaroxaban, or a salt, isomer, solvate, analog, derivative, metabolite, or prodrugs thereof.

7. The method of claim 5, wherein the direct factor Xa inhibitor comprises apixaban, or a salt, isomer, solvate, analog, derivative, metabolite, or prodrugs thereof.

8. The method of claim 1, wherein a delivered dose of the direct factor Xa is sufficient to generate a tissue concentration of the direct factor Xa inhibitor in the patient's lung alveoli of at least 0.2 ng/mg tissue measured 5 minutes after delivery of the therapeutic composition is completed.

9. The method of claim 8, wherein the delivered dose of the direct factor Xa inhibitor is sufficient to generate a tissue concentration of the direct factor Xa inhibitor of at least 0.5 ng/mg tissue.

10. The method of claim 8, wherein the delivered dose of the direct factor Xa inhibitor is sufficient to generate a tissue concentration of the direct factor Xa inhibitor of at least 1 ng/mg tissue.

11. The method of claim 8, wherein the patient's blood concentration of the direct factor Xa inhibitor is less than 200 ng/ml measured 5 minutes after delivery of the therapeutic composition is completed.

12. The method of claim 11, wherein the patient's blood concentration of the direct factor Xa inhibitor is less than 100 ng/ml.

13. The method of claim 11, wherein the patient's blood concentration of the direct factor Xa inhibitor is less than 50 ng/ml.

14. The method of claim 11, wherein the patient's blood concentration of the direct factor Xa inhibitor is less than 40 ng/ml.

15. The method of claim 11, wherein the patient's blood concentration of the direct factor Xa inhibitor is less than 10 ng/ml.

16. The method of claim 1, wherein the direct factor IIa inhibitor is selected from the group consisting of argatroban, dabigatran, ximelagatran, melagatran, efegatran, inogatran, atecegatran metoxil (AZD-0837), hirudin, hirudin analogs, bivalirudin, desirudin, or lepirudin.

17. The method of claim 16, wherein the direct factor IIa inhibitor comprises argatroban or a salt, isomer, solvate, analog, derivative, metabolite, or prodrugs thereof.

18. The method of claim 17, wherein the direct factor Xa inhibitor comprises apixaban and the direct factor IIa inhibitor comprises argatroban.

19. The method of claim 16, wherein a delivered dose of the direct factor IIa inhibitor is sufficient to generate a tissue concentration of the direct factor IIa inhibitor of at least 0.1 ng/mg tissue measured 5 minutes after delivery of the therapeutic composition is completed.

20. The method of claim 19, wherein the delivered dose of the direct factor IIa inhibitor is sufficient to generate a tissue concentration of the direct factor IIa inhibitor of at least 0.2 ng/mg tissue.

21. The method of claim 19, wherein the delivered dose of the direct factor IIa inhibitor is sufficient to generate a tissue concentration of the direct factor IIa inhibitor of at least 0.5 ng/mg tissue.

22. The method of claim 19, wherein the delivered dose of the direct factor IIa inhibitor is sufficient to generate a tissue concentration of the direct factor IIa inhibitor of at least 1 ng/mg tissue.

23. The method of any one of claim 19, wherein the patient's blood concentration of the direct factor IIa inhibitor is less than 100 ng/ml measured 30 minutes after delivery of the therapeutic composition is completed.

24. The method of claim 23, wherein the patient's blood concentration of the direct factor IIa inhibitor is less than 50 ng/ml.

25. The method of claim 23, wherein the patient's blood concentration of the direct factor IIa inhibitor is less than 30 ng/ml.

26. The method of claim 23, wherein the patient's blood concentration of the direct factor IIa inhibitor is less than 10 ng/ml.

27. The method of claim 1, wherein the dose is sufficient to generate a blood concentration of the direct factor Xa inhibitor which is smaller than a median maximum serum concentration (Cmax) of the direct factor Xa inhibitor generated by systemic delivery of the direct factor Xa inhibitor to achieve the same tissue concentration at the site of the inflammatory pulmonary disease.

28. The method of claim 1, wherein the therapeutically effective dose is sufficient to generate a blood concentration of the direct factor Xa inhibitor which does not exceed a median maximum serum concentration (Cmax) of the direct factor Xa inhibitor generated by systemic delivery of the direct factor Xa inhibitor to achieve the same tissue concentration at the site of the inflammatory pulmonary disease for more than about 26 hours to about 4 hours.

29. The method of claim 1, wherein the dose is sufficient to generate a blood concentration of the direct factor IIa inhibitor which is smaller than a median maximum serum concentration (Cmax) of the direct factor IIa inhibitor generated by systemic delivery of the direct factor IIa inhibitor to achieve the same tissue concentration at the site of the inflammatory pulmonary disease.

30. The method of claim 1, wherein the dose is sufficient to generate a blood concentration of the direct factor IIa inhibitor which does not exceed a median maximum serum concentration (Cmax) of the direct factor IIa inhibitor generated by systemic delivery of the direct factor IIa inhibitor to achieve the same tissue concentration at the site of the inflammatory pulmonary disease for more than about 2 hours to about 4 hours.

31. The method of claim 1, wherein a weight ratio of the direct factor Xa inhibitor to the direct factor IIa inhibitor in the therapeutic composition is within a range of 3:1 to 1:10.

32. The method of claim 31, wherein the weight compositional ratio of the direct factor Xa inhibitor to the direct factor IIa inhibitor in the therapeutic composition is about 1:5.

33. The method of claim 1, wherein the therapeutic composition comprises one or more additional pharmaceutical agents.

34. The method of claim 39, wherein the one or more additional pharmaceutical agents comprises one or more anti-fibrotic agents, anti-platelet, antihistamine, anti-viral agents, anti-bacterial agents, metformin or its salt, steroids, interferons, anti-proliferative, anti-angiogenic, anti-VEGF, or combinations thereof.

35. The method of claim 1, wherein delivering the therapeutic composition to the patient's lung alveoli comprises dispersing liquid droplets comprising the therapeutic composition into a breathing gas which is delivered to or inhaled by the patient.

36. The method of claim 35 wherein the droplets have a mean droplet size in a range from 1 μm to 10 μm.

37. The method of claim 1, wherein delivering the therapeutic composition to the patient's lung alveoli comprises dispersing dry particles comprising the therapeutic composition into a breathing gas which is delivered to or inhaled by the patient.

38. The method of claim 37, wherein the particles have a mean particle size in a range from 1 μm to 10 μm.

39. The method of any one of claim 1, wherein a total dosage of apixaban from 1 mg to 5 mg and a total dosage of argatroban from 20 mg to 40 mg is delivered per day.

40.-82. (canceled)
